(19) United States
(12) Patent Application Publication
(10) Pub. No.: US 2026/0255871 A1
(43) Pub. Date: Aug. 27, 2026

(54) HETEROCYCLIC COMPOUND, ORGANIC LIGHT-EMITTING ELEMENT COMPRISING SAME, AND COMPOSITION FOR ORGANIC LAYER

(71) Applicant: LT MATERIALS CO., LTD., Yongin-si, Gyeonggi-do (KR)

(72) Inventors: Seung Woo LEE, Yongin-si (KR); Yu Jin HEO, Yongin-si (KR); Jun Tae MO, Yongin-si (KR); Dong Jun KIM, Yongin-si (KR)

(73) Assignee: LT MATERIALS CO., LTD., Yongin-si, Gyeonggi-do (KR)

(21) Appl. No.: 18/852,928

(22) PCT Filed: Feb. 20, 2023
(86) PCT No.: PCT/KR2023/095006

§ 371 Date: Sep. 30, 2024

(30) Foreign Application Priority Data
Apr. 18, 2022 (KR) 10-2022-0047680

(51) Int. Cl. H10K 85/60; C07B 59/00; C07D 241/44; C07D 405/12; C07D 495/04; C09K 11/02; H10K 50/11

(52) U.S. Cl. CPC H10K 85/633; C07B 59/002; C07D 241/44; C07D 405/12; C07D 495/04; C09K 11/02; H10K 85/636; H10K 85/654; H10K 85/657; H10K 50/11; H10K 85/615; H10K 85/622; H10K 85/626; H10K 85/6572; H10K 85/6574

(57) ABSTRACT

The present invention relates to a heterocyclic compound represented by Chemical Formula 1, an organic light-emitting element comprising same, and a composition for an organic layer.

### TECHNICAL FIELD

This application claims priority to and the benefits of Korean Patent Application No. 10-2022-0047680, filed with the Korean Intellectual Property Office on Apr. 18, 2022, the entire contents of which are incorporated herein by reference.

The present invention relates to a heterocyclic compound, an organic light-emitting element including the same, and a composition for an organic material layer.

### BACKGROUND ART

An organic light-emitting device is one type of self-emissive display devices, and has advantages of having a wide viewing angle and a high response speed as well as having an excellent contrast.

The organic light-emitting device has a structure of disposing an organic thin film between two electrodes. When a voltage is applied to the organic light-emitting device having such a structure, electrons and holes injected from the two electrodes bind and pair in the organic thin film, and then light emits as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of serving as a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light-emitting device.

### PRIOR ART DOCUMENTS

### Patent Documents

U.S. Pat. No. 4,356,429

### DISCLOSURE

### Technical Problem

An object of the present invention is to provide a heterocyclic compound, an organic light-emitting device including the same, and a composition for an organic material layer.

### Technical Solution

In order to object,
one embodiment of the present invention provides a heterocyclic compound represented by the following Chemical Formula 1.

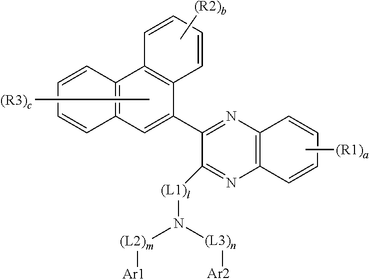

In Chemical Formula 1,
R1 to R3 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring; or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
a is an integer of 0 to 4, and when a is 2 or greater, R1s are the same as or different from each other,
b is an integer of 0 to 4, and when b is 2 or greater, R2s are the same as or different from each other,
c is an integer of 0 to 5, and when c is 2 or greater, R3s are the same as or different from each other,
Ar1 and Ar2 are the same as or different from each other, and each independently a substituted or unsubstituted C6 to C60 aryl group; or a substituted or unsubstituted C2 to C60 heteroaryl group including one or more of O; or S as a heteroatom,
L1 to L3 are the same as or different from each other, and each independently a direct bond; a substituted or unsubstituted C6 to C60 arylene group; or a substituted or unsubstituted C2 to C60 heteroarylene group including one or more of O; or S as a heteroatom, and
l, m and n are an integer of 0 to 5, and when 1 is 2 or greater, L1s are the same as or different from each other, when m is 2 or greater, L2s are the same as or different from each other, and when n is 2 or greater, L3s are the same as or different from each other.

In addition, the present invention provides an organic light-emitting device comprising: a first electrode;
a second electrode provided opposite to the first electrode; and
one or more organic material layers provided between the first electrode and the second electrode,
wherein at least one or more organic material layers comprises the heterocyclic compound represented by Chemical Formula 1.

In addition, the present invention provides an organic light-emitting device, wherein the organic material layer further comprises a heterocyclic compound represented by the following Chemical Formula 2.

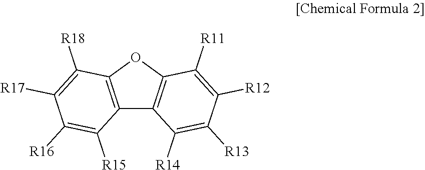

In Chemical Formula 2,
R11 to R18 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; —NR201R202; and the following Chemical Formula 3, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
at least one of R11 to R18 is the following Chemical Formula 3,

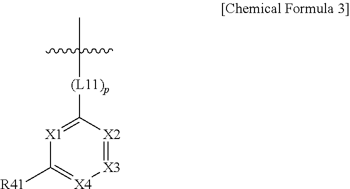

in Chemical Formula 3,
X1 is N; or CRa,
X2 is N; or CRb,
X3 is N; or CRc,
X4 is N; or CRd,
at least two of X1 to X4 are N,
R41 and Ra to Rd are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R301R302; —SiR301R302R303; and —NR301R302, or two or more groups adjacent to each other bond to each other to form a substituted or unsubstituted C6 to C60 aromatic hydrocarbon ring; or a substituted or unsubstituted C2 to C60 heteroring, and R301, R302 and R303 are the same as or different from each other and each independently a substituted or unsubstituted C1 to C60 alkyl group; a substituted or unsubstituted C6 to C60 aryl group; or a substituted or unsubstituted C2 to C60 heteroaryl group;
L11 is a direct bond; a substituted or unsubstituted C6 to C60 arylene group; or a substituted or unsubstituted C2 to C60 heteroarylene group; and
p is an integer of 0 to 5, and when p is 2 or greater, L11s are the same as or different from each other.

In addition, the present invention provides a composition for an organic material layer, the composition comprising the heterocyclic compound represented by Chemical Formula 1 above and the heterocyclic compound represented by Chemical Formula 2 above.

### Advantageous Effects

The compound described in the present specification can be used as an organic material layer material of an organic light-emitting device. The compound is capable of performing roles of a hole injection layer material, a hole transport layer material, a light emitting layer material, an electron transport layer material, an electron injection layer material and the like in an organic light-emitting device. Particularly, the compound can be used as a light emitting layer material of an organic light-emitting device, and the compound can be used as a light emitting material alone, or as a host material or a dopant material of a light emitting layer.

Specifically, the compound can be used as a light emitting material alone, or as a host material or a dopant material of a light emitting layer. When the heterocyclic compound represented by Chemical Formula 1 is used in an organic material layer, it is possible to lower a driving voltage of an organic light-emitting device, enhance light emission efficiency thereof, and enhance lifetime properties thereof.

### DESCRIPTION OF DRAWINGS

FIGS. 1 to 3 are views schematically showing of an organic light emitting device according to an embodiment of the present invention.

### MODE FOR DISCLOSURE

Hereinafter, the present invention will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; halogen; a cyano group; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent in which two or more substituents selected from among the substituents exemplified above are linked or being unsubstituted.

In the present specification, the halogen may be fluorine, chlorine, bromine or iodine.

In the present specification, the alkyl group includes a linear or branched form having 1 to 60 carbon atoms, and may be further substituted with other substituents. The number of carbon atoms of the alkyl group may be from 1 to 60, specifically from 1 to 40 and more specifically from 1 to 20. Specific examples of the alkyl group may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group and the like, but are not limited thereto.

In the present specification, the alkenyl group includes a linear or branched form having 2 to 60 carbon atoms, and may be further substituted with other substituents. The number of carbon atoms of the alkenyl group may be from 2 to 60, specifically from 2 to 40 and more specifically from 2 to 20. Specific examples of the alkenyl group may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes a linear or branched form having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples of the alkoxy group may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes a monocyclic or polycyclic group having 3 to 60 carbon atoms, and may be further substituted with other substituents. Herein, the polycyclic group means a group in which the cycloalkyl group is directly linked to or fused with another cyclic group. Herein, the another cyclic group may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples of the cycloalkyl group may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes a monocyclic or polycyclic group having 2 to 60 carbon atoms, and may be further substituted with other substituents. Herein, the polycyclic group means a group in which the heterocycloalkyl group is directly linked to or fused with another cyclic group. Herein, the another cyclic group may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes a monocyclic or polycyclic group having 6 to 60 carbon atoms, and may be further substituted with other substituents. Herein, the polycyclic group means a group in which the aryl group is directly linked to or fused with another cyclic group. Herein, the another cyclic group may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

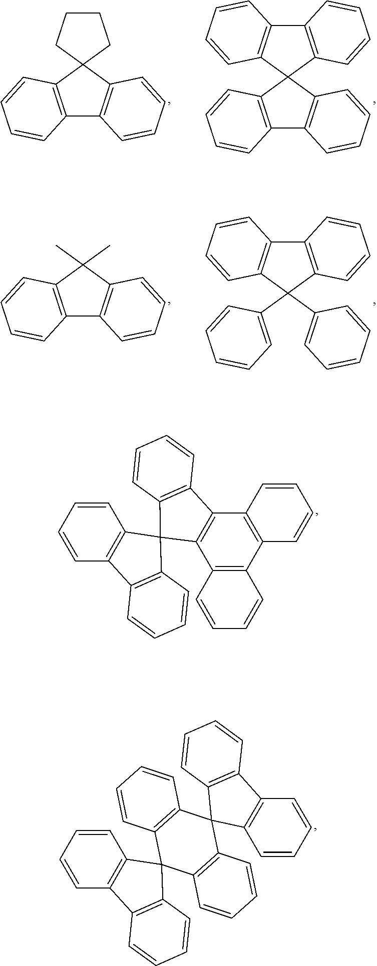

and the like may be included, however, the structure is not limited thereto.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

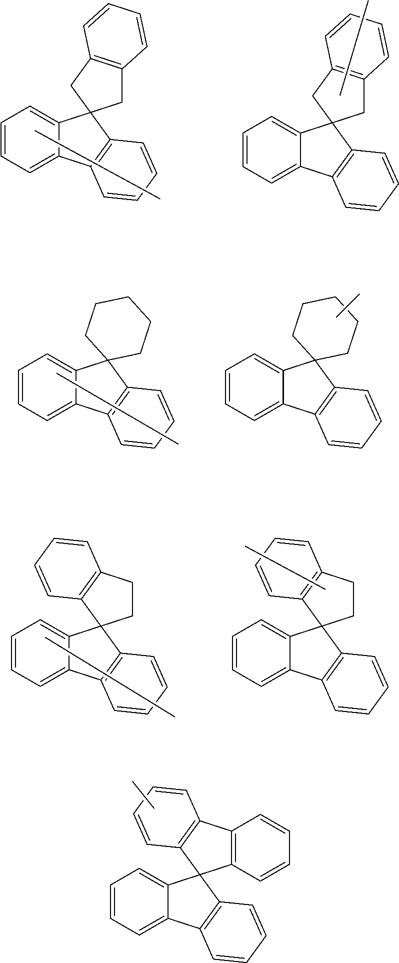

In the present specification, the heteroaryl group includes S, O, Se, N or Si as a heteroatom, includes a monocyclic or polycyclic group having 2 to 60 carbon atoms, and may be further substituted with other substituents. Herein, the polycyclic group means a group in which the heteroaryl group is directly linked to or fused with another cyclic group. Herein, the another cyclic group may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenanthridinyl group, an imidazopyridinyl group, a diazanaphthalenyl group, a triazaindenyl group, a 2-indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilole group, a spirobi(dibenzosilole) group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present invention, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present invention, a “case of a substituent being not indicated in a chemical formula or compound structure” may mean that positions to which substituents may come are all hydrogen or deuterium. In other words, since deuterium is an isotope of hydrogen, some hydrogen atoms may be deuterium that is an isotope, and herein, a content of the deuterium may be from 0% to 100%.

In one embodiment of the present invention, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present invention, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present invention, an isotope means an atom with the same atomic number (Z) but with a different mass number (A), and may also be interpreted as an element with the same number of protons but with a different number of neutrons.

In one embodiment of the present invention, a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

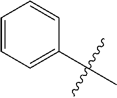 [SMILES: *c1ccccc1]

may mean that the total number of substituents that the phenyl group may have is 5 (T1 in the formula), and the number of deuterium atoms among these is 1 (T2 in the formula). In other words, having a deuterium content of 20% in a phenyl group may be represented by the following structural formulae.

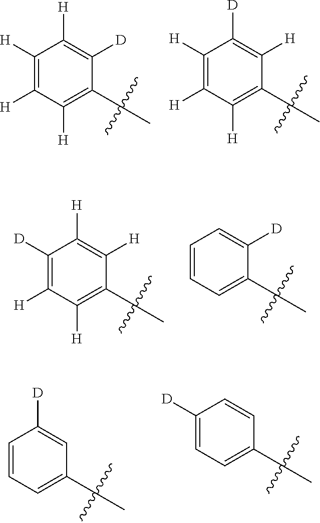 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present invention, “a phenyl group having a deuterium content of 0%” may mean a phenyl group that does not include a deuterium atom, that is, a phenyl group that has 5 hydrogen atoms.

In the present invention, the C6 to C60 aromatic hydrocarbon ring means a compound including an aromatic ring formed with C6 to C60 carbons and hydrogens. Examples thereof may include phenyl, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art and satisfying the above-mentioned number of carbon atoms.

One embodiment of the present invention provides a heterocyclic compound represented by the following Chemical Formula 1.

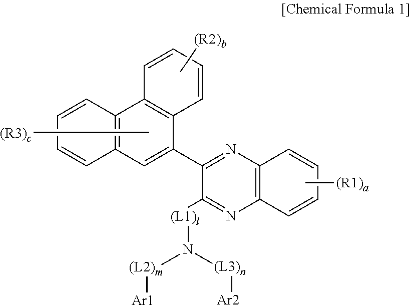

In Chemical Formula 1,
R1 to R3 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring; or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
a is an integer of 0 to 4, and when a is 2 or greater, R1s are the same as or different from each other,
b is an integer of 0 to 4, and when b is 2 or greater, R2s are the same as or different from each other,
c is an integer of 0 to 5, and when c is 2 or greater, R3s are the same as or different from each other,
Ar1 and Ar2 are the same as or different from each other, and each independently a substituted or unsubstituted C6 to C60 aryl group; or a substituted or unsubstituted C2 to C60 heteroaryl group including one or more of O; or S as a heteroatom,
L1 to L3 are the same as or different from each other, and each independently a direct bond; a substituted or unsubstituted C6 to C60 arylene group; or a substituted or unsubstituted C2 to C60 heteroarylene group including one or more of O; or S as a heteroatom, and
l, m and n are an integer of 0 to 5, and when 1 is 2 or greater, L1s are the same as or different from each other, when m is 2 or greater, L2s are the same as or different from each other, and when n is 2 or greater, L3s are the same as or different from each other.

In one embodiment of the present invention, R1 to R3 are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R11R102; —SiR101R102R103; or —NR101R102, or two or more groups adjacent to each other bond to each other to form a substituted or unsubstituted C6 to C30 aromatic hydrocarbon ring; or a substituted or unsubstituted C2 to C30 heteroring, and R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present invention, R1 to R3 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or —NR101R102, or two or more groups adjacent to each other bond to each other to form a substituted or unsubstituted C6 to C20 aromatic hydrocarbon ring; or a substituted or unsubstituted C2 to C20 heteroring, and R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present invention, R1 to R3 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or —NR101R102.

In another embodiment of the present invention, R1 to R3 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present invention, R1 to R3 are the same as or different from each other, and may be each independently hydrogen; or deuterium.

In one embodiment of the present invention, L1 to L3 are the same as or different from each other, and may be each independently a direct bond; a substituted or unsubstituted C6 to C30 arylene group; or a substituted or unsubstituted C2 to C30 heteroarylene group including one or more of O; or S as a heteroatom.

In another embodiment of the present invention, L1 to L3 are the same as or different from each other, and may be each independently a direct bond; a substituted or unsubstituted C6 to C20 arylene group; or a substituted or unsubstituted C2 to C20 heteroarylene group including one or more of O; or S as a heteroatom.

In another embodiment of the present invention, L1 to L3 are the same as or different from each other, and may be each independently a direct bond; or a substituted or unsubstituted C6 to C20 arylene group.

In another embodiment of the present invention, L1 may be a direct bond; a substituted or unsubstituted phenylene group; a substituted or unsubstituted biphenylene group; a substituted or unsubstituted terphenylene group; a substituted or unsubstituted naphthylene group; a substituted or unsubstituted anthracene group; or a substituted or unsubstituted triphenylene group.

In another embodiment of the present invention, L2 and L3 are the same as or different from each other, and may be each independently a direct bond; a substituted or unsubstituted phenylene group; a substituted or unsubstituted biphenylene group; a substituted or unsubstituted terphenylene group; a substituted or unsubstituted naphthylene group; a substituted or unsubstituted anthracene group; or a substituted or unsubstituted phenanthrene group.

In one embodiment of the present invention, Ar1 and Ar2 are the same as or different from each other, and may be each independently a substituted or unsubstituted C6 to C30 aryl group; or a substituted or unsubstituted C2 to C30 heteroaryl group including one or more of O; or S as a heteroatom.

In another embodiment of the present invention, Ar1 and Ar2 are the same as or different from each other, and may be each independently a substituted or unsubstituted C6 to C20 aryl group; or a substituted or unsubstituted C2 to C20 heteroaryl group including one or more of O; or S as a heteroatom.

In another embodiment of the present invention, Ar1 and Ar2 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted anthracenyl group; a substituted or unsubstituted triphenylenyl group; a substituted or unsubstituted fluorenyl group; a substituted or unsubstituted benzofluorenyl group; a substituted or unsubstituted chrysenyl group; a substituted or unsubstituted dibenzofuranyl group; a substituted or unsubstituted naphthobenzofuranyl group; a substituted or unsubstituted dibenzothiophenyl group; or a substituted or unsubstituted naphthobenzothiophenyl group.

In one embodiment of the present invention, the heterocyclic compound represented by Chemical Formula 1 may not include deuterium as a substituent, or may have a deuterium content of, for example, greater than 0%, 1% or greater % or greater, 10% or greater, 20% or greater, 30% or greater, 40% or greater or 50% or greater, or 100% or less, 90% or less, 80% or less, 70% or less or 60% or less with respect to the total number of hydrogen atoms and deuterium atoms.

In another embodiment of the present invention, the heterocyclic compound represented by Chemical Formula 1 may not include deuterium as a substituent, or may have a deuterium content of 1% to 100% with respect to the total number of hydrogen atoms and deuterium atoms.

In another embodiment of the present invention, the heterocyclic compound represented by Chemical Formula 1 may not include deuterium as a substituent, or may have a deuterium content of 20% to 90% with respect to the total number of hydrogen atoms and deuterium atoms.

In another embodiment of the present invention, the heterocyclic compound represented by Chemical Formula 1 may not include deuterium as a substituent, or may have a deuterium content of 30% to 80% with respect to the total number of hydrogen atoms and deuterium atoms.

In another embodiment of the present invention, the heterocyclic compound represented by Chemical Formula 1 may not include deuterium as a substituent, or may have a deuterium content of 50% to 70% with respect to the total number of hydrogen atoms and deuterium atoms.

In one embodiment of the present invention, the heterocyclic compound represented by Chemical Formula 1 may be represented by any one of the following compounds.

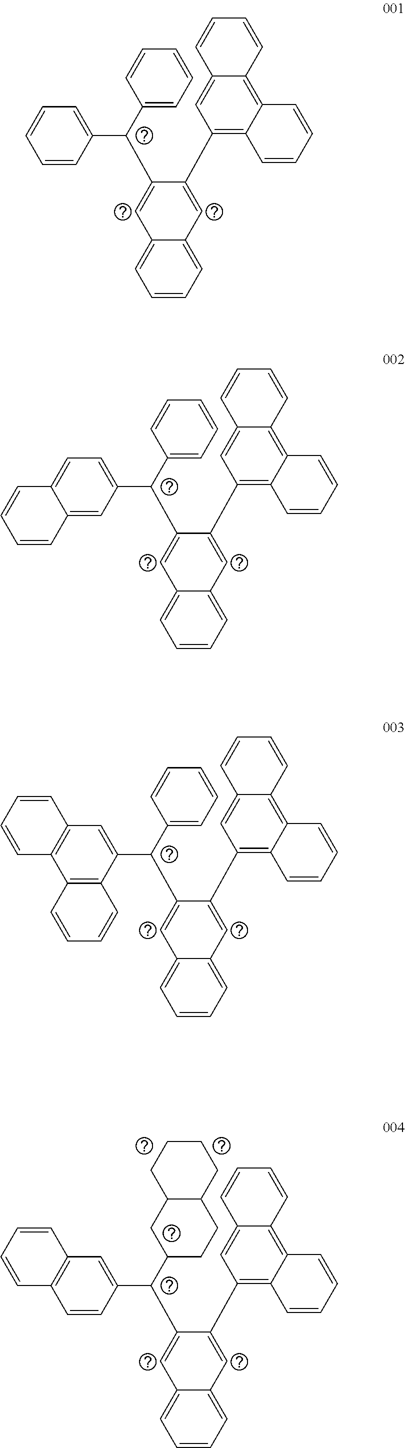

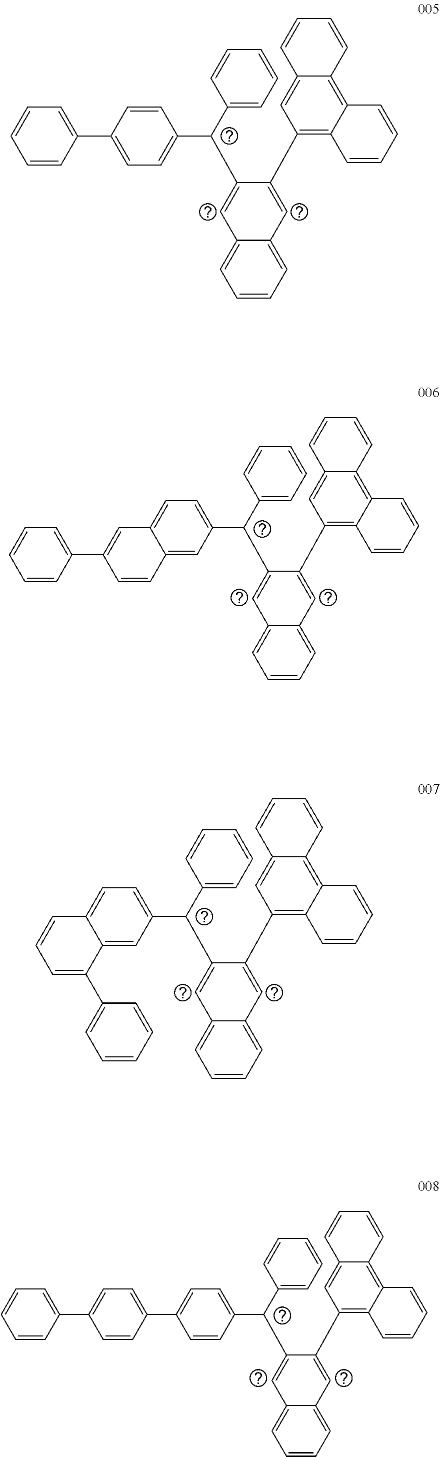

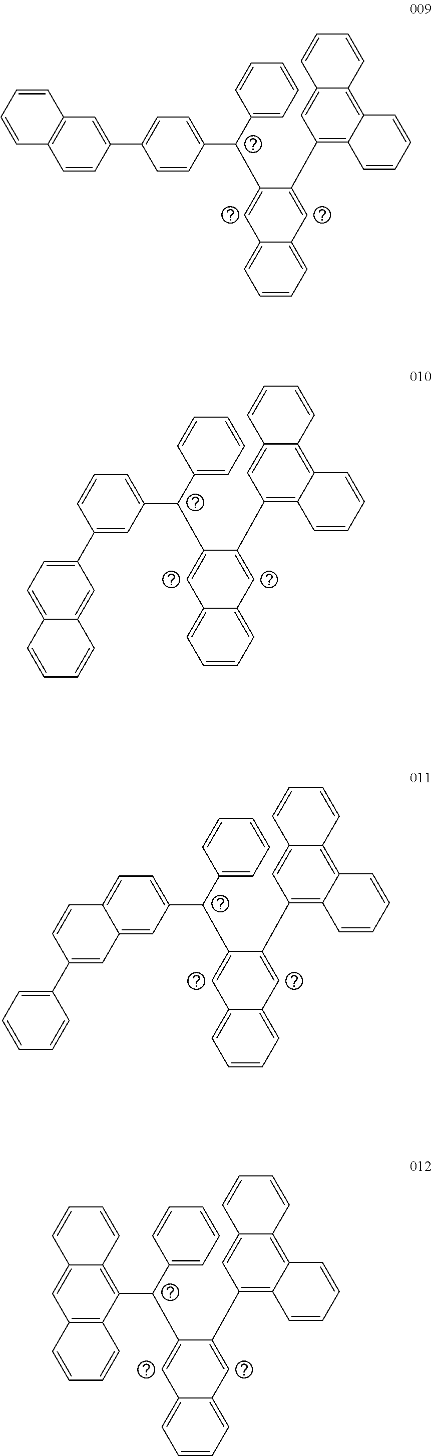

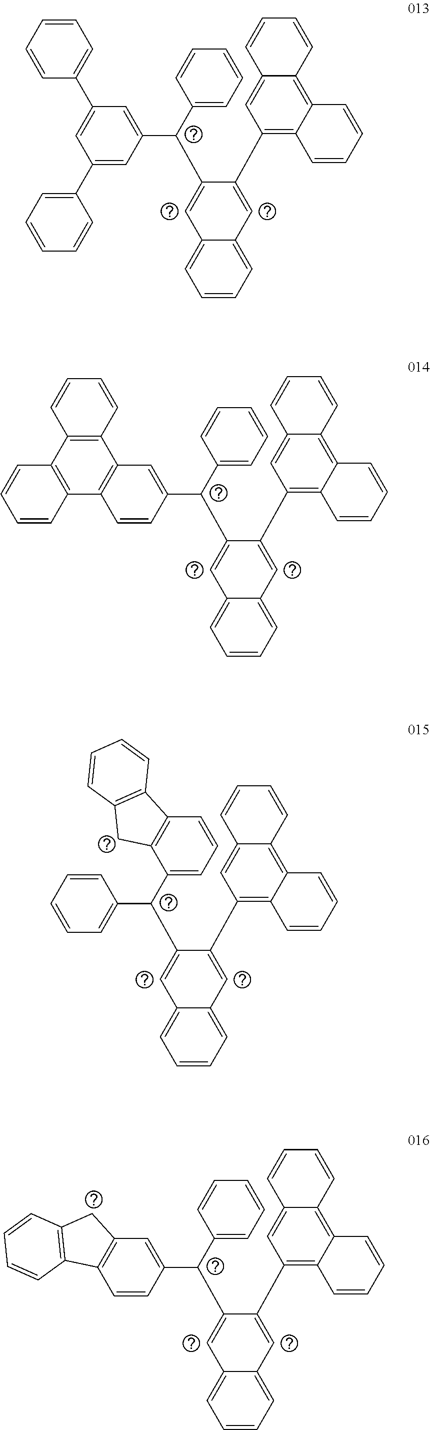

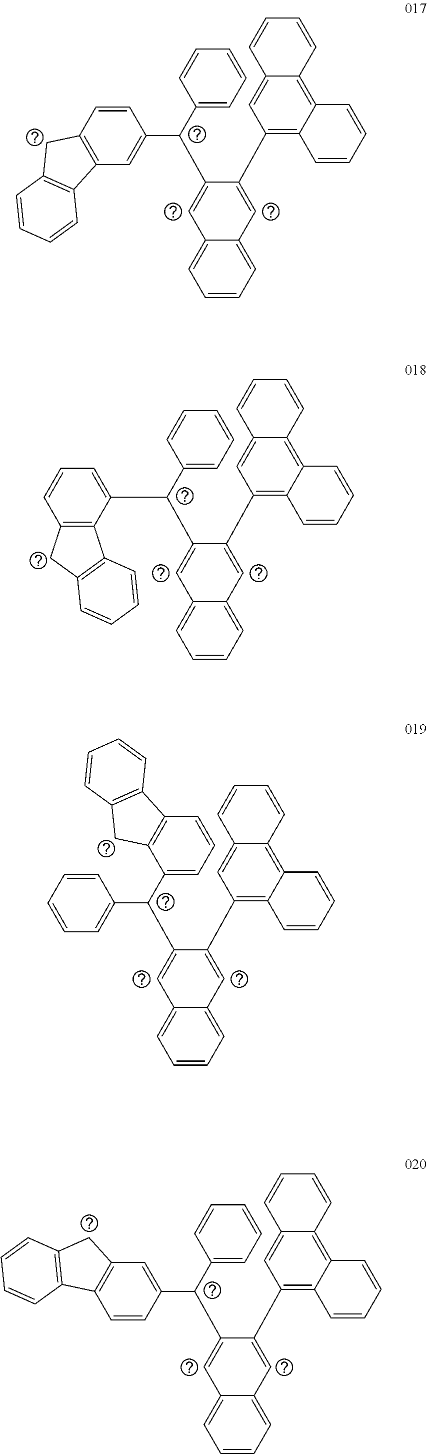

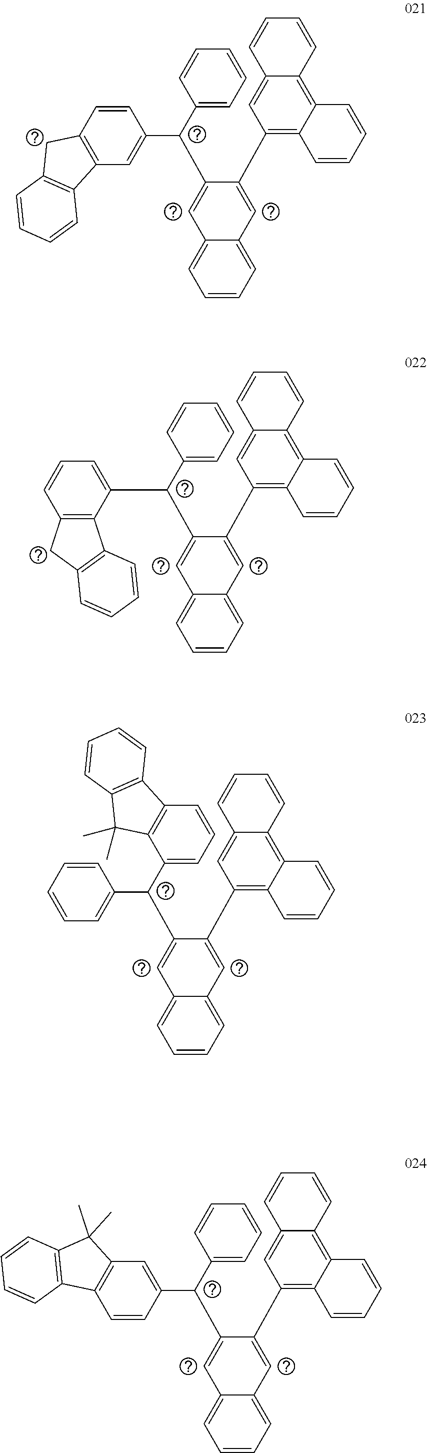

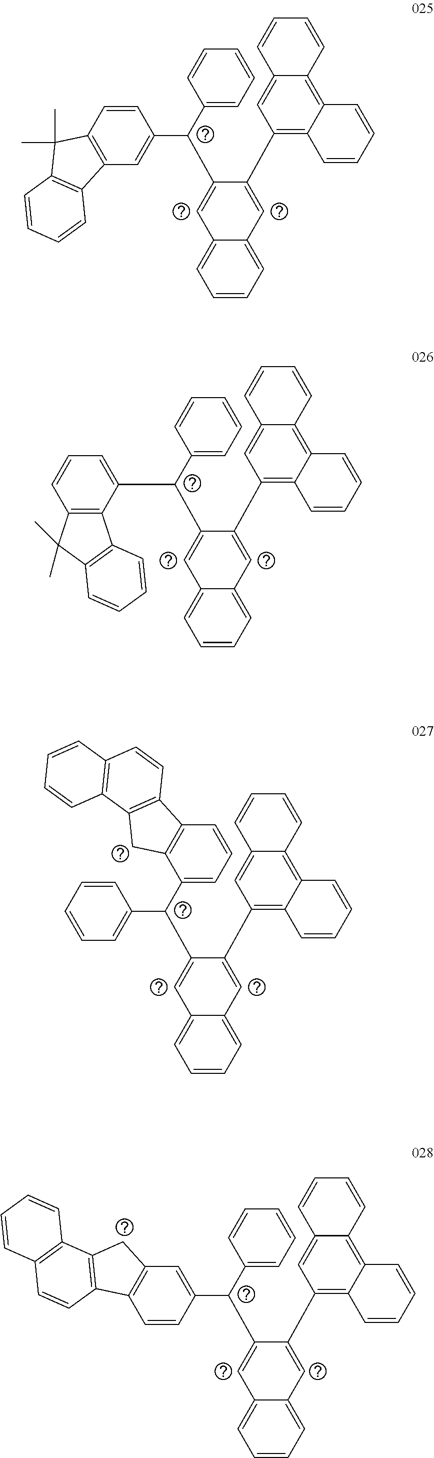

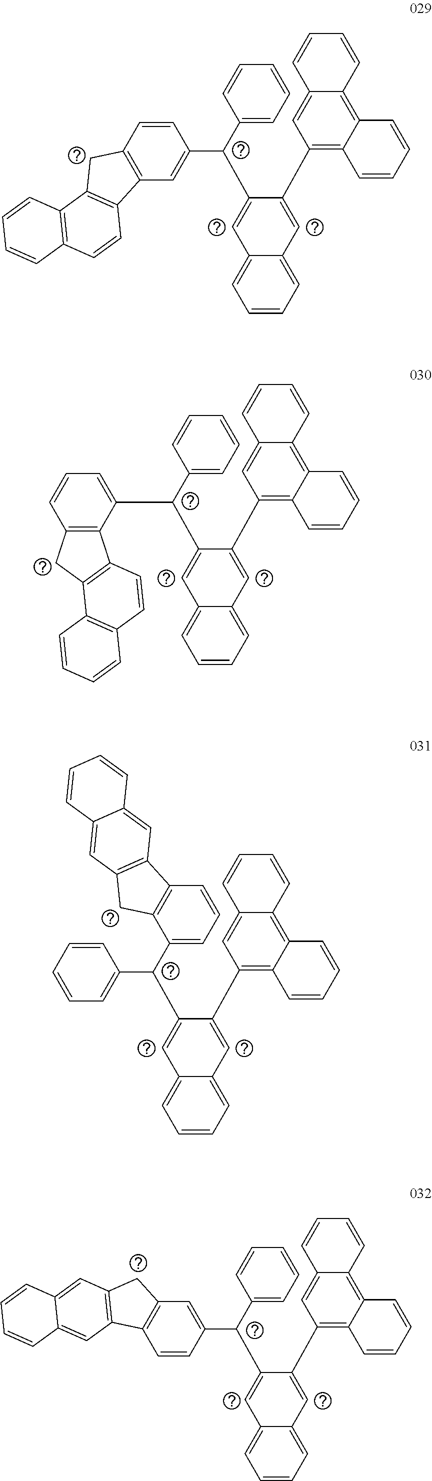

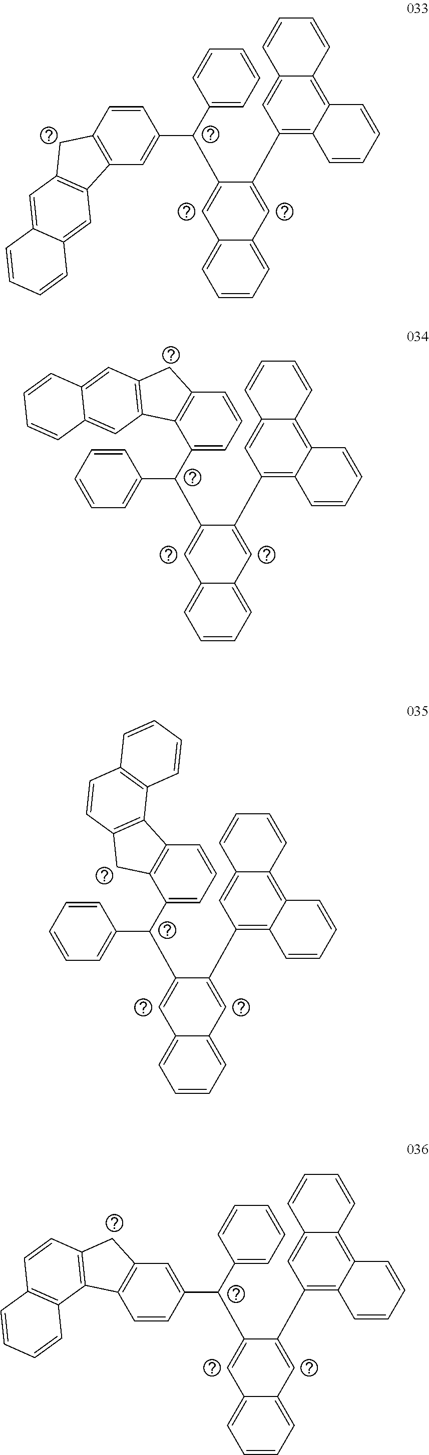

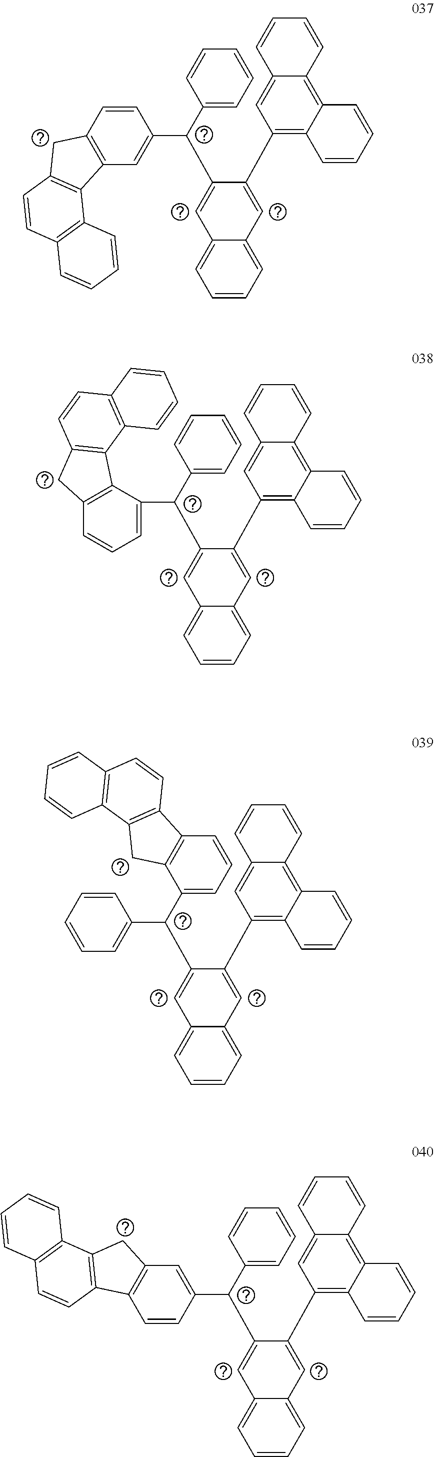

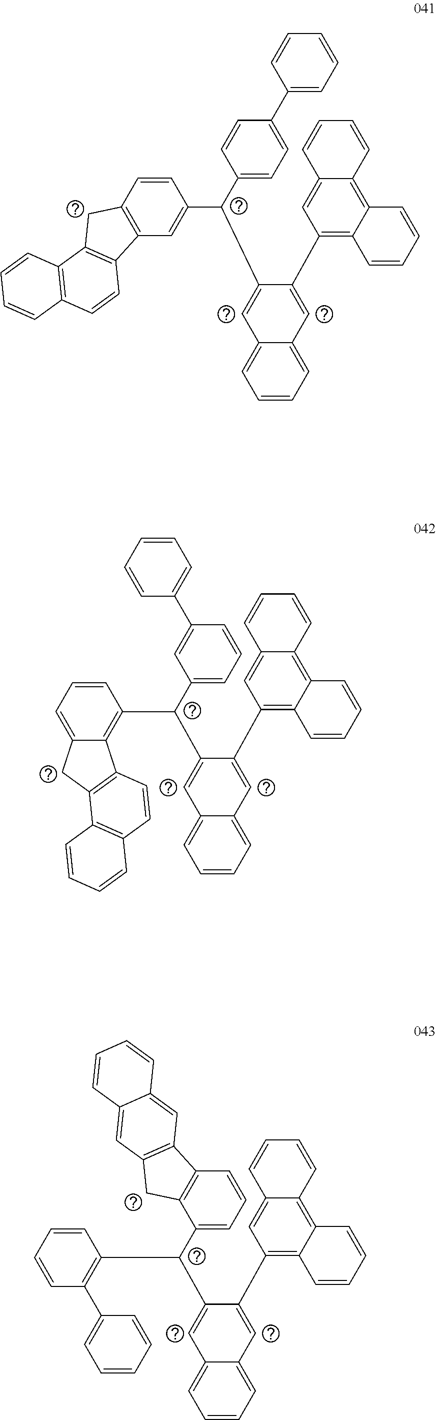

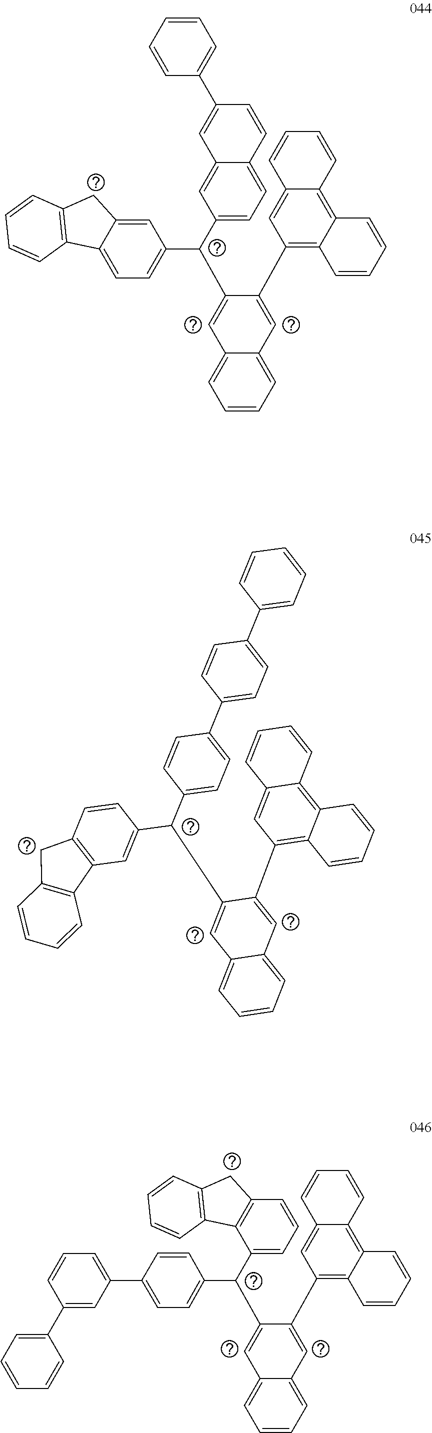

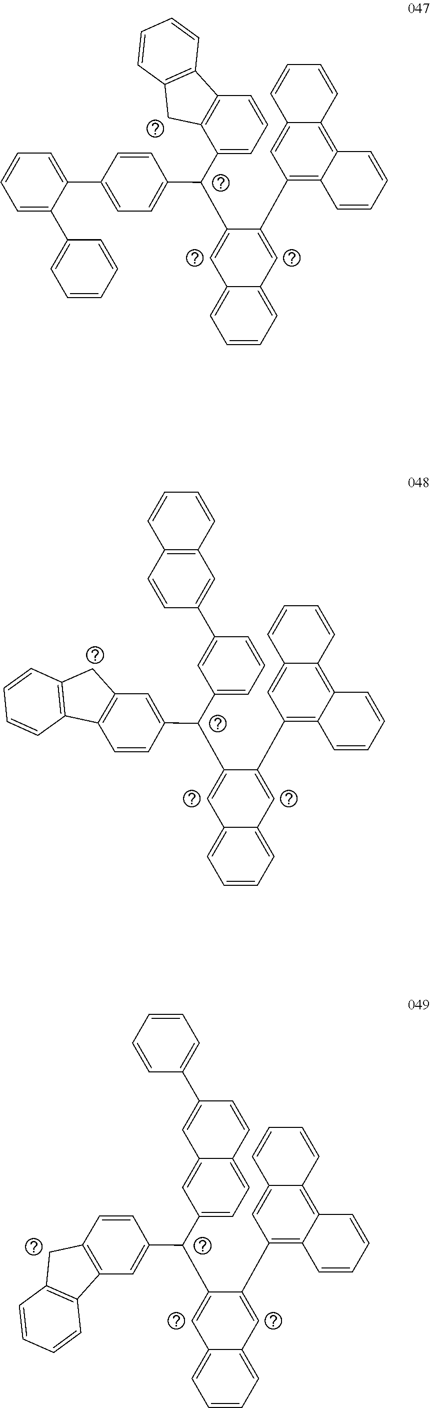

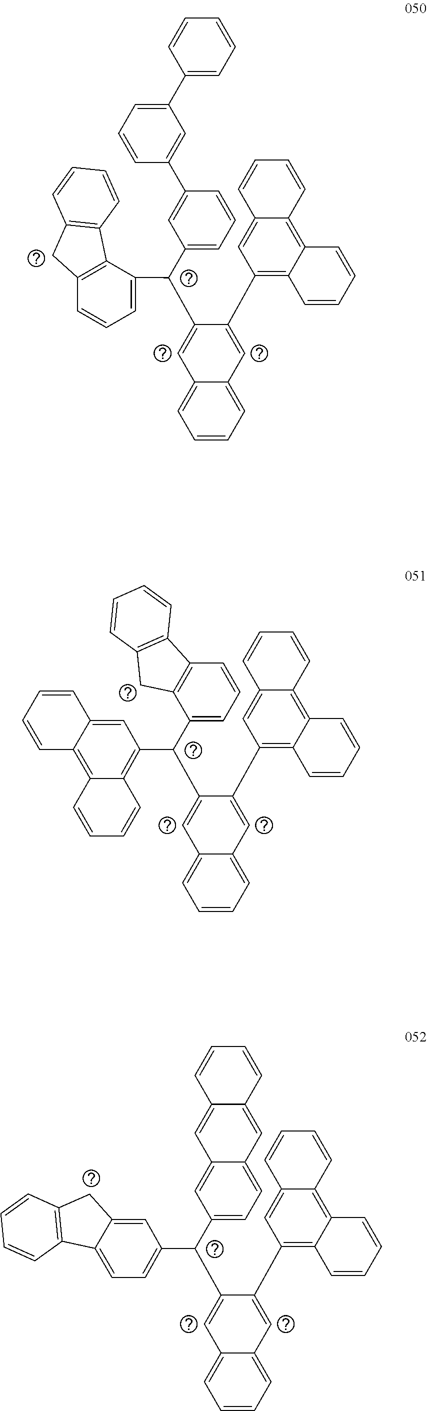

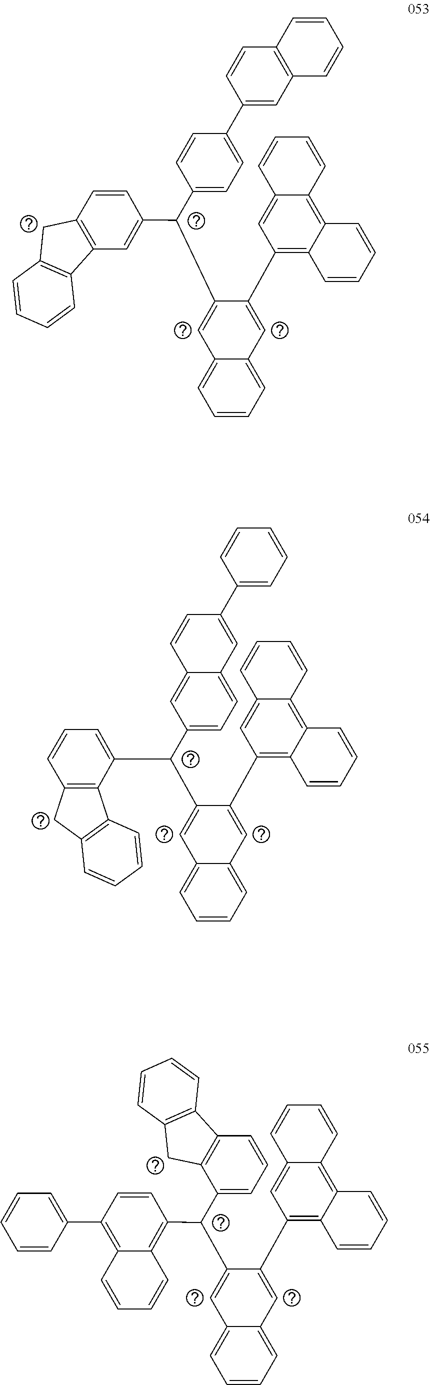

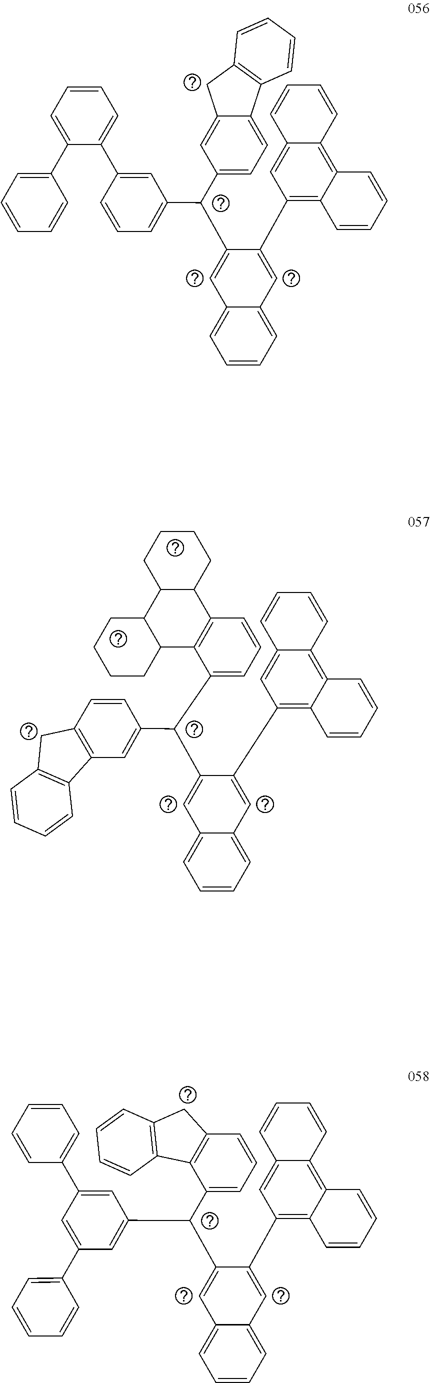

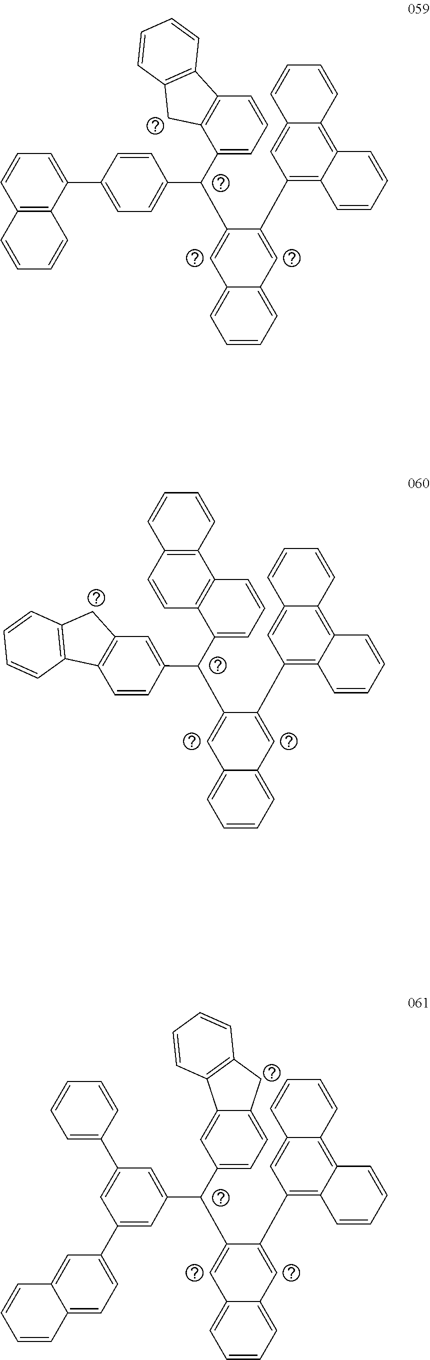

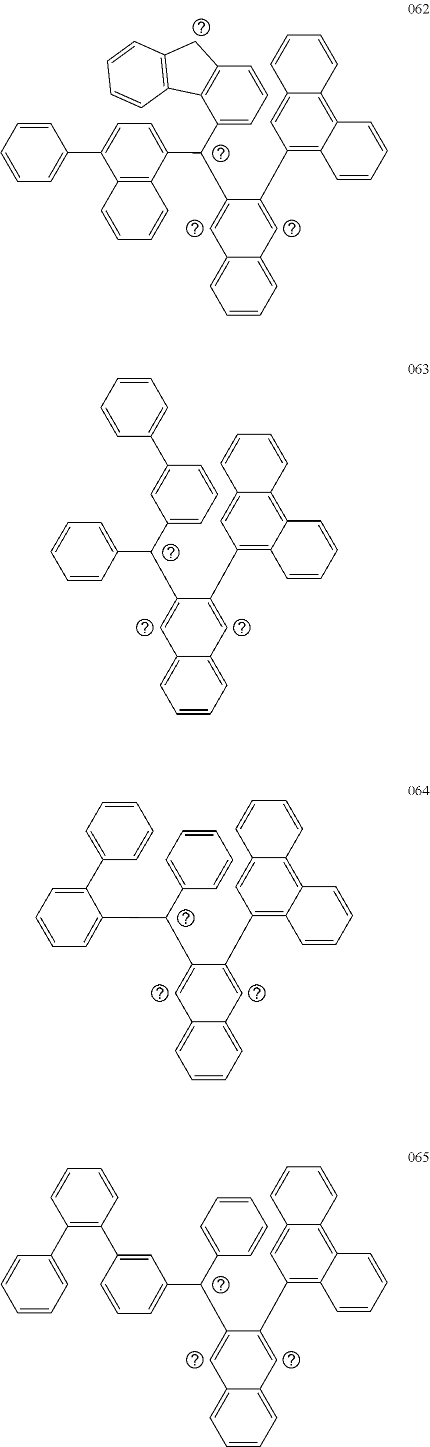

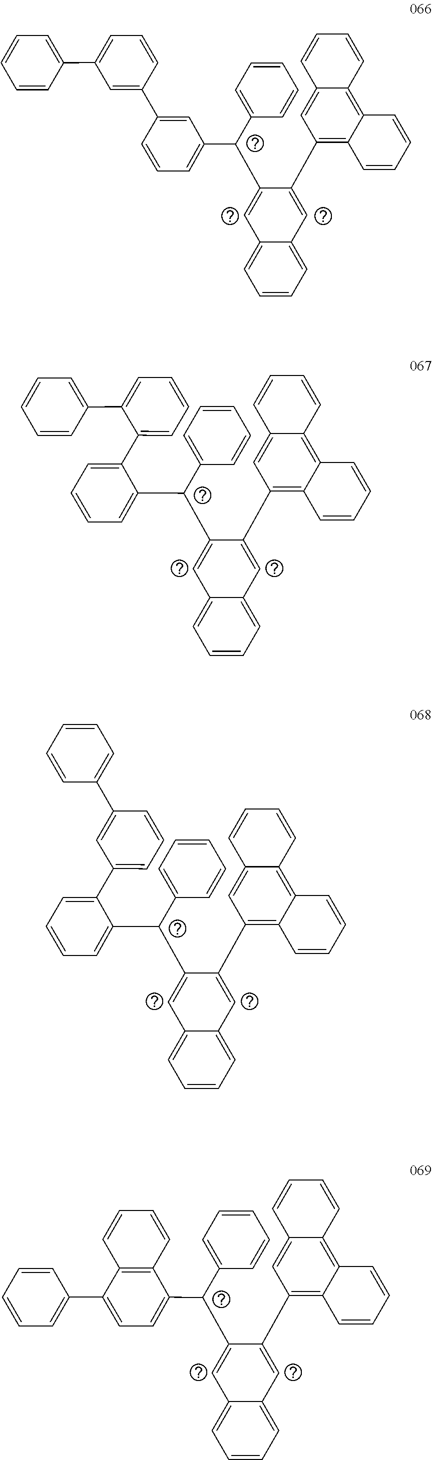

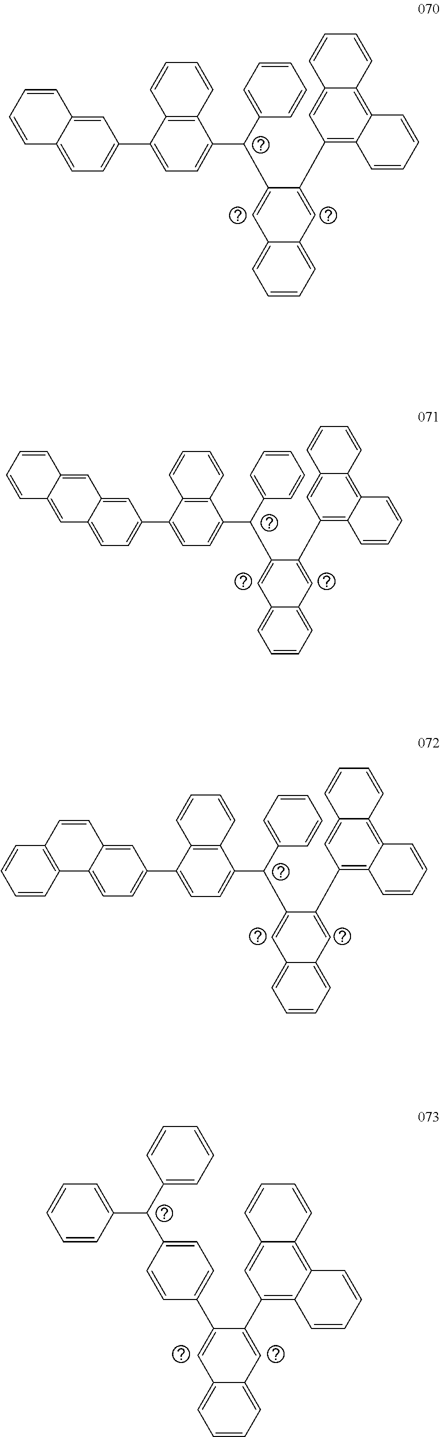

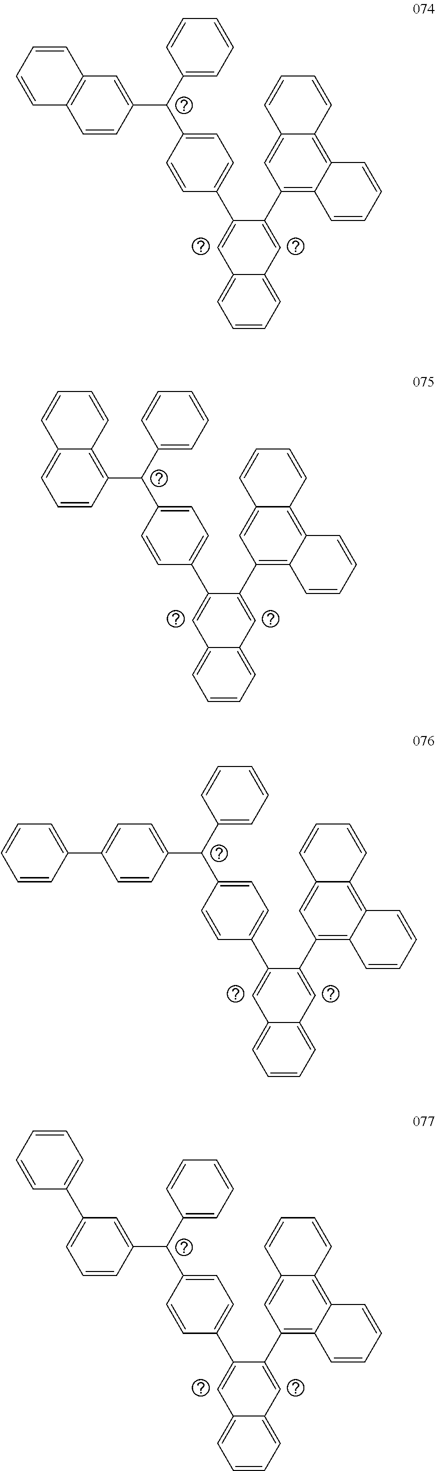

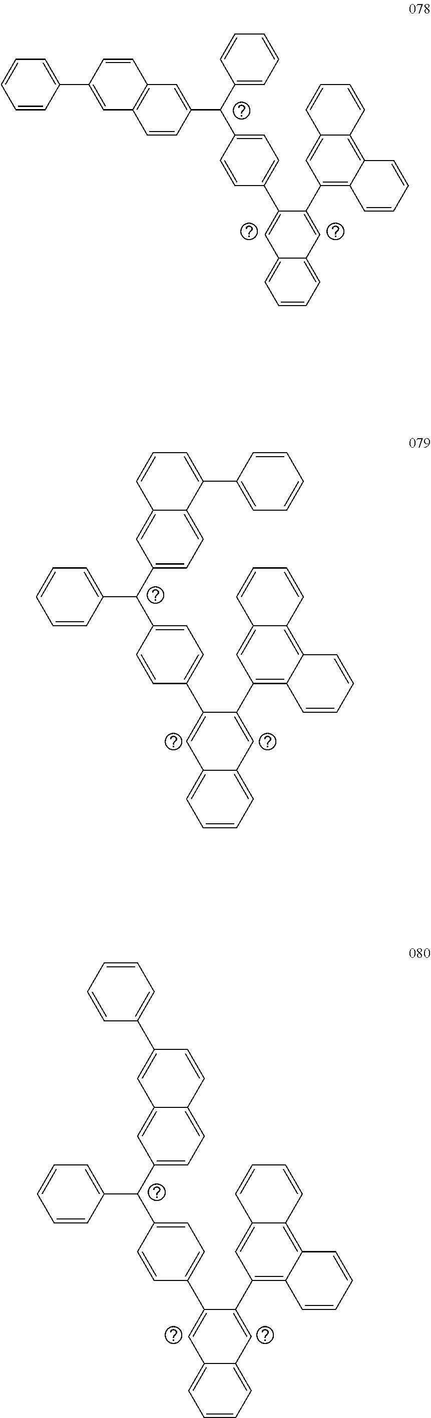

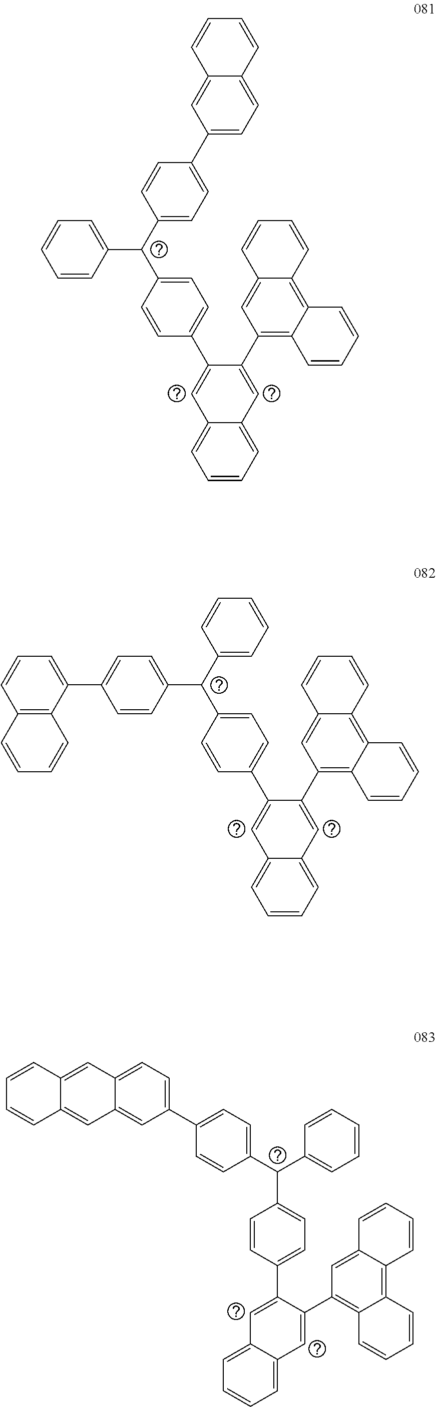

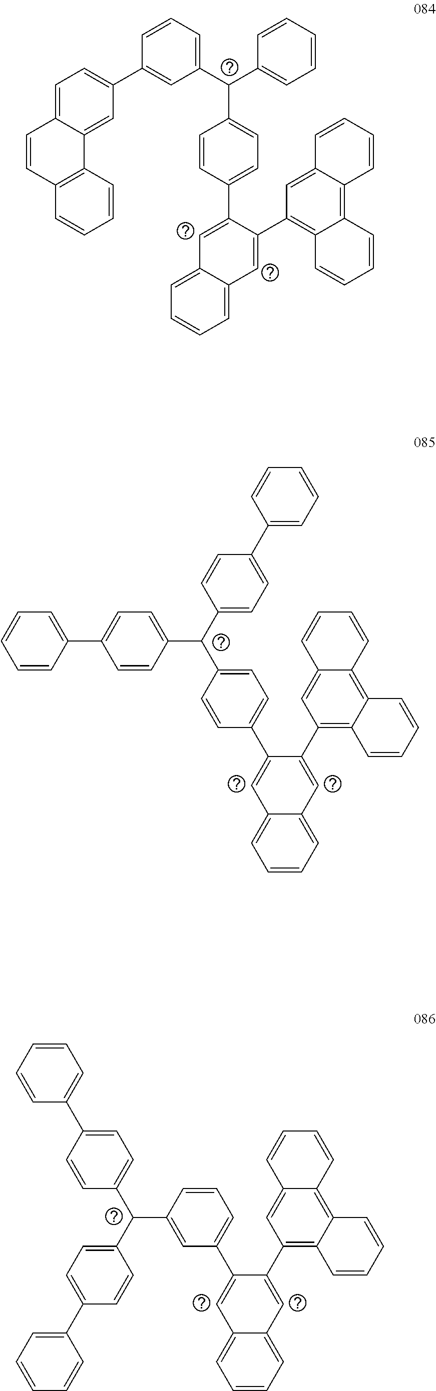

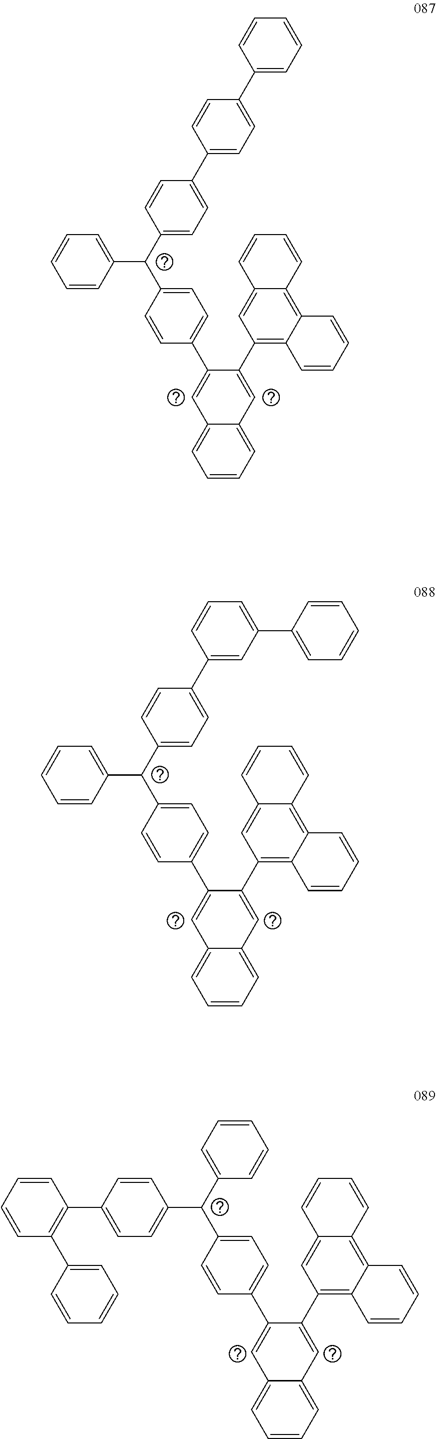

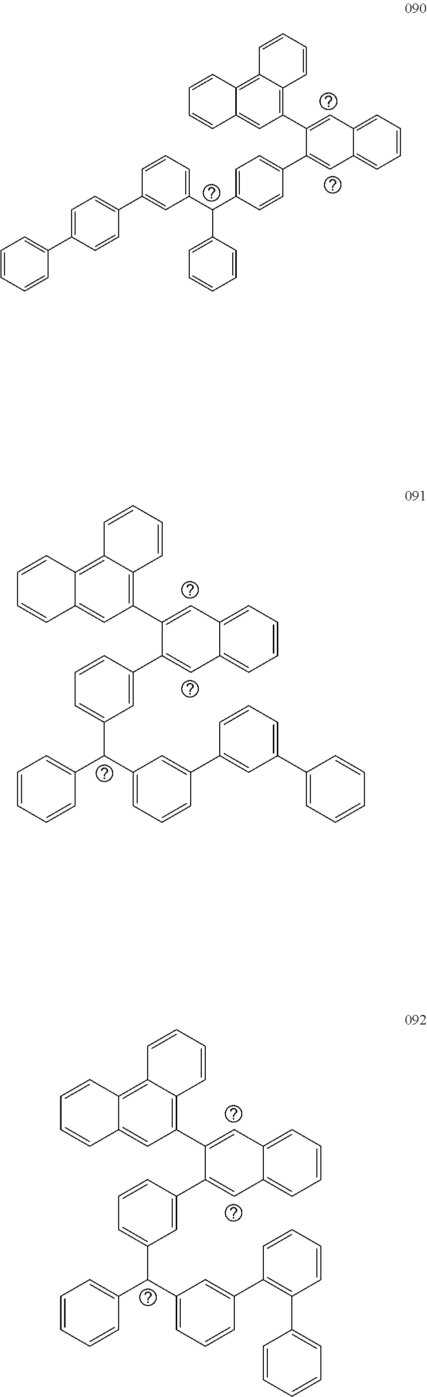

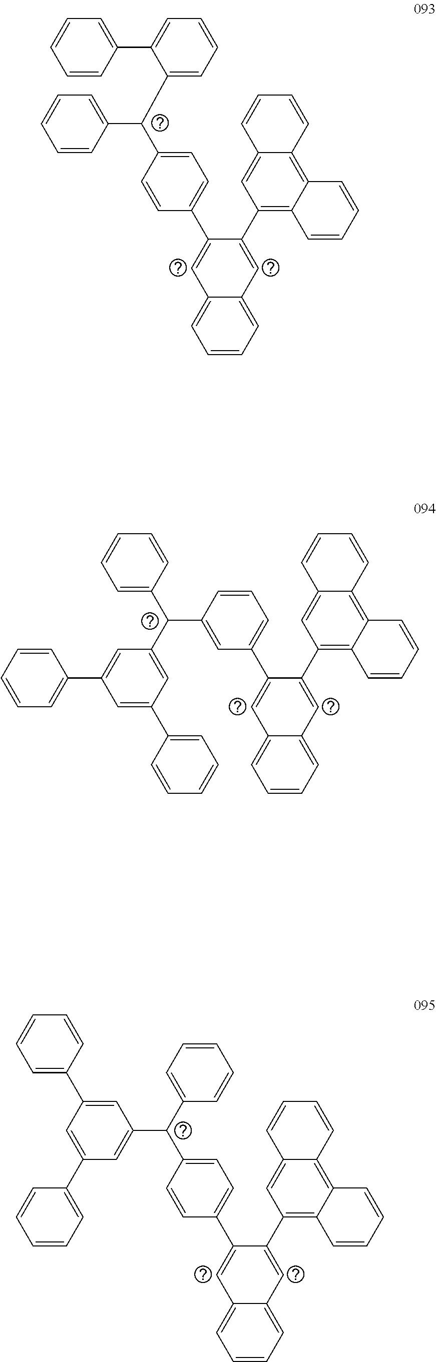

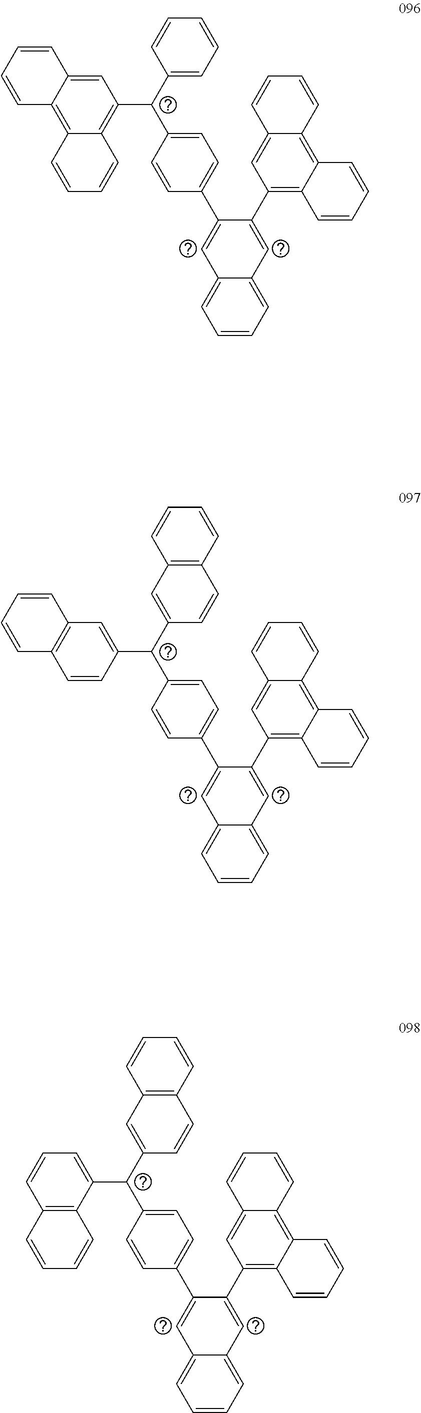

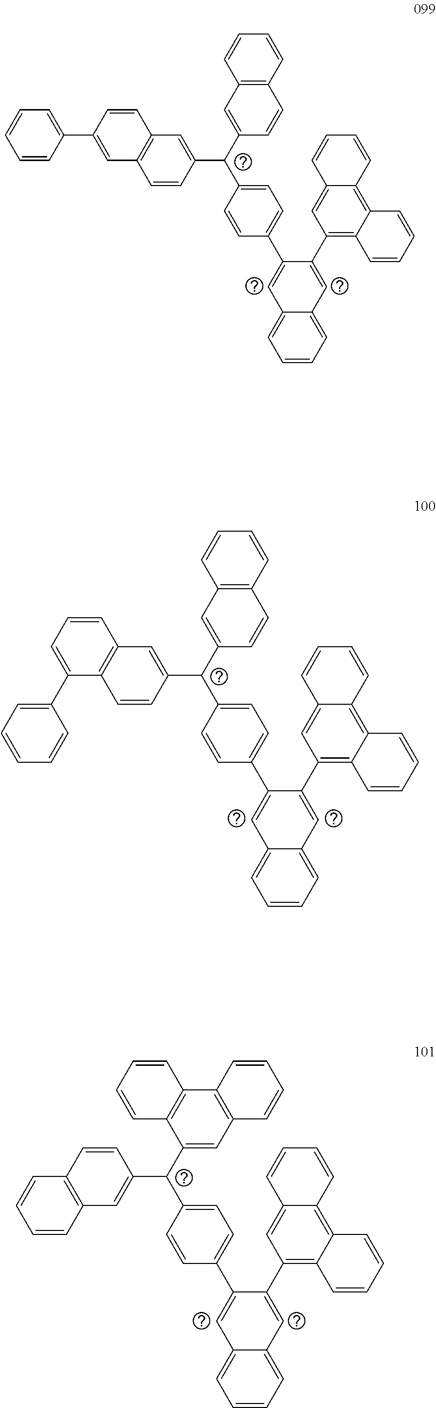

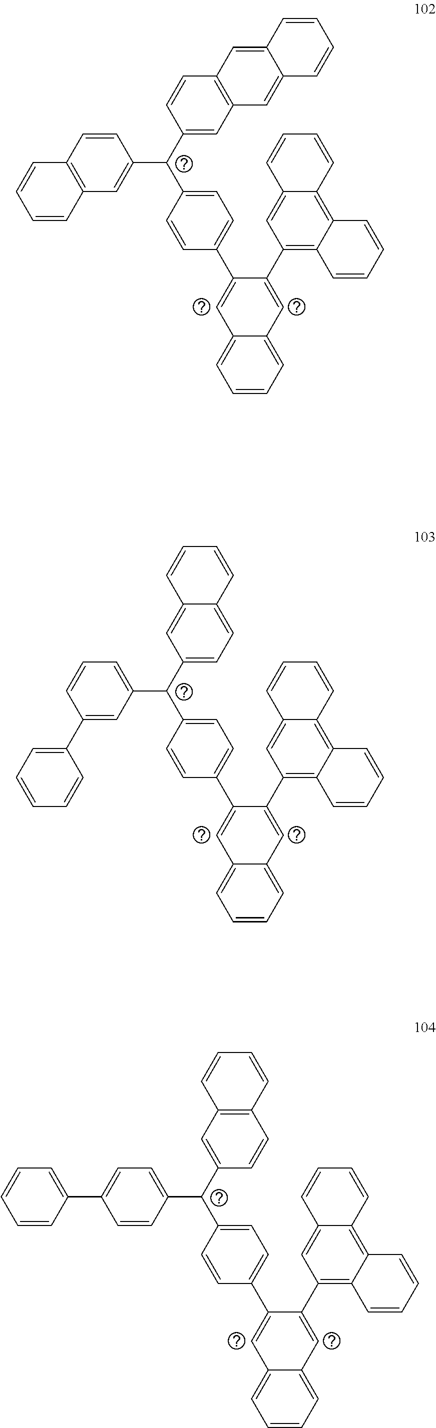

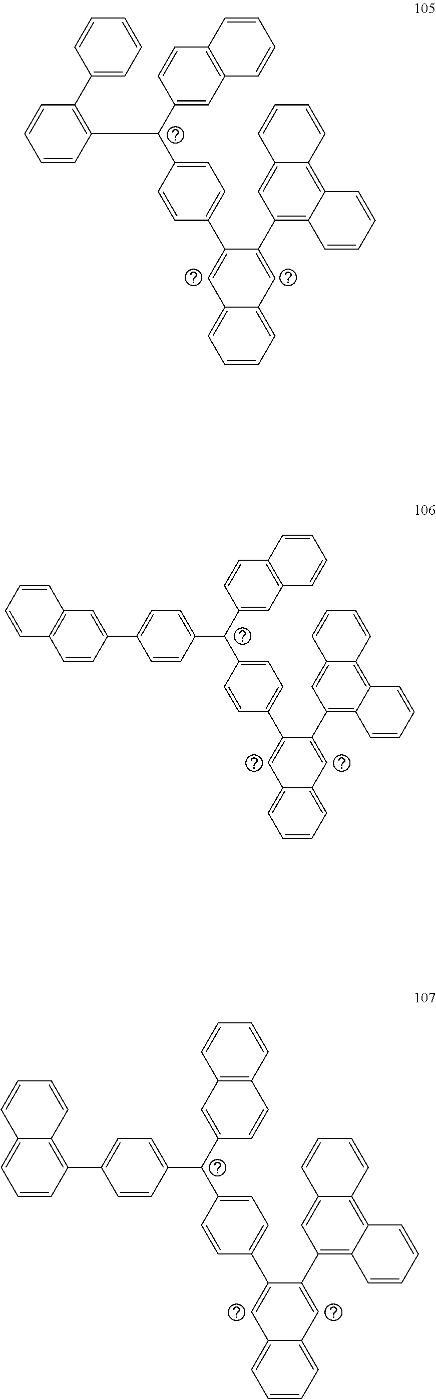

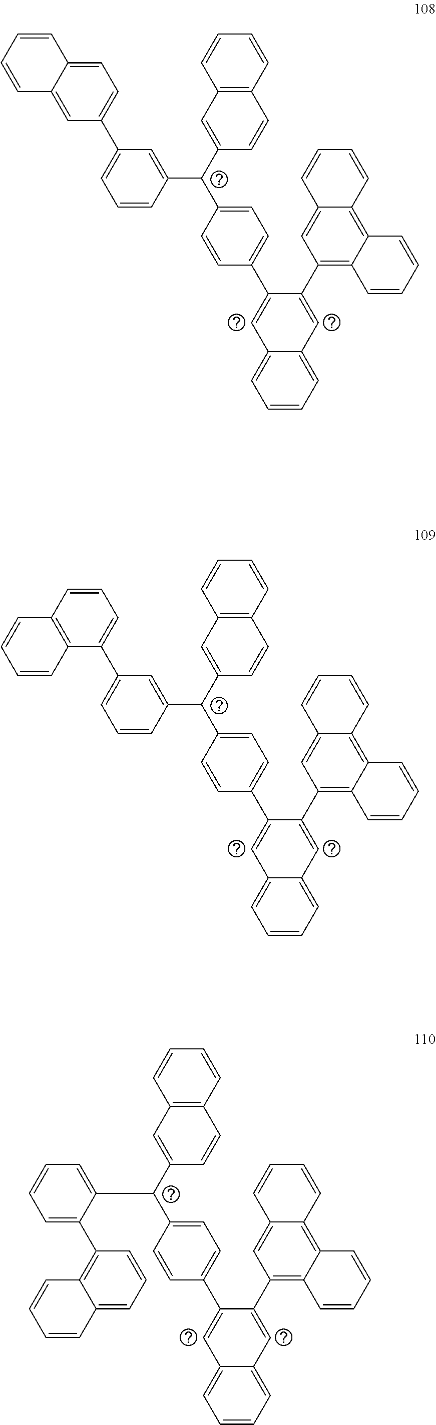

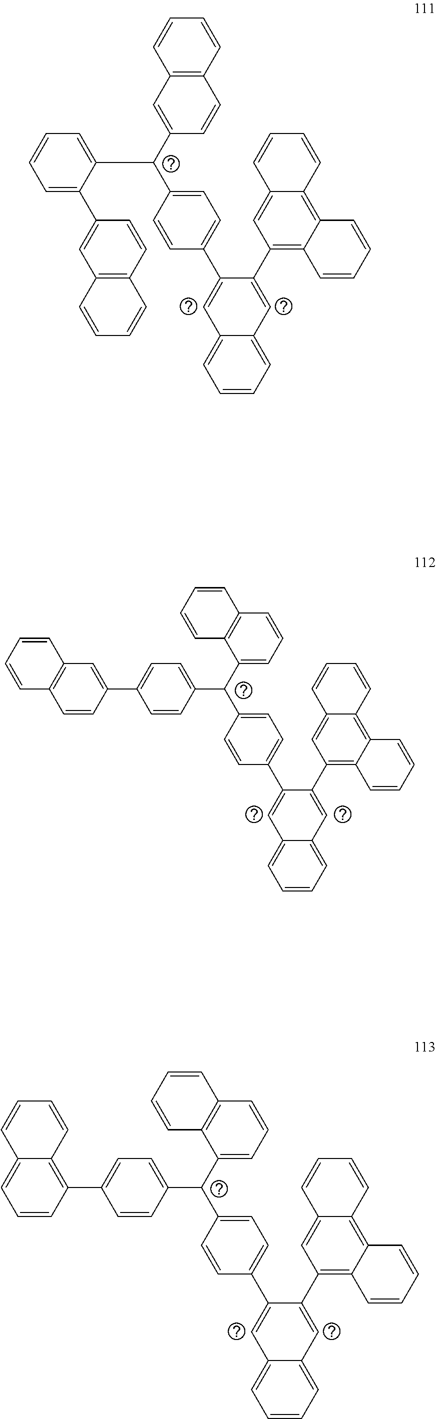

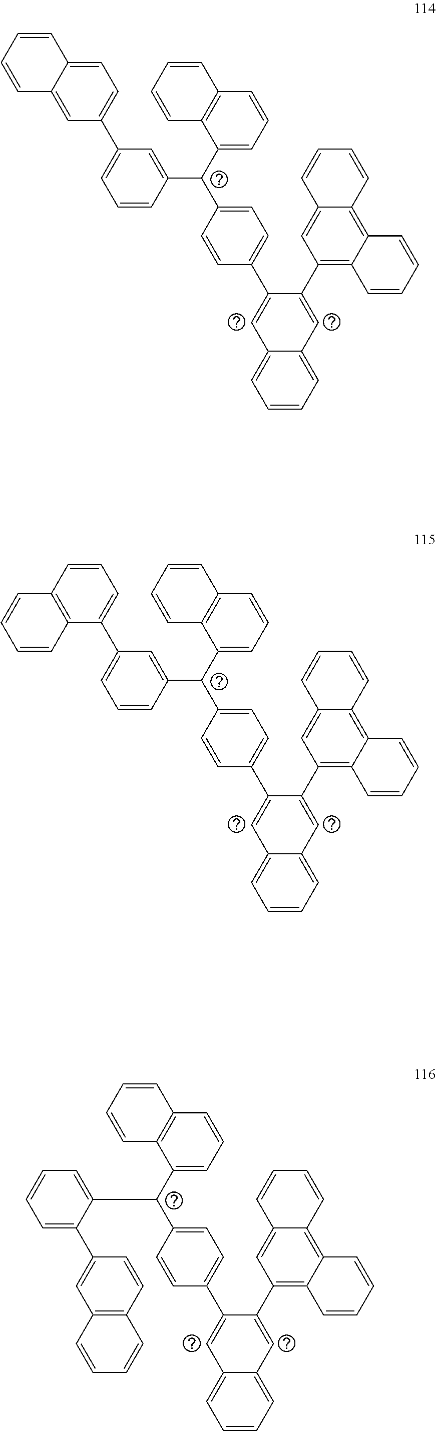

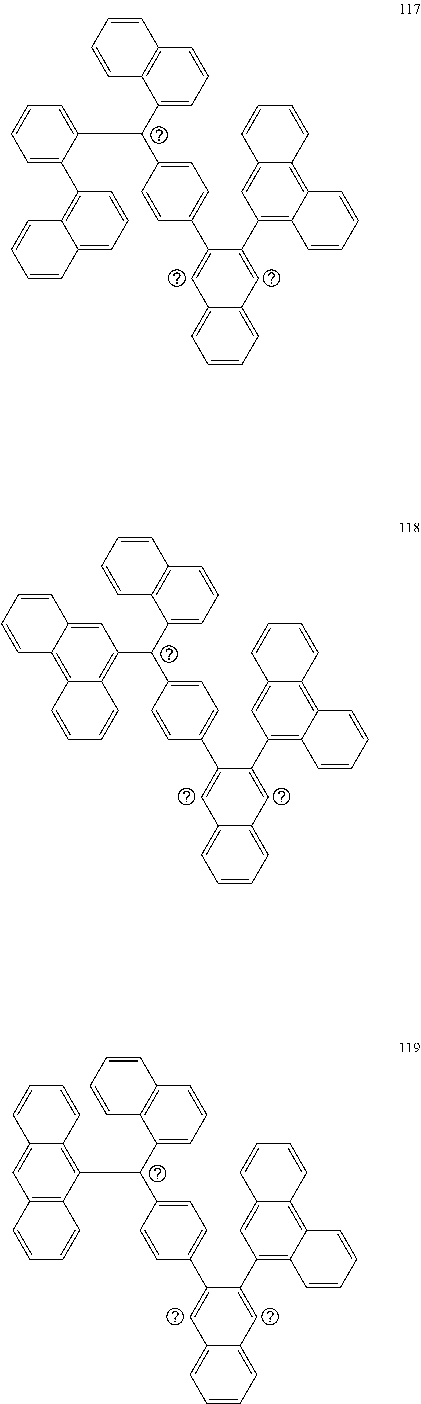

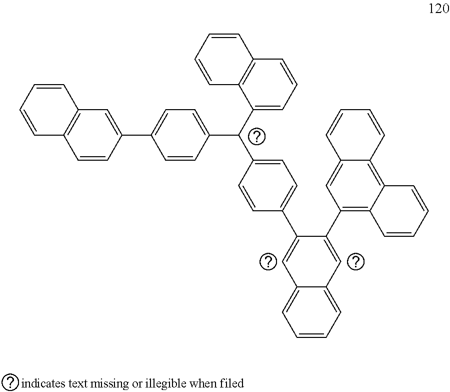 [SMILES: c1ccc2cc(-c3ccc(C(c4ccc(-c5cc6ccccc6cc5-c5cc6ccccc6c6ccccc56)cc4)c4cccc5ccccc45)cc3)ccc2c1]

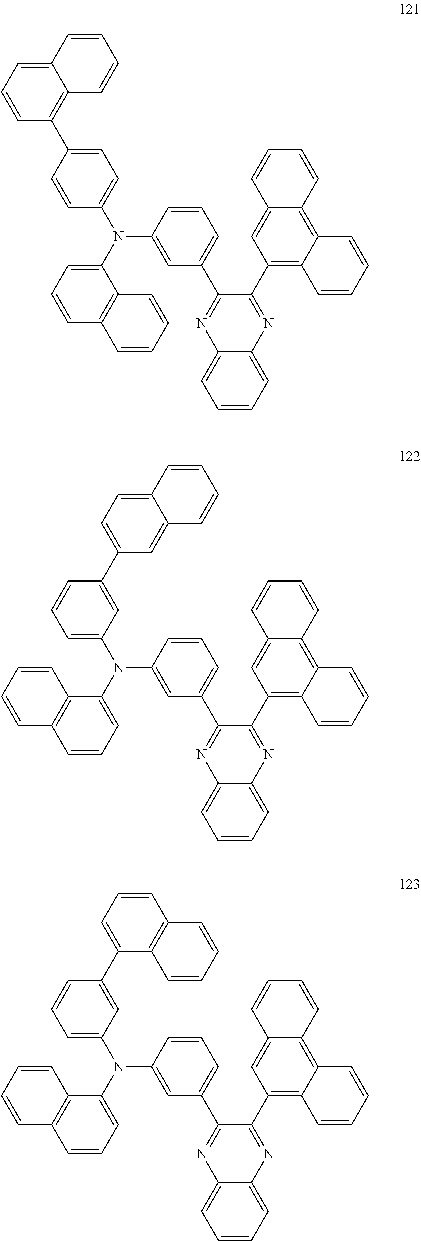 [SMILES: c1cc(-c2nc3ccccc3nc2-c2cc3ccccc3c3ccccc23)cc(N(c2ccc(-c3cccc4ccccc34)cc2)c2cccc3ccccc23)c1 ; c1cc(-c2ccc3ccccc3c2)cc(N(c2cccc(-c3nc4ccccc4nc3-c3cc4ccccc4c4ccccc34)c2)c2cccc3ccccc23)c1 ; c1cc(-c2nc3ccccc3nc2-c2cc3ccccc3c3ccccc23)cc(N(c2cccc(-c3cccc4ccccc34)c2)c2cccc3ccccc23)c1]

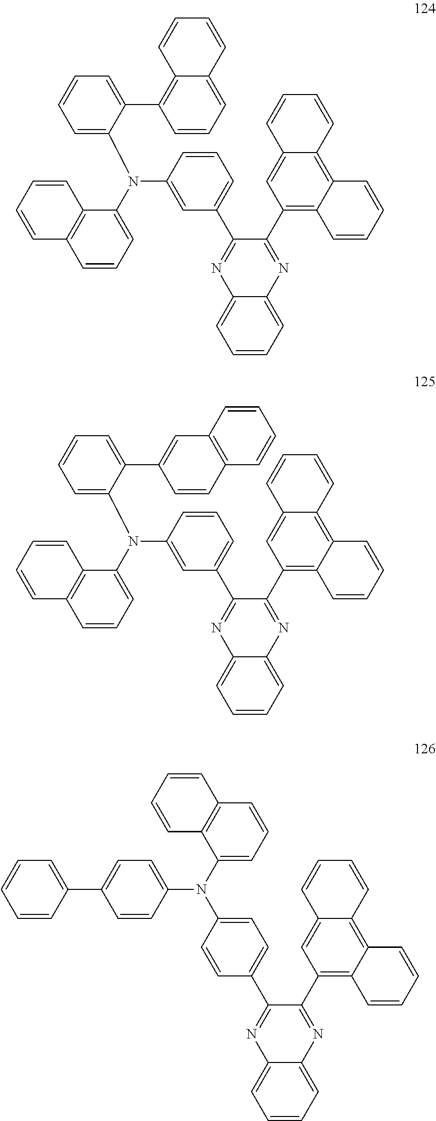 [SMILES: c1cc(-c2nc3ccccc3nc2-c2cc3ccccc3c3ccccc23)cc(N(c2ccccc2-c2cccc3ccccc23)c2cccc3ccccc23)c1 ; c1cc(-c2nc3ccccc3nc2-c2cc3ccccc3c3ccccc23)cc(N(c2ccccc2-c2ccc3ccccc3c2)c2cccc3ccccc23)c1 ; c1ccc(-c2ccc(N(c3ccc(-c4nc5ccccc5nc4-c4cc5ccccc5c5ccccc45)cc3)c3cccc4ccccc34)cc2)cc1]

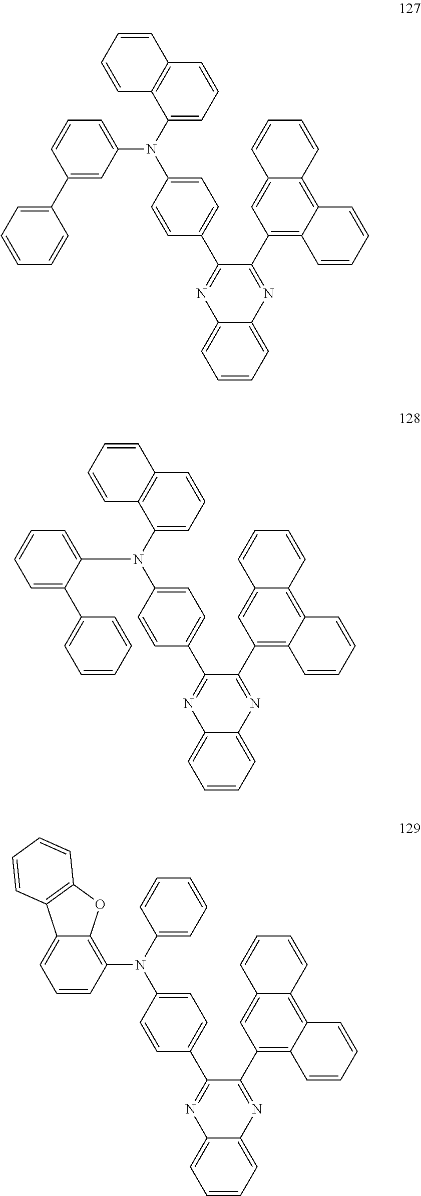 [SMILES: c1ccc(-c2cccc(N(c3ccc(-c4nc5ccccc5nc4-c4cc5ccccc5c5ccccc45)cc3)c3cccc4ccccc34)c2)cc1 ; c1ccc(-c2ccccc2N(c2ccc(-c3nc4ccccc4nc3-c3cc4ccccc4c4ccccc34)cc2)c2cccc3ccccc23)cc1 ; c1ccc(N(c2ccc(-c3nc4ccccc4nc3-c3cc4ccccc4c4ccccc34)cc2)c2cccc3c2oc2ccccc23)cc1]

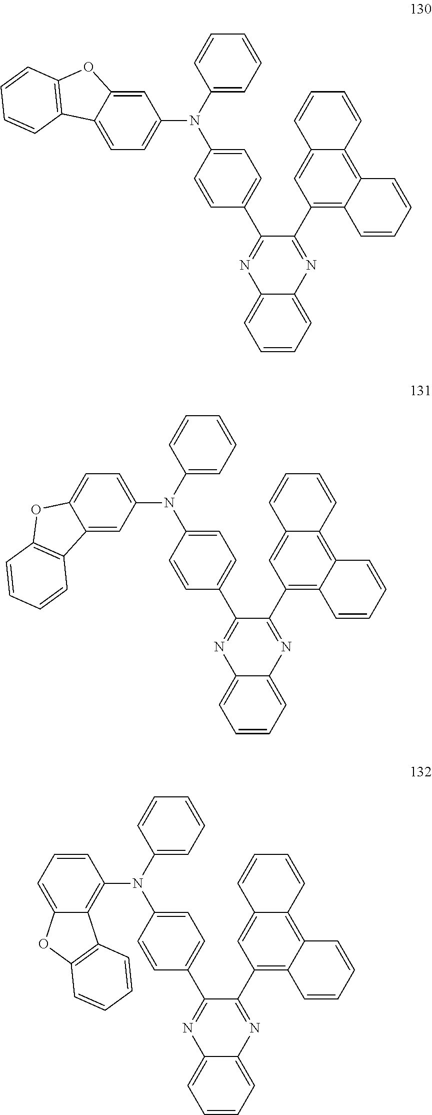 [SMILES: c1ccc(N(c2ccc(-c3nc4ccccc4nc3-c3cc4ccccc4c4ccccc34)cc2)c2ccc3c(c2)oc2ccccc23)cc1 ; c1ccc(N(c2ccc(-c3nc4ccccc4nc3-c3cc4ccccc4c4ccccc34)cc2)c2ccc3oc4ccccc4c3c2)cc1 ; c1ccc(N(c2ccc(-c3nc4ccccc4nc3-c3cc4ccccc4c4ccccc34)cc2)c2cccc3oc4ccccc4c23)cc1]

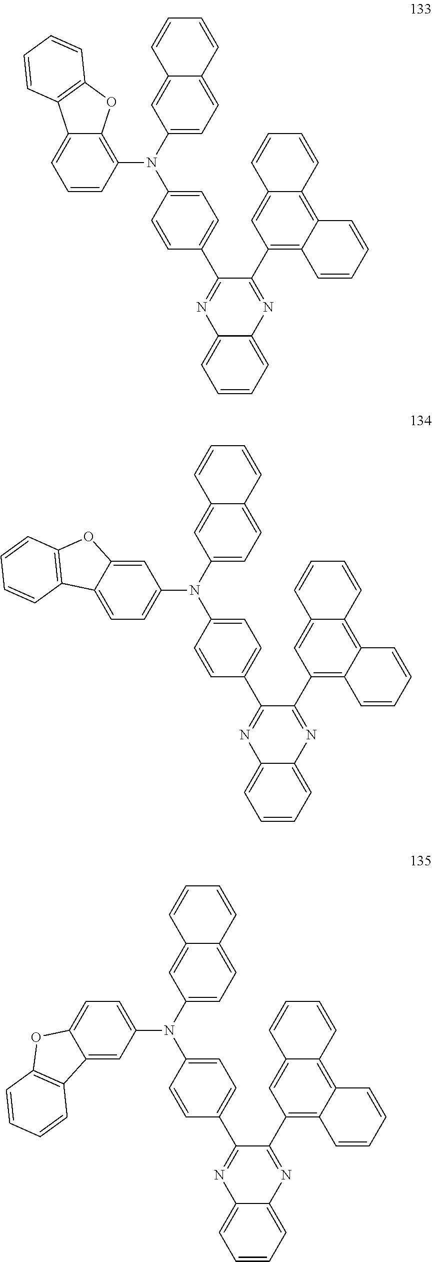 [SMILES: c1ccc2cc(N(c3ccc(-c4nc5ccccc5nc4-c4cc5ccccc5c5ccccc45)cc3)c3cccc4c3oc3ccccc34)ccc2c1 ; c1ccc2cc(N(c3ccc(-c4nc5ccccc5nc4-c4cc5ccccc5c5ccccc45)cc3)c3ccc4c(c3)oc3ccccc34)ccc2c1 ; c1ccc2cc(N(c3ccc(-c4nc5ccccc5nc4-c4cc5ccccc5c5ccccc45)cc3)c3ccc4oc5ccccc5c4c3)ccc2c1]

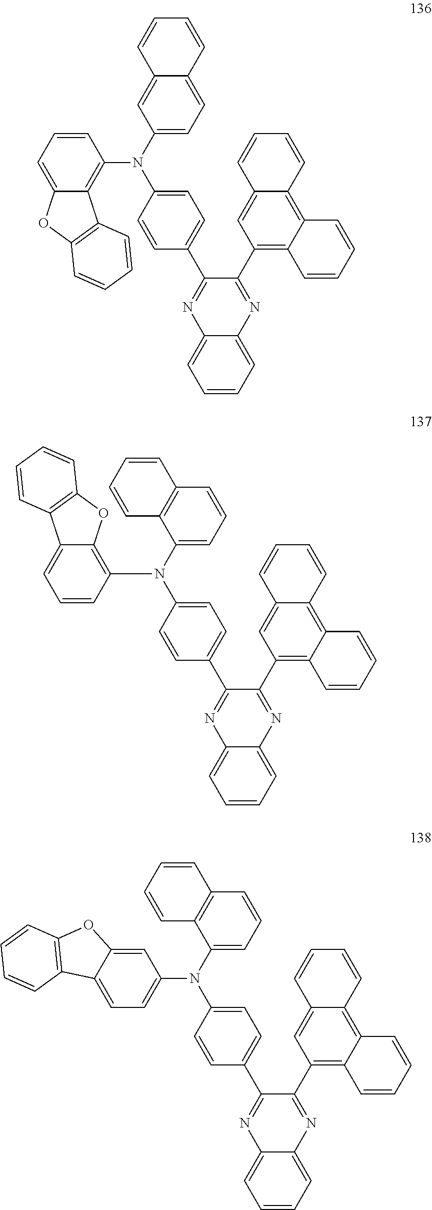 [SMILES: c1ccc2cc(N(c3ccc(-c4nc5ccccc5nc4-c4cc5ccccc5c5ccccc45)cc3)c3cccc4oc5ccccc5c34)ccc2c1 ; c1ccc2c(N(c3ccc(-c4nc5ccccc5nc4-c4cc5ccccc5c5ccccc45)cc3)c3cccc4c3oc3ccccc34)cccc2c1 ; c1ccc2c(N(c3ccc(-c4nc5ccccc5nc4-c4cc5ccccc5c5ccccc45)cc3)c3ccc4c(c3)oc3ccccc34)cccc2c1]

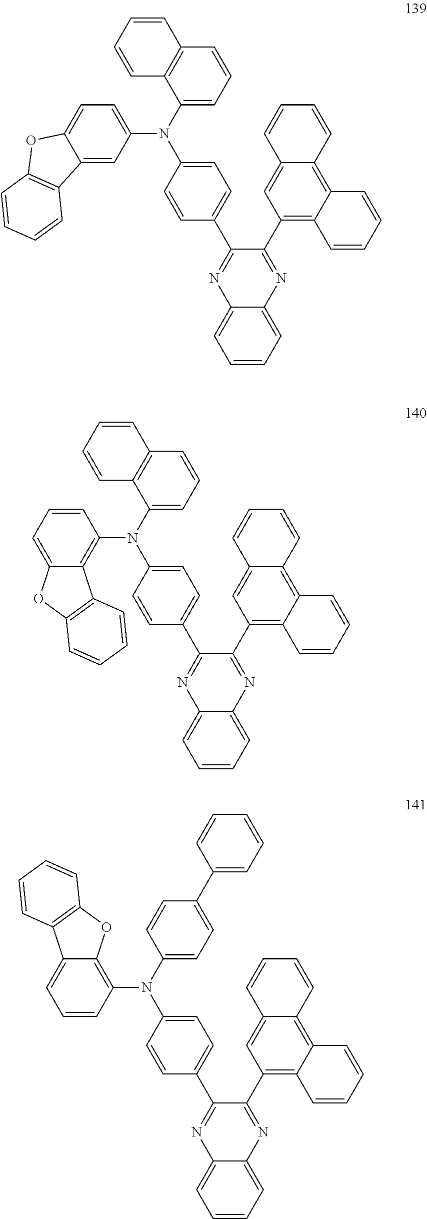 [SMILES: c1ccc2c(N(c3ccc(-c4nc5ccccc5nc4-c4cc5ccccc5c5ccccc45)cc3)c3ccc4oc5ccccc5c4c3)cccc2c1 ; c1ccc2c(N(c3ccc(-c4nc5ccccc5nc4-c4cc5ccccc5c5ccccc45)cc3)c3cccc4oc5ccccc5c34)cccc2c1 ; c1ccc(-c2ccc(N(c3ccc(-c4nc5ccccc5nc4-c4cc5ccccc5c5ccccc45)cc3)c3cccc4c3oc3ccccc34)cc2)cc1]

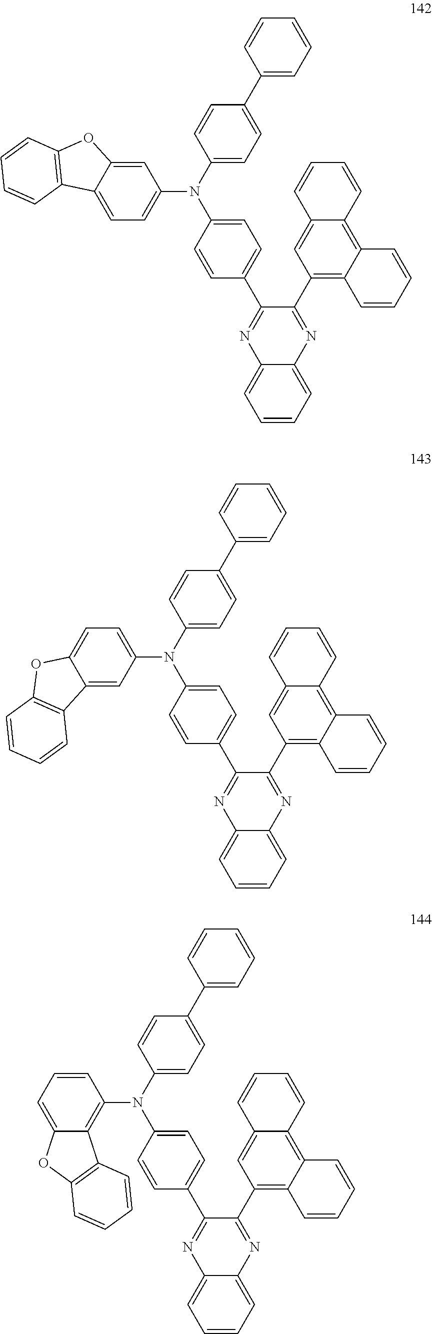

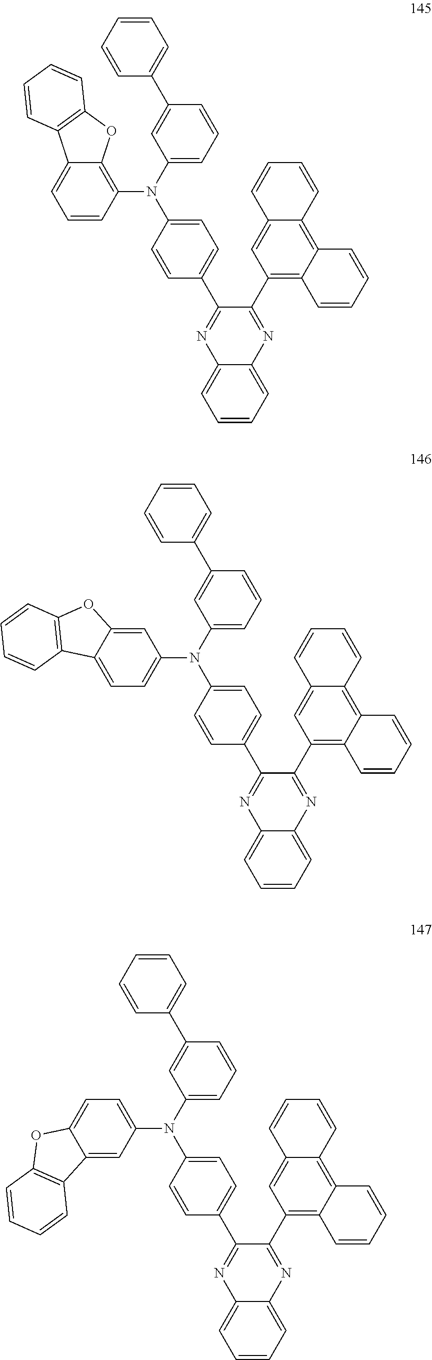 [SMILES: c1ccc(-c2cccc(N(c3ccc(-c4nc5ccccc5nc4-c4cc5ccccc5c5ccccc45)cc3)c3cccc4c3oc3ccccc34)c2)cc1 ; c1ccc(-c2cccc(N(c3ccc(-c4nc5ccccc5nc4-c4cc5ccccc5c5ccccc45)cc3)c3ccc4c(c3)oc3ccccc34)c2)cc1 ; c1ccc(-c2cccc(N(c3ccc(-c4nc5ccccc5nc4-c4cc5ccccc5c5ccccc45)cc3)c3ccc4oc5ccccc5c4c3)c2)cc1]

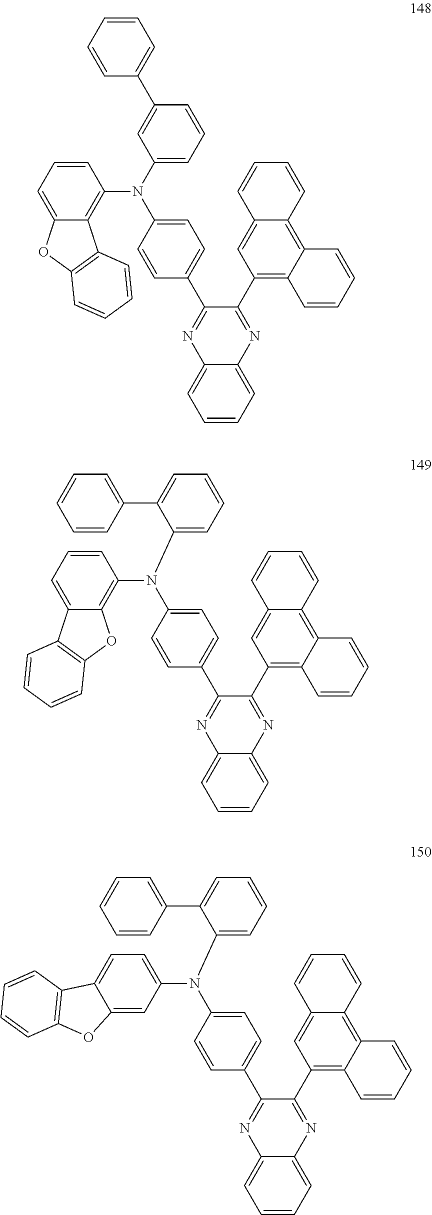 [SMILES: c1ccc(-c2cccc(N(c3ccc(-c4nc5ccccc5nc4-c4cc5ccccc5c5ccccc45)cc3)c3cccc4oc5ccccc5c34)c2)cc1 ; c1ccc(-c2ccccc2N(c2ccc(-c3nc4ccccc4nc3-c3cc4ccccc4c4ccccc34)cc2)c2cccc3c2oc2ccccc23)cc1 ; c1ccc(-c2ccccc2N(c2ccc(-c3nc4ccccc4nc3-c3cc4ccccc4c4ccccc34)cc2)c2ccc3c(c2)oc2ccccc23)cc1]

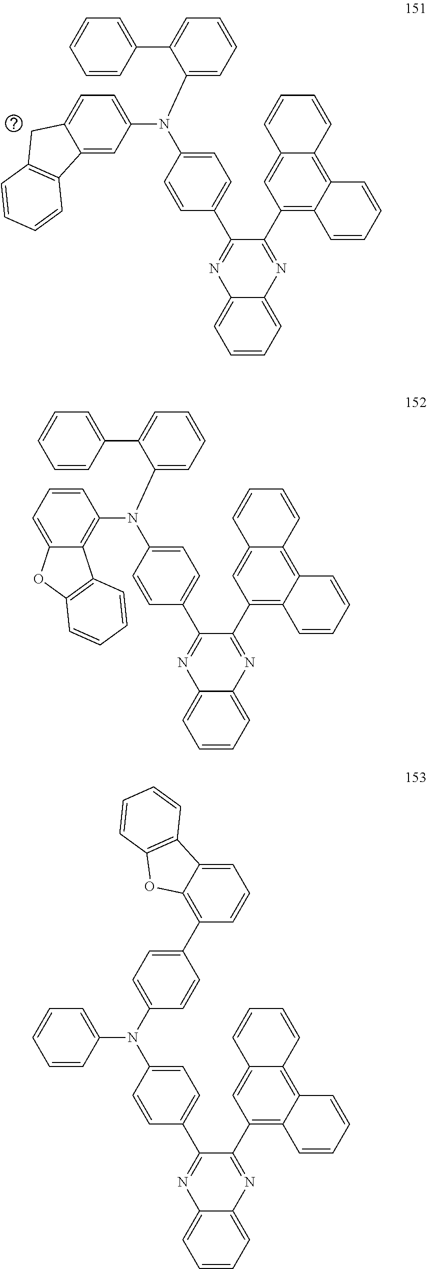 [SMILES: c1ccc(-c2ccccc2N(c2ccc(-c3nc4ccccc4nc3-c3cc4ccccc4c4ccccc34)cc2)c2ccc3c(c2)-c2ccccc2C3)cc1 ; c1ccc(-c2ccccc2N(c2ccc(-c3nc4ccccc4nc3-c3cc4ccccc4c4ccccc34)cc2)c2cccc3oc4ccccc4c23)cc1 ; c1ccc(N(c2ccc(-c3nc4ccccc4nc3-c3cc4ccccc4c4ccccc34)cc2)c2ccc(-c3cccc4c3oc3ccccc34)cc2)cc1]

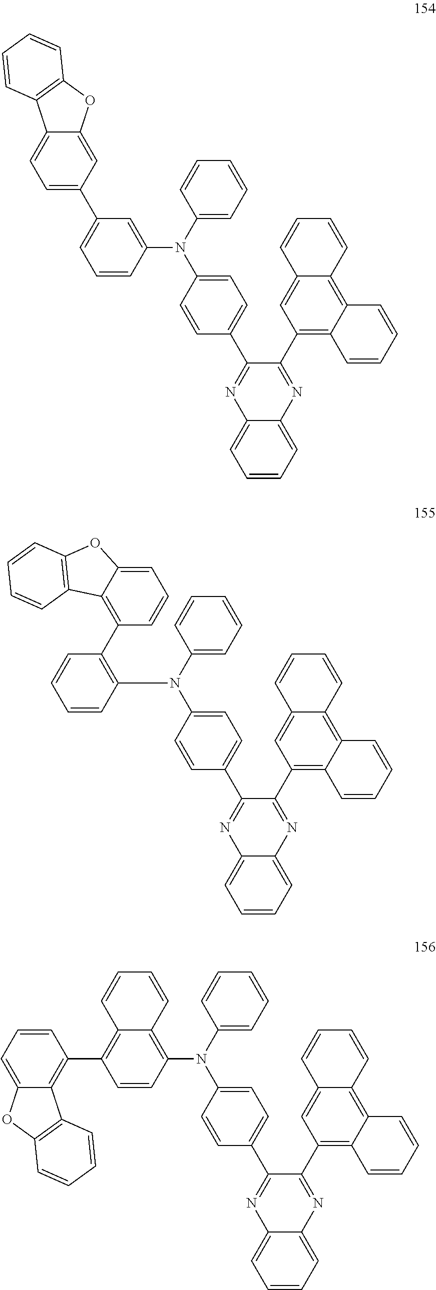 [SMILES: c1ccc(N(c2ccc(-c3nc4ccccc4nc3-c3cc4ccccc4c4ccccc34)cc2)c2cccc(-c3ccc4c(c3)oc3ccccc34)c2)cc1 ; c1ccc(N(c2ccc(-c3nc4ccccc4nc3-c3cc4ccccc4c4ccccc34)cc2)c2ccccc2-c2cccc3oc4ccccc4c23)cc1 ; c1ccc(N(c2ccc(-c3nc4ccccc4nc3-c3cc4ccccc4c4ccccc34)cc2)c2ccc(-c3cccc4oc5ccccc5c34)c3ccccc23)cc1]

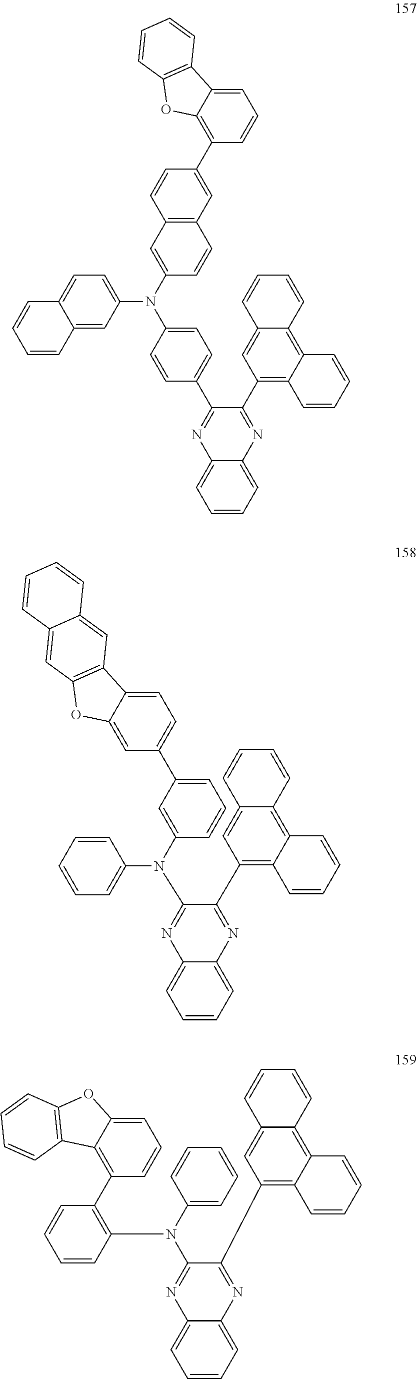 [SMILES: c1ccc2cc(N(c3ccc(-c4nc5ccccc5nc4-c4cc5ccccc5c5ccccc45)cc3)c3ccc4cc(-c5cccc6c5oc5ccccc56)ccc4c3)ccc2c1 ; c1ccc(N(c2cccc(-c3ccc4c(c3)oc3cc5ccccc5cc34)c2)c2nc3ccccc3nc2-c2cc3ccccc3c3ccccc23)cc1 ; c1ccc(N(c2ccccc2-c2cccc3oc4ccccc4c23)c2nc3ccccc3nc2-c2cc3ccccc3c3ccccc23)cc1]

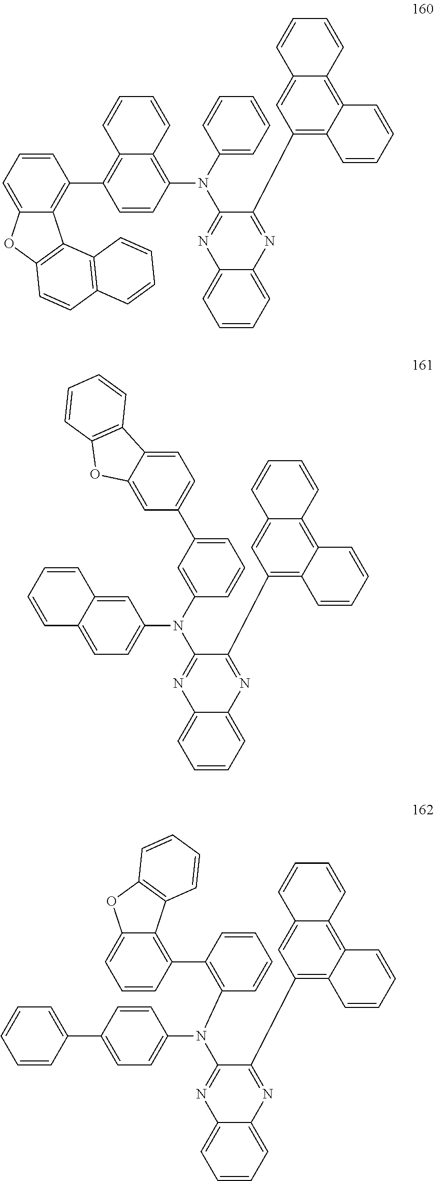 [SMILES: c1ccc(N(c2nc3ccccc3nc2-c2cc3ccccc3c3ccccc23)c2ccc(-c3cccc4oc5ccc6ccccc6c5c34)c3ccccc23)cc1 ; c1cc(-c2ccc3c(c2)oc2ccccc23)cc(N(c2ccc3ccccc3c2)c2nc3ccccc3nc2-c2cc3ccccc3c3ccccc23)c1 ; c1ccc(-c2ccc(N(c3ccccc3-c3cccc4oc5ccccc5c34)c3nc4ccccc4nc3-c3cc4ccccc4c4ccccc34)cc2)cc1]

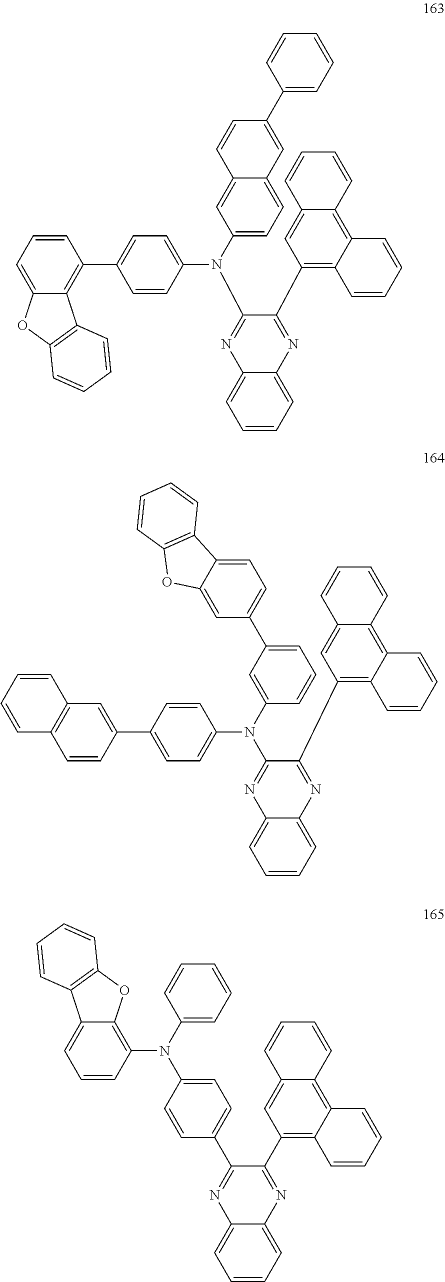 [SMILES: c1ccc(-c2ccc3cc(N(c4ccc(-c5cccc6oc7ccccc7c56)cc4)c4nc5ccccc5nc4-c4cc5ccccc5c5ccccc45)ccc3c2)cc1 ; c1cc(-c2ccc3c(c2)oc2ccccc23)cc(N(c2ccc(-c3ccc4ccccc4c3)cc2)c2nc3ccccc3nc2-c2cc3ccccc3c3ccccc23)c1 ; c1ccc(N(c2ccc(-c3nc4ccccc4nc3-c3cc4ccccc4c4ccccc34)cc2)c2cccc3c2oc2ccccc23)cc1]

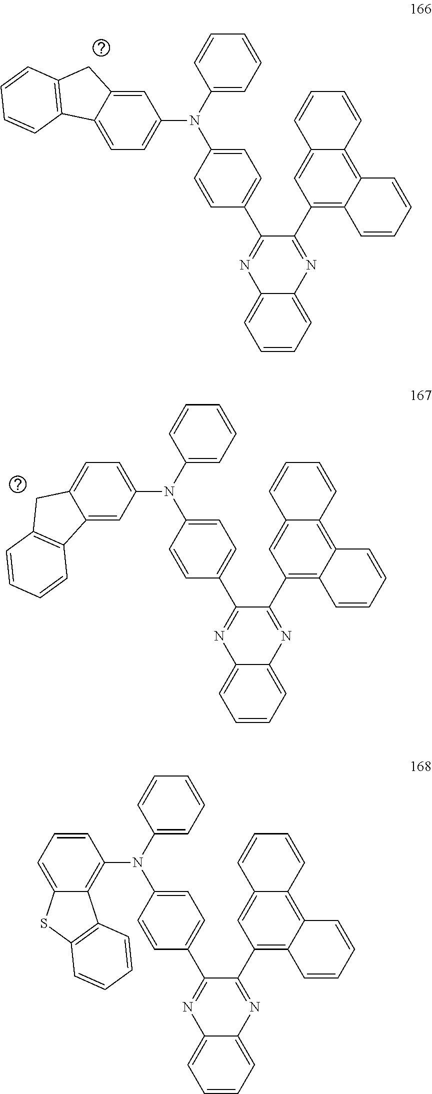 [SMILES: c1ccc(N(c2ccc(-c3nc4ccccc4nc3-c3cc4ccccc4c4ccccc34)cc2)c2ccc3c(c2)Cc2ccccc2-3)cc1 ; c1ccc(N(c2ccc(-c3nc4ccccc4nc3-c3cc4ccccc4c4ccccc34)cc2)c2ccc3c(c2)-c2ccccc2C3)cc1 ; c1ccc(N(c2ccc(-c3nc4ccccc4nc3-c3cc4ccccc4c4ccccc34)cc2)c2cccc3sc4ccccc4c23)cc1]

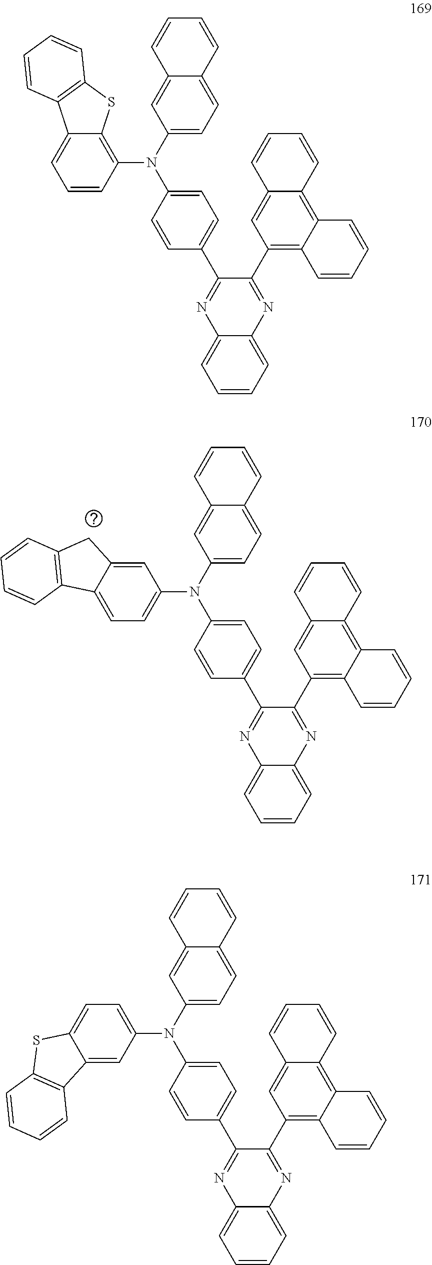 [SMILES: c1ccc2cc(N(c3ccc(-c4nc5ccccc5nc4-c4cc5ccccc5c5ccccc45)cc3)c3cccc4c3sc3ccccc34)ccc2c1 ; c1ccc2c(c1)Cc1cc(N(c3ccc(-c4nc5ccccc5nc4-c4cc5ccccc5c5ccccc45)cc3)c3ccc4ccccc4c3)ccc1-2 ; c1ccc2cc(N(c3ccc(-c4nc5ccccc5nc4-c4cc5ccccc5c5ccccc45)cc3)c3ccc4sc5ccccc5c4c3)ccc2c1]

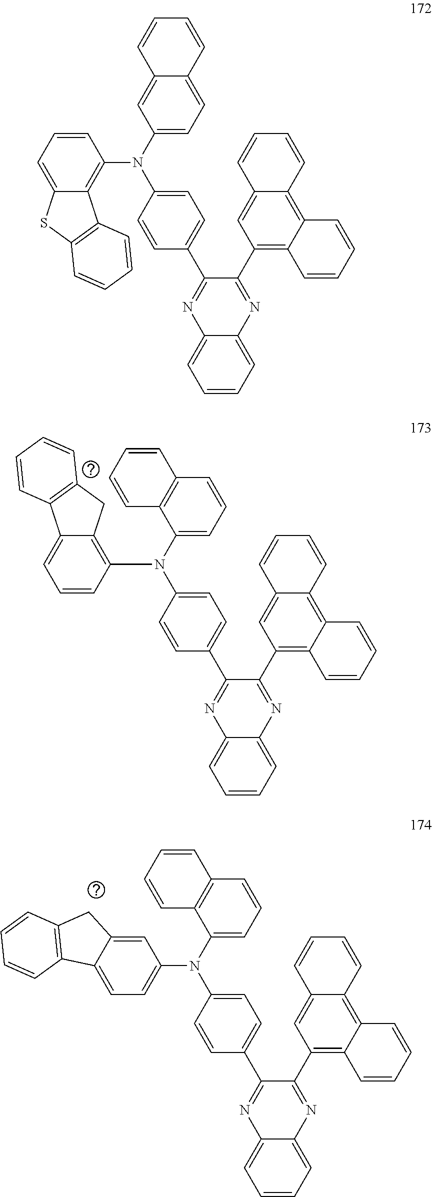 [SMILES: c1ccc2cc(N(c3ccc(-c4nc5ccccc5nc4-c4cc5ccccc5c5ccccc45)cc3)c3cccc4sc5ccccc5c34)ccc2c1 ; c1ccc2c(c1)Cc1c-2cccc1N(c1ccc(-c2nc3ccccc3nc2-c2cc3ccccc3c3ccccc23)cc1)c1cccc2ccccc12 ; c1ccc2c(c1)Cc1cc(N(c3ccc(-c4nc5ccccc5nc4-c4cc5ccccc5c5ccccc45)cc3)c3cccc4ccccc34)ccc1-2]

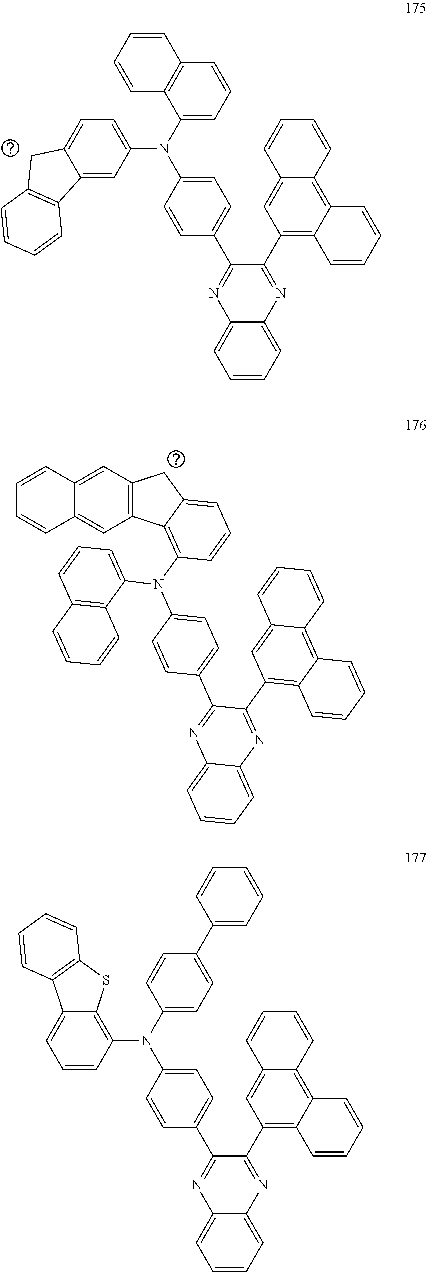

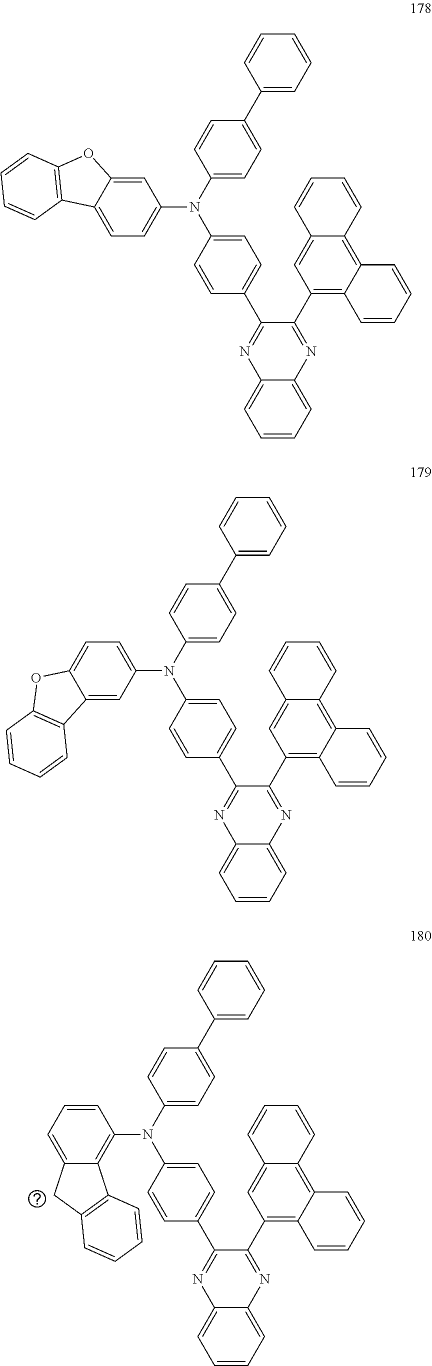 [SMILES: c1ccc(-c2ccc(N(c3ccc(-c4nc5ccccc5nc4-c4cc5ccccc5c5ccccc45)cc3)c3ccc4c(c3)oc3ccccc34)cc2)cc1 ; c1ccc(-c2ccc(N(c3ccc(-c4nc5ccccc5nc4-c4cc5ccccc5c5ccccc45)cc3)c3ccc4oc5ccccc5c4c3)cc2)cc1 ; c1ccc(-c2ccc(N(c3ccc(-c4nc5ccccc5nc4-c4cc5ccccc5c5ccccc45)cc3)c3cccc4c3-c3ccccc3C4)cc2)cc1]

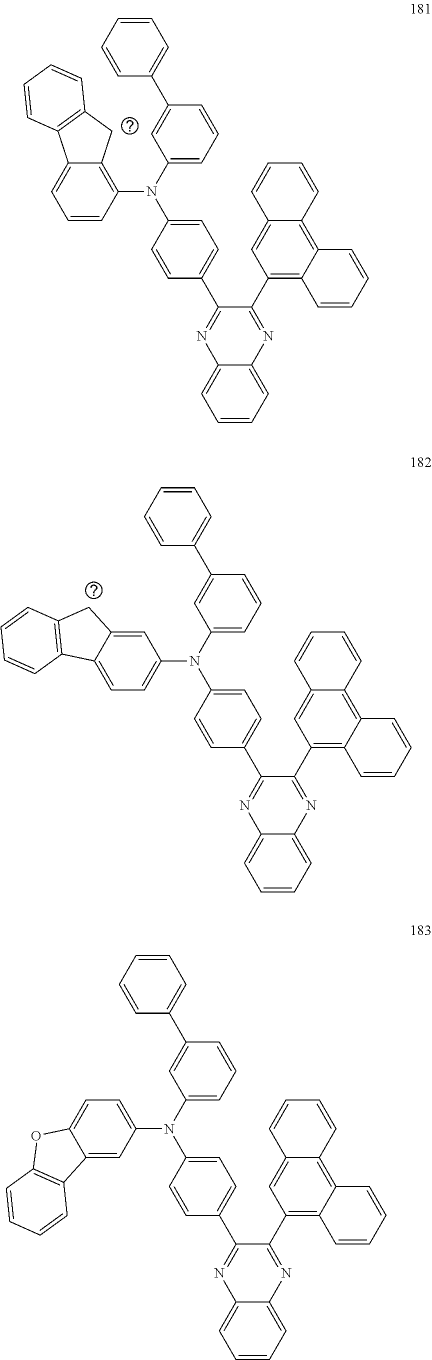 [SMILES: c1ccc(-c2cccc(N(c3ccc(-c4nc5ccccc5nc4-c4cc5ccccc5c5ccccc45)cc3)c3cccc4c3Cc3ccccc3-4)c2)cc1 ; c1ccc(-c2cccc(N(c3ccc(-c4nc5ccccc5nc4-c4cc5ccccc5c5ccccc45)cc3)c3ccc4c(c3)Cc3ccccc3-4)c2)cc1 ; c1ccc(-c2cccc(N(c3ccc(-c4nc5ccccc5nc4-c4cc5ccccc5c5ccccc45)cc3)c3ccc4oc5ccccc5c4c3)c2)cc1]

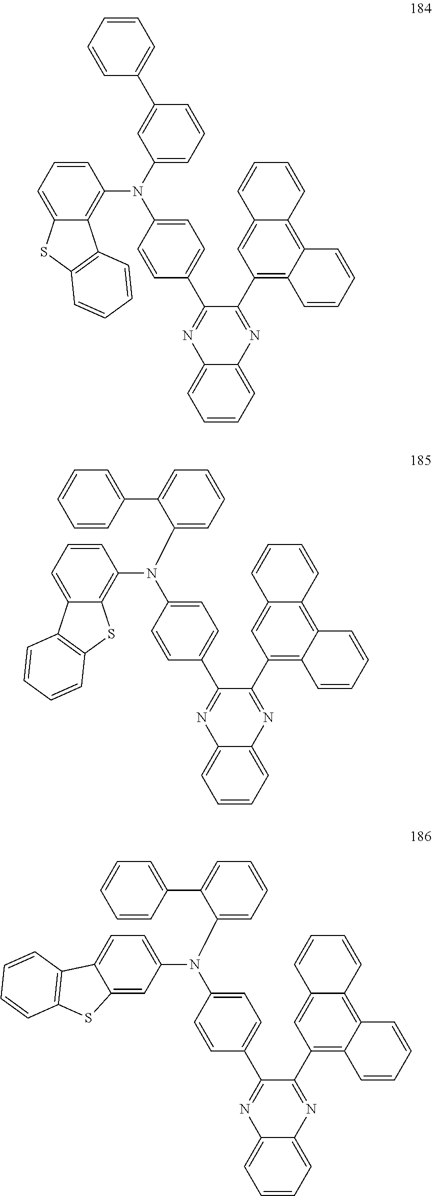 [SMILES: c1ccc(-c2cccc(N(c3ccc(-c4nc5ccccc5nc4-c4cc5ccccc5c5ccccc45)cc3)c3cccc4sc5ccccc5c34)c2)cc1 ; c1ccc(-c2ccccc2N(c2ccc(-c3nc4ccccc4nc3-c3cc4ccccc4c4ccccc34)cc2)c2cccc3c2sc2ccccc23)cc1 ; c1ccc(-c2ccccc2N(c2ccc(-c3nc4ccccc4nc3-c3cc4ccccc4c4ccccc34)cc2)c2ccc3c(c2)sc2ccccc23)cc1]

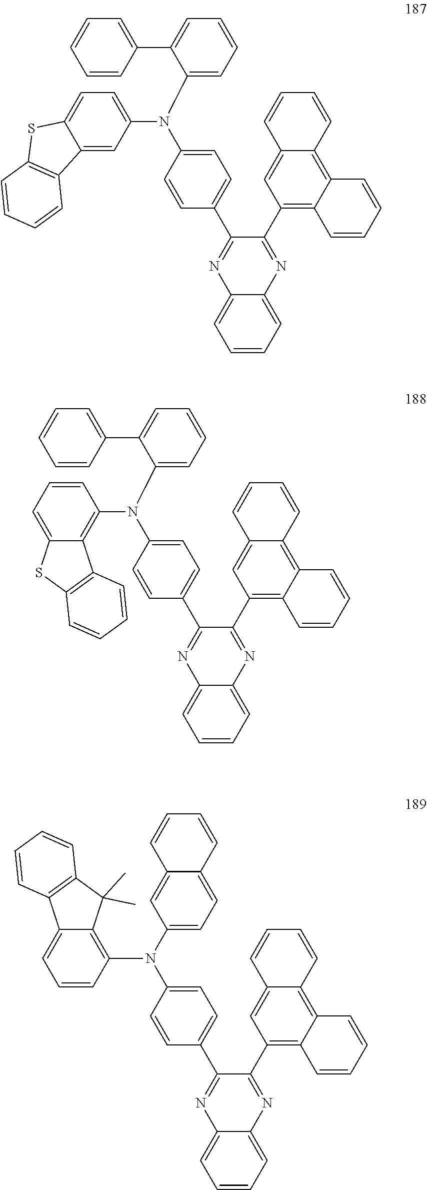 [SMILES: c1ccc(-c2ccccc2N(c2ccc(-c3nc4ccccc4nc3-c3cc4ccccc4c4ccccc34)cc2)c2ccc3sc4ccccc4c3c2)cc1 ; c1ccc(-c2ccccc2N(c2ccc(-c3nc4ccccc4nc3-c3cc4ccccc4c4ccccc34)cc2)c2cccc3sc4ccccc4c23)cc1 ; CC1(C)c2ccccc2-c2cccc(N(c3ccc(-c4nc5ccccc5nc4-c4cc5ccccc5c5ccccc45)cc3)c3ccc4ccccc4c3)c21]

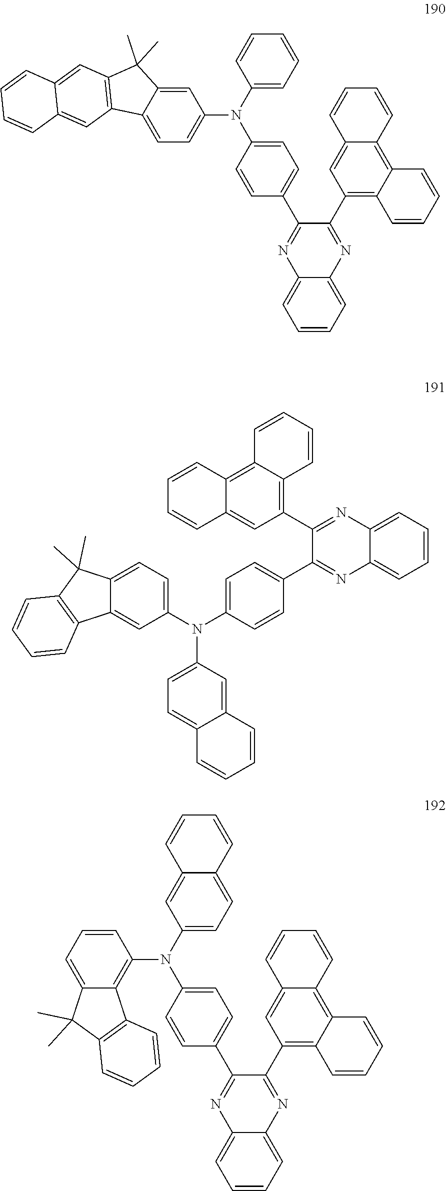

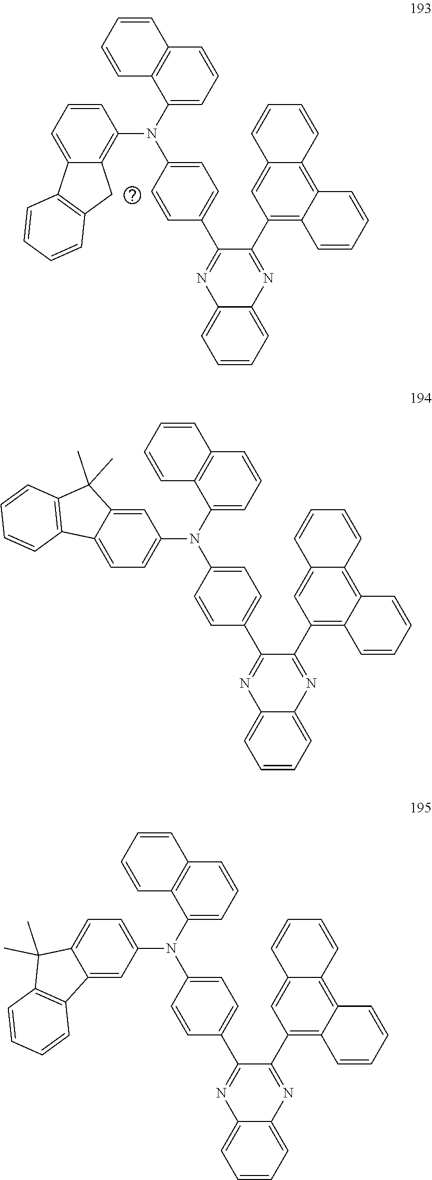 [SMILES: c1ccc2c(c1)Cc1c-2cccc1N(c1ccc(-c2nc3ccccc3nc2-c2cc3ccccc3c3ccccc23)cc1)c1cccc2ccccc12 ; CC1(C)c2ccccc2-c2ccc(N(c3ccc(-c4nc5ccccc5nc4-c4cc5ccccc5c5ccccc45)cc3)c3cccc4ccccc34)cc21 ; CC1(C)c2ccccc2-c2cc(N(c3ccc(-c4nc5ccccc5nc4-c4cc5ccccc5c5ccccc45)cc3)c3cccc4ccccc34)ccc21]

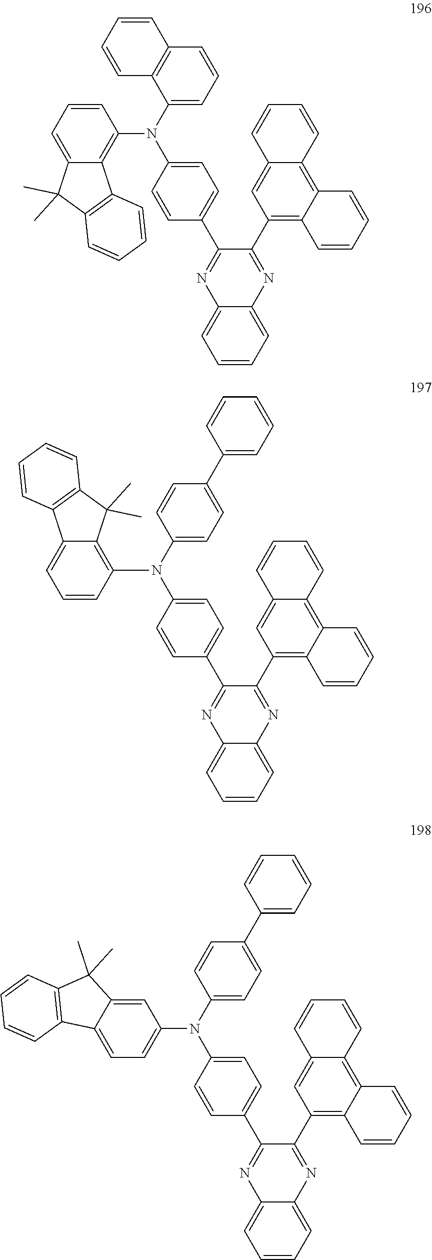

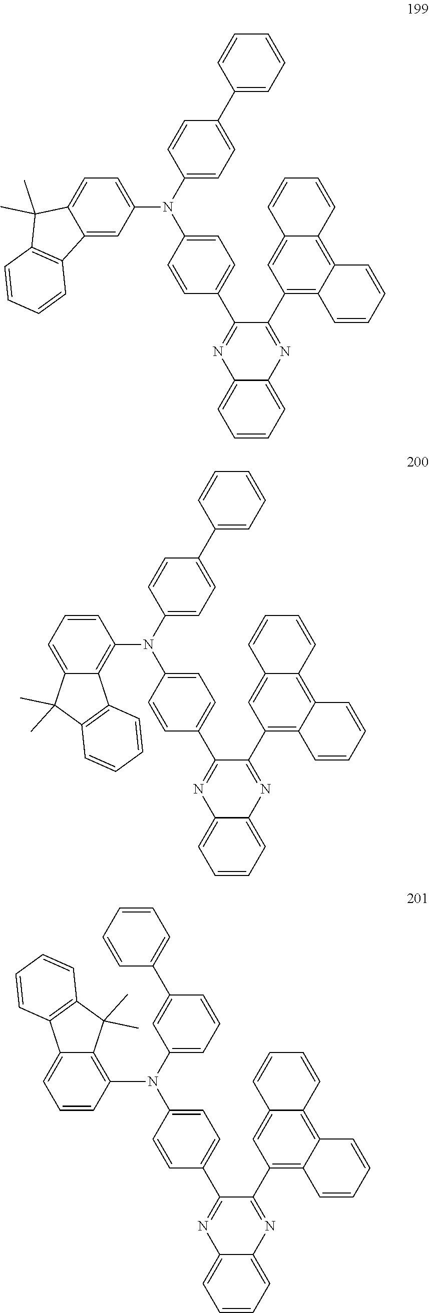

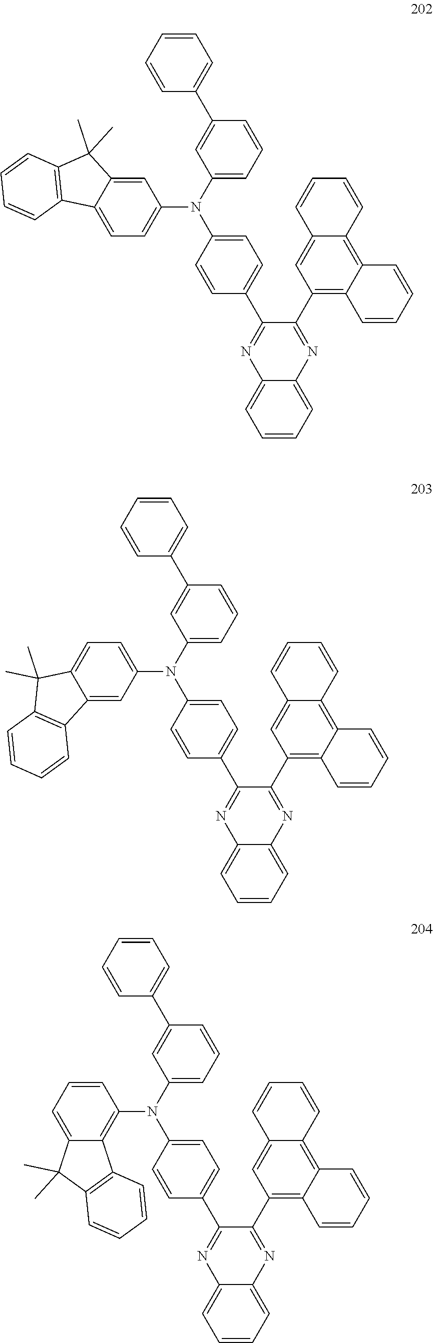 [SMILES: CC1(C)c2ccccc2-c2ccc(N(c3ccc(-c4nc5ccccc5nc4-c4cc5ccccc5c5ccccc45)cc3)c3cccc(-c4ccccc4)c3)cc21 ; CC1(C)c2ccccc2-c2cc(N(c3ccc(-c4nc5ccccc5nc4-c4cc5ccccc5c5ccccc45)cc3)c3cccc(-c4ccccc4)c3)ccc21 ; CC1(C)c2ccccc2-c2c(N(c3ccc(-c4nc5ccccc5nc4-c4cc5ccccc5c5ccccc45)cc3)c3cccc(-c4ccccc4)c3)cccc21]

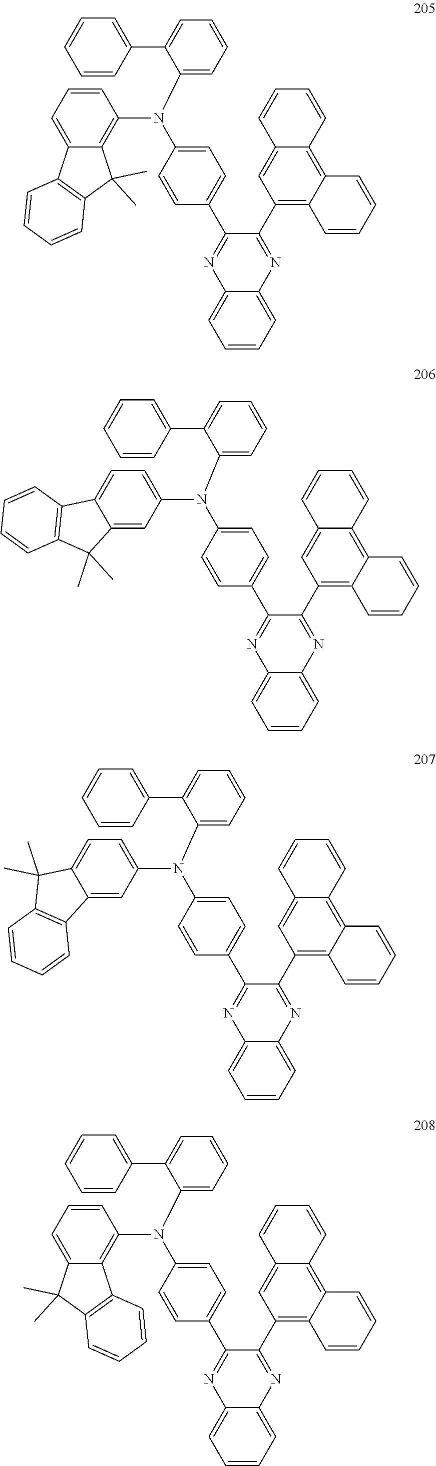 [SMILES: CC1(C)c2ccccc2-c2cccc(N(c3ccc(-c4nc5ccccc5nc4-c4cc5ccccc5c5ccccc45)cc3)c3ccccc3-c3ccccc3)c21 ; CC1(C)c2ccccc2-c2ccc(N(c3ccc(-c4nc5ccccc5nc4-c4cc5ccccc5c5ccccc45)cc3)c3ccccc3-c3ccccc3)cc21 ; CC1(C)c2ccccc2-c2cc(N(c3ccc(-c4nc5ccccc5nc4-c4cc5ccccc5c5ccccc45)cc3)c3ccccc3-c3ccccc3)ccc21 ; CC1(C)c2ccccc2-c2c(N(c3ccc(-c4nc5ccccc5nc4-c4cc5ccccc5c5ccccc45)cc3)c3ccccc3-c3ccccc3)cccc21]

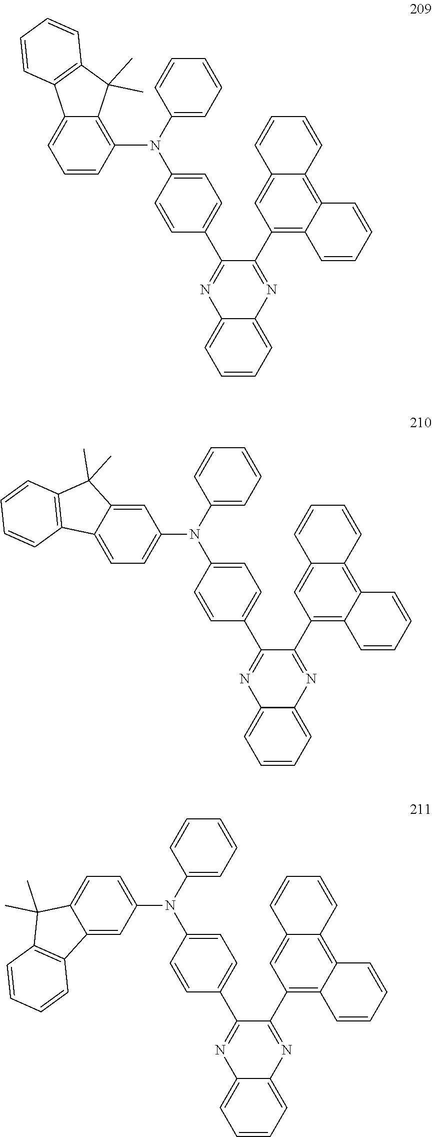 [SMILES: CC1(C)c2ccccc2-c2cccc(N(c3ccccc3)c3ccc(-c4nc5ccccc5nc4-c4cc5ccccc5c5ccccc45)cc3)c21 ; CC1(C)c2ccccc2-c2ccc(N(c3ccccc3)c3ccc(-c4nc5ccccc5nc4-c4cc5ccccc5c5ccccc45)cc3)cc21 ; CC1(C)c2ccccc2-c2cc(N(c3ccccc3)c3ccc(-c4nc5ccccc5nc4-c4cc5ccccc5c5ccccc45)cc3)ccc21]

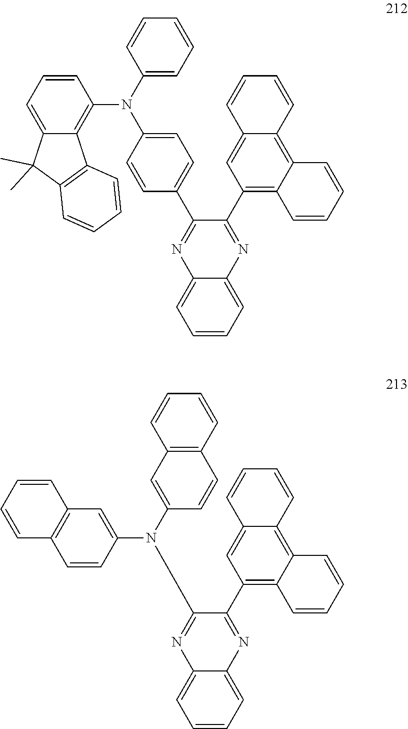 [SMILES: CC1(C)c2ccccc2-c2c(N(c3ccccc3)c3ccc(-c4nc5ccccc5nc4-c4cc5ccccc5c5ccccc45)cc3)cccc21 ; c1ccc2cc(N(c3ccc4ccccc4c3)c3nc4ccccc4nc3-c3cc4ccccc4c4ccccc34)ccc2c1]

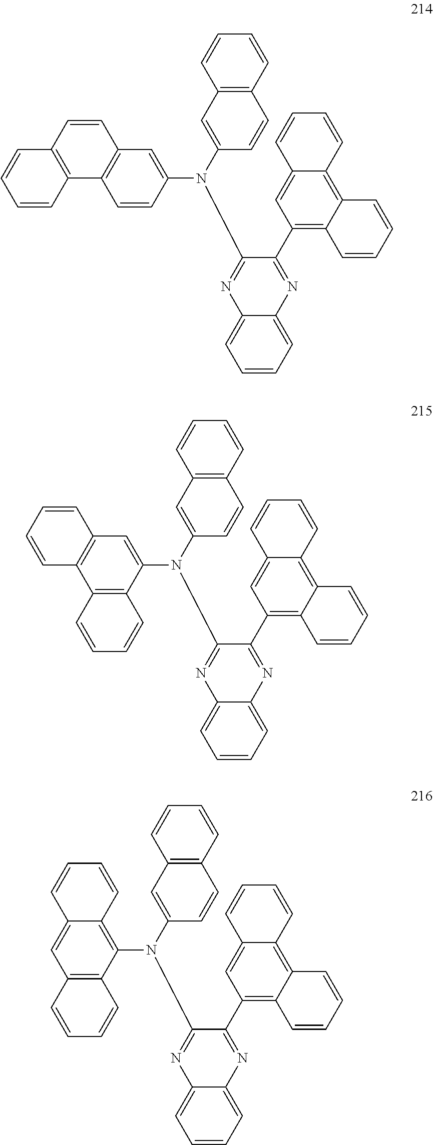 [SMILES: c1ccc2cc(N(c3ccc4c(ccc5ccccc54)c3)c3nc4ccccc4nc3-c3cc4ccccc4c4ccccc34)ccc2c1 ; c1ccc2cc(N(c3nc4ccccc4nc3-c3cc4ccccc4c4ccccc34)c3cc4ccccc4c4ccccc34)ccc2c1 ; c1ccc2cc(N(c3nc4ccccc4nc3-c3cc4ccccc4c4ccccc34)c3c4ccccc4cc4ccccc34)ccc2c1]

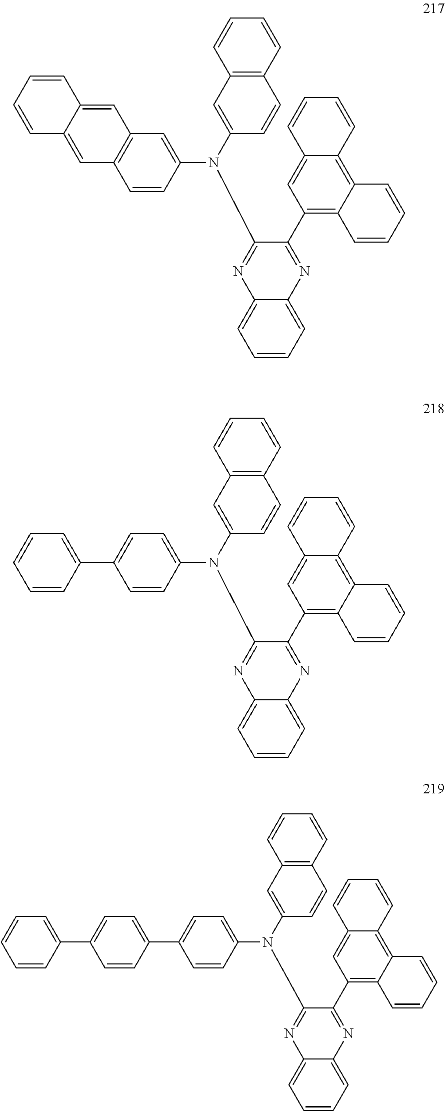 [SMILES: c1ccc2cc(N(c3ccc4cc5ccccc5cc4c3)c3nc4ccccc4nc3-c3cc4ccccc4c4ccccc34)ccc2c1 ; c1ccc(-c2ccc(N(c3ccc4ccccc4c3)c3nc4ccccc4nc3-c3cc4ccccc4c4ccccc34)cc2)cc1 ; c1ccc(-c2ccc(-c3ccc(N(c4ccc5ccccc5c4)c4nc5ccccc5nc4-c4cc5ccccc5c5ccccc45)cc3)cc2)cc1]

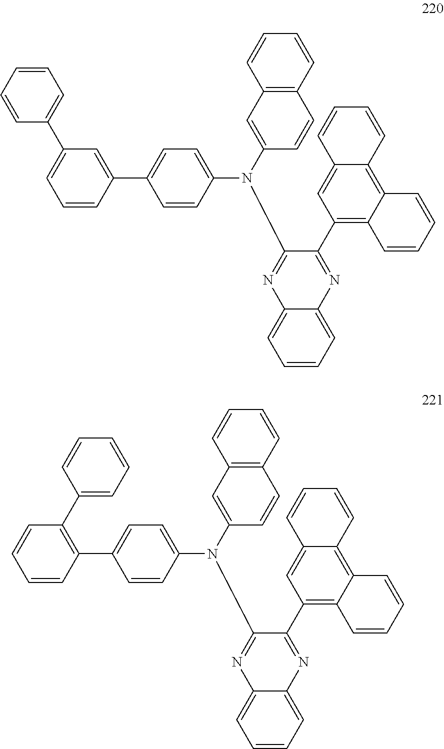 [SMILES: c1ccc(-c2cccc(-c3ccc(N(c4ccc5ccccc5c4)c4nc5ccccc5nc4-c4cc5ccccc5c5ccccc45)cc3)c2)cc1 ; c1ccc(-c2ccccc2-c2ccc(N(c3ccc4ccccc4c3)c3nc4ccccc4nc3-c3cc4ccccc4c4ccccc34)cc2)cc1]

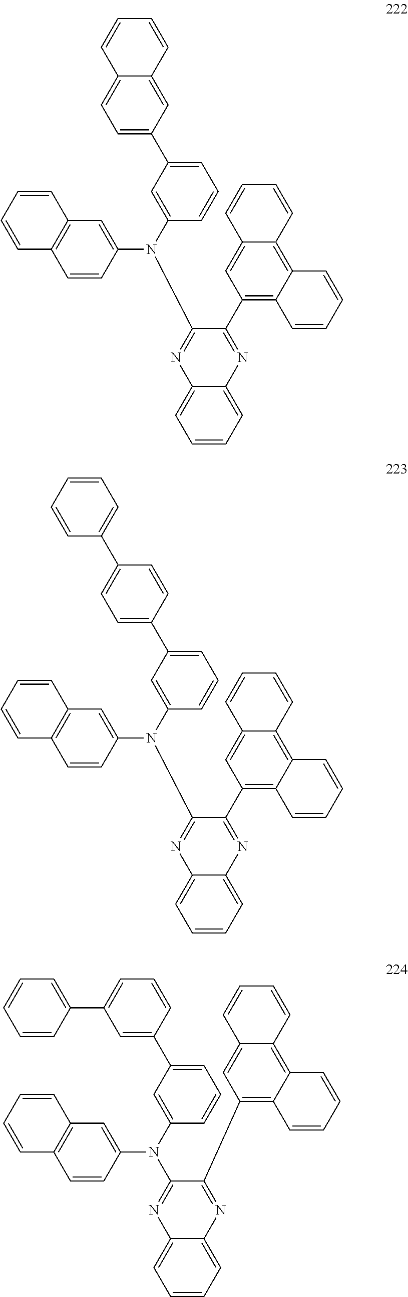 [SMILES: c1cc(-c2ccc3ccccc3c2)cc(N(c2ccc3ccccc3c2)c2nc3ccccc3nc2-c2cc3ccccc3c3ccccc23)c1 ; c1ccc(-c2ccc(-c3cccc(N(c4ccc5ccccc5c4)c4nc5ccccc5nc4-c4cc5ccccc5c5ccccc45)c3)cc2)cc1 ; c1ccc(-c2cccc(-c3cccc(N(c4ccc5ccccc5c4)c4nc5ccccc5nc4-c4cc5ccccc5c5ccccc45)c3)c2)cc1]

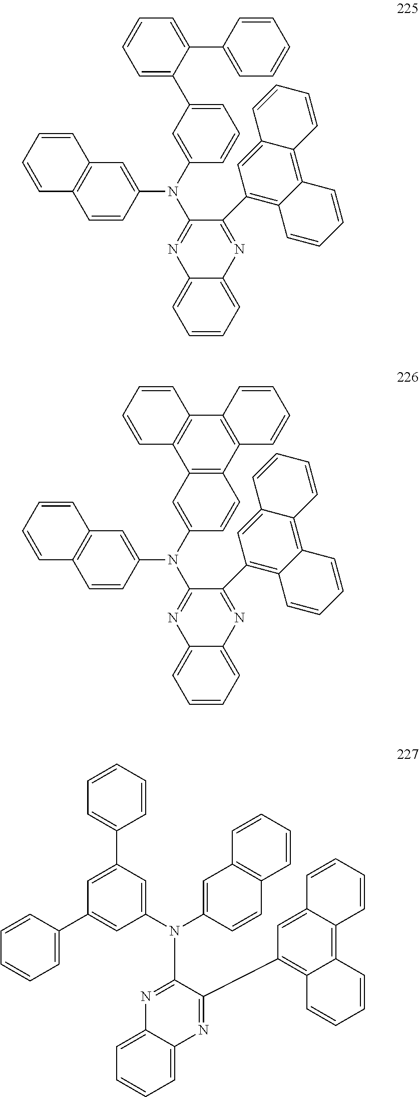 [SMILES: c1ccc(-c2ccccc2-c2cccc(N(c3ccc4ccccc4c3)c3nc4ccccc4nc3-c3cc4ccccc4c4ccccc34)c2)cc1 ; c1ccc2cc(N(c3ccc4c5ccccc5c5ccccc5c4c3)c3nc4ccccc4nc3-c3cc4ccccc4c4ccccc34)ccc2c1 ; c1ccc(-c2cc(-c3ccccc3)cc(N(c3ccc4ccccc4c3)c3nc4ccccc4nc3-c3cc4ccccc4c4ccccc34)c2)cc1]

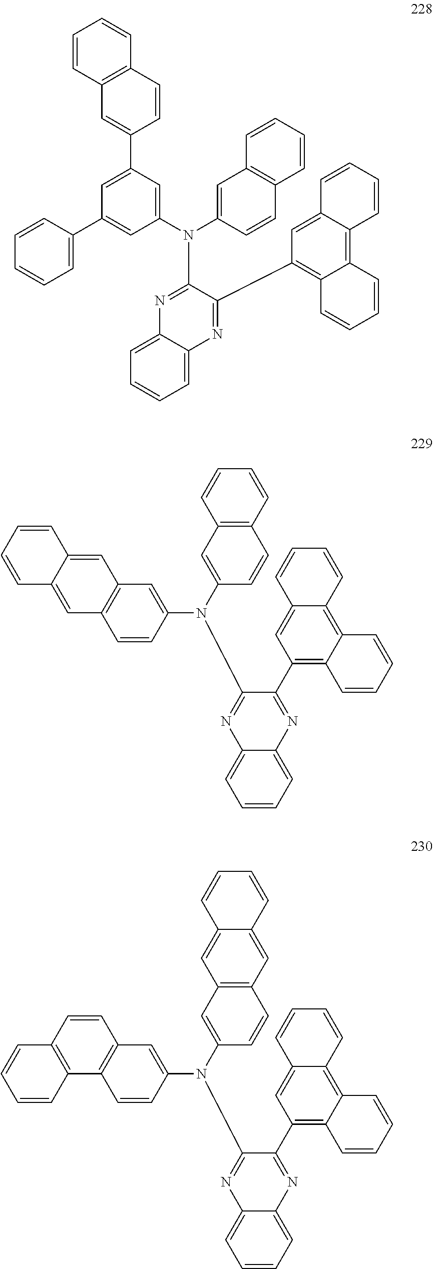 [SMILES: c1ccc(-c2cc(-c3ccc4ccccc4c3)cc(N(c3ccc4ccccc4c3)c3nc4ccccc4nc3-c3cc4ccccc4c4ccccc34)c2)cc1 ; c1ccc2cc(N(c3ccc4cc5ccccc5cc4c3)c3nc4ccccc4nc3-c3cc4ccccc4c4ccccc34)ccc2c1 ; c1ccc2cc3cc(N(c4ccc5c(ccc6ccccc65)c4)c4nc5ccccc5nc4-c4cc5ccccc5c5ccccc45)ccc3cc2c1]

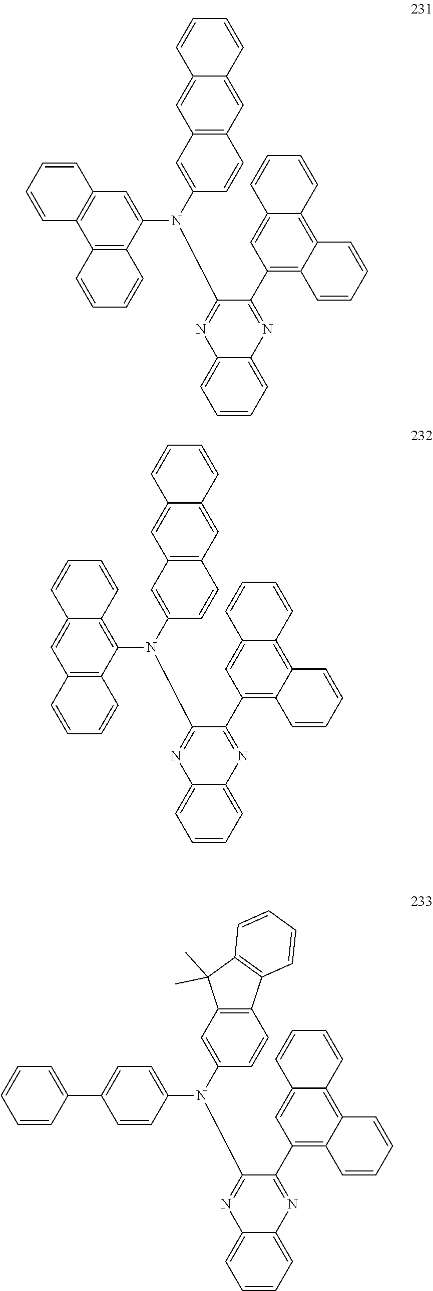

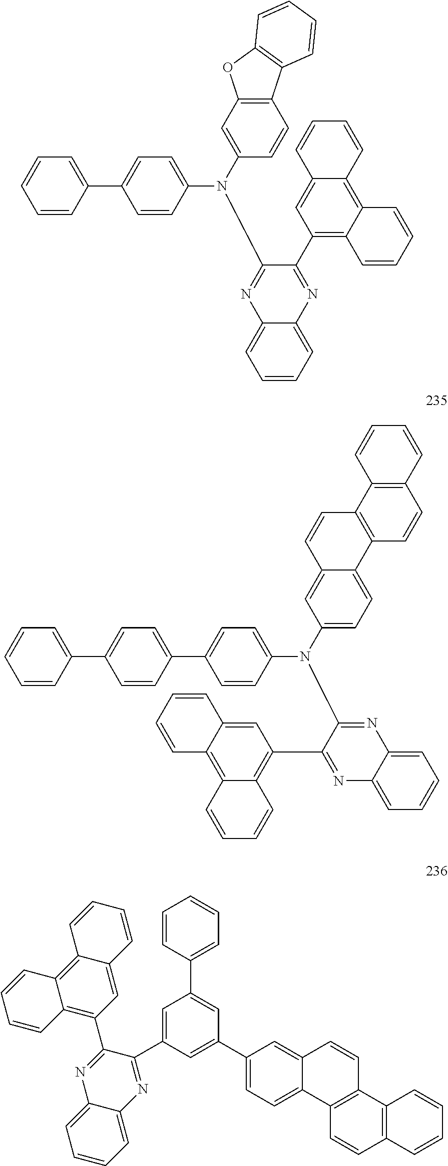 [SMILES: c1ccc(-c2ccc(N(c3ccc4c(c3)oc3ccccc34)c3nc4ccccc4nc3-c3cc4ccccc4c4ccccc34)cc2)cc1 ; c1ccc(-c2ccc(-c3ccc(N(c4ccc5c(ccc6c7ccccc7ccc56)c4)c4nc5ccccc5nc4-c4cc5ccccc5c5ccccc45)cc3)cc2)cc1 ; c1ccc(-c2cc(-c3ccc4c(ccc5c6ccccc6ccc45)c3)cc(-c3nc4ccccc4nc3-c3cc4ccccc4c4ccccc34)c2)cc1]

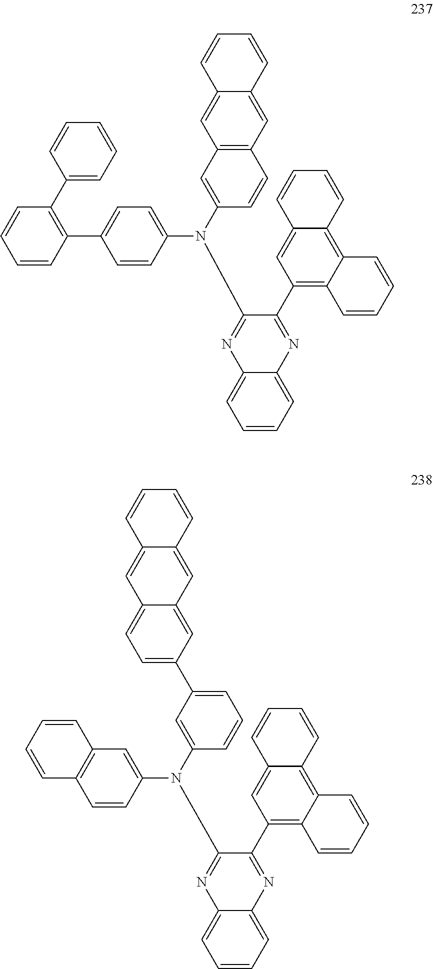 [SMILES: c1ccc(-c2ccccc2-c2ccc(N(c3ccc4cc5ccccc5cc4c3)c3nc4ccccc4nc3-c3cc4ccccc4c4ccccc34)cc2)cc1 ; c1cc(-c2ccc3cc4ccccc4cc3c2)cc(N(c2ccc3ccccc3c2)c2nc3ccccc3nc2-c2cc3ccccc3c3ccccc23)c1]

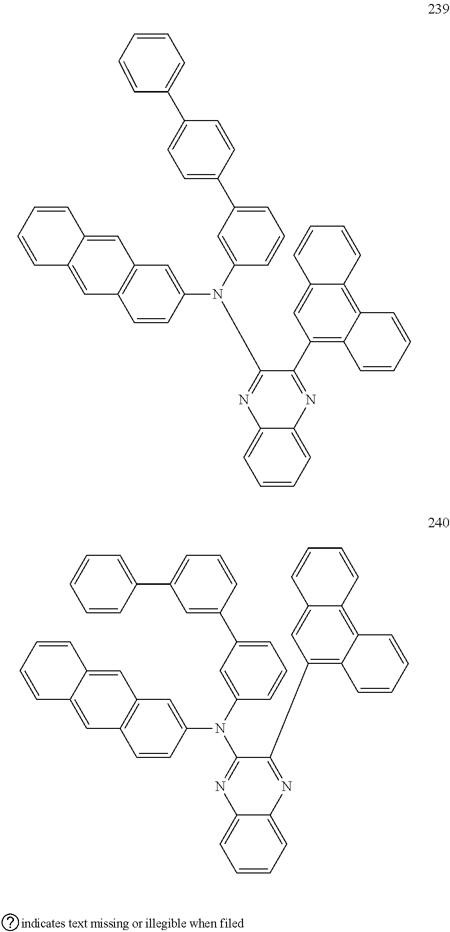 [SMILES: c1ccc(-c2ccc(-c3cccc(N(c4ccc5cc6ccccc6cc5c4)c4nc5ccccc5nc4-c4cc5ccccc5c5ccccc45)c3)cc2)cc1 ; c1ccc(-c2cccc(-c3cccc(N(c4ccc5cc6ccccc6cc5c4)c4nc5ccccc5nc4-c4cc5ccccc5c5ccccc45)c3)c2)cc1]

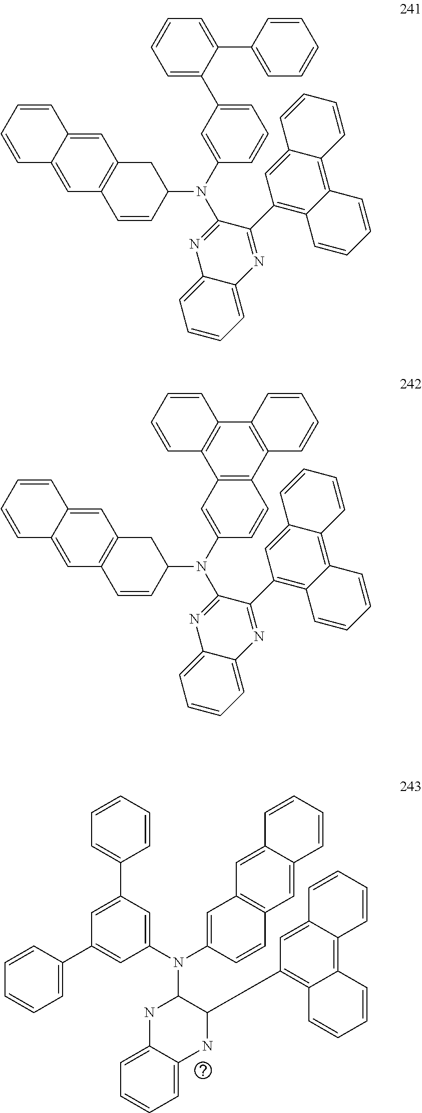 [SMILES: C1=CC(N(c2cccc(-c3ccccc3-c3ccccc3)c2)c2nc3ccccc3nc2-c2cc3ccccc3c3ccccc23)Cc2cc3ccccc3cc21 ; C1=CC(N(c2ccc3c4ccccc4c4ccccc4c3c2)c2nc3ccccc3nc2-c2cc3ccccc3c3ccccc23)Cc2cc3ccccc3cc21 ; c1ccc(-c2cc(-c3ccccc3)cc(N(c3ccc4cc5ccccc5cc4c3)C3Nc4ccccc4NC3c3cc4ccccc4c4ccccc34)c2)cc1]

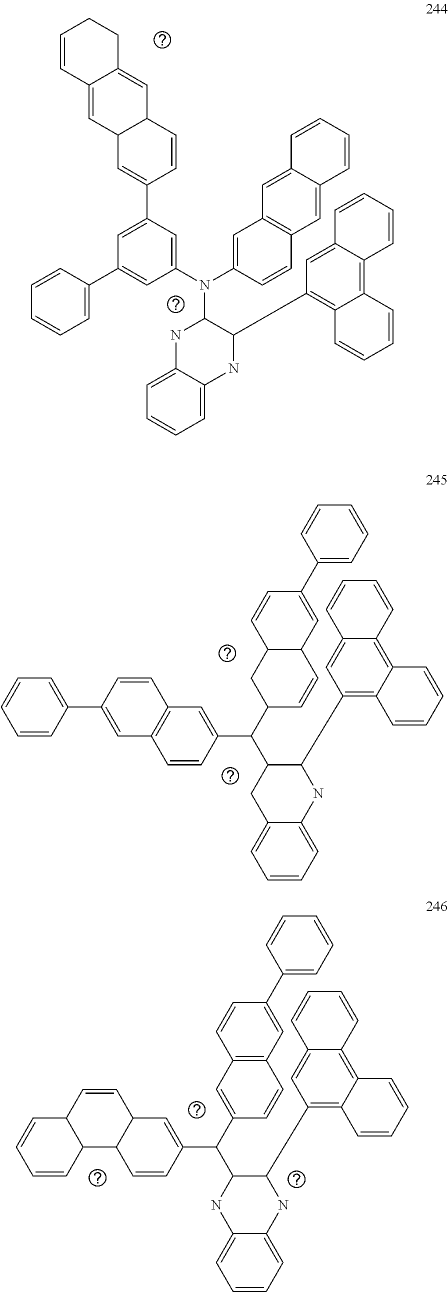

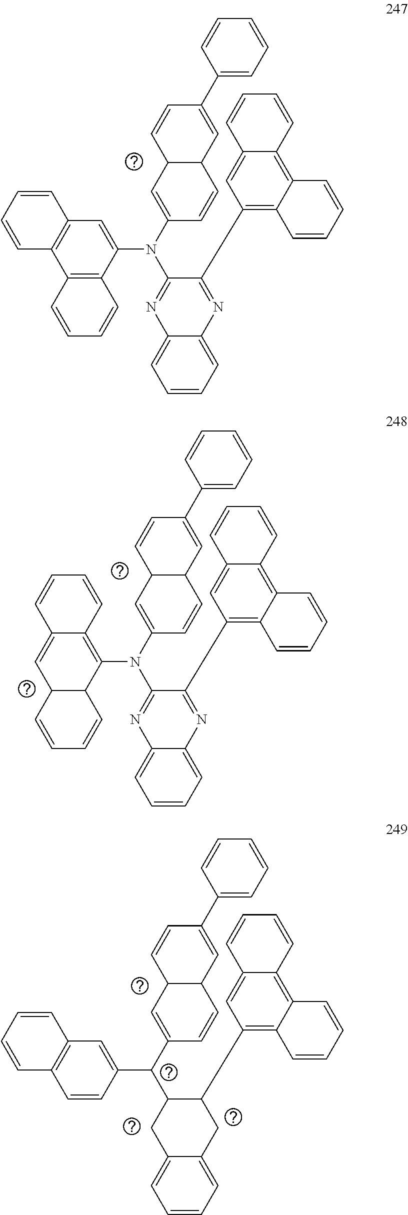

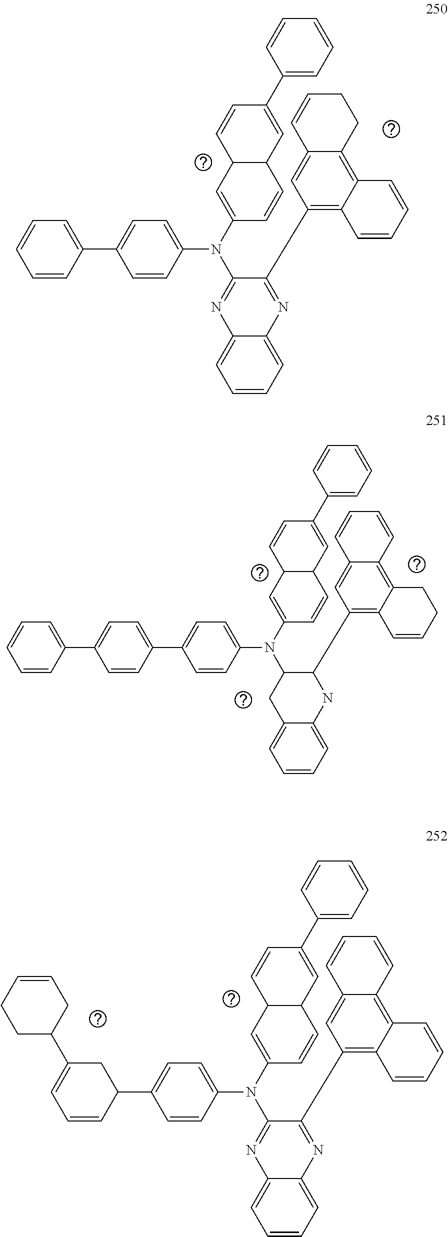 [SMILES: C1=Cc2cc(-c3nc4ccccc4nc3N(C3=CC4C=CC(c5ccccc5)=CC4C=C3)c3ccc(-c4ccccc4)cc3)c3ccccc3c2CC1 ; C1=Cc2c(C3Nc4ccccc4CC3N(C3=CC4C=CC(c5ccccc5)=CC4C=C3)c3ccc(-c4ccc(-c5ccccc5)cc4)cc3)cc3ccccc3c2CC1 ; C1=CC(c2ccc(N(C3=CC4C=CC(c5ccccc5)=CC4C=C3)c3nc4ccccc4nc3-c3cc4ccccc4c4ccccc34)cc2)CC(C2CC=CCC2)=C1]

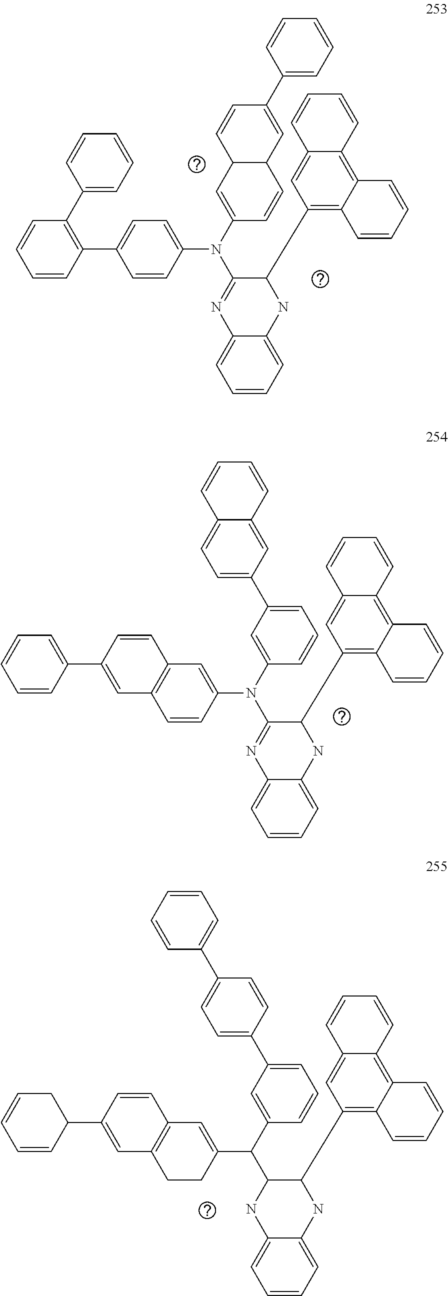 [SMILES: C1=CC2C=C(N(C3=Nc4ccccc4NC3c3cc4ccccc4c4ccccc34)c3ccc(-c4ccccc4-c4ccccc4)cc3)C=CC2C=C1c1ccccc1 ; c1ccc(-c2ccc3cc(N(C4=Nc5ccccc5NC4c4cc5ccccc5c5ccccc45)c4cccc(-c5ccc6ccccc6c5)c4)ccc3c2)cc1 ; C1=CCC(c2ccc3c(c2)CCC(C(c2cccc(-c4ccc(-c5ccccc5)cc4)c2)C2Nc4ccccc4NC2c2cc4ccccc4c4ccccc24)=C3)C=C1]

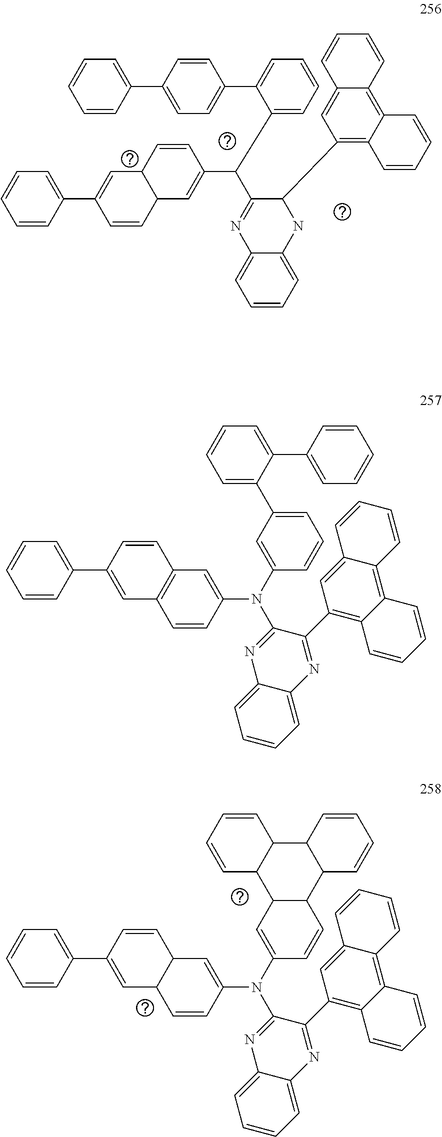 [SMILES: C1=CC2C=C(C(C3=Nc4ccccc4NC3c3cc4ccccc4c4ccccc34)c3ccccc3-c3ccc(-c4ccccc4)cc3)C=CC2C=C1c1ccccc1 ; c1ccc(-c2ccc3cc(N(c4cccc(-c5ccccc5-c5ccccc5)c4)c4nc5ccccc5nc4-c4cc5ccccc5c5ccccc45)ccc3c2)cc1 ; C1=CC2C3C=CC=CC3C3C=C(N(C4=CC5C=CC(c6ccccc6)=CC5C=C4)c4nc5ccccc5nc4-c4cc5ccccc5c5ccccc45)C=CC3C2C=C1]

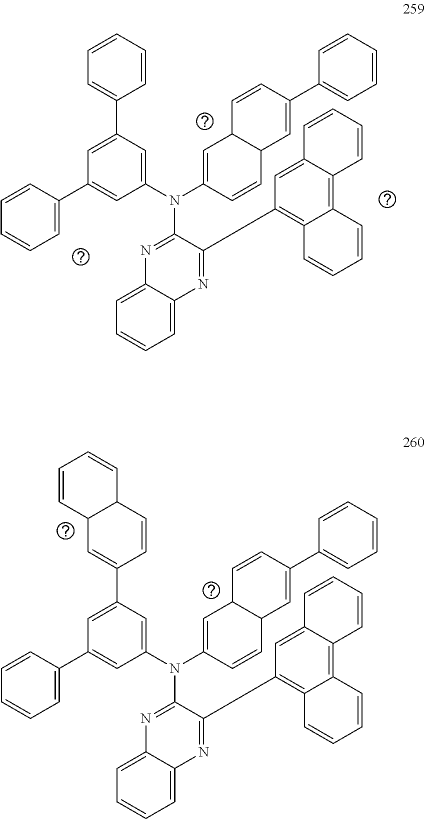 [SMILES: C1=CC2C=C(N(c3cc(-c4ccccc4)cc(-c4ccccc4)c3)c3nc4ccccc4nc3-c3cc4ccccc4c4ccccc34)C=CC2C=C1c1ccccc1 ; C1=CC2C=CC(c3cc(-c4ccccc4)cc(N(C4=CC5C=CC(c6ccccc6)=CC5C=C4)c4nc5ccccc5nc4-c4cc5ccccc5c5ccccc45)c3)=CC2C=C1]

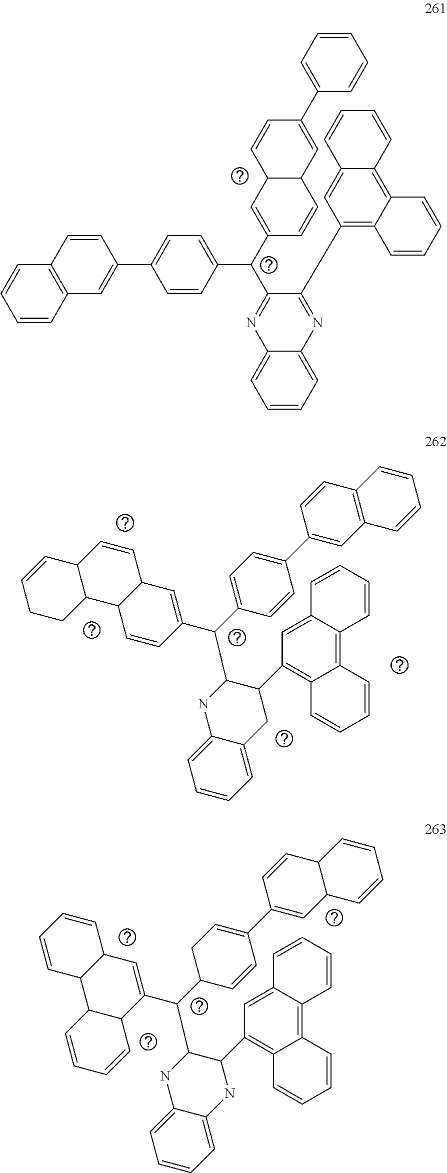 [SMILES: C1=CC2C=C(C(c3ccc(-c4ccc5ccccc5c4)cc3)c3nc4ccccc4nc3-c3cc4ccccc4c4ccccc34)C=CC2C=C1c1ccccc1 ; C1=CC2C=CC3C=C(C(c4ccc(-c5ccc6ccccc6c5)cc4)C4Nc5ccccc5CC4c4cc5ccccc5c5ccccc45)C=CC3C2CC1 ; C1=CC2C=CC(C3=CCC(C(C4=CC5C=CC=CC5C5C=CC=CC45)C4Nc5ccccc5NC4c4cc5ccccc5c5ccccc45)C=C3)=CC2C=C1]

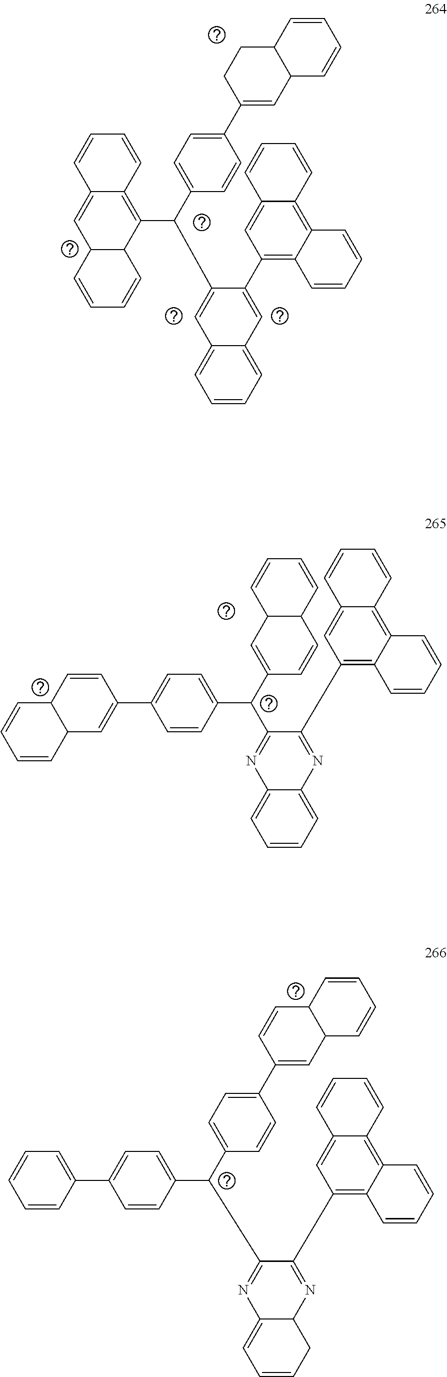

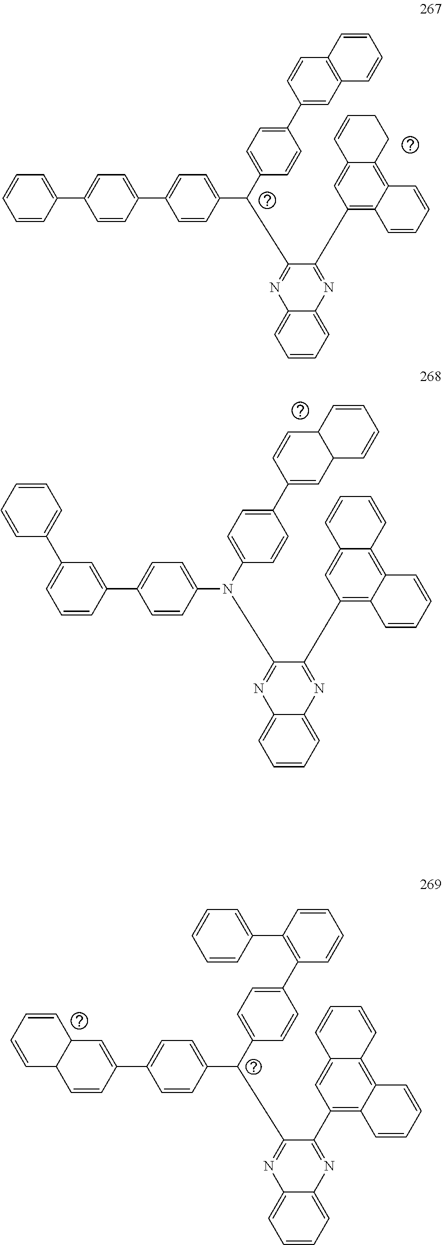 [SMILES: C1=Cc2cc(-c3nc4ccccc4nc3C(c3ccc(-c4ccc(-c5ccccc5)cc4)cc3)c3ccc(-c4ccc5ccccc5c4)cc3)c3ccccc3c2CC1 ; C1=CC2C=CC(c3ccc(N(c4ccc(-c5cccc(-c6ccccc6)c5)cc4)c4nc5ccccc5nc4-c4cc5ccccc5c5ccccc45)cc3)=CC2C=C1 ; C1=CC2C=CC(c3ccc(C(c4ccc(-c5ccccc5-c5ccccc5)cc4)c4nc5ccccc5nc4-c4cc5ccccc5c5ccccc45)cc3)=CC2C=C1]

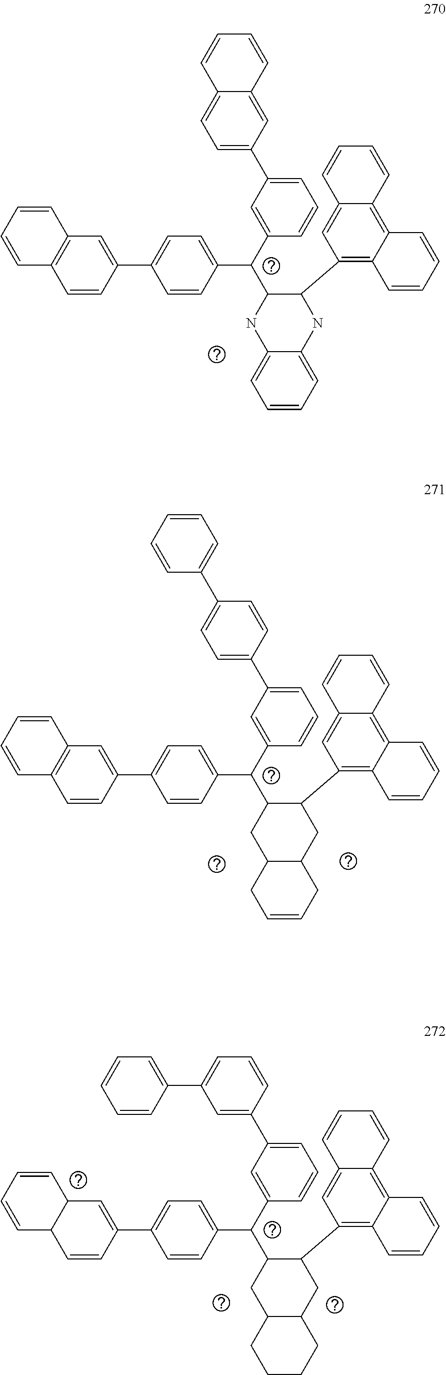

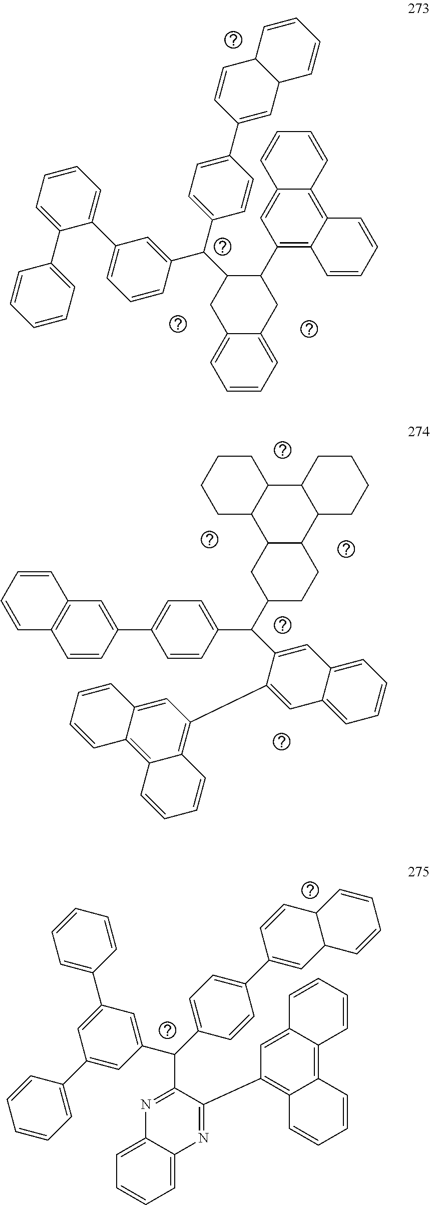

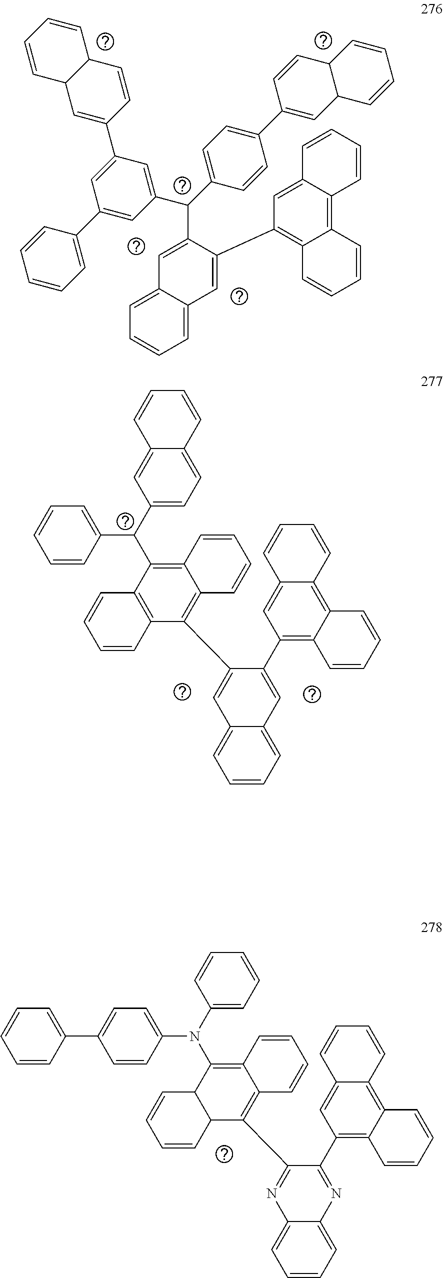

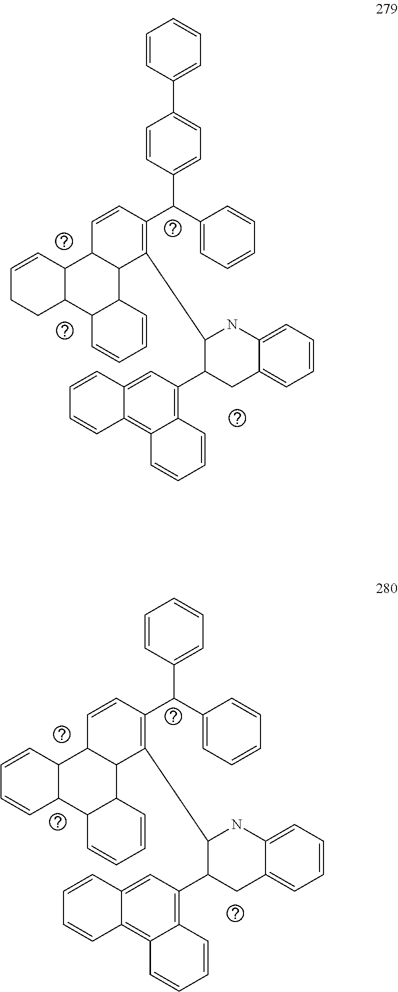 [SMILES: C1=CC2C(C=C1)C1C(C3Nc4ccccc4CC3c3cc4ccccc4c4ccccc34)=C(C(c3ccccc3)c3ccc(-c4ccccc4)cc3)C=CC1C1C=CCCC21 ; C1=CC2C3C=CC=CC3C3C(C4Nc5ccccc5CC4c4cc5ccccc5c5ccccc45)=C(C(c4ccccc4)c4ccccc4)C=CC3C2C=C1]

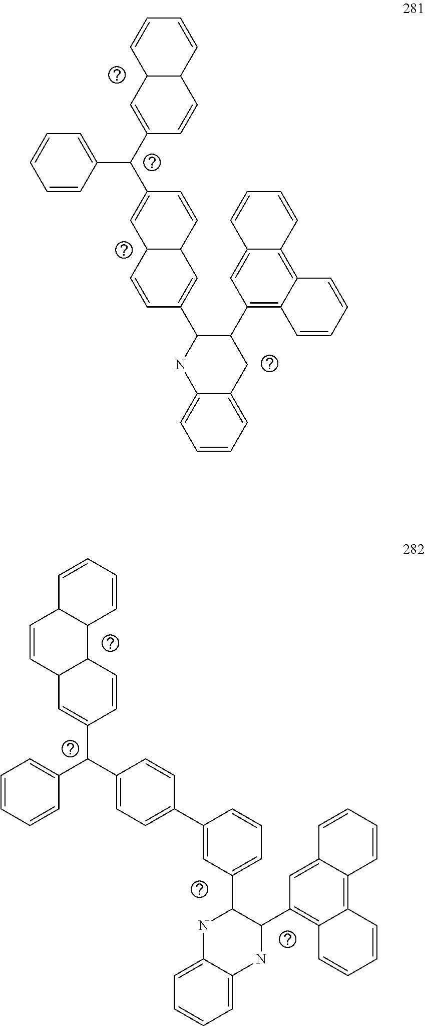 [SMILES: C1=CC2C=CC(C(C3=CC4C=CC(C5Nc6ccccc6CC5c5cc6ccccc6c6ccccc56)=CC4C=C3)c3ccccc3)=CC2C=C1 ; C1=CC2C=CC3C=C(C(c4ccccc4)c4ccc(-c5cccc(C6Nc7ccccc7NC6c6cc7ccccc7c7ccccc67)c5)cc4)C=CC3C2C=C1]

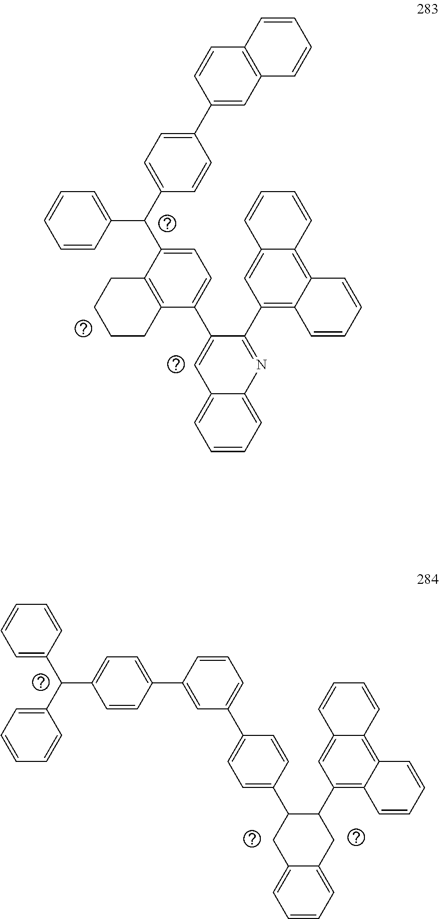

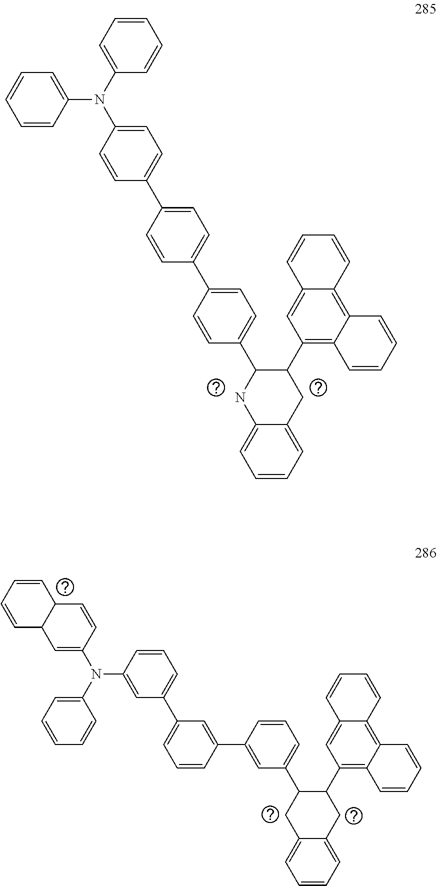 [SMILES: c1ccc(N(c2ccccc2)c2ccc(-c3ccc(-c4ccc(C5Nc6ccccc6CC5c5cc6ccccc6c6ccccc56)cc4)cc3)cc2)cc1 ; C1=CC2C=CC(N(c3ccccc3)c3cccc(-c4cccc(-c5cccc(C6Cc7ccccc7CC6c6cc7ccccc7c7ccccc67)c5)c4)c3)=CC2C=C1]

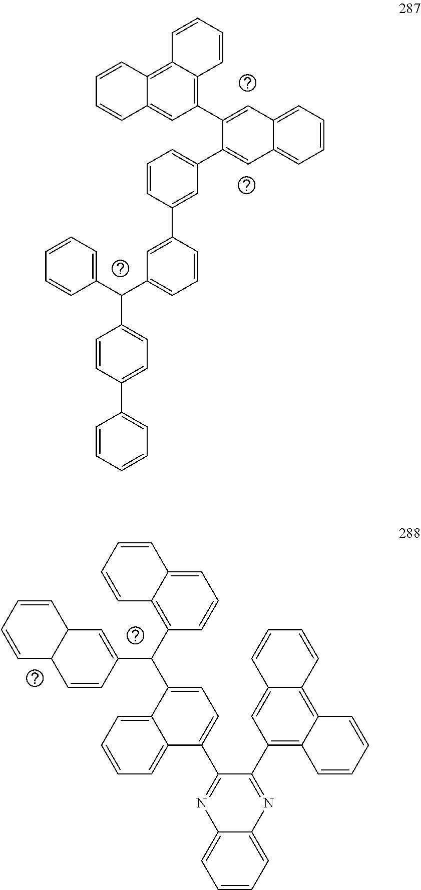

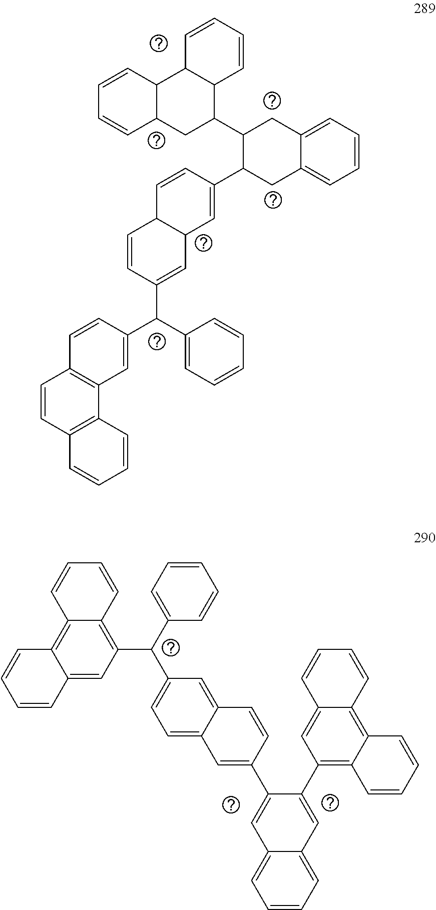

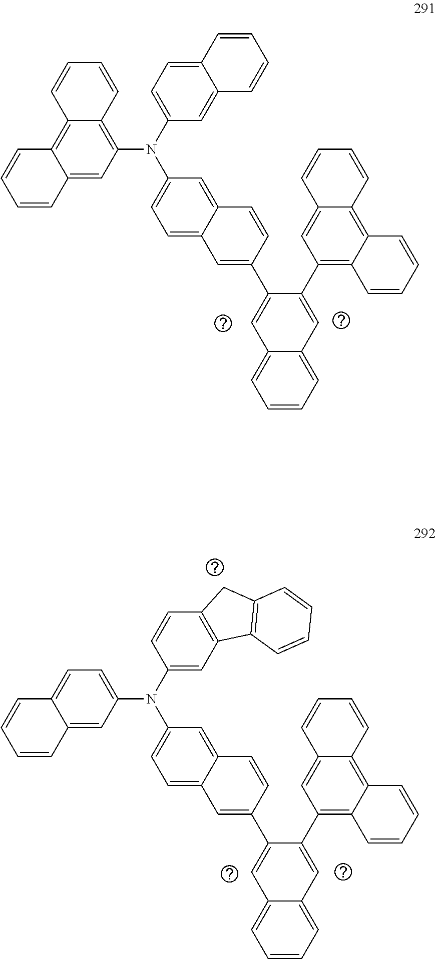 [SMILES: c1ccc2cc(N(c3ccc4cc(-c5cc6ccccc6cc5-c5cc6ccccc6c6ccccc56)ccc4c3)c3cc4ccccc4c4ccccc34)ccc2c1 ; c1ccc2c(c1)Cc1ccc(N(c3ccc4ccccc4c3)c3ccc4cc(-c5cc6ccccc6cc5-c5cc6ccccc6c6ccccc56)ccc4c3)cc1-2]

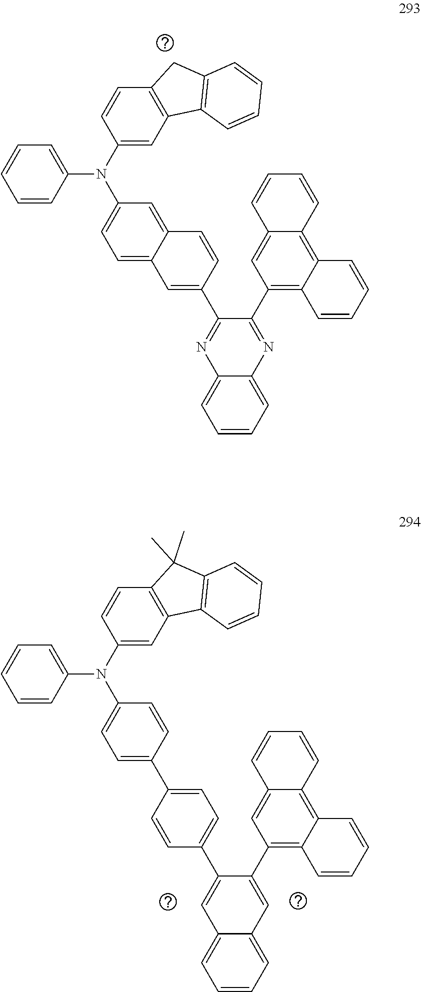 [SMILES: c1ccc(N(c2ccc3c(c2)-c2ccccc2C3)c2ccc3cc(-c4nc5ccccc5nc4-c4cc5ccccc5c5ccccc45)ccc3c2)cc1 ; CC1(C)c2ccccc2-c2cc(N(c3ccccc3)c3ccc(-c4ccc(-c5cc6ccccc6cc5-c5cc6ccccc6c6ccccc56)cc4)cc3)ccc21]

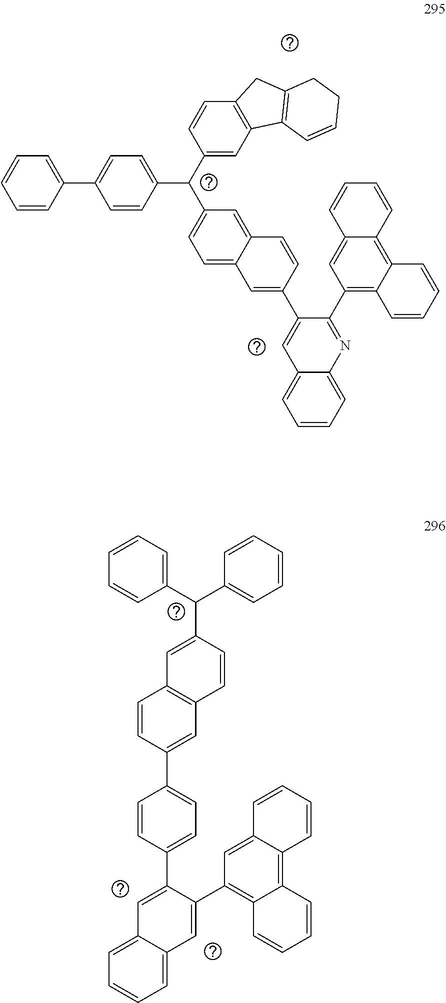

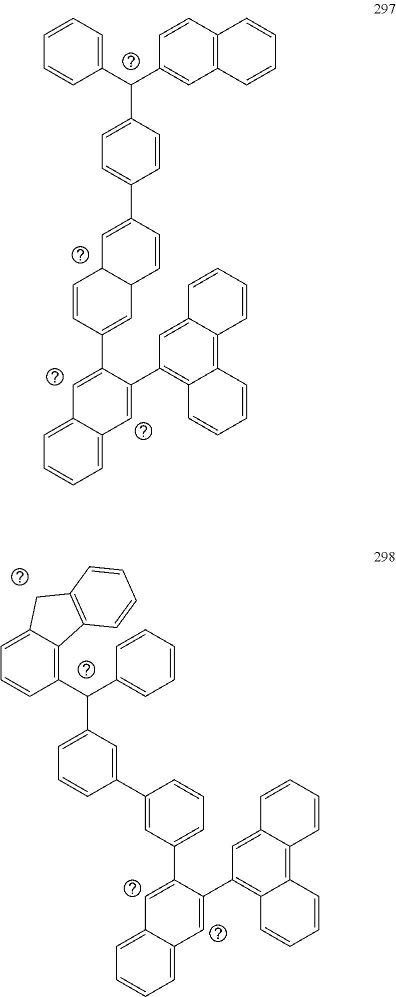

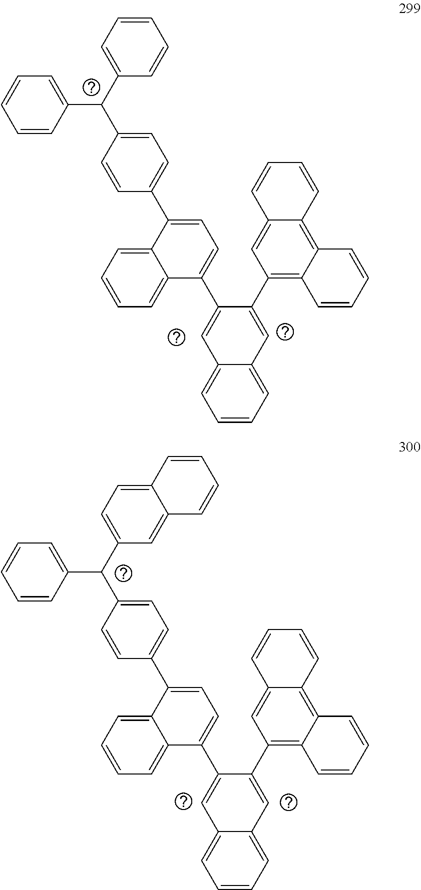

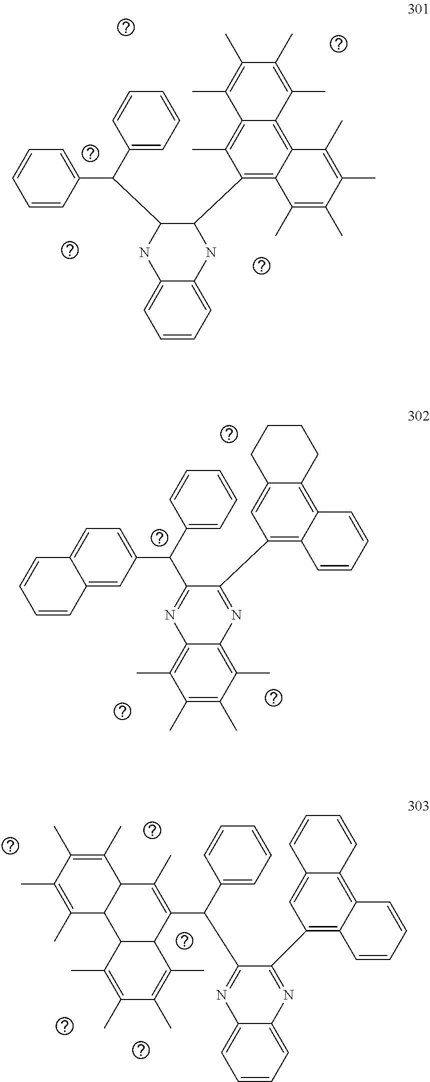

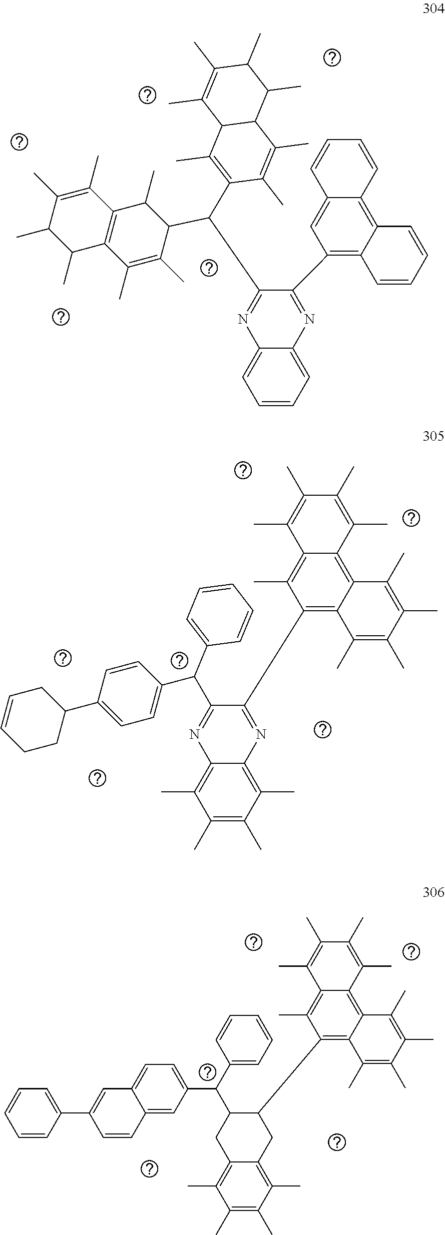

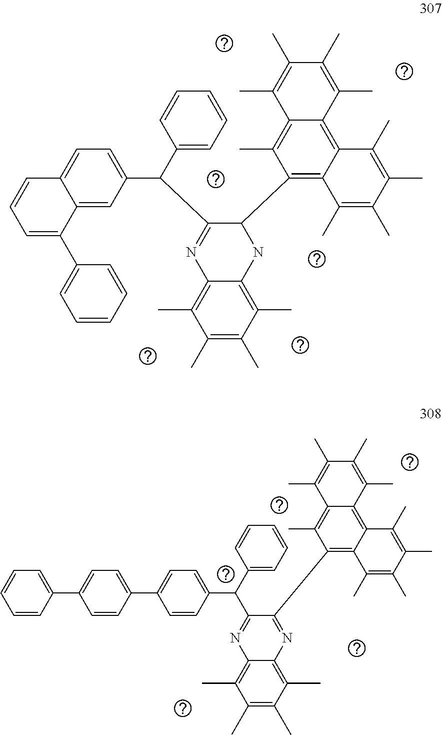 [SMILES: Cc1c(C)c(C)c2c(c1C)N=C(C(c1ccccc1)c1ccc3cccc(-c4ccccc4)c3c1)C(c1c(C)c3c(C)c(C)c(C)c(C)c3c3c(C)c(C)c(C)c(C)c13)N2 ; Cc1c(C)c(C)c2nc(C(c3ccccc3)c3ccc(-c4ccc(-c5ccccc5)cc4)cc3)c(-c3c(C)c4c(C)c(C)c(C)c(C)c4c4c(C)c(C)c(C)c(C)c34)nc2c1C]

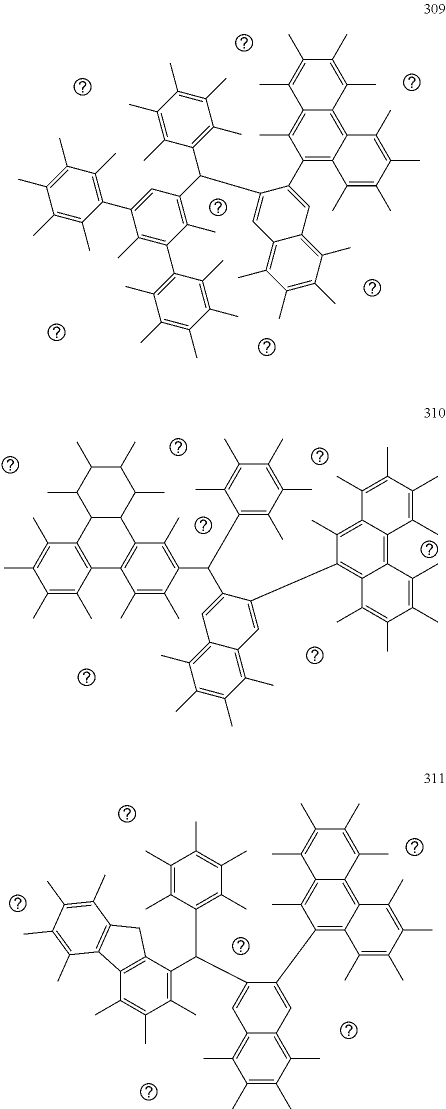

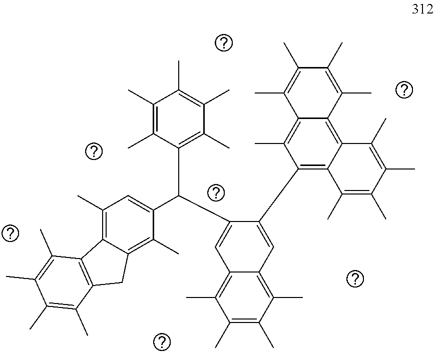

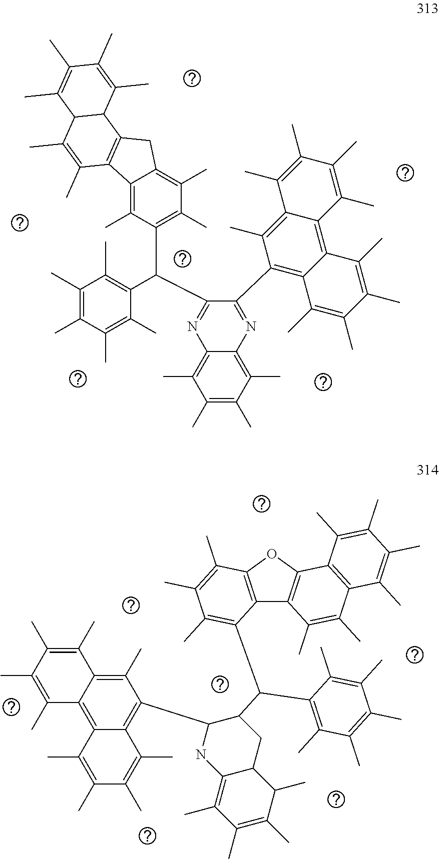

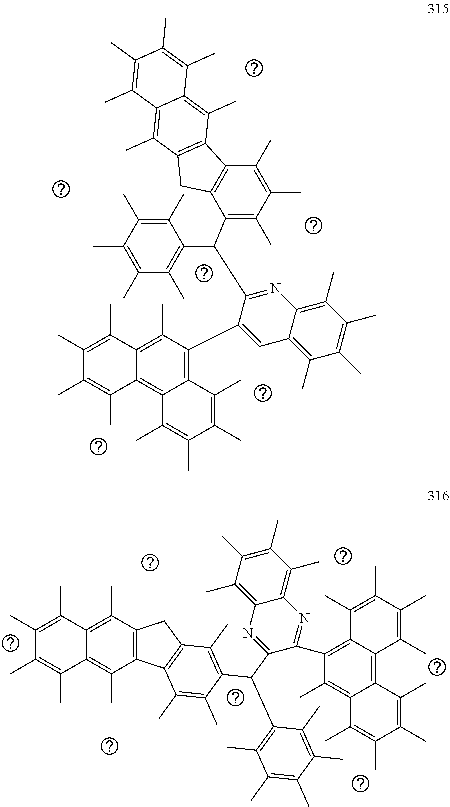

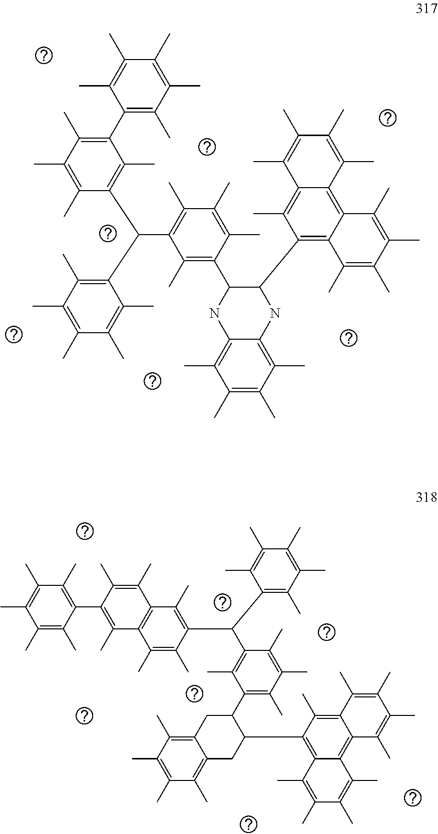

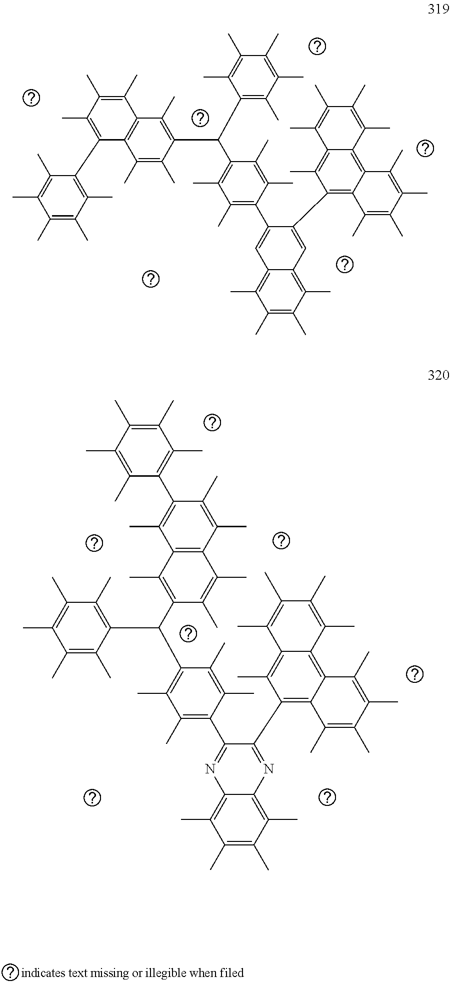

In addition, by introducing various substituents to the structure of Chemical Formula 1, compounds having unique properties of the introduced substituents may be synthesized. For example, by introducing substituents normally used for a hole injection layer material, a hole transport layer material, a light emitting layer material, an electron transport layer material and a charge generation layer material used when manufacturing an organic light-emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials may be enhanced, and material applications may become diverse.

Meanwhile, the heterocyclic compound has a high glass transition temperature (Tg), thereby having excellent thermal stability. Such an increase in the thermal stability becomes an important factor providing driving stability to a device.

The heterocyclic compound according to one embodiment of the present invention may be prepared using a multi-step chemical reaction. Some intermediate compounds are prepared first, and from the intermediate compounds, the heterocyclic compound represented by Chemical Formula 1 may be prepared. More specifically, the heterocyclic compound according to one embodiment of the present invention may be prepared based on preparation examples to be described later.

Another embodiment of the present invention provides an organic light-emitting device including the heterocyclic compound represented by Chemical Formula 1. The “organic light-emitting device” may be expressed in terms such as an “organic light emitting diode”, an “OLED”, an “OLED device” and an “organic electroluminescent device”.

In addition, the present invention provides to an organic light-emitting device comprising:
a first electrode;
a second electrode provided opposite to the first electrode; and
one or more organic material layers provided between the first electrode and the second electrode,
wherein at least one or more organic material layers comprise the heterocyclic compound represented by Chemical Formula 1.

In one embodiment of the present invention, the first electrode may be a positive electrode, and the second electrode may be a negative electrode.

In another embodiment, the first electrode may be a negative electrode, and the second electrode may be a positive electrode.

In one embodiment of the present invention, the organic light-emitting device may be a red organic light-emitting device, and the heterocyclic compound represented by Chemical Formula 1 may be used as a material of the red organic light-emitting device.

In another embodiment of the present invention, the organic light-emitting device may be a blue organic light-emitting device, and the heterocyclic compound represented by Chemical Formula 1 may be used as a material of the blue organic light-emitting device.

In another embodiment of the present invention, the organic light-emitting device may be a green organic light-emitting device, and the heterocyclic compound represented by Chemical Formula 1 may be used as a material of the green organic light-emitting device.

In one embodiment of the present invention, the organic light-emitting device may be a red organic light-emitting device, and the heterocyclic compound represented by Chemical Formula 1 may be used as a light emitting layer material of the red organic light-emitting device.

In another embodiment of the present invention, the organic light-emitting device may be a blue organic light-emitting device, and the heterocyclic compound represented by Chemical Formula 1 may be used as a light emitting layer material of the blue organic light-emitting device.

In another embodiment of the present invention, the organic light-emitting device may be a green organic light-emitting device, and the heterocyclic compound represented by Chemical Formula 1 may be used as a light emitting layer material of the green organic light-emitting device.

Specific descriptions on the heterocyclic compound represented by Chemical Formula 1 are the same as the descriptions provided above.

The organic light-emitting device of the present invention may be manufactured using common organic light-emitting device manufacturing methods and materials except that one or more organic material layers are formed using the heterocyclic compound described above.

The heterocyclic compound may be formed into the organic material layer using a solution coating method as well as a vacuum deposition method when the organic light-emitting device is manufactured. Herein, the solution coating method means spin coating, dip coating, inkjet printing, screen printing, a spray method, roll coating and the like, but is not limited thereto.

The organic material layer of the organic light-emitting device of the present invention may be formed in a single layer structure, but may also be formed in a multilayer structure in which two or more organic material layers are laminated. For example, the organic light-emitting device of the present invention may have a structure including a hole injection layer, an electron blocking layer, a hole transport layer, a light emitting layer, an electron transport layer, a hole blocking layer, an electron injection layer and the like as the organic material layer. However, the structure of the organic light-emitting device is not limited thereto, and may include a smaller number of organic material layers.

In the organic light-emitting device of the present invention, the organic material layer includes a light emitting layer, and the light emitting layer may include the heterocyclic compound represented by Chemical Formula 1. When the heterocyclic compound is used in the light emitting layer, HOMO (highest occupied molecular orbital) and LUMO (lowest unoccupied molecular orbital) are spatially separated, enabling strong charge transfer, and therefore, driving efficiency and lifetime of the organic light-emitting device may become superior.

In the organic light-emitting device according to one embodiment of the present invention, there is provided an organic light-emitting device in which the organic material layer comprising the heterocyclic compound represented by Chemical Formula 1 further includes a heterocyclic compound represented by the following Chemical Formula 2.

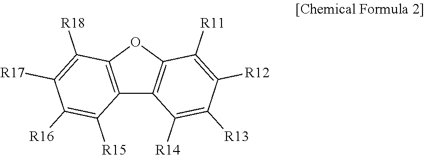

In Chemical Formula 2,
R11 to R18 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; —NR201R202; and the following Chemical Formula 3, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
at least one of R11 to R18 is the following Chemical Formula 3,

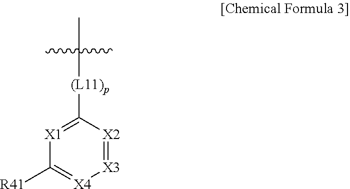

in Chemical Formula 3,
X1 is N; or CRa,
X2 is N; or CRb,
X3 is N; or CRc,
X4 is N; or CRd,
at least two of X1 to X4 are N,
R41 and Ra to Rd are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R301R302; —SiR301R302R303; and —NR301R302, or two or more groups adjacent to each other bond to each other to form a substituted or unsubstituted C6 to C60 aromatic hydrocarbon ring; or a substituted or unsubstituted C2 to C60 heteroring, and R301, R302 and R303 are the same as or different from each other and each independently a substituted or unsubstituted C1 to C60 alkyl group; a substituted or unsubstituted C6 to C60 aryl group; or a substituted or unsubstituted C2 to C60 heteroaryl group,
L11 is a direct bond; a substituted or unsubstituted C6 to C60 arylene group; or a substituted or unsubstituted C2 to C60 heteroarylene group, and
p is an integer of 0 to 5, and when p is 2 or greater, L11s are the same as or different from each other.

In one embodiment of the present invention, R11 to R18 are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R201R202; —SiR201R202R203; —NR201R202; or Chemical Formula 3, or two or more groups adjacent to each other bond to each other to form a substituted or unsubstituted C6 to C30 aromatic hydrocarbon ring; or a substituted or unsubstituted C2 to C30 heteroring, and R201, R202 and R203 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present invention, R11 to R18 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R201R202; —SiR201R202R203; —NR201R202; or Chemical Formula 3, or two or more groups adjacent to each other bond to each other to form a substituted or unsubstituted C6 to C20 aromatic hydrocarbon ring; or a substituted or unsubstituted C2 to C20 heteroring, and R201, R202 and R203 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present invention, R11 to R18 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; or Chemical Formula 3, or two or more groups adjacent to each other may bond to each other to form a substituted or unsubstituted C6 to C20 aromatic hydrocarbon ring; or a substituted or unsubstituted C2 to C20 heteroring.

In another embodiment of the present invention, R11 to R18 are the same as or different from each other, and each independently hydrogen; deuterium; a substituted or unsubstituted C1 to C20 alkyl group; a substituted or unsubstituted C6 to C20 aryl group; a substituted or unsubstituted C2 to C20 heteroaryl group; or Chemical Formula 3, or two or more groups adjacent to each other may bond to each other to form a substituted or unsubstituted C6 to C20 aromatic hydrocarbon ring; or a substituted or unsubstituted C2 to C20 heteroring.

In another embodiment of the present invention, R11 to R18 are the same as or different from each other, and each independently hydrogen; deuterium; a substituted or unsubstituted phenyl group; a substituted or unsubstituted biphenyl group; a substituted or unsubstituted naphthyl group; a substituted or unsubstituted dibenzothiophenyl group; or Chemical Formula 3, or two or more groups adjacent to each other may bond to each other to form a substituted or unsubstituted C6 to C20 aromatic hydrocarbon ring; or a substituted or unsubstituted C2 to C20 heteroring.

In one embodiment of the present invention, the heterocyclic compound represented by Chemical Formula 2 may be represented by any one of the following Chemical Formulae 2-1 to 2-4.

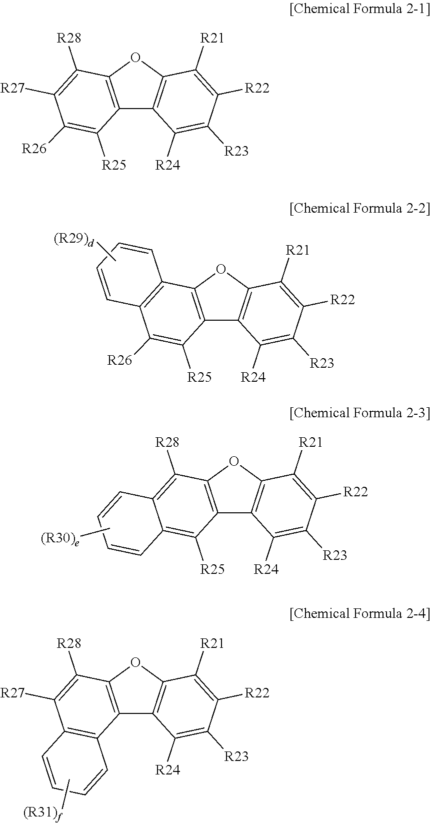

In Chemical Formulae 2-1 to 2-4,
R21 to R28 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; —NR201R202; and Chemical Formula 3, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group,
at least one of R21 to R28 is Chemical Formula 3,
R29 to R31 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group,
d is an integer of 0 to 4, and when d is 2 or greater, R29s are the same as or different from each other,
e is an integer of 0 to 4, and when e is 2 or greater, R30s are the same as or different from each other, and
f is an integer of 0 to 4, and when f is 2 or greater, R31s are the same as or different from each other.

In one embodiment of the present invention, R21 to R28 are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R201R202; —SiR201R202R203; —NR201R202; or Chemical Formula 3, and R201, R202 and R203 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present invention, R21 to R28 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R201R202; —SiR201R202R203; —NR201R202; or Chemical Formula 3, and R201, R202 and R203 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present invention, R21 to R28 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; or Chemical Formula 3.

In another embodiment of the present invention, R21 to R28 are the same as or different from each other, and may be each independently hydrogen; deuterium; a substituted or unsubstituted C1 to C20 alkyl group; a substituted or unsubstituted C6 to C20 aryl group; a substituted or unsubstituted C2 to C20 heteroaryl group; or Chemical Formula 3.

In another embodiment of the present invention, R21 to R28 are the same as or different from each other, and may be each independently hydrogen; deuterium; a substituted or unsubstituted phenyl group; a substituted or unsubstituted biphenyl group; a substituted or unsubstituted naphthyl group; a substituted or unsubstituted dibenzothiophenyl group; or Chemical Formula 3.

In one embodiment of the present invention, R29 to R31 are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R201R202; —SiR201R202R203; or —NR201R202.

In another embodiment of the present invention, R29 to R31 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R201R202; —SiR201R202R203; or —NR201R202.

In another embodiment of the present invention, R29 to R31 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present invention, R29 to R31 are the same as or different from each other, and may be each independently hydrogen; or deuterium.

In one embodiment of the present invention, R41 and Ra to Rd are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R301R302; —SiR301R302R303; or —NR301R302, or two or more groups adjacent to each other bond to each other to form a substituted or unsubstituted C6 to C30 aromatic hydrocarbon ring; or a substituted or unsubstituted C2 to C30 heteroring, and R301, R302 and R303 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present invention, R41 and Ra to Rd are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R301R302; —SiR301R302R303; or —NR301R302, or two or more groups adjacent to each other bond to each other to form a substituted or unsubstituted C6 to C20 aromatic hydrocarbon ring; or a substituted or unsubstituted C2 to C20 heteroring, and R301, R302 and R303 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present invention, R41 and Ra to Rd are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group, or two or more groups adjacent to each other may bond to each other to form a substituted or unsubstituted C6 to C20 aromatic hydrocarbon ring; or a substituted or unsubstituted C2 to C20 heteroring.

In another embodiment of the present invention, R41 and Ra to Rd are the same as or different from each other, and each independently hydrogen; deuterium; a substituted or unsubstituted C1 to C20 alkyl group; a substituted or unsubstituted C6 to C20 aryl group; or a substituted or unsubstituted C2 to C20 heteroaryl group, or two or more groups adjacent to each other may bond to each other to form a substituted or unsubstituted C6 to C20 aromatic hydrocarbon ring; or a substituted or unsubstituted C2 to C20 heteroring.

In another embodiment of the present invention, R41 and Ra to Rd are the same as or different from each other, and each independently hydrogen; deuterium; a substituted or unsubstituted phenyl group; a substituted or unsubstituted biphenyl group; a substituted or unsubstituted naphthyl group; a substituted or unsubstituted chrysenyl group; or a substituted or unsubstituted dibenzofuranyl group, or two or more groups adjacent to each other may bond to each other to form a substituted or unsubstituted C6 to C20 aromatic hydrocarbon ring; or a substituted or unsubstituted C2 to C20 heteroring.

In one embodiment of the present invention, L11 may be a direct bond; a substituted or unsubstituted C6 to C30 arylene group; or a substituted or unsubstituted C2 to C30 heteroarylene group.

In another embodiment of the present invention, L11 may be a direct bond; a substituted or unsubstituted C6 to C20 arylene group; or a substituted or unsubstituted C2 to C20 heteroarylene group.

In another embodiment of the present invention, L11 may be a direct bond; a substituted or unsubstituted phenylene group; or a substituted or unsubstituted naphthylene group.

In one embodiment of the present invention, Chemical Formula 3 may be represented by any one of the following Chemical Formulae 3-1 to 3-6.

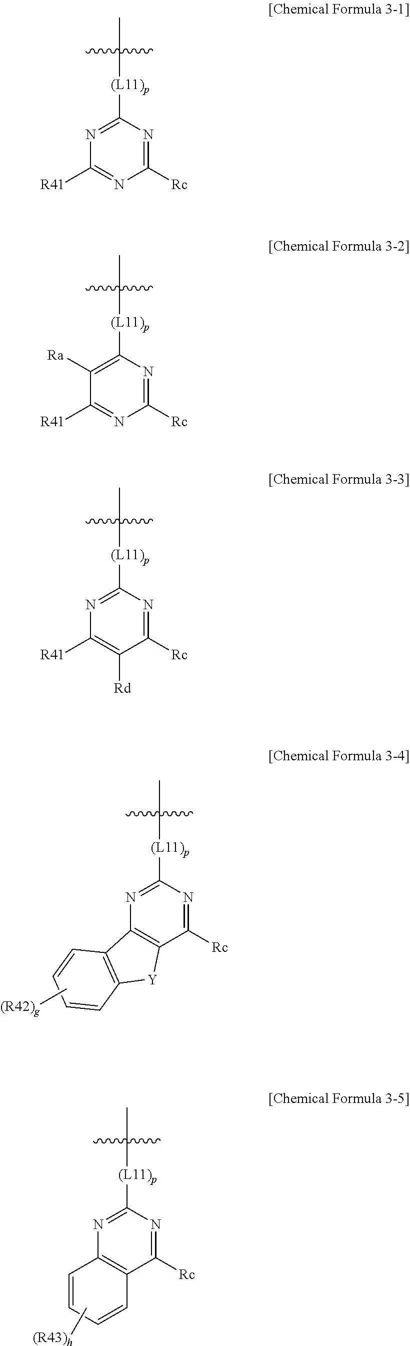

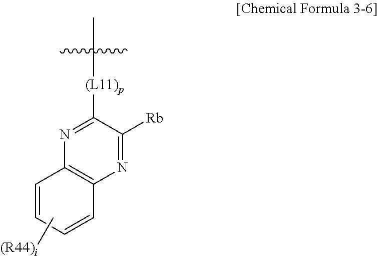

In Chemical Formulae 3-1 to 3-6,
Y is O; or S,
R42 to R44 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R301R302; —SiR301R302R303; and —NR301R302, and R301, R302 and R303 are the same as or different from each other and each independently a substituted or unsubstituted C1 to C60 alkyl group; a substituted or unsubstituted C6 to C60 aryl group; or a substituted or unsubstituted C2 to C60 heteroaryl group,
g is an integer of 0 to 4, and when g is 2 or greater, R42s are the same as or different from each other,
h is an integer of 0 to 4, and when h is 2 or greater, R43s are the same as or different from each other,
i is an integer of 0 to 4, and when i is 2 or greater, R44s are the same as or different from each other, and
R41, Ra to Rd, L11 and p have the same definitions as in Chemical Formula 3.

In one embodiment of the present invention, R42 to R44 are the same as or different from each other and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R301R302; —SiR301R302R303; or —NR301R302, and R301, R302 and R303 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present invention, R42 to R44 are the same as or different from each other and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R301R302; —SiR301R302R303; or —NR301R302, and R301, R302 and R303 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present invention, R42 to R44 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In another embodiment of the present invention, R42 to R44 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present invention, R42 to R44 are the same as or different from each other, and may be each independently hydrogen; or deuterium.

In one embodiment of the present invention, the heterocyclic compound represented by Chemical Formula 2 may not include deuterium as a substituent, or may have a deuterium content of, for example, greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present invention, the heterocyclic compound represented by Chemical Formula 2 may not include deuterium as a substituent, or may have a deuterium content of 1% to 100% with respect to the total number of hydrogen atoms and deuterium atoms.

In another embodiment of the present invention, the heterocyclic compound represented by Chemical Formula 2 may not include deuterium as a substituent, or may have a deuterium content of 20% to 90% with respect to the total number of hydrogen atoms and deuterium atoms.

In another embodiment of the present invention, the heterocyclic compound represented by Chemical Formula 2 may not include deuterium as a substituent, or may have a deuterium content of 30% to 80% with respect to the total number of hydrogen atoms and deuterium atoms.

In another embodiment of the present invention, the heterocyclic compound represented by Chemical Formula 2 may not include deuterium as a substituent, or may have a deuterium content of 50% to 70% with respect to the total number of hydrogen atoms and deuterium atoms.

When the heterocyclic compound represented by Chemical Formula 1 and the heterocyclic compound represented by Chemical Formula 2 are included at the same time, effects of more superior efficiency and lifetime are obtained. From this, it may be expected that an exciplex phenomenon occurs when the two compounds are included at the same time.

The exciplex phenomenon is a phenomenon of releasing energy having sizes of a donor (p-host) HOMO energy level and an acceptor (n-host) LUMO energy level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and a driving voltage may be lowered, which resultantly helps with enhancement in the lifetime. In other words, when the compound represented by Chemical Formula 1 is used as the donor and the compound represented by Chemical Formula 2 is used as the acceptor, excellent device properties are obtained.

In one embodiment of the present invention, when the heterocyclic compound represented by Chemical Formula 1 and the heterocyclic compound represented by Chemical Formula 2 are included at the same time, at least one of the compounds does not include deuterium as a substituent, or may have a deuterium content of greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present invention, at least one of the compounds may not include deuterium as a substituent, or may have a deuterium content of 1% to 100% with respect to the total number of hydrogen atoms and deuterium atoms.

In another embodiment of the present invention, at least one of the compounds may not include deuterium as a substituent, or may have a deuterium content of 20% to 90% with respect to the total number of hydrogen atoms and deuterium atoms.

In another embodiment of the present invention, at least one of the compounds may not include deuterium as a substituent, or may have a deuterium content of 30% to 80% with respect to the total number of hydrogen atoms and deuterium atoms.

In another embodiment of the present invention, at least one of the compounds may not include deuterium as a substituent, or may have a deuterium content of 50% to 70% with respect to the total number of hydrogen atoms and deuterium atoms.

In one embodiment of the present invention, the heterocyclic compound represented by Chemical Formula 2 may be represented by any one of the following compounds.

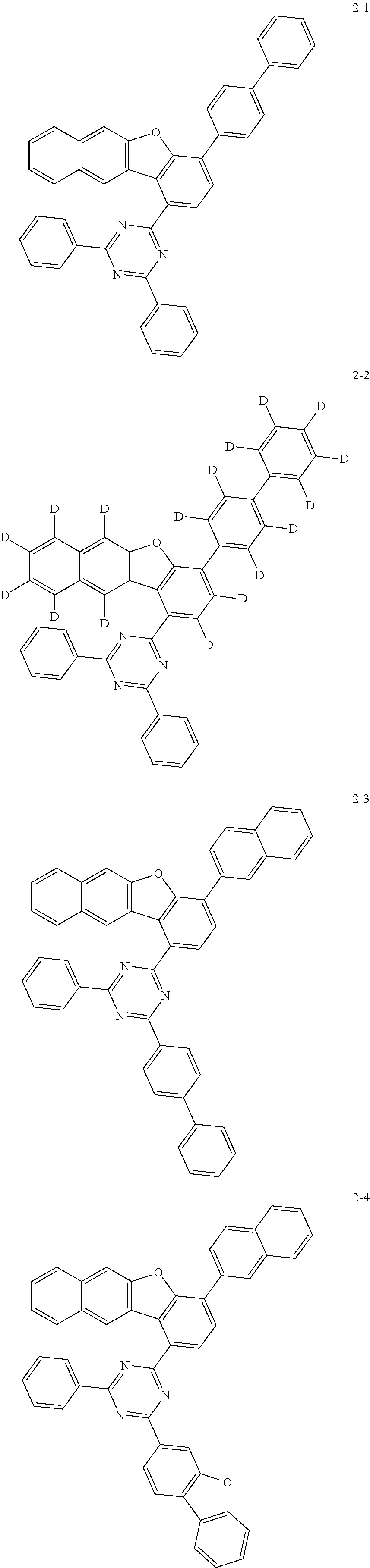 [SMILES: c1ccc(-c2ccc(-c3ccc(-c4nc(-c5ccccc5)nc(-c5ccccc5)n4)c4c3oc3cc5ccccc5cc34)cc2)cc1 ; [2H]c1c([2H])c([2H])c(-c2c([2H])c([2H])c(-c3c([2H])c([2H])c(-c4nc(-c5ccccc5)nc(-c5ccccc5)n4)c4c3oc3c([2H])c5c([2H])c([2H])c([2H])c([2H])c5c([2H])c34)c([2H])c2[2H])c([2H])c1[2H] ; c1ccc(-c2ccc(-c3nc(-c4ccccc4)nc(-c4ccc(-c5ccc6ccccc6c5)c5oc6cc7ccccc7cc6c45)n3)cc2)cc1 ; c1ccc(-c2nc(-c3ccc4c(c3)oc3ccccc34)nc(-c3ccc(-c4ccc5ccccc5c4)c4oc5cc6ccccc6cc5c34)n2)cc1]

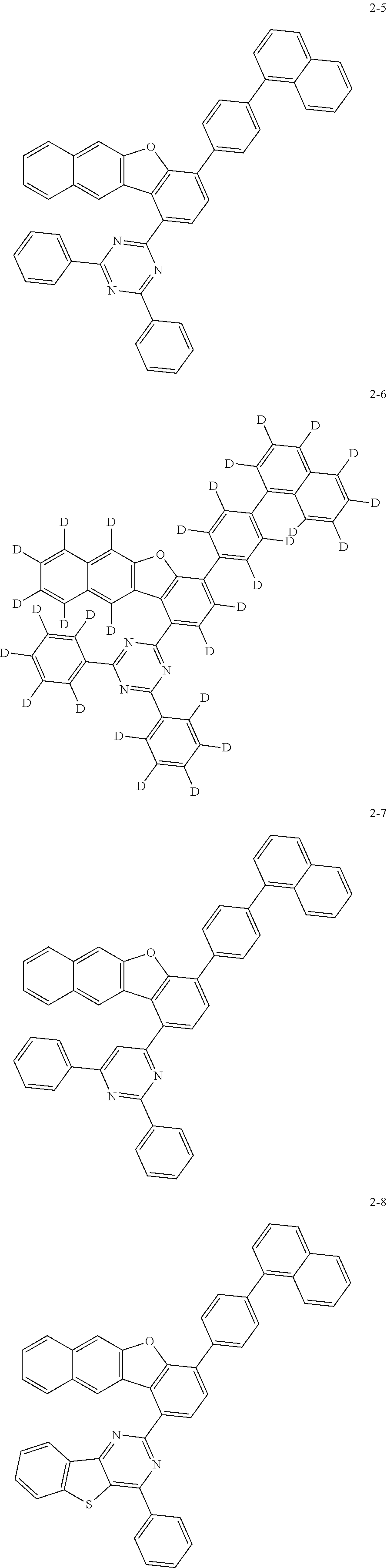

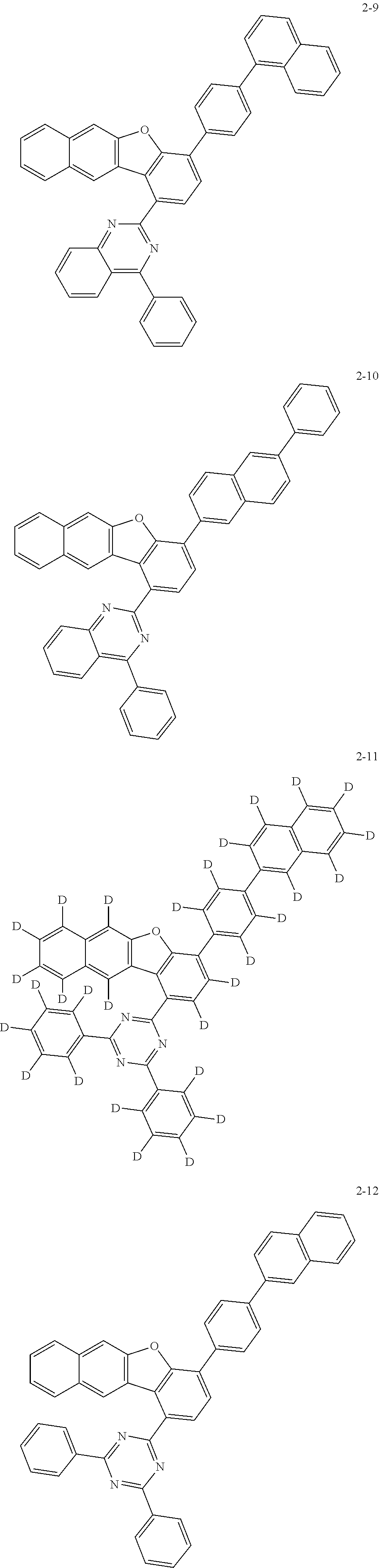

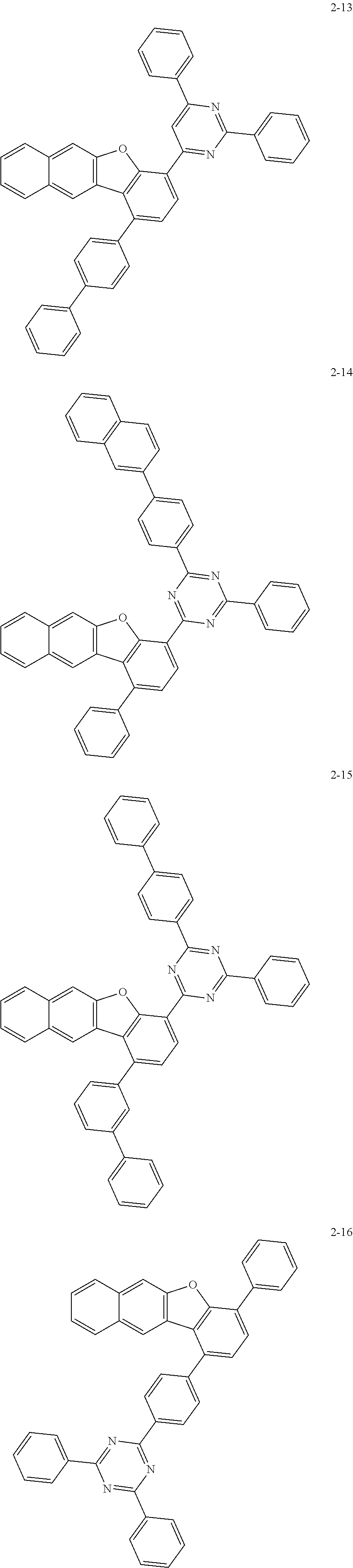 [SMILES: c1ccc(-c2ccc(-c3ccc(-c4cc(-c5ccccc5)nc(-c5ccccc5)n4)c4oc5cc6ccccc6cc5c34)cc2)cc1 ; c1ccc(-c2nc(-c3ccc(-c4ccc5ccccc5c4)cc3)nc(-c3ccc(-c4ccccc4)c4c3oc3cc5ccccc5cc34)n2)cc1 ; c1ccc(-c2ccc(-c3nc(-c4ccccc4)nc(-c4ccc(-c5cccc(-c6ccccc6)c5)c5c4oc4cc6ccccc6cc45)n3)cc2)cc1 ; c1ccc(-c2nc(-c3ccccc3)nc(-c3ccc(-c4ccc(-c5ccccc5)c5oc6cc7ccccc7cc6c45)cc3)n2)cc1]

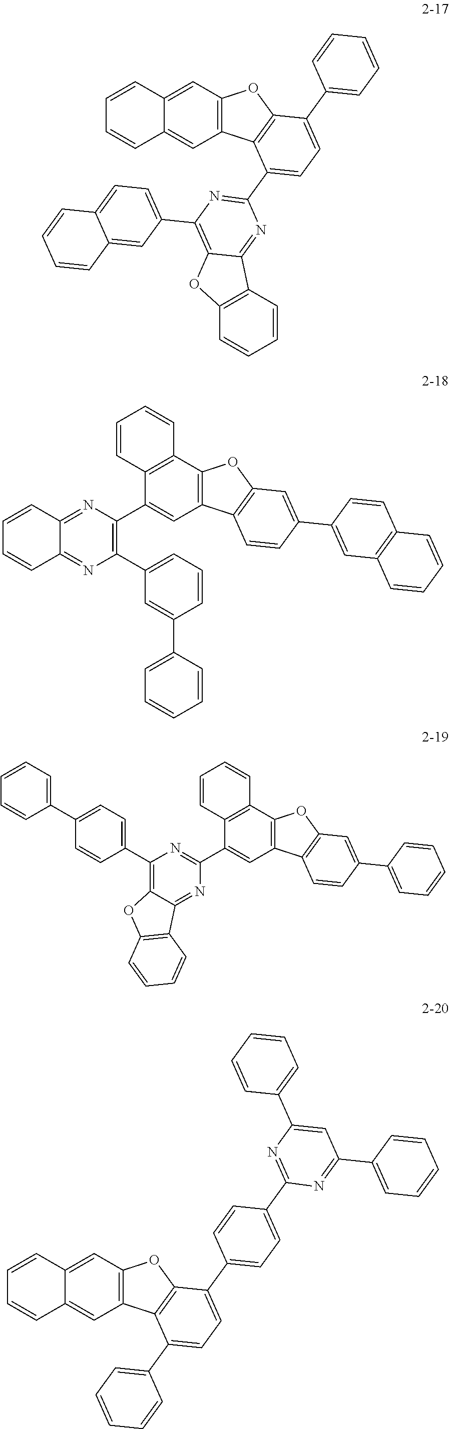 [SMILES: c1ccc(-c2ccc(-c3nc(-c4ccc5ccccc5c4)c4oc5ccccc5c4n3)c3c2oc2cc4ccccc4cc23)cc1 ; c1ccc(-c2cccc(-c3nc4ccccc4nc3-c3cc4c5ccc(-c6ccc7ccccc7c6)cc5oc4c4ccccc34)c2)cc1 ; c1ccc(-c2ccc(-c3nc(-c4cc5c6ccc(-c7ccccc7)cc6oc5c5ccccc45)nc4c3oc3ccccc34)cc2)cc1 ; c1ccc(-c2cc(-c3ccccc3)nc(-c3ccc(-c4ccc(-c5ccccc5)c5c4oc4cc6ccccc6cc45)cc3)n2)cc1]

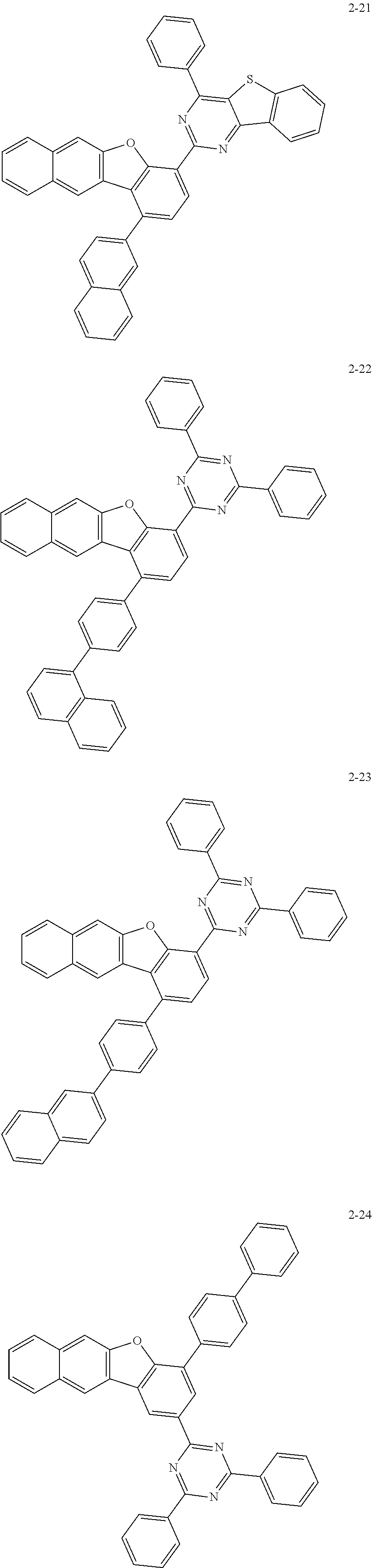 [SMILES: c1ccc(-c2nc(-c3ccc(-c4ccc5ccccc5c4)c4c3oc3cc5ccccc5cc34)nc3c2sc2ccccc23)cc1 ; c1ccc(-c2nc(-c3ccccc3)nc(-c3ccc(-c4ccc(-c5cccc6ccccc56)cc4)c4c3oc3cc5ccccc5cc34)n2)cc1 ; c1ccc(-c2nc(-c3ccccc3)nc(-c3ccc(-c4ccc(-c5ccc6ccccc6c5)cc4)c4c3oc3cc5ccccc5cc34)n2)cc1 ; c1ccc(-c2ccc(-c3cc(-c4nc(-c5ccccc5)nc(-c5ccccc5)n4)cc4c3oc3cc5ccccc5cc34)cc2)cc1]

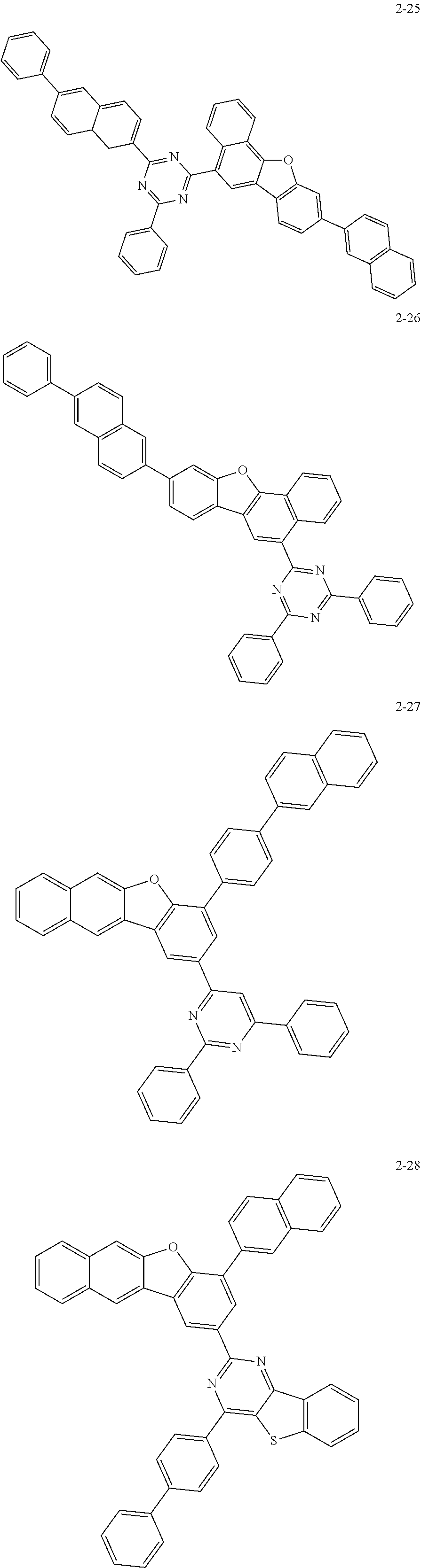 [SMILES: C1=CC2CC(c3nc(-c4ccccc4)nc(-c4cc5c6ccc(-c7ccc8ccccc8c7)cc6oc5c5ccccc45)n3)=CC=C2C=C1c1ccccc1 ; c1ccc(-c2ccc3cc(-c4ccc5c(c4)oc4c6ccccc6c(-c6nc(-c7ccccc7)nc(-c7ccccc7)n6)cc54)ccc3c2)cc1 ; c1ccc(-c2cc(-c3cc(-c4ccc(-c5ccc6ccccc6c5)cc4)c4oc5cc6ccccc6cc5c4c3)nc(-c3ccccc3)n2)cc1 ; c1ccc(-c2ccc(-c3nc(-c4cc(-c5ccc6ccccc6c5)c5oc6cc7ccccc7cc6c5c4)nc4c3sc3ccccc34)cc2)cc1]

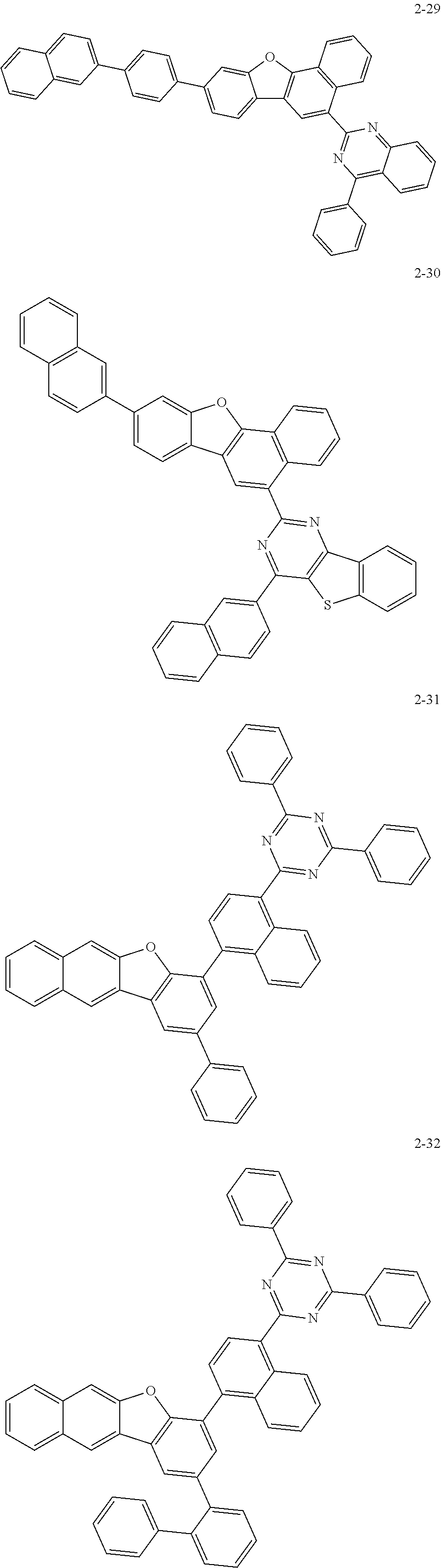

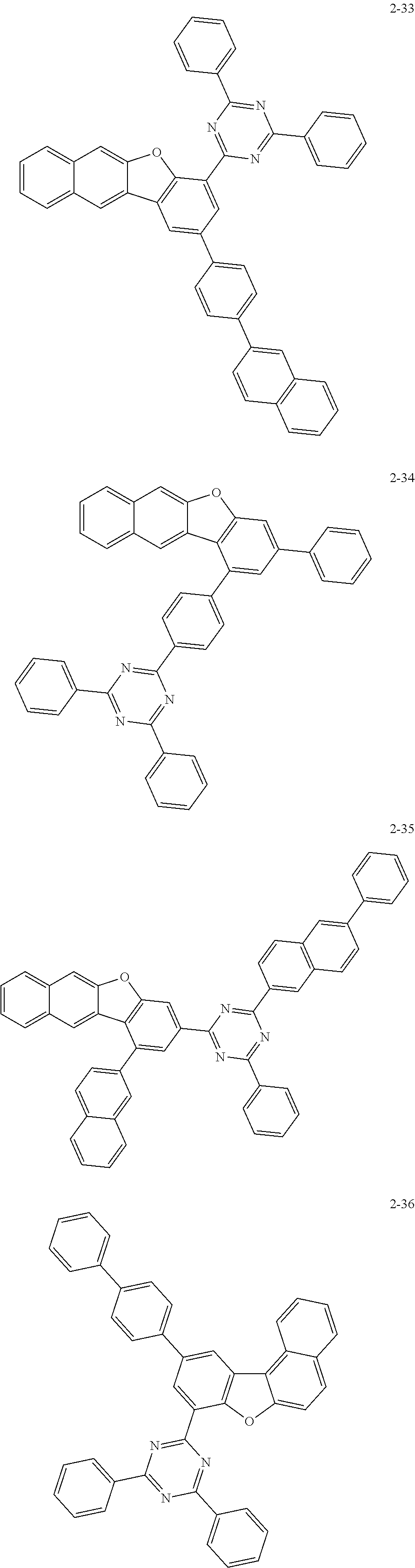 [SMILES: c1ccc(-c2nc(-c3ccccc3)nc(-c3cc(-c4ccc(-c5ccc6ccccc6c5)cc4)cc4c3oc3cc5ccccc5cc34)n2)cc1 ; c1ccc(-c2cc(-c3ccc(-c4nc(-c5ccccc5)nc(-c5ccccc5)n4)cc3)c3c(c2)oc2cc4ccccc4cc23)cc1 ; c1ccc(-c2ccc3cc(-c4nc(-c5ccccc5)nc(-c5cc(-c6ccc7ccccc7c6)c6c(c5)oc5cc7ccccc7cc56)n4)ccc3c2)cc1 ; c1ccc(-c2ccc(-c3cc(-c4nc(-c5ccccc5)nc(-c5ccccc5)n4)c4oc5ccc6ccccc6c5c4c3)cc2)cc1]

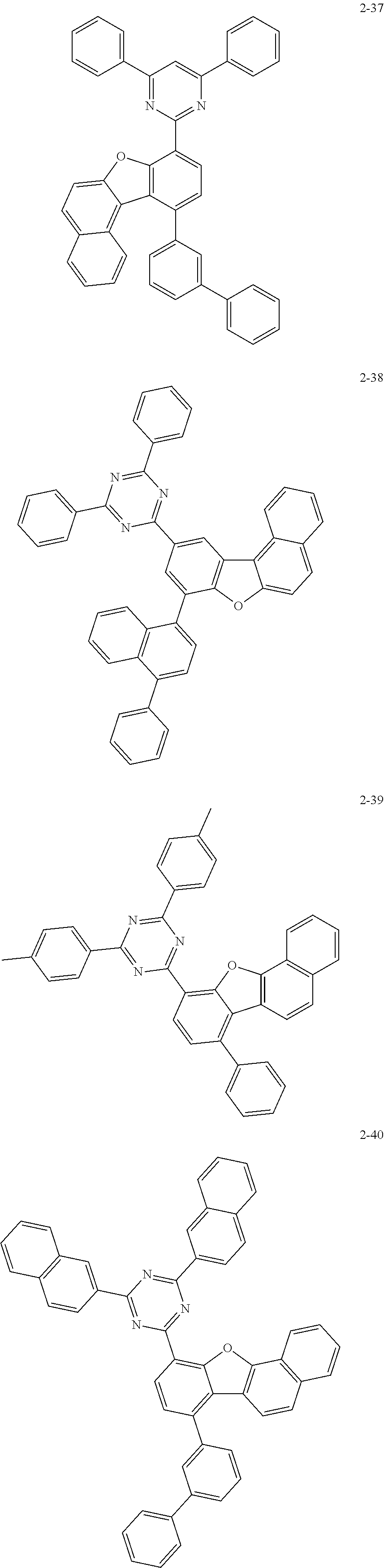 [SMILES: c1ccc(-c2cccc(-c3ccc(-c4nc(-c5ccccc5)cc(-c5ccccc5)n4)c4oc5ccc6ccccc6c5c34)c2)cc1 ; c1ccc(-c2nc(-c3ccccc3)nc(-c3cc(-c4ccc(-c5ccccc5)c5ccccc45)c4oc5ccc6ccccc6c5c4c3)n2)cc1 ; Cc1ccc(-c2nc(-c3ccc(C)cc3)nc(-c3ccc(-c4ccccc4)c4c3oc3c5ccccc5ccc34)n2)cc1 ; c1ccc(-c2cccc(-c3ccc(-c4nc(-c5ccc6ccccc6c5)nc(-c5ccc6ccccc6c5)n4)c4oc5c6ccccc6ccc5c34)c2)cc1]

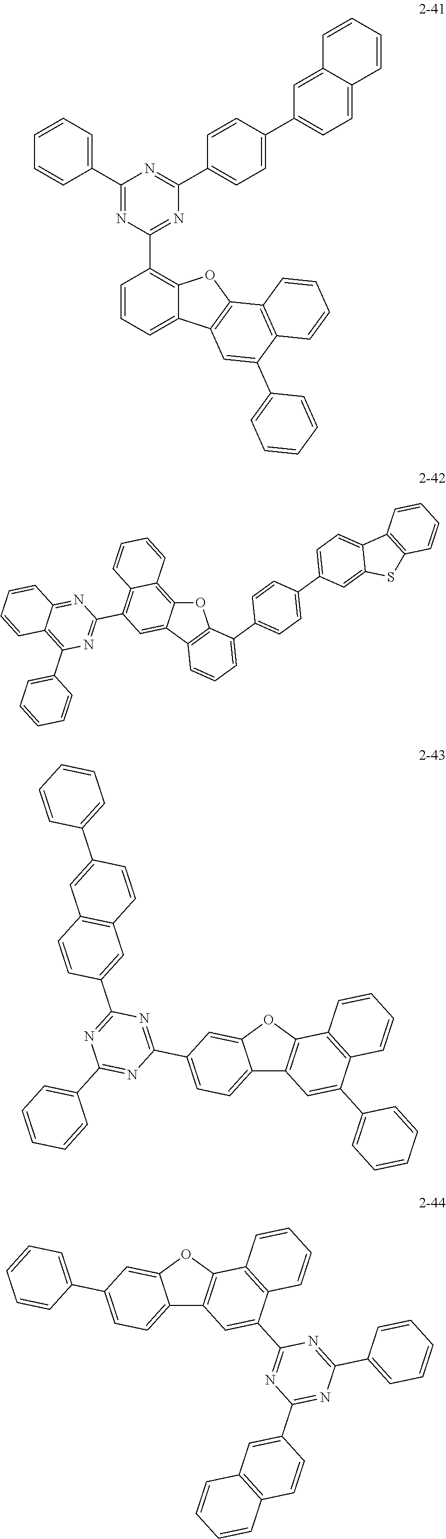

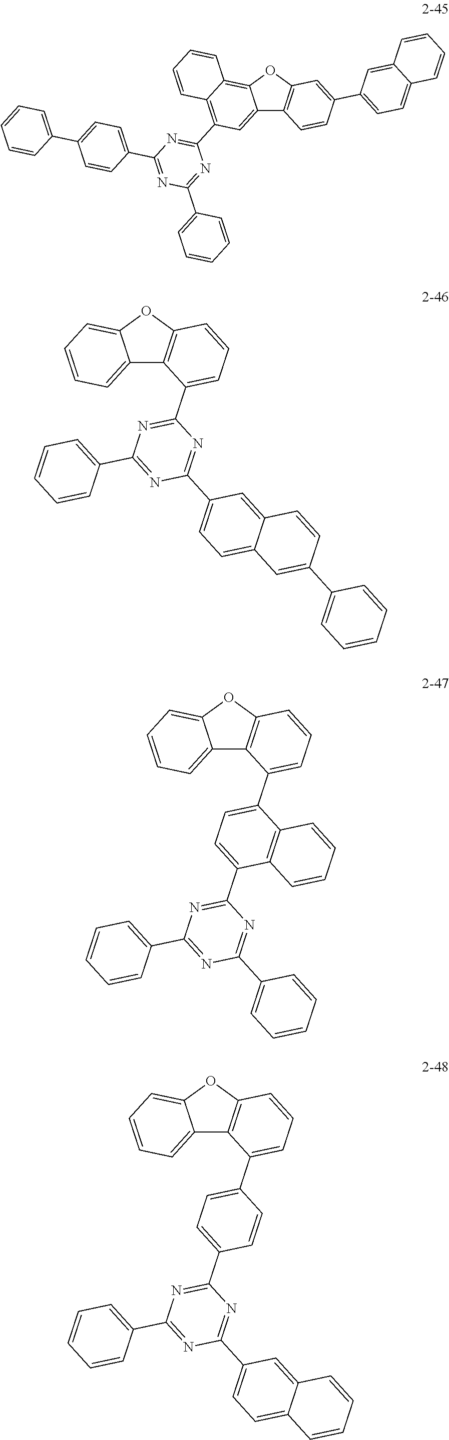 [SMILES: c1ccc(-c2ccc(-c3nc(-c4ccccc4)nc(-c4cc5c6ccc(-c7ccc8ccccc8c7)cc6oc5c5ccccc45)n3)cc2)cc1 ; c1ccc(-c2ccc3cc(-c4nc(-c5ccccc5)nc(-c5cccc6oc7ccccc7c56)n4)ccc3c2)cc1 ; c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1]

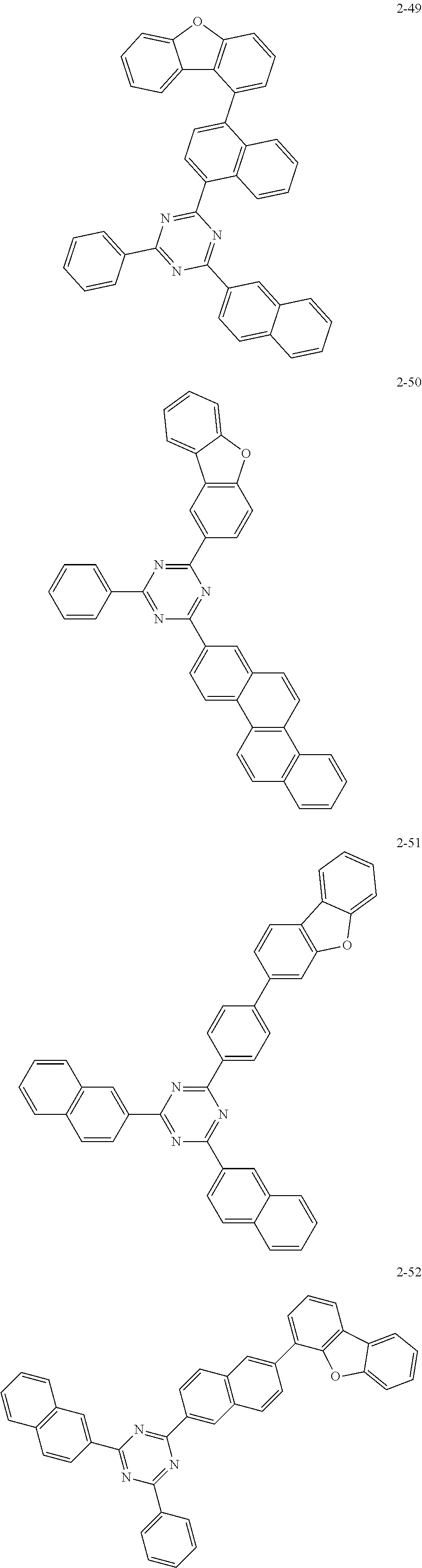 [SMILES: c1ccc(-c2nc(-c3ccc4ccccc4c3)nc(-c3ccc(-c4cccc5oc6ccccc6c45)c4ccccc34)n2)cc1 ; c1ccc(-c2nc(-c3ccc4c(ccc5c6ccccc6ccc45)c3)nc(-c3ccc4oc5ccccc5c4c3)n2)cc1 ; c1ccc2cc(-c3nc(-c4ccc(-c5ccc6c(c5)oc5ccccc56)cc4)nc(-c4ccc5ccccc5c4)n3)ccc2c1 ; c1ccc(-c2nc(-c3ccc4ccccc4c3)nc(-c3ccc4cc(-c5cccc6c5oc5ccccc56)ccc4c3)n2)cc1]

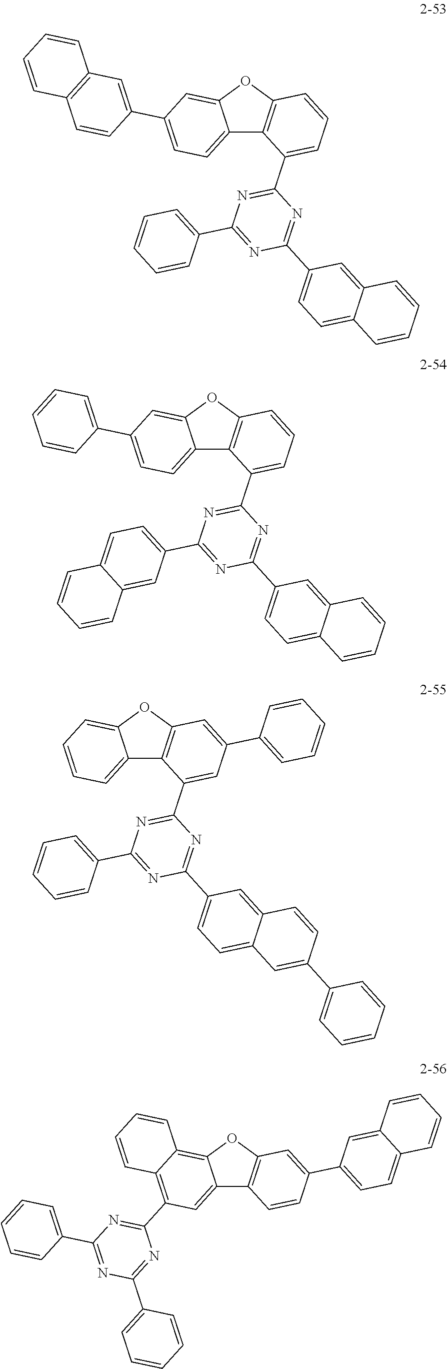 [SMILES: c1ccc(-c2nc(-c3ccc4ccccc4c3)nc(-c3cccc4oc5cc(-c6ccc7ccccc7c6)ccc5c34)n2)cc1 ; c1ccc(-c2ccc3c(c2)oc2cccc(-c4nc(-c5ccc6ccccc6c5)nc(-c5ccc6ccccc6c5)n4)c23)cc1 ; c1ccc(-c2ccc3cc(-c4nc(-c5ccccc5)nc(-c5cc(-c6ccccc6)cc6oc7ccccc7c56)n4)ccc3c2)cc1 ; c1ccc(-c2nc(-c3ccccc3)nc(-c3cc4c5ccc(-c6ccc7ccccc7c6)cc5oc4c4ccccc34)n2)cc1]

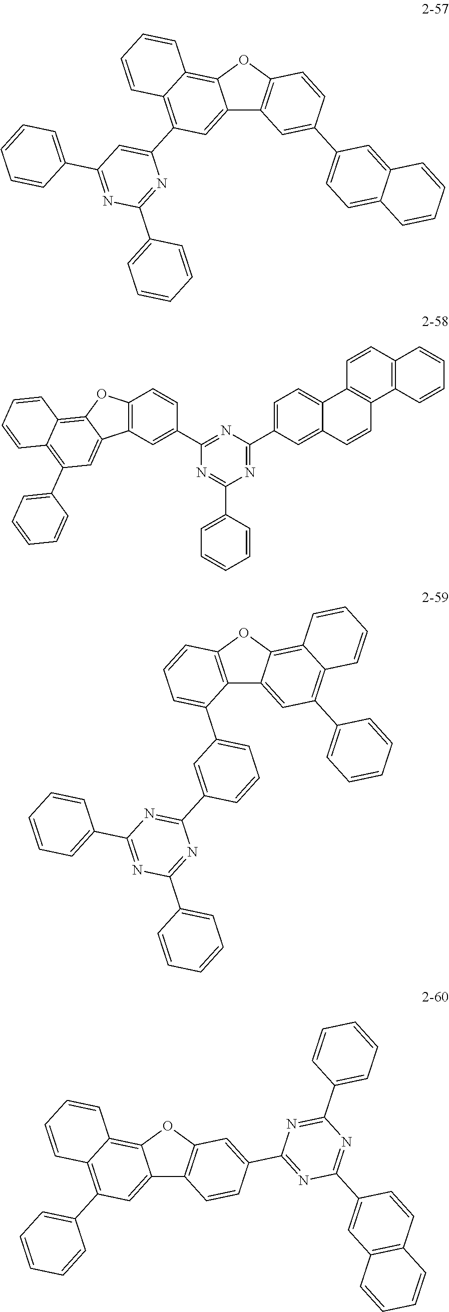 [SMILES: c1ccc(-c2cc(-c3cc4c5cc(-c6ccc7ccccc7c6)ccc5oc4c4ccccc34)nc(-c3ccccc3)n2)cc1 ; c1ccc(-c2nc(-c3ccc4c(ccc5c6ccccc6ccc45)c3)nc(-c3ccc4oc5c6ccccc6c(-c6ccccc6)cc5c4c3)n2)cc1 ; c1ccc(-c2nc(-c3ccccc3)nc(-c3cccc(-c4cccc5oc6c7ccccc7c(-c7ccccc7)cc6c45)c3)n2)cc1 ; c1ccc(-c2nc(-c3ccc4ccccc4c3)nc(-c3ccc4c(c3)oc3c5ccccc5c(-c5ccccc5)cc43)n2)cc1]

In addition, one embodiment of the present invention provides a composition for an organic material layer of an organic light-emitting device, the composition comprising: the heterocyclic compound represented by Chemical Formula 1 and the heterocyclic compound represented by Chemical Formula 2.

Specific descriptions on the heterocyclic compound represented by Chemical Formula 1 and the heterocyclic compound represented by Chemical Formula 2 are the same as the descriptions provided above.

In one embodiment of the present invention, the heterocyclic compound represented by Chemical Formula 1 and the heterocyclic compound represented by Chemical Formula 2 in the composition for an organic material layer may have a weight ratio of 1:9 to 9:1, 1:9 to 5:5, or 2:8 to 5:5, however, the ratio is not limited thereto.

The composition for an organic material layer may be used when forming an organic material of an organic light-emitting device, and particularly, may be more preferably used when forming a host of a light emitting layer.

In one embodiment of the present invention, the organic material layer includes the heterocyclic compound represented by Chemical Formula 1 and the heterocyclic compound represented by Chemical Formula 2, and a phosphorescent dopant may be used therewith.

As the phosphorescent dopant material, those known in the art may be used. For example, phosphorescent dopant materials represented by LL′MX′, LL′L″M, LMX′X″, L₂MX′ and L₃M may be used, however, the scope of the present invention is not limited by these examples.
M may be iridium, platinum, osmium or the like.
L is an anionic bidentate ligand coordinated to M by sp² carbon and heteroatom, and X may function to trap electrons or holes. Nonlimiting examples of L may include 2-(1-naphthyl)benzoxazole, (2-phenylbenzoxazole), (2-phenylbenzothiazole), (7,8-benzoquinoline), phenylpyridine, benzothiophenylpyrizine, 3-methoxy-2-phenylpyridine, thiophenylpyrizine, tolylpyridine and the like. Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are shown below, however, the phosphorescent dopant is not limited to these examples.

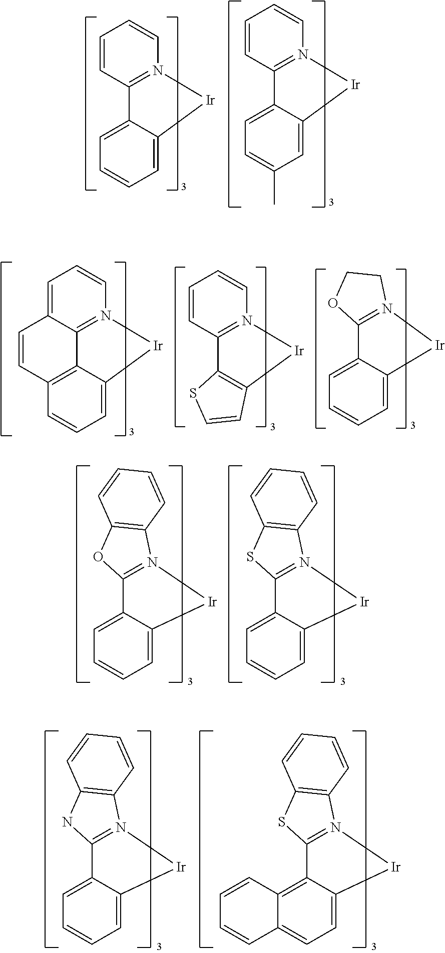

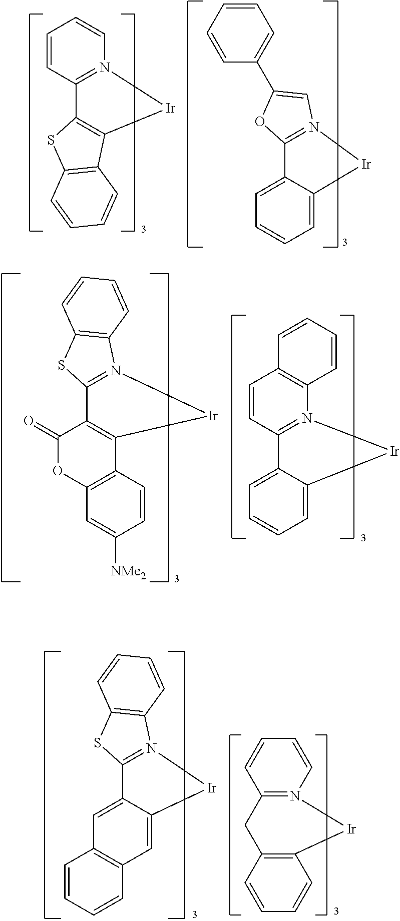

In one embodiment of the present invention, the organic material layer includes the heterocyclic compound represented by Chemical Formula 1 and the heterocyclic compound represented by Chemical Formula 2, and an iridium-based dopant may be used therewith.

In one embodiment of the present invention, as the iridium-based dopant, (piq)₂(Ir) (acac) may be used as a red phosphorescent dopant or Ir(ppy)₃ may be used as a green phosphorescent dopant.

In one embodiment of the present invention, a content of the dopant may be from 1% to 15%, preferably from 2% to 10% and more preferably from 3% to 7% based on the total weight of the light emitting layer.

In the organic light-emitting device according to one embodiment of the present invention, the organic material layer includes an electron injection layer or an electron transport layer, and the electron injection layer or the electron transport layer may include the heterocyclic compound represented by Chemical Formula 1.

In the organic light-emitting device according to another embodiment of the present invention, the organic material layer includes an electron blocking layer or a hole blocking layer, and the electron blocking layer or the hole blocking layer may include the heterocyclic compound represented by Chemical Formula 1.

In the organic light-emitting device according to another embodiment of the present invention, the organic material layer includes an electron transport layer, a light emitting layer or a hole blocking layer, and the electron transport layer, the light emitting layer or the hole blocking layer may include the heterocyclic compound represented by Chemical Formula 1.

In the organic light-emitting device according to another embodiment of the present invention, the organic material layer includes a light emitting layer, and the light emitting layer may include the heterocyclic compound represented by Chemical Formula 1.

In the organic light-emitting device according to another embodiment of the present invention, the organic material layer includes a light emitting layer, the light emitting layer includes a host material, and the host material may include the heterocyclic compound represented by Chemical Formula 1.

In the organic light-emitting device according to another embodiment of the present invention, the light emitting layer may include two or more host materials, and at least one of the host materials may include the heterocyclic compound represented by Chemical Formula 1 and another one may include the heterocyclic compound represented by Chemical Formula 2.

In the organic light-emitting device according to another embodiment of the present invention, two or more host materials may be pre-mixed and used in the light emitting layer, and at least one of the two or more host materials may include the heterocyclic compound represented by Chemical Formula 1 and another one may include the heterocyclic compound represented by Chemical Formula 2.

The pre-mixing means, before depositing the two or more host materials on the organic material layer, putting and mixing the materials first in one source of supply.

The organic light-emitting device according to one embodiment of the present invention may further include, one, or two or more layers selected from the group consisting of a light emitting layer, a hole injection layer, a hole transport layer, an electron injection layer, an electron transport layer, an electron blocking layer and a hole blocking layer.

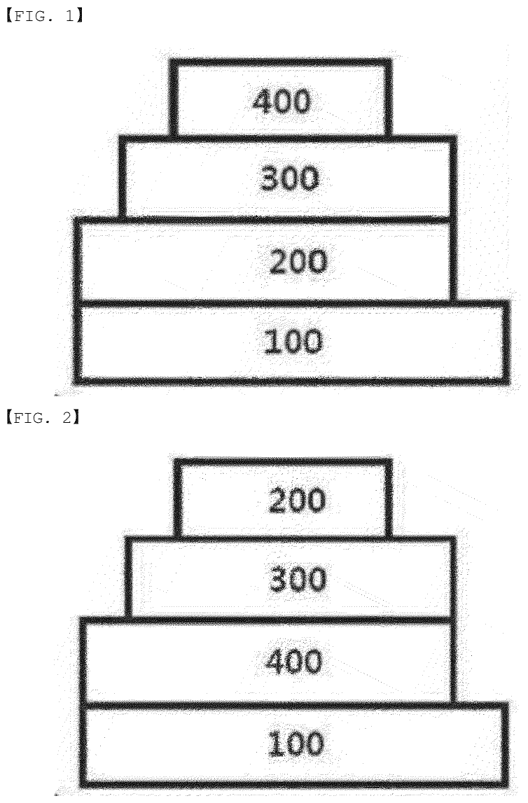

FIGS. 1 to 3 illustrate a lamination order of electrodes and organic material layers of the organic light-emitting device according to one embodiment of the present invention. However, it is not intended that the scope of the present application be limited by these drawings, and structures of organic light-emitting devices known in the art may also be applied to the present application.

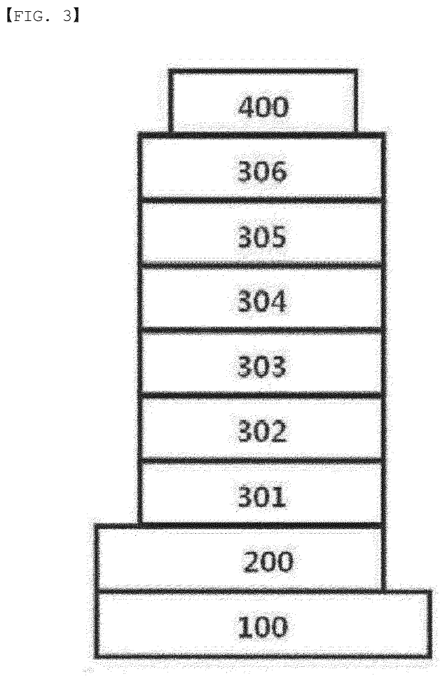

FIG. 1 illustrates an organic light-emitting device in which a positive electrode 200, an organic material layer 300 and a negative electrode 400 are sequentially laminated on a substrate 100. However, the structure is not limited only to such a structure, and as illustrated in FIG. 2, an organic light-emitting device in which a negative electrode, an organic material layer and a positive electrode are sequentially laminated on a substrate may also be obtained.

FIG. 3 illustrates a case of the organic material layer being a multilayer. An organic light-emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

One embodiment of the present invention provides a method for manufacturing an organic light-emitting device, the method including:
preparing a substrate;
forming a first electrode on the substrate;
forming one or more organic material layers on the first electrode; and
forming a second electrode on the one or more organic material layers, wherein the forming of one or more organic material layers includes forming the one or more organic material layers using the composition for an organic material layer of an organic light-emitting device according to one embodiment of the present invention.

In one embodiment of the present invention, the forming of organic material layers may be forming the organic material layers using a thermal vacuum deposition method after pre-mixing the heterocyclic compound represented by Chemical Formula 1 and the heterocyclic compound represented by Chemical Formula 2.

The pre-mixing means, before depositing the heterocyclic compound represented by Chemical Formula 1 and the heterocyclic compound represented by Chemical Formula 2 on the organic material layer, putting and mixing the materials first in one source of supply.

The pre-mixed material may be referred to as the composition for an organic material layer according to one embodiment of the present application.

The organic material layer comprising the heterocyclic compound represented by Chemical Formula 1 may further include other materials as necessary.

The organic material layer comprising the heterocyclic compound represented by Chemical Formula 1 and the heterocyclic compound represented by Chemical Formula 2 at the same time may further include other materials as necessary.

In the organic light-emitting device according to one embodiment of the present invention, materials other than the heterocyclic compound represented by Chemical Formula 1 or the heterocyclic compound represented by Chemical Formula 2 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene](PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tris[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involved in light emission together may also be used.

When hosts of the light emitting layer material are mixed and used, same series hosts may be mixed and used, or different series hosts may be mixed and used. For example, any two or more types of materials among n-type host materials and p-type host materials may be selected and used as a host material of a light emitting layer.

The organic light-emitting device according to one embodiment of the present invention may be a top-emission type, a bottom-emission type or a dual-emission type depending on the materials used.

The heterocyclic compound according to one embodiment of the present invention may also be used in an organic electronic device including an organic solar cell, an organic photo conductor, an organic transistor and the like under a principle similar to that in the organic light-emitting device.

Hereinafter, preferred examples are provided to help to understand the present invention, however, the following examples are only provided to more readily understand the present invention, and the present invention is not limited thereto.

### PREPARATION EXAMPLE

### Preparation Example 1. Preparation of Compound 5

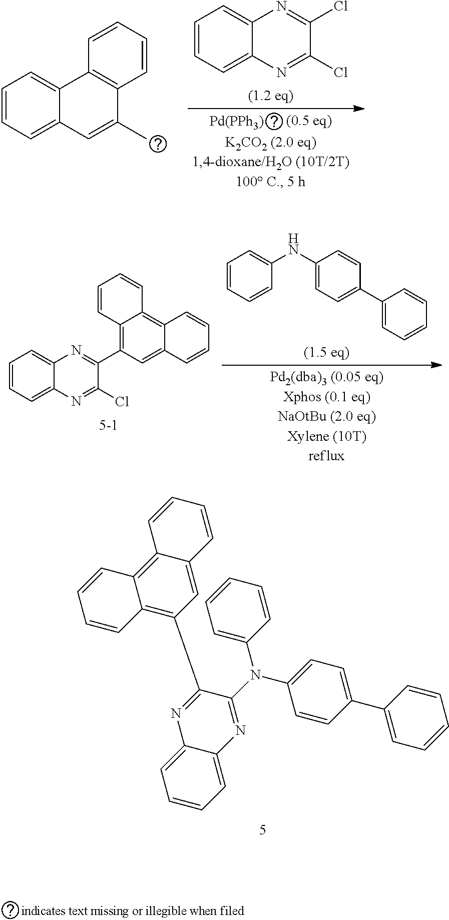

### Preparation Example 1-1. Preparation of Compound 5-1

Phenanthren-9-ylboronic acid (20 g, 90.07 mmol), 2,3-dichloroquinoxaline (21.73 g, 108.08 mmol), tetrakis(triphenylphosphine)palladium(0) (Pd(PPh₃)₄) (5.2 g, 4.5 mmol) and potassium carbonate (K₂CO₃) (31.12 g, 225.17 mmol) were dissolved in 1,4-dioxane (200 mL) and water (40 mL), and then the mixture was refluxed for 5 hours at 100° C.

After the reaction was completed, the result was cooled to room temperature, and then precipitated solids were filtered. The obtained solids were dissolved in dichloromethane (DCM), and after removing moisture with MgSO₄, the result was purified using a column to obtain Compound 5-1 (20 g, yield 65.2%).

### Preparation Example 1-2. Preparation of Compound 5

Compound 5-1 (7 g, 20.54 mmol), N-phenyl-(1,1′-biphenyl)-4-amine (7.61 g, 30.81 mmol), tris(dibenzylideneacetone)dipalladium(0) (Pd₂dba₃) (0.94 g, 1.03 mmol), Xphos (0.98 g, 2.05 mmol) and sodium tert-butoxide (NaOtBu) (4.93 g, 51.35 mmol) were dissolved in xylene, and then the mixture was refluxed.

After the reaction was completed, the result was cooled to room temperature, and then the solvent was removed under reduced pressure. The reaction material was purified by column chromatography (dichloromethane:hexane=1:1 (volume ratio)) to obtain Compound 5 (9.14 g, yield 81%).

Target compounds were prepared as shown in the following Table 1 in the same manner as in Preparation Example 1, except that, in preparing Compound 5, Intermediate A of the following Table 1 was used instead of N-phenyl-(1,1′-biphenyl)-4-amine.

TABLE 1

| Com- | | | |
|---|---|---|---|
| pound | | | |
| No. | Intermediate A | Target Compound | Yield |
| 8 | 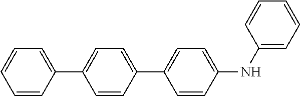 [SMILES: c1ccc(Nc2ccc(-c3ccc(-c4ccccc4)cc3)cc2)cc1] | 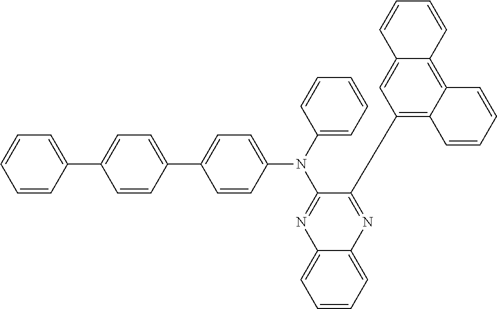 [SMILES: c1ccc(-c2ccc(-c3ccc(N(c4ccccc4)c4nc5ccccc5nc4-c4cc5ccccc5c5ccccc45)cc3)cc2)cc1] | 79% |
| 13 | 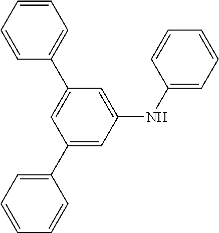 [SMILES: c1ccc(Nc2cc(-c3ccccc3)cc(-c3ccccc3)c2)cc1] | 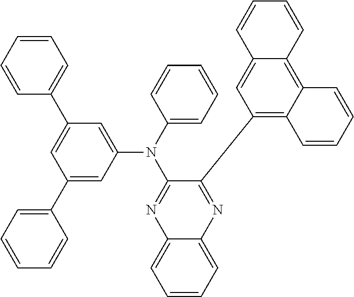 [SMILES: c1ccc(-c2cc(-c3ccccc3)cc(N(c3ccccc3)c3nc4ccccc4nc3-c3cc4ccccc4c4ccccc34)c2)cc1] | 78% |
| 14 | 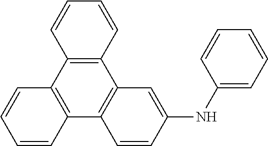 [SMILES: c1ccc(Nc2ccc3c4ccccc4c4ccccc4c3c2)cc1] | 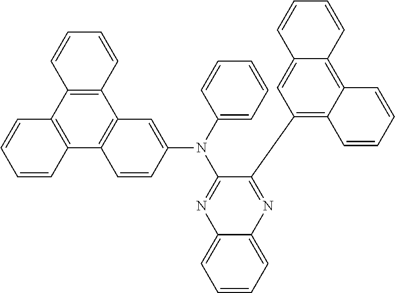 [SMILES: c1ccc(N(c2ccc3c4ccccc4c4ccccc4c3c2)c2nc3ccccc3nc2-c2cc3ccccc3c3ccccc23)cc1] | 71% |
| 15 | 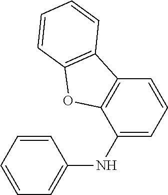 [SMILES: c1ccc(Nc2cccc3c2oc2ccccc23)cc1] | 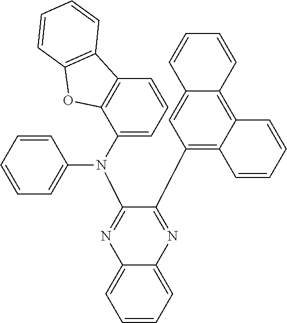 [SMILES: c1ccc(N(c2nc3ccccc3nc2-c2cc3ccccc3c3ccccc23)c2cccc3c2oc2ccccc23)cc1] | 84% |
| 45 | 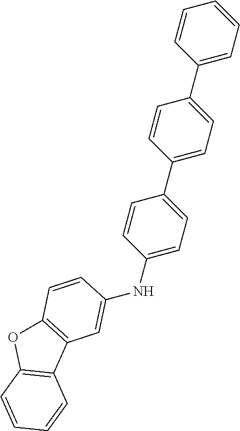 [SMILES: c1ccc(-c2ccc(-c3ccc(Nc4ccc5oc6ccccc6c5c4)cc3)cc2)cc1] | 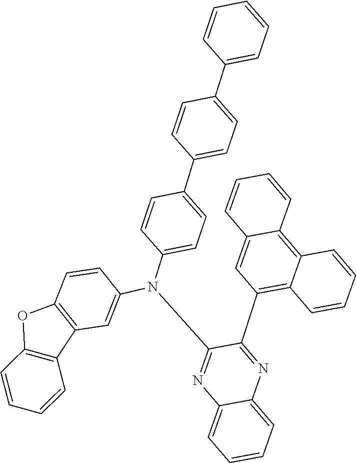 [SMILES: c1ccc(-c2ccc(-c3ccc(N(c4ccc5oc6ccccc6c5c4)c4nc5ccccc5nc4-c4cc5ccccc5c5ccccc45)cc3)cc2)cc1] | 82% |
| 221 | 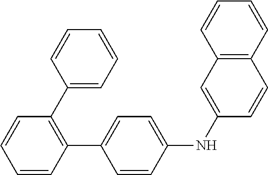 [SMILES: c1ccc(-c2ccccc2-c2ccc(Nc3ccc4ccccc4c3)cc2)cc1] | 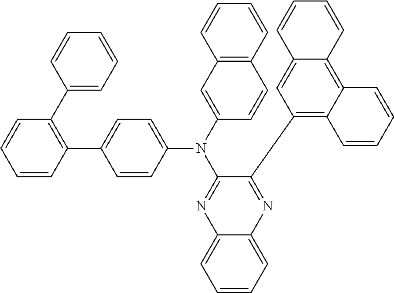 [SMILES: c1ccc(-c2ccccc2-c2ccc(N(c3ccc4ccccc4c3)c3nc4ccccc4nc3-c3cc4ccccc4c4ccccc34)cc2)cc1] | 79% |
| 226 | 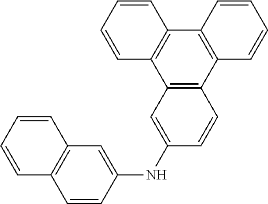 [SMILES: c1ccc2cc(Nc3ccc4c5ccccc5c5ccccc5c4c3)ccc2c1] | 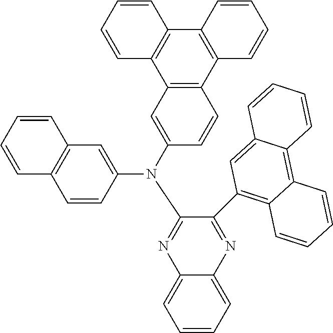 [SMILES: c1ccc2cc(N(c3ccc4c5ccccc5c5ccccc5c4c3)c3nc4ccccc4nc3-c3cc4ccccc4c4ccccc34)ccc2c1] | 88% |
| 233 | 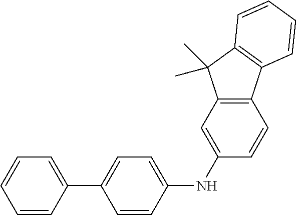 [SMILES: CC1(C)c2ccccc2-c2ccc(Nc3ccc(-c4ccccc4)cc3)cc21] | 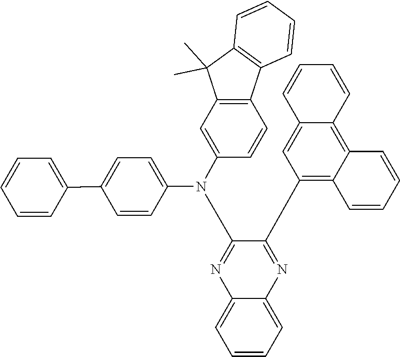 [SMILES: CC1(C)c2ccccc2-c2ccc(N(c3ccc(-c4ccccc4)cc3)c3nc4ccccc4nc3-c3cc4ccccc4c4ccccc34)cc21] | 72% |
| 244 | 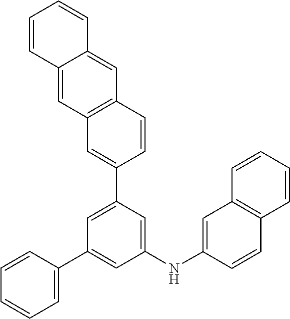 [SMILES: c1ccc(-c2cc(Nc3ccc4ccccc4c3)cc(-c3ccc4cc5ccccc5cc4c3)c2)cc1] | 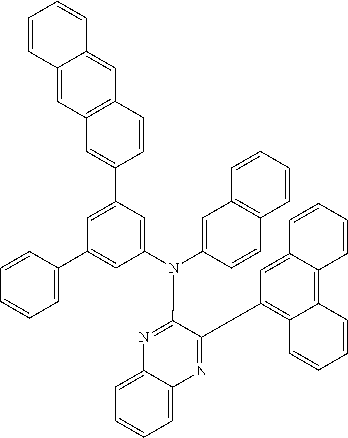 [SMILES: c1ccc(-c2cc(-c3ccc4cc5ccccc5cc4c3)cc(N(c3ccc4ccccc4c3)c3nc4ccccc4nc3-c3cc4ccccc4c4ccccc34)c2)cc1] | 71% |

### Preparation Example 2. Preparation of Compound 76

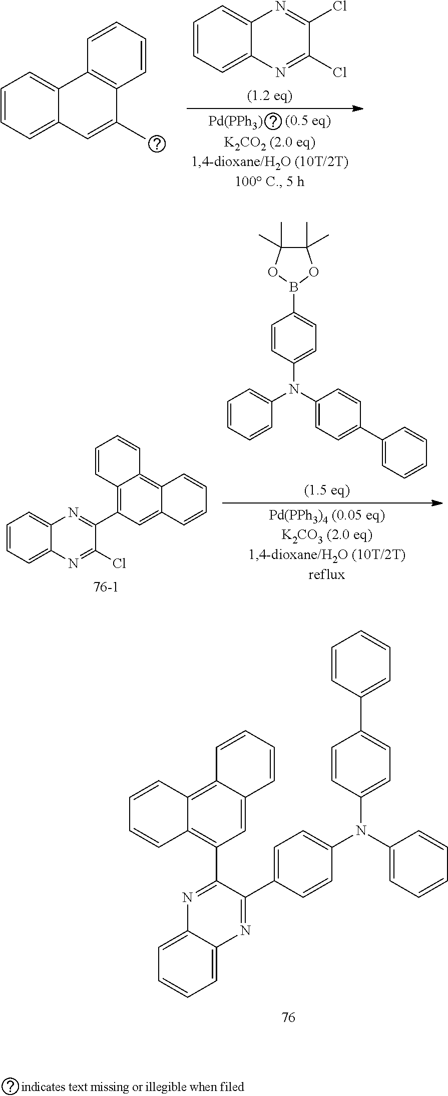

### Preparation Example 2-1. Preparation of Compound 76-1

Phenanthren-9-ylboronic acid (20 g, 90.07 mmol), 2,3-dichloroquinoxaline (21.73 g, 108.08 mmol), tetrakis(triphenylphosphine)palladium(0) (Pd(PPh₃)₄) (5.2 g, 4.5 mmol) and potassium carbonate (K₂CO₃) (31.12 g, 225.17 mmol) were dissolved in 1,4-dioxane (200 mL) and water (40 mL), and then the mixture was refluxed for 5 hours at 100° C.

After the reaction was completed, the result was cooled to room temperature, and then precipitated solids were filtered. The obtained solids were dissolved in dichloromethane (DCM), and after removing moisture with MgSO₄, the result was purified using a column to obtain Compound 76-1 (20 g, yield 65.2%).

### Preparation Example 2-2. Preparation of Compound 76

Compound 76-1 (5.1 g, 14.96 mmol), N-phenyl-N-(4-(4,4,5,5-tetramethyl-1,3,2-dioxaborolan-2-yl)phenyl)-[1,1′-biphenyl]-4-amine (7.83 g, 14.96 mmol), tetrakis(triphenylphosphine)palladium(0) (Pd(PPh₃)₄) (0.86 g, 0.75 mmol) and potassium carbonate (K₂CO₃) (4.14 g, 29.93 mmol) were dissolved in 1,4-dioxane (80 mL) and water (16 mL), and then the mixture was refluxed for 3 hours at 100° C.

After the reaction was completed, the result was cooled to room temperature, and then the solvent was removed under reduced pressure. The reaction material was purified by column chromatography (dichloromethane:hexane=1:3 (volume ratio)) to obtain Compound 76 (6.55 g, yield 70%).

Target compounds were prepared as in the following Table 2 in the same manner as in Preparation Example 2, except that, in preparing Compound 76, Intermediate B of the following Table 2 was used instead of N-phenyl-N-(4-(4,4,5,5-tetramethyl-1,3,2-dioxaborolan-2-yl)phenyl)-[1,1′-biphenyl]-4-amine.

TABLE 2

| Com- | | | |
|---|---|---|---|
| pound | | | |
| No. | Intermediate B | Target Compound | Yield |
| 94 | 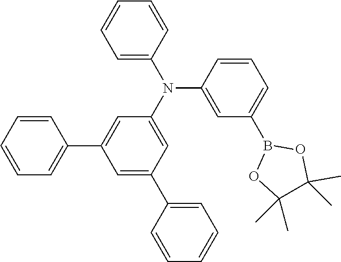 [SMILES: CC1(C)OB(c2cccc(N(c3ccccc3)c3cc(-c4ccccc4)cc(-c4ccccc4)c3)c2)OC1(C)C] | 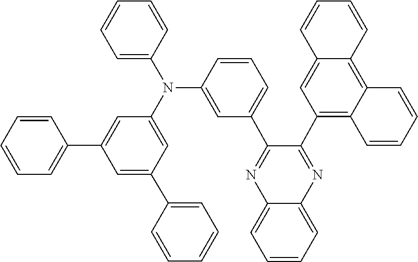 [SMILES: c1ccc(-c2cc(-c3ccccc3)cc(N(c3ccccc3)c3cccc(-c4nc5ccccc5nc4-c4cc5ccccc5c5ccccc45)c3)c2)cc1] | 70% |
| 140 | 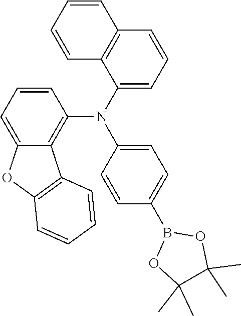 [SMILES: CC1(C)OB(c2ccc(N(c3cccc4ccccc34)c3cccc4oc5ccccc5c34)cc2)OC1(C)C] | 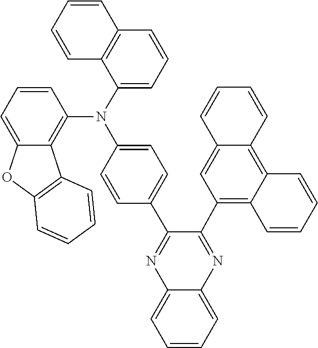 [SMILES: c1ccc2c(N(c3ccc(-c4nc5ccccc5nc4-c4cc5ccccc5c5ccccc45)cc3)c3cccc4oc5ccccc5c34)cccc2c1] | 82% |
| 156 | 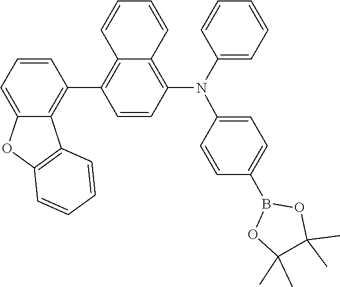 [SMILES: CC1(C)OB(c2ccc(N(c3ccccc3)c3ccc(-c4cccc5oc6ccccc6c45)c4ccccc34)cc2)OC1(C)C] | 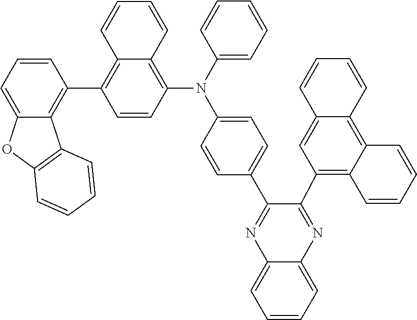 [SMILES: c1ccc(N(c2ccc(-c3nc4ccccc4nc3-c3cc4ccccc4c4ccccc34)cc2)c2ccc(-c3cccc4oc5ccccc5c34)c3ccccc23)cc1] | 88% |
| 157 | 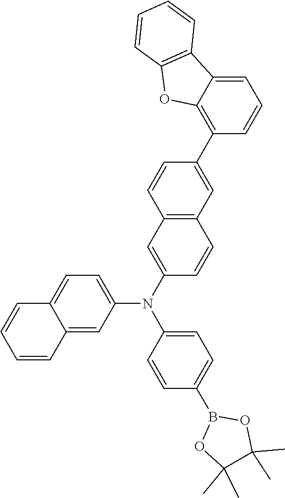 [SMILES: CC1(C)OB(c2ccc(N(c3ccc4ccccc4c3)c3ccc4cc(-c5cccc6c5oc5ccccc56)ccc4c3)cc2)OC1(C)C] | 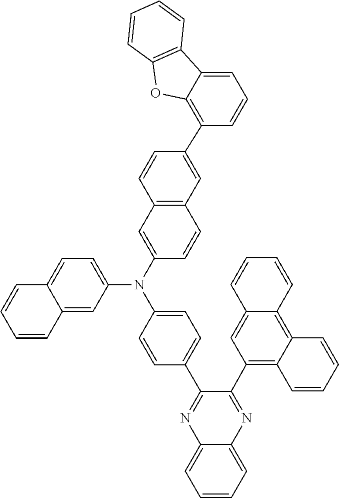 [SMILES: c1ccc2cc(N(c3ccc(-c4nc5ccccc5nc4-c4cc5ccccc5c5ccccc45)cc3)c3ccc4cc(-c5cccc6c5oc5ccccc56)ccc4c3)ccc2c1] | 77% |
| 281 | 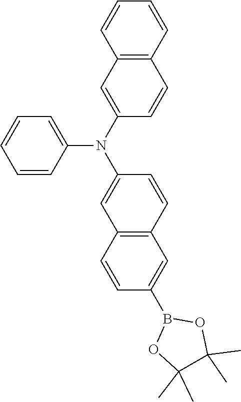 [SMILES: CC1(C)OB(c2ccc3cc(N(c4ccccc4)c4ccc5ccccc5c4)ccc3c2)OC1(C)C] | 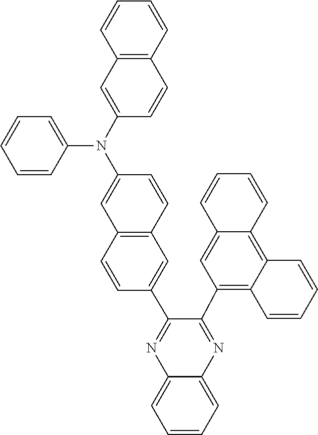 [SMILES: c1ccc(N(c2ccc3ccccc3c2)c2ccc3cc(-c4nc5ccccc5nc4-c4cc5ccccc5c5ccccc45)ccc3c2)cc1] | 71% |
| 289 | 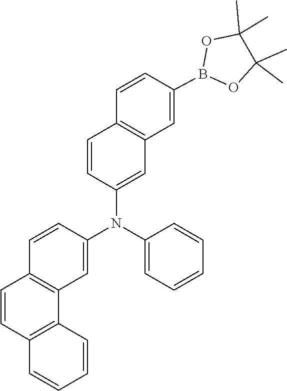 [SMILES: CC1(C)OB(c2ccc3ccc(N(c4ccccc4)c4ccc5ccc6ccccc6c5c4)cc3c2)OC1(C)C] | 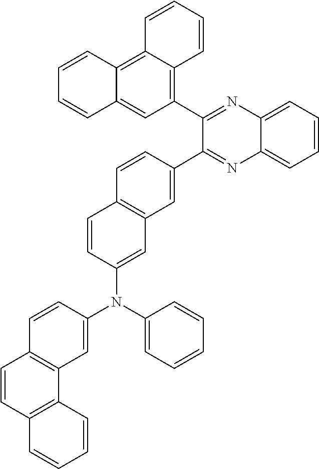 [SMILES: c1ccc(N(c2ccc3ccc(-c4nc5ccccc5nc4-c4cc5ccccc5c5ccccc45)cc3c2)c2ccc3ccc4ccccc4c3c2)cc1] | 81% |
| 292 | 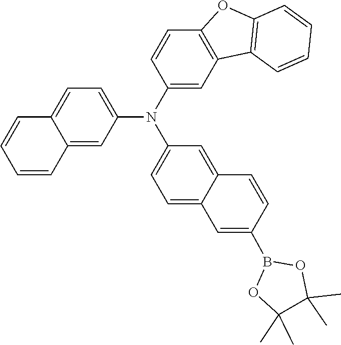 [SMILES: CC1(C)OB(c2ccc3cc(N(c4ccc5ccccc5c4)c4ccc5oc6ccccc6c5c4)ccc3c2)OC1(C)C] | 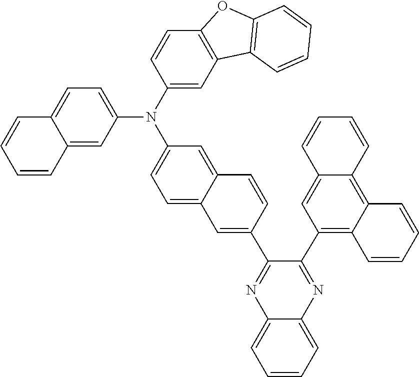 [SMILES: c1ccc2cc(N(c3ccc4cc(-c5nc6ccccc6nc5-c5cc6ccccc6c6ccccc56)ccc4c3)c3ccc4oc5ccccc5c4c3)ccc2c1] | 78% |
| 294 | 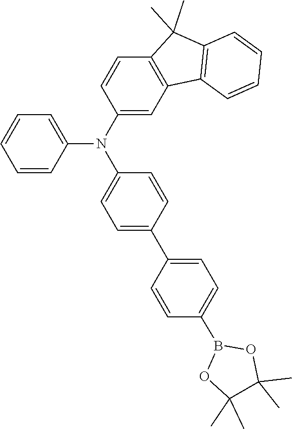 [SMILES: CC1(C)c2ccccc2-c2cc(N(c3ccccc3)c3ccc(-c4ccc(B5OC(C)(C)C(C)(C)O5)cc4)cc3)ccc21] | 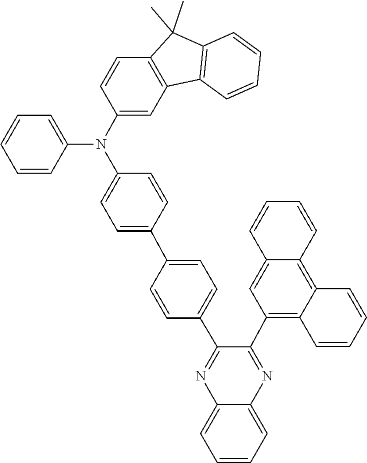 [SMILES: CC1(C)c2ccccc2-c2cc(N(c3ccccc3)c3ccc(-c4ccc(-c5nc6ccccc6nc5-c5cc6ccccc6c6ccccc56)cc4)cc3)ccc21] | 83% |
| 296 | 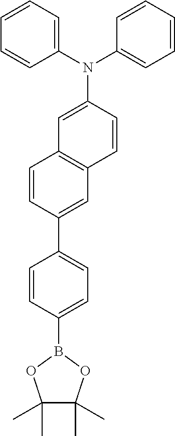 [SMILES: CC1(C)OB(c2ccc(-c3ccc4cc(N(c5ccccc5)c5ccccc5)ccc4c3)cc2)OC1(C)C] | 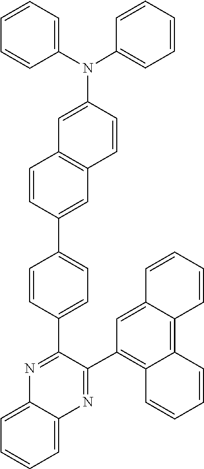 [SMILES: c1ccc(N(c2ccccc2)c2ccc3cc(-c4ccc(-c5nc6ccccc6nc5-c5cc6ccccc6c6ccccc56)cc4)ccc3c2)cc1] | 74% |
| 298 | 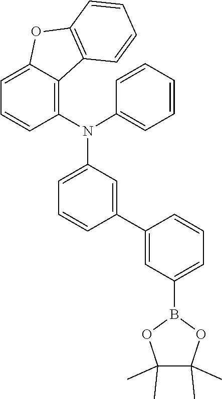 [SMILES: CC1(C)OB(c2cccc(-c3cccc(N(c4ccccc4)c4cccc5oc6ccccc6c45)c3)c2)OC1(C)C] | 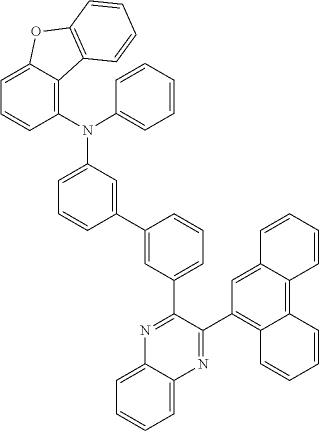 [SMILES: c1ccc(N(c2cccc(-c3cccc(-c4nc5ccccc5nc4-c4cc5ccccc5c5ccccc45)c3)c2)c2cccc3oc4ccccc4c23)cc1] | 79% |

### Preparation Example 3. Preparation of Compound 309

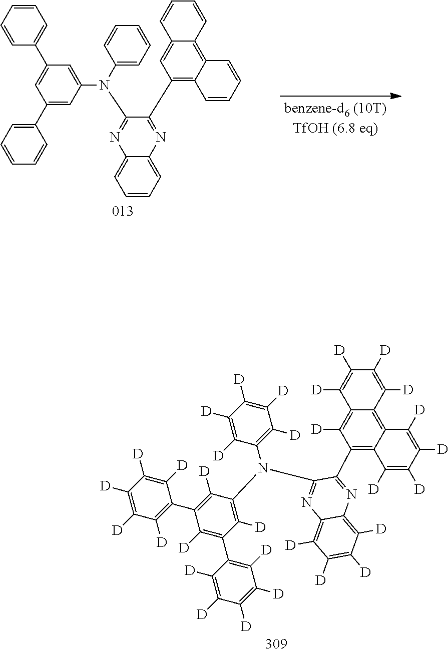 [SMILES: c1ccc(-c2cc(-c3ccccc3)cc(N(c3ccccc3)c3nc4ccccc4nc3-c3cc4ccccc4c4ccccc34)c2)cc1 ; [2H]c1c([2H])c([2H])c(-c2c([2H])c(-c3c([2H])c([2H])c([2H])c([2H])c3[2H])c([2H])c(N(c3nc4c([2H])c([2H])c([2H])c([2H])c4nc3-c3c([2H])c4c([2H])c([2H])c([2H])c([2H])c4c4c([2H])c([2H])c([2H])c([2H])c34)c3c([2H])c([2H])c([2H])c([2H])c3[2H])c2[2H])c([2H])c1[2H]]

Compound 13 (10 g, 15.98 mmol) was dissolved in benzene-d₆ (100 mL), and after adjusting the temperature of the mixture to 0° C. using an ice bath, trifluoromethanesulfonic acid (10 mL, 114.02 mmol) was slowly added dropwise thereto. After that, the mixture was stirred for 1 hour at a temperature of 60° C.

After the reaction was completed, the mixture solution was cooled to room temperature, and an ice bath was installed. After that, the mixture solution was neutralized using an aqueous K₃PO₄ solution, and solids were precipitated using methanol and filtered to obtain Compound 309 (7.35 g, yield 70%).

Target compounds were prepared as in the following Table 3 in the same manner as in Preparation Example 3, except that, in preparing Compound 309, Intermediate C of the following Table 3 was used instead of Compound 13.

TABLE 3

| Com- | | | |
|---|---|---|---|
| pound | | | |
| No. | Intermediate C | Target Compound | Yield |
| 310 | 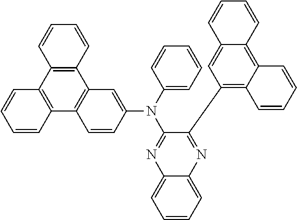 [SMILES: c1ccc(N(c2ccc3c4ccccc4c4ccccc4c3c2)c2nc3ccccc3nc2-c2cc3ccccc3c3ccccc23)cc1] | 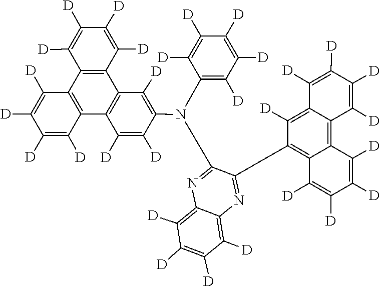 [SMILES: [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c3c4c([2H])c([2H])c([2H])c([2H])c4c4c([2H])c([2H])c([2H])c([2H])c4c3c2[2H])c([2H])c1[2H]] | 73% |
| 311 | 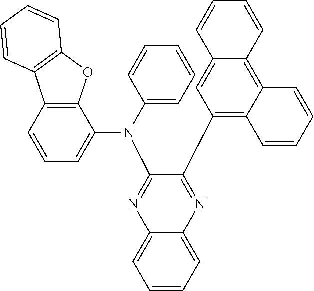 [SMILES: c1ccc(N(c2nc3ccccc3nc2-c2cc3ccccc3c3ccccc23)c2cccc3c2oc2ccccc23)cc1] | 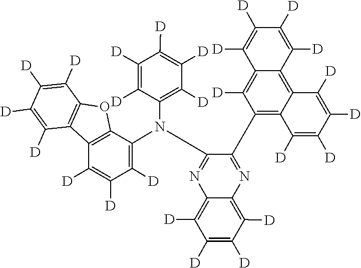 [SMILES: [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c([2H])c3c2oc2c([2H])c([2H])c([2H])c([2H])c23)c([2H])c1[2H]] | 72% |
| 312 | 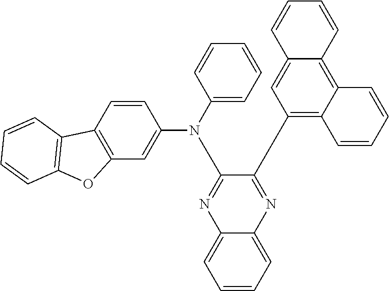 [SMILES: c1ccc(N(c2ccc3c(c2)oc2ccccc23)c2nc3ccccc3nc2-c2cc3ccccc3c3ccccc23)cc1] | 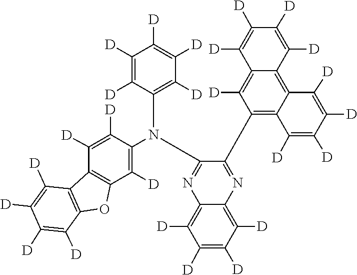 [SMILES: [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c3c(oc4c([2H])c([2H])c([2H])c([2H])c43)c2[2H])c([2H])c1[2H]] | 82% |
| 313 | 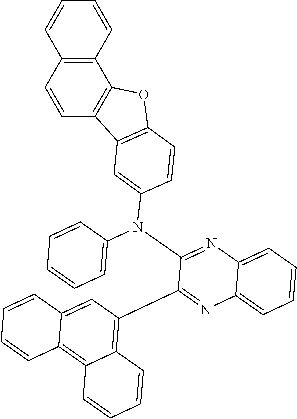 [SMILES: c1ccc(N(c2ccc3oc4c5ccccc5ccc4c3c2)c2nc3ccccc3nc2-c2cc3ccccc3c3ccccc23)cc1] | 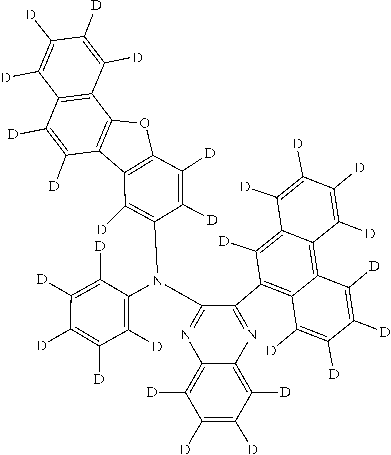 [SMILES: [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c3oc4c5c([2H])c([2H])c([2H])c([2H])c5c([2H])c([2H])c4c3c2[2H])c([2H])c1[2H]] | 80% |
| 315 | 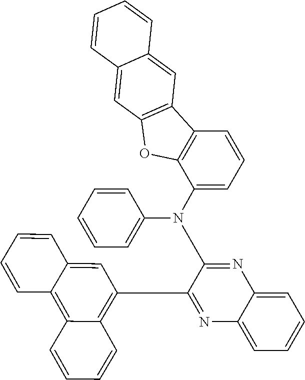 [SMILES: c1ccc(N(c2nc3ccccc3nc2-c2cc3ccccc3c3ccccc23)c2cccc3c2oc2cc4ccccc4cc23)cc1] | 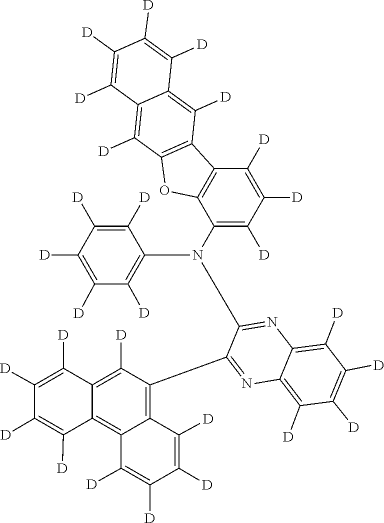 [SMILES: [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c([2H])c3c2oc2c([2H])c4c([2H])c([2H])c([2H])c([2H])c4c([2H])c23)c([2H])c1[2H]] | 78% |
| 317 | 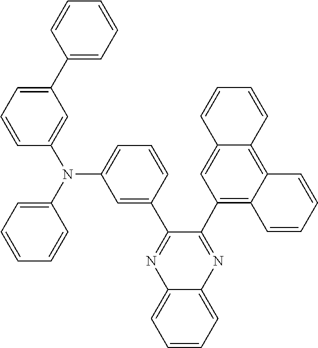 [SMILES: c1ccc(-c2cccc(N(c3ccccc3)c3cccc(-c4nc5ccccc5nc4-c4cc5ccccc5c5ccccc45)c3)c2)cc1] | 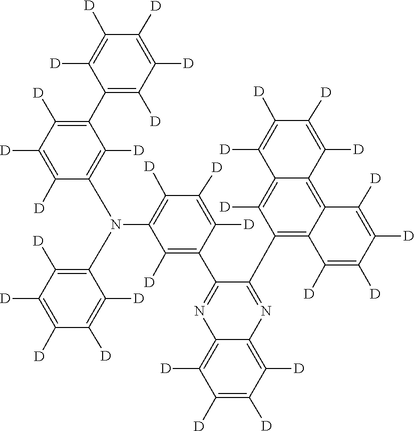 [SMILES: [2H]c1c([2H])c([2H])c(-c2c([2H])c([2H])c([2H])c(N(c3c([2H])c([2H])c([2H])c([2H])c3[2H])c3c([2H])c([2H])c([2H])c(-c4nc5c([2H])c([2H])c([2H])c([2H])c5nc4-c4c([2H])c5c([2H])c([2H])c([2H])c([2H])c5c5c([2H])c([2H])c([2H])c([2H])c45)c3[2H])c2[2H])c([2H])c1[2H]] | 72% |
| 318 | 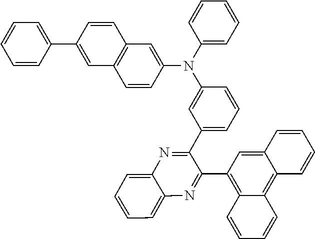 [SMILES: c1ccc(-c2ccc3cc(N(c4ccccc4)c4cccc(-c5nc6ccccc6nc5-c5cc6ccccc6c6ccccc56)c4)ccc3c2)cc1] | 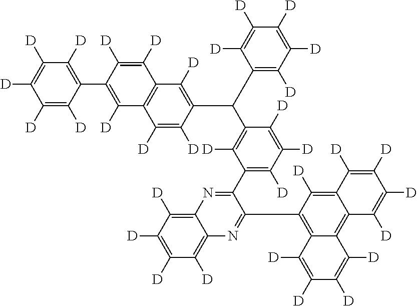 [SMILES: [2H]c1c([2H])c([2H])c(-c2c([2H])c([2H])c3c([2H])c(C(c4c([2H])c([2H])c([2H])c([2H])c4[2H])c4c([2H])c([2H])c([2H])c(-c5nc6c([2H])c([2H])c([2H])c([2H])c6nc5-c5c([2H])c6c([2H])c([2H])c([2H])c([2H])c6c6c([2H])c([2H])c([2H])c([2H])c56)c4[2H])c([2H])c([2H])c3c2[2H])c([2H])c1[2H]] | 85% |
| 319 | 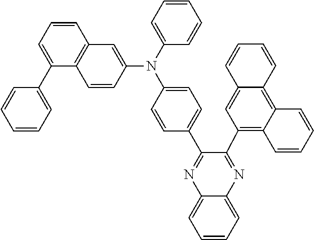 [SMILES: c1ccc(-c2cccc3cc(N(c4ccccc4)c4ccc(-c5nc6ccccc6nc5-c5cc6ccccc6c6ccccc56)cc4)ccc23)cc1] | 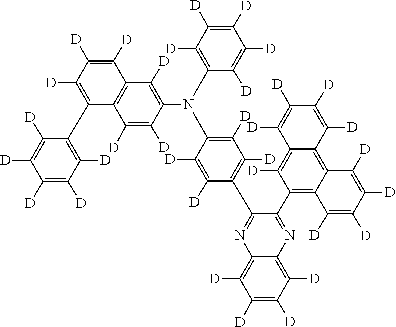 [SMILES: [2H]c1c([2H])c([2H])c(-c2c([2H])c([2H])c([2H])c3c([2H])c(N(c4c([2H])c([2H])c([2H])c([2H])c4[2H])c4c([2H])c([2H])c(-c5nc6c([2H])c([2H])c([2H])c([2H])c6nc5-c5c([2H])c6c([2H])c([2H])c([2H])c([2H])c6c6c([2H])c([2H])c([2H])c([2H])c56)c([2H])c4[2H])c([2H])c([2H])c23)c([2H])c1[2H]] | 74% |
| 320 | 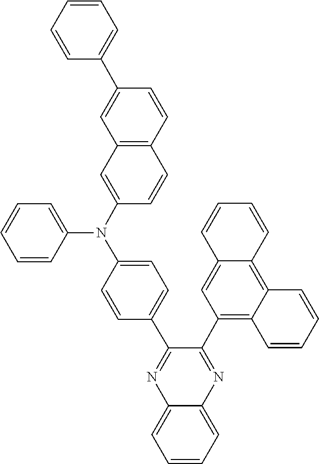 [SMILES: c1ccc(-c2ccc3ccc(N(c4ccccc4)c4ccc(-c5nc6ccccc6nc5-c5cc6ccccc6c6ccccc56)cc4)cc3c2)cc1] | 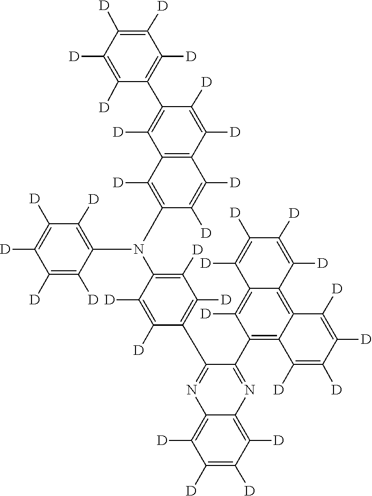 [SMILES: [2H]c1c([2H])c([2H])c(-c2c([2H])c([2H])c3c([2H])c([2H])c(N(c4c([2H])c([2H])c([2H])c([2H])c4[2H])c4c([2H])c([2H])c(-c5nc6c([2H])c([2H])c([2H])c([2H])c6nc5-c5c([2H])c6c([2H])c([2H])c([2H])c([2H])c6c6c([2H])c([2H])c([2H])c([2H])c56)c([2H])c4[2H])c([2H])c3c2[2H])c([2H])c1[2H]] | 71% |

### Preparation Example 4. Preparation of Compound 304

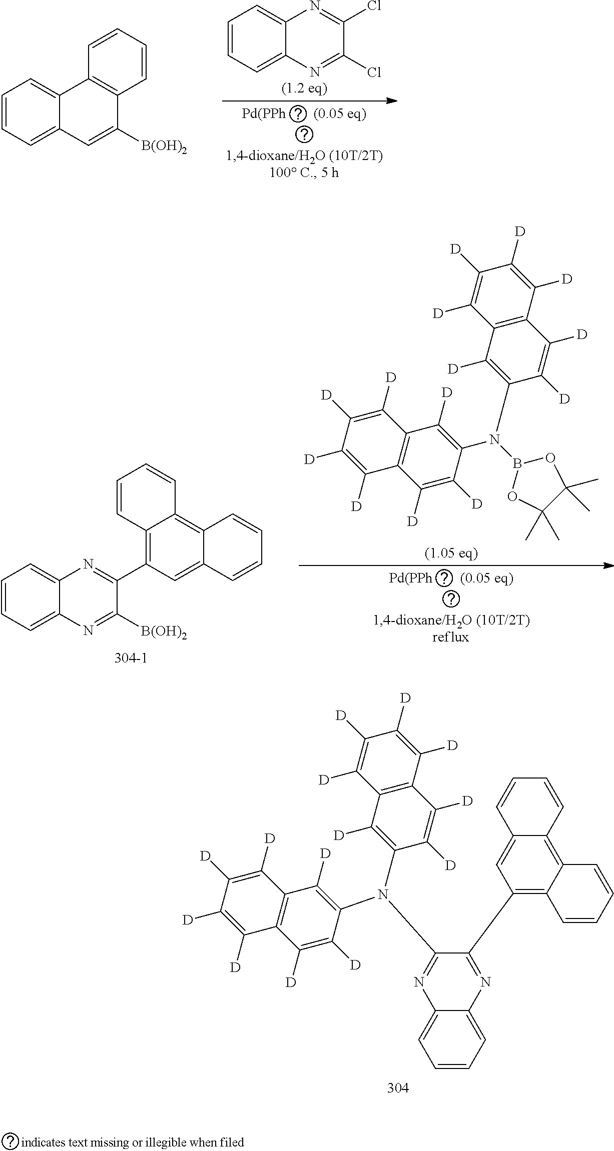 [SMILES: OB(O)c1cc2ccccc2c2ccccc12 ; Clc1nc2ccccc2nc1Cl ; OB(O)c1nc2ccccc2nc1-c1cc2ccccc2c2ccccc12 ; [2H]c1c([2H])c([2H])c2c([2H])c(N(B3OC(C)(C)C(C)(C)O3)c3c([2H])c([2H])c4c([2H])c([2H])c([2H])c([2H])c4c3[2H])c([2H])c([2H])c2c1[2H] ; [2H]c1c([2H])c([2H])c2c([2H])c(N(c3nc4ccccc4nc3-c3cc4ccccc4c4ccccc34)c3c([2H])c([2H])c4c([2H])c([2H])c([2H])c([2H])c4c3[2H])c([2H])c([2H])c2c1[2H]]

### Preparation Example 4-1. Preparation of Compound 304-1

Phenanthren-9-ylboronic acid (20 g, 90.07 mmol), 2,3-dichloroquinoxaline (21.73 g, 108.08 mmol), tetrakis(triphenylphosphine)palladium(0) (Pd(PPh₃)₄) (5.2 g, 4.5 mmol) and potassium carbonate (K₂CO₃) (31.12 g, 225.17 mmol) were dissolved in 1,4-dioxane (200 mL) and water (40 mL), and then the mixture was refluxed for 5 hours at 100° C.

After the reaction was completed, the result was cooled to room temperature, and then precipitated solids were filtered. The obtained solids were dissolved in dichloromethane (DCM), and after removing moisture with MgSO₄, the result was purified using a column to obtain Compound 304-1 (20 g, yield 65.2%).

### Preparation Example 4-2. Preparation of Compound 304

Compound 304-1 (5.1 g, 14.96 mmol), 4,4,5,5-tetramethyl-N, N-di(naphthalen-2-yl)-1,3,2-dioxaborolan-2-amine-d9 (6.12 g, 14.96 mmol), tetrakis(triphenylphosphine)palladium(0) (Pd(PPh₃)₄) (0.86 g, 0.75 mmol) and potassium carbonate (K₂CO₃) (4.14 g, 29.93 mmol) were dissolved in 1,4-dioxane (80 mL) and water (16 mL), and then the mixture was refluxed for 3 hours at 100° C.

After the reaction was completed, the result was cooled to room temperature, and then the solvent was removed under reduced pressure. The reaction material was purified by column chromatography (dichloromethane:hexane=1:3 (volume ratio)) to obtain Compound 304 (6.55 g, yield 70%).

Target compounds were prepared as in the following Table 4 in the same manner as in Preparation Example 4, except that Intermediate D of the following Table 4 was used instead of phenanthren-9-ylboronic acid, Intermediate E of the following Table 4 was used instead of 2,3-dichloroquinoxaline, and Intermediate F of the following Table 4 was used instead of 4,4,5,5-tetramethyl-N,N-di(naphthalen-2-yl)-1,3,2-dioxaborolan-2-amine-d9.

TABLE 4

| Com- | | | |
|---|---|---|---|
| pound | Intermediate | Intermediate | Intermediate |
| No. | D | E | F |
| 308 | 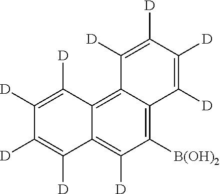 [SMILES: [2H]c1c([2H])c([2H])c2c(c1[2H])c([2H])c(B(O)O)c1c([2H])c([2H])c([2H])c([2H])c12] | 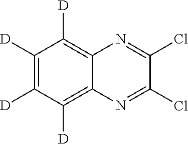 [SMILES: [2H]c1c([2H])c([2H])c2nc(Cl)c(Cl)nc2c1[2H]] | 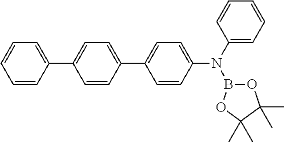 [SMILES: CC1(C)OB(N(c2ccccc2)c2ccc(-c3ccc(-c4ccccc4)cc3)cc2)OC1(C)C] |

| Com- | | |
|---|---|---|
| pound | | |
| No. | Target Compound | Yield |
| 308 | 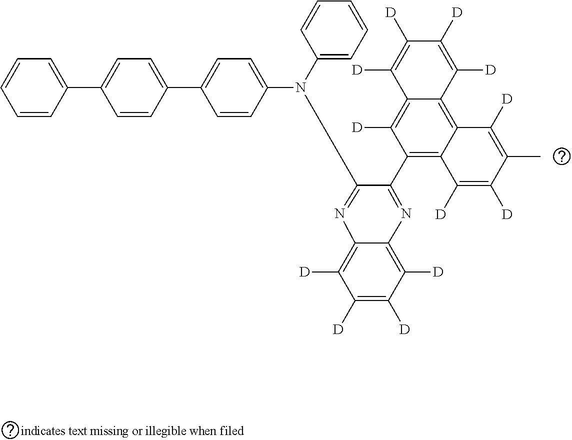 [SMILES: [2H]c1c([2H])c([2H])c2nc(N(c3ccccc3)c3ccc(-c4ccc(-c5ccccc5)cc4)cc3)c(-c3c([2H])c4c([2H])c([2H])c([2H])c([2H])c4c4c([2H])c(C)c([2H])c([2H])c34)nc2c1[2H]] | 88% |

Synthesis results for the compounds described in Preparation Examples 1 to 4 and Tables 1 to 4 are shown in the following Tables 5 and 6.

The following Table 5 shows measurement values of 1H NMR (CDCl₃, 400 MHz), and the following Table 6 shows measurement values of FD-mass spectrometry (FD-MS: field desorption mass spectrometry).

TABLE 5

| Com- | |
|---|---|
| pound | |
| No. | ¹H NMR (CDCl₃, 400 MHZ) |
| 5 | δ = 8.93 (d, 2H), 8.12 (d, 2H), 7.93 (m, 1H), 7.88 (t, 2H) |
| | 7.82 (m, 2H), 7.80 (m, 2H), 7.67 (t, 2H), 7.54 (m, 2H) |
| | 7.52 (m, 2H), 7.51 (m, 2H), 7.41 (m, 1H), 7.20 (t, 2H), |
| | 6.81 (m, 1H), 6.93 (d, 2H), 6.63 (d, 2H) |
| 8 | δ = 8.93 (d, 2H), 8.12 (d, 2H), 7.93 (m, 1H), 7.88 (t, 2H), |
| | 7.82 (m, 2H), 7.80 (m, 2H), 7.67 (t, 2H), 7.25 (s, 4H), |
| | 7.20 (t, 2H), 6.18 (t, 1H), 6.69 (d, 2H), 6.63 (d, 2H) |
| 13 | δ = 8.93 (d, 2H), 8.12 (d, 2H), 7.93 (m, 1H), 7.88 (t, 2H) |
| | 7.82 (m, 2H), 7.80 (m, 2H), 7.67(t, 2H), 7.52 (t, 4H), |
| | 7.51 (t, 4H), 7.41 (m, 2H), 7.20 (t, 2H), 7.06 (t, 1H), |
| | 6.85 (s, 2H), 6.82 (s, 1H), 6.63 (d, 2H) |
| 14 | δ = 8.93 (d, 4H), 8.12 (d, 4H), 7.93 (m, 1H), 7.88 (m, |
| | 4H), 7.82 (m, 4H), 7.80 (m 2H), 7.67 (t, 2H), 7,20 (t, |
| | 2H), 7.02 (d, 1H), 6.81 (t, 1H), 6.63 (d, 2H) |
| 15 | δ = 8.93 (d, 2H), 8.12 (d, 2H), 7.93 (m, 1H), 7.89 (s, |
| | 1H), 7.88 (t, 2H) 7.82 (m, 2H), 7.80 (m, 2H), 7.67 (t, |
| | 2H), 7.38 (s, 1H), 7.32 (m, 1H), 7.20 (m, 2H), 7.07 (t, |
| | 1H), 6.81 (t, 1H), 6.63 (d, 2H), 6.39 (d, 2H) |
| 45 | δ = 8.93 (d, 2H), 8.12 (d, 2H), 7.93 (m, 1H), 7.89 (s, |
| | 1H), 7.88 (t, 2H) 7.82 (m, 2H), 7.80 (m, 2H), 7.67 (t, |
| | 2H), 7.66 (m, 1H), 7.65 (m, 1H), 7.54 (t, 2H) 7.52 (t, |
| | 2H), 7.51 (m, 2H), 7.41 (m, 2H), 7.38 (m, 1H), 7.32 |
| | (m, 1H), 7.25 (s, 4H), 6.69 (d, 2H), 6.39 (d, 1H) |
| 76 | δ = 8.93 (d, 2H), 8.12 (d, 2H), 7.93 (s, 1H), 7.88 (m, |
| | 2H), 7.82 (m, 12), 7.80 (m, 2H), 7.67 (m, 2H), 7.54 |
| | (m, 4H), 7.52 (m, 2H), 7.51 (m, 2H), 7.41 (m, 1H), |
| | 7.20 (t, 2H), 6.81 (t, 1H), 6.69 (d, 4H), 6.63 (d, 2H) |
| 94 | δ = 8.93 (d, 2H), 8.12 (d, 2H), 7.93 (m, 1H), 7.88 (t, |
| | 2H) 7.82 (m, 2H), 7.80 (m, 2H), 7.67 (t, 2H), 7.52 |
| | (t, 4H), 7.51 (t, 4H), 7.44 (m, 1H), 7.41 (m, 2H), |
| | 7.20 (m, 2H), 7.15 (m, 1H), 7.06 (s, 1H), 6.89 (s, |
| | 1H), 6.85 (s, 2H), 6.81 (m, 1H), 6.63 (m, 2H), 6.59 |
| | (m, 1H) |
| 140 | δ = 8.93 (d, 2H), 8.12 (d, 2H), 8.02 (m, 2H), 7.89 (m, |
| | 1H), 7.88 (m, 2H), 7.82 (m, 2H), 7.80 (m, 2H), 7.67 |
| | (m, 2H), 7.66 (m, 1H), 7.57 (m, 1H), 7.54 (m, 2H), |
| | 7.53 (m, 2H), 7.38 (m, 2H), 7.32 (m, 1H), 7.13 (t, |
| | 1H), 7.02 (m, 1H), 6.98 (m, 1H), 6.69 (d, 2H), 6.33 |
| | (d, 1H) |
| 156 | δ = 8.93 (d, 2H), 8.49 (m, 1H), 8.12 (d, 2H), 8.07 (m, |
| | 1H), 7.89 (m, 1H), 7.88 (m, 2H), 7.82 (m, 2H), 7.80 |
| | (m, 2H), 7.78 (m, 1H), 7.75 (m, 1H), 7.67 (m, 2H), |
| | 7.66 (m, 1H), 7.62 (m, 1H), 7.54 (t, 3H), 7.53 (t, 1H), |
| | 7.44 (t, 1H), 7.38 (t, 1H), 7.32 (t, 1H), 7.20 (t, 2H), |
| | 7.04 (d, 1H), 6.81 (t, 1H), 6.69 (d, 2H), 6.63 (d, 2H) |
| 157 | δ = 8.93 (d, 2H), 8.12 (d, 2H), 7.89 (m, 1H), 7.88 (m, |
| | 3H), 7.85 (m, 1H) 7.84 (m, 2H), 7.82 (m, 2H), 7.81 |
| | (m, 1H), 7.80 (m, 2H), 7.77 (m, 1H), 7.74 (m, 2H), |
| | 7.69 (m, 1H), 7.67 (m, 2H), 7.66 (m, 1H), 7.64 (m, |
| | 1H), 7.54 (t, 2H), 7.50 (m, 1H), 7.49 (m, 2H), 7.46 |
| | (d, 1H), 7.38 (m, 2H), 7.36 (m, 1H), 7.32(m, 1H), |
| | 6.69 (d, 2H) |
| 221 | δ = 8.93 (d, 2H), 8.12 (d, 2H), 7.93 (s, 1H), 7.88 (m, |
| | 3H), 7.85 (m, 2H), 7.84 (m, 1H), 7.82 (m, 2H), 7.79 |
| | (m, 2H), 7.77 (m, 1H), 7.74 (m, 1H), 7.67 (m, 2H), |
| | 7.54 (m, 2h), 7.51 (m, 2H), 7.50 (m, 1H), 7.49 (m, |
| | 1H), 7.47 (m, 2H), 7.41 (t, 1H), 7.36 (t, 1H), 6.69 |
| | (d, 2H) |
| 226 | δ = 8.93 (d, 3H), 8.13 (d, 1H), 8.12 (d, 4H), 7.93 (s, |
| | 1H), 7.88 (m, 5H), 7.87 (m, 1H), 7.84 (m, 1H), 7.82 |
| | (m, 4H), 7.80 (m, 2H), 7.77 (m, 1H), 7.74 (m, 1H), |
| | 7.67 (m, 2H), 7.49 (t, 2H), .36 (t, 1H), 7.02 (d, 1H) |
| 233 | δ = 8.93 (d, 2H), 8.12 (d, 2H), 7.93 (s, 1H), 7.88 (m, |
| | 2H), 7.82 (m, 2H), 7.80 (m, 2H), 7.67 (m, 2H), 7.62 |
| | (S, 1H), 7.55 (m, 1H), 7.54 (m, 2H), 7.52 (m, 2H), |
| | 7.50 (m, 2H), 7.41 (m, 1H), 7.38 (m, 1H), 7.28 (t, |
| | 1H), 6.75 (s, 1H), 6.69 (d, 2H), 6.58 (d, 1H), 1.72 |
| | (s, 6H) |
| 244 | δ = 8.93 (d, 2H), 8.31 (s, 2H), 8.13 (d, 1H), 8.12 (d, |
| | 2H), 7.97 (d, 1H), 7.93 (m, 1H), 7.91 (m, 2H), 7.88 |
| | (m, 2H), 7.84 (m, 1H), 7.82 (m, 2H), 7.80 (m, 2H), |
| | 7.77 (m, 1H), 7.74 (m, 1H), 7.67 (m, 2H), 7.52 |
| | (m, 2H), 7.51 (m, 2H), 7.50 (m, 1H), 7.49 (m, 1H), |
| | 7.41 (m, 1H), 7.39 (m, 2H), 7.06 (s, 1H), 6.85 (s, |
| | 2H) |
| 281 | δ = 8.93 (d, 2H), 8.22 (s, 1H), 8.12 (d, 2H), 7.93 |
| | (s, 1H), 7.88 (m, 3H), 7.84 (m, 2H), 7.82 (m, 2H), |
| | 7.80 (m, 2H), 7.78 (m, 1H), 7.77 (m, 1H), 7.74 (s, |
| | 2H), 7.69 (m, 1H), 7.67 (m, 2H), 7.50 (t, 1H), 7.49 |
| | (t, 2H), 7.36 (t, 1H), 7.20 (t, 2H), 6.81 (t, 1H), 6.63 |
| | (d, 2H) |
| 289 | δ = 8.93 (d, 2H), 8.12 (d, 3H), 7.88 (m, 2H), 7.87 |
| | (m, 1H), 7.84 (m, 1H), 7.82 (m, 3H) 7.80 (m, 3H), |
| | 7.74 (s, 1H), 7.71 (m, 2H), 7.67 (m, 2H), 7.49 (d, |
| | 1H), 7.20 (t, 2H), 7.02 (d, 1H), 6.81 (t, 1H), 6.63 |
| | (d, 2H) |
| 292 | δ = 8.93 (d, 2H), 8.22 (s, 1H), 8.12 (d, 2H), 7.93 |
| | (s, 1H), 7.89 (m, 1H), 7.88 (m, 3H), 7.84 (m, |
| | 2H), 7.82 (m, 2H), 7.80 (m, 2H), 7.78 (m, 1H), |
| | 7.77 (m, 1H), 7.74 (m, 2H), 7.69 (m, 1H) 7.67 |
| | (t, 2H), 7.66 (t, 1H), 7.65 (t, 1H), 7.50 (m, 1H), |
| | 7.49 (m, 2H), 7.41 (m, 1H), 7.38 (m, 1H), 7.36 |
| | (m, 1H) 7.32 (m, 1H), 6.39 (d, 1H) |
| 294 | δ = 8.93 (d, 2H), 8.30 (d, 2H), 8.12 (d, 2H), 7.93 |
| | (s, 1H), 7.88 (m, 2H), 7.85 (m, 2H), 7.82 (m, |
| | 2H), 7.80 (m, 2H), 7.67 (m, 2H), 7.55 (m, 1H), |
| | 7.54 (m, 2H), 7.38 (t, 1H), 7.30 (m, 1H), 7.28 |
| | (m, 1H), 7.20 (t, 2H), 7.04 (s, 1H), 6.81 (t, 1H), |
| | 6.69 (d, 2H), 6.63 (d, 2H), 6.48 (d, 1H), 1.72 |
| | (s, 6H) |
| 296 | δ = 8.93 (d, 2H), 8.30 (d, 2H), 8.12 (d, 2H), 7.93 |
| | (s, 1H), 7.88 (m, 2H), 7.85 (m, 2H), 7.84 (m, |
| | 1H), 7.82 (m, 2H), 7.80 (m, 2H), 7.74 (s, 1H), |
| | 7.69 (m, 1H), 7.67 (m, 2H), 7.64 (m, 1H), 7.49 |
| | (s, 1H), 7.46 (d, 1H), 7.20 (t, 4H), 6.81 (t, 2H), |
| | 6. 63 (d, 4H), |
| 298 | δ = 8.93 (d, 2H), 8.12 (d, 2H), 7.93 (s, 1H), 7.88 |
| | (m, 2H), 7.82 (m, 2H), 7.80 (m, 2H), 7.75 (m, |
| | 1H), 7. 70 (m, 1H), 7.67 (m, 2H), 7.66 (m, 1H), |
| | 7.57 (t, 1H), 7.48 (t, 1H) 7.44 (m, 1H), 7.38 |
| | (m, 1H), 7.20 (t, 2H), 7.13 (t, 1H), 7.02 (t, 1H), |
| | 6.89 (d, 1H), 6.88 (d, 1H), 6.81 (t, 1H), 6.63 |
| | (t, 2H), 6.59 (m, 1H), 6.33 (d, 1H) |
| 304 | δ = 8.93 (d, 2H), 8.12 (d, 2H), 7.93 (s, 1H), 7.88 |
| | (m, 2H), 7.82 (m, 2H), 7.80 (m, 2H), 7.67 (m, |
| 308 | δ = 7.54 (m, 2H), 7.52 (m, 2H), 7.51 (m, 2H), |
| | 2H) 7.41 (m, 1H), 7.25 (s, 4H), 7.20 (t, 2H), 6.81 |
| | (t, 1H), 6.69 (d, 2H), 6.63 (d, 2H) |

TABLE 6

| Compound | FD-Mass |
|---|---|
| 5 | m/z = 549.22 (C₄₀H₂₇N₃, 549.66) |
| 8 | m/z = 625.25 (C₄₆H₃₁N₃, 625.76) |
| 13 | m/z = 625.25 (C₄₆H₃₁N₃, 625.76) |
| 14 | m/z = 623.24 (C₄₆H₂₉N₃, 623.74) |
| 15 | m/z = 563.20 (C₄₀H₂₅N₃O, 563.65) |
| 45 | m/z = 715.26 (C₅₂H₃₃N₃O, 715.84) |
| 76 | m/z = 625.25 (C46H31N3, 625.76) |
| 94 | m/z = 701.28 (C₅₂H₃₅N₃, 701.85) |
| 140 | m/z = 689.25 (C₅₀H₃₁N₃O, 689.80) |
| 156 | m/z = 765.28 (C₅₆H₃₅N₃O, 765.90) |
| 157 | m/z = 815.29 (C₆₀H₃₇N₃O, 815.96) |
| 221 | m/z = 675.27 (C₅₀H₃₃N₃, 675.82) |
| 226 | m/z = 673.25 (C₅₀H₃₁N₃, 673.80) |
| 233 | m/z = 665.28 (C₄₉H₃₅N₃, 665.82) |
| 244 | m/z = 775.30 (C₅₈H₃₇N₃, 775.93) |
| 281 | m/z = 649.25 (C₄₈H₃₁N₃, 649.78) |
| 289 | m/z = 699.27 (C₅₂H₃₃N₃, 699.84) |
| 292 | m/z = 739.26 (C₅₄H₃₃N₃O, 739.86) |
| 294 | m/z = 741.31 (C₅₅H₃₉N₃, 741.92) |
| 296 | m/z = 675.27 (C₅₀H₃₃N₃, 675.82) |
| 298 | m/z = 715.26 (C₅₂H₃₃N₃O, 715.84) |
| 309 | m/z = 656.45 (C₄₆D₃₁N₃, 656.95) |
| 310 | m/z = 652.42 (C₄₆D₂₉N₃, 652.92) |
| 311 | m/z = 588.36 (C₄₀D₂₅N₃O, 588.80) |
| 312 | m/z = 588.36 (C₄₀D₂₅N₃O, 588.80) |
| 313 | m/z = 640.38 (C₄₄D₂₇N₃O, 640.87) |
| 315 | m/z = 640.38 (C₄₄D₂₇N₃O, 640.87) |
| 317 | m/z = 656.45 (C₄₆D₃₁N₃, 656.95) |
| 318 | m/z = 708.47 (C5₀D₃₃N₃, 709.02) |
| 319 | m/z = 708.47 (C₅₀D₃₃N₃, 709.02) |
| 320 | m/z = 708.47 (C₅₀D₃₃N₃, 709.02) |
| 304 | m/z = 587.31 (C₄₂H₁₃D₁₄N₃, 587.77) |
| 308 | m/z = 638.33 (C₄₆H₁₈D₁₃N₃, 638.84) |

### Preparation Example 5. Preparation of Compound 2-1

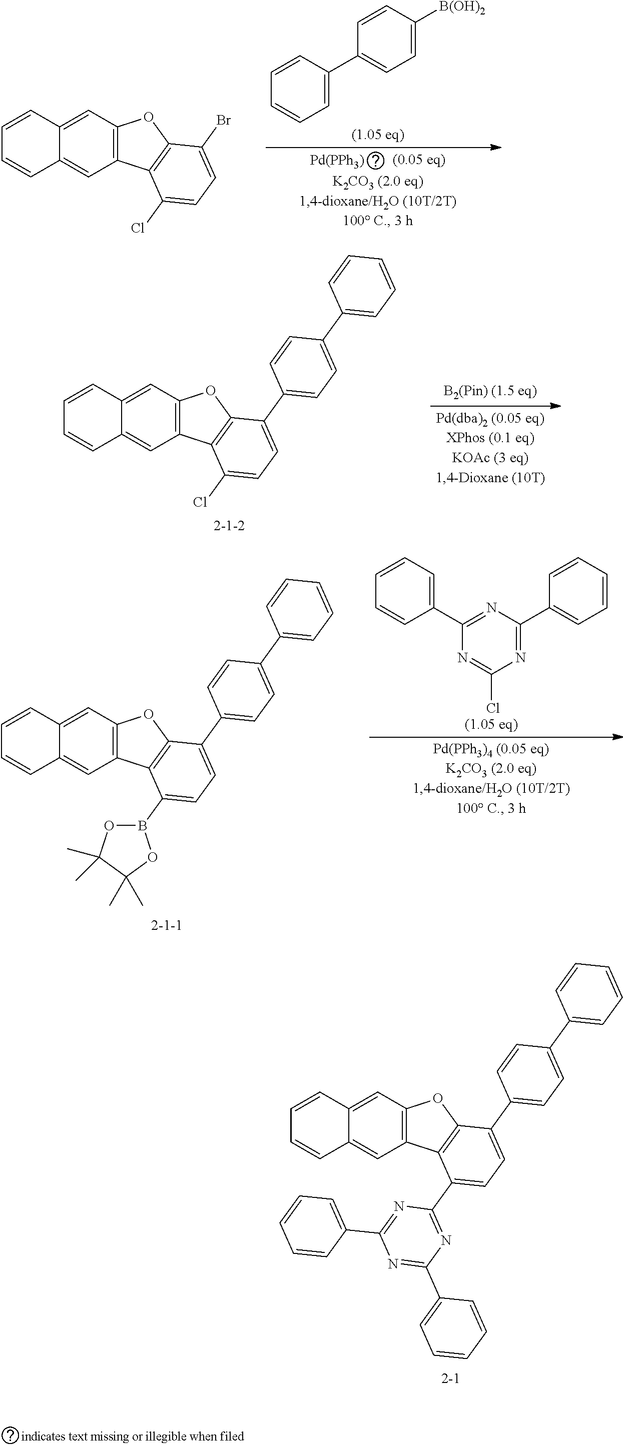

### Preparation Example 5-1. Preparation of Compound 2-1-2

4-Bromo-1-chloronaphtho[2,3-b]benzofuran (20 g, 60.62 mmol), [1,1′-biphenyl]-4-ylboronic acid (12.61 g, 63.65 mmol), tetrakis(triphenylphosphine)palladium(0) (Pd(PPh₃)₄) (3.5 g, 3.03 mmol) and potassium carbonate (K₂CO₃) (16.76 g, 121.24 mmol) were dissolved in 1,4-dioxane (200 mL) and water (40 mL), and then the mixture was refluxed for 3 hours at 100° C.

After the reaction was completed, the result was cooled to room temperature, and then precipitated solids were filtered. The obtained solids were dissolved in dichloromethane (DCM), and after removing moisture with MgSO₄, the result was purified using a column to obtain Compound 2-1-2 (21.8 g, yield 89%).

### Preparation Example 5-2. Preparation of Compound 2-1-1

Compound 2-1-2 (10 g, 24.75 mmol), bis(pinacolato)diboron (9.43 g, 37.12 mmol), bis(dibenzylideneacetone)palladium(0) (Pd(dba)₂) (0.71 g, 1.24 mmol), Xphos (1.18 g, 2.475 mmol) and potassium acetate (7.29 g, 74.25 mmol) were dissolved in 1,4-dioxane (100 mL), and then the mixture was refluxed for 3 hours at 100° C.

After the reaction was completed, the result was cooled to room temperature, and then the solvent was removed under reduced pressure. The reaction material was purified by column chromatography (dichloromethane:hexane=1:3 (volume ratio)) to obtain Compound 2-1-1 (8.35 g, yield 68%).

### Preparation Example 5-3. Preparation of Compound 2-1

Compound 2-1-1 (8 g, 16.12 mmol), 2-chloro-4,6-diphenyl-1,3,5-triazine (4.22 g, 15.80 mmol), tetrakis(triphenylphosphine)palladium(0) (Pd(PPh₃)₄) (0.93 g, 0.806 mmol) and potassium carbonate (K₂CO₃) (4.26 g, 48.36 mmol) were dissolved in 1,4-dioxane (80 mL) and water (16 mL), and then the mixture was refluxed for 3 hours at 100° C.

After the reaction was completed, the result was cooled to room temperature, and then precipitated solids were filtered. The obtained solids were dissolved in dichloromethane (DCM), and after removing moisture with MgSO₄, the result was purified using a column to obtain Compound 2-1 (7.66 g, yield 89%).

The following target compounds were synthesized in the same manner as in Preparation Example 5, except that Intermediate G of the following Table 7 was used instead of 4-bromo-1-chloronaphtho[2,3-b]benzofuran, Intermediate H of the following Table 7 was used instead of [1,1′-biphenyl]-4-ylboronic acid, and Intermediate I of the following Table 7 was used instead of 2-chloro-4,6-diphenyl-1,3,5-triazine.

TABLE 7

| Com- | | | | | |
|---|---|---|---|---|---|
| pound | Intermediate | Intermediate | Intermediate | | |
| No. | G | H | I | Target Compound | Yield |
| 2-2 | 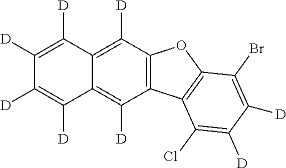 [SMILES: [2H]c1c([2H])c(Cl)c2c(oc3c([2H])c4c([2H])c([2H])c([2H])c([2H])c4c([2H])c32)c1Br] | 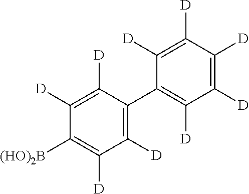 [SMILES: [2H]c1c([2H])c([2H])c(-c2c([2H])c([2H])c(B(O)O)c([2H])c2[2H])c([2H])c1[2H]] | 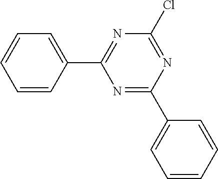 [SMILES: Clc1nc(-c2ccccc2)nc(-c2ccccc2)n1] | 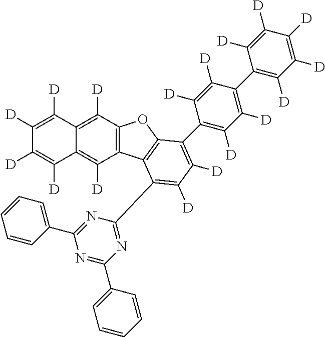 [SMILES: [2H]c1c([2H])c([2H])c(-c2c([2H])c([2H])c(-c3c([2H])c([2H])c(-c4nc(-c5ccccc5)nc(-c5ccccc5)n4)c4c3oc3c([2H])c5c([2H])c([2H])c([2H])c([2H])c5c([2H])c34)c([2H])c2[2H])c([2H])c1[2H]] | 82% |
| 2-12 | 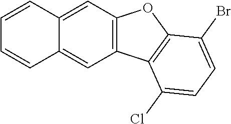 [SMILES: Clc1ccc(Br)c2oc3cc4ccccc4cc3c12] | 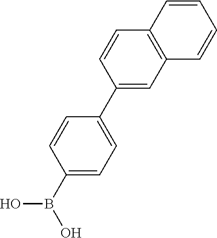 [SMILES: OB(O)c1ccc(-c2ccc3ccccc3c2)cc1] | 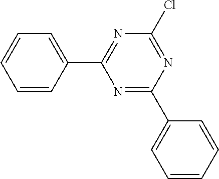 [SMILES: Clc1nc(-c2ccccc2)nc(-c2ccccc2)n1] | 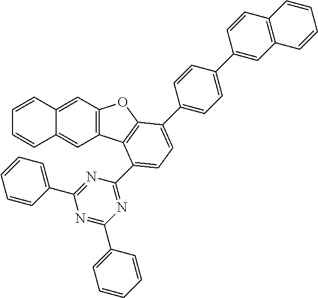 [SMILES: c1ccc(-c2nc(-c3ccccc3)nc(-c3ccc(-c4ccc(-c5ccc6ccccc6c5)cc4)c4oc5cc6ccccc6cc5c34)n2)cc1] | 80% |
| 2-18 | 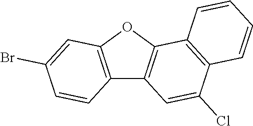 [SMILES: Clc1cc2c3ccc(Br)cc3oc2c2ccccc12] | 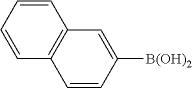 [SMILES: OB(O)c1ccc2ccccc2c1] | 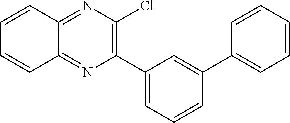 [SMILES: Clc1nc2ccccc2nc1-c1cccc(-c2ccccc2)c1] | 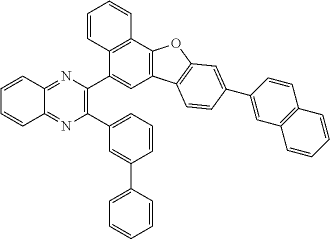 [SMILES: c1ccc(-c2cccc(-c3nc4ccccc4nc3-c3cc4c5ccc(-c6ccc7ccccc7c6)cc5oc4c4ccccc34)c2)cc1] | 88% |
| 2-20 | 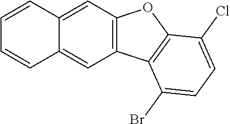 [SMILES: Clc1ccc(Br)c2c1oc1cc3ccccc3cc12] | 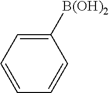 | 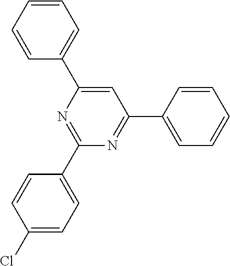 [SMILES: Clc1ccc(-c2nc(-c3ccccc3)cc(-c3ccccc3)n2)cc1] | 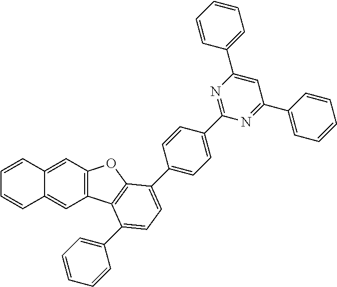 [SMILES: c1ccc(-c2cc(-c3ccccc3)nc(-c3ccc(-c4ccc(-c5ccccc5)c5c4oc4cc6ccccc6cc45)cc3)n2)cc1] | 81% |
| 2-21 | 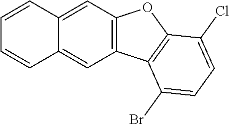 [SMILES: Clc1ccc(Br)c2c1oc1cc3ccccc3cc12] | 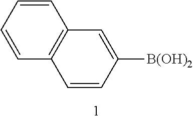 [SMILES: OB(O)c1ccc2ccccc2c1] | 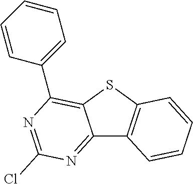 [SMILES: Clc1nc(-c2ccccc2)c2sc3ccccc3c2n1] | 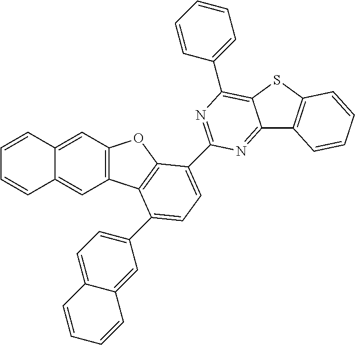 [SMILES: c1ccc(-c2nc(-c3ccc(-c4ccc5ccccc5c4)c4c3oc3cc5ccccc5cc34)nc3c2sc2ccccc23)cc1] | 78% |
| 2-36 | 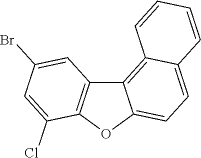 [SMILES: Clc1cc(Br)cc2c1oc1ccc3ccccc3c12] | 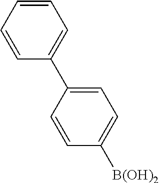 [SMILES: OB(O)c1ccc(-c2ccccc2)cc1] | 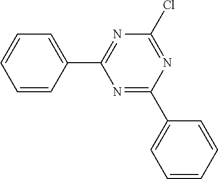 [SMILES: Clc1nc(-c2ccccc2)nc(-c2ccccc2)n1] | 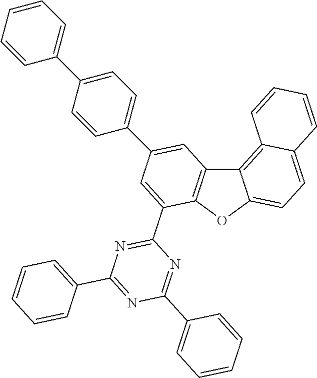 [SMILES: c1ccc(-c2ccc(-c3cc(-c4nc(-c5ccccc5)nc(-c5ccccc5)n4)c4oc5ccc6ccccc6c5c4c3)cc2)cc1] | 88% |
| 2-53 | 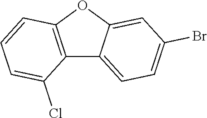 [SMILES: Clc1cccc2oc3cc(Br)ccc3c12] | 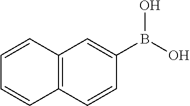 [SMILES: OB(O)c1ccc2ccccc2c1] | 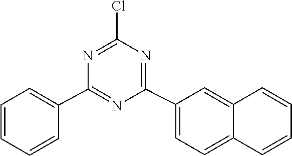 [SMILES: Clc1nc(-c2ccccc2)nc(-c2ccc3ccccc3c2)n1] | 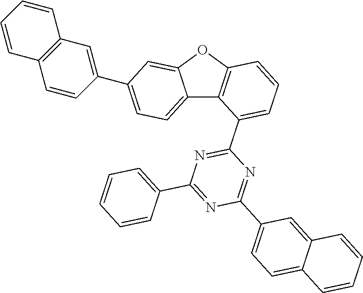 [SMILES: c1ccc(-c2nc(-c3ccc4ccccc4c3)nc(-c3cccc4oc5cc(-c6ccc7ccccc7c6)ccc5c34)n2)cc1] | 78% |
| 2-54 | 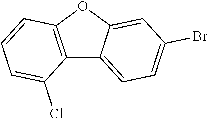 [SMILES: Clc1cccc2oc3cc(Br)ccc3c12] | 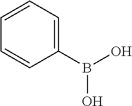 | 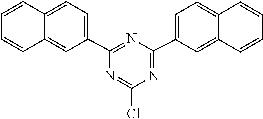 [SMILES: Clc1nc(-c2ccc3ccccc3c2)nc(-c2ccc3ccccc3c2)n1] | 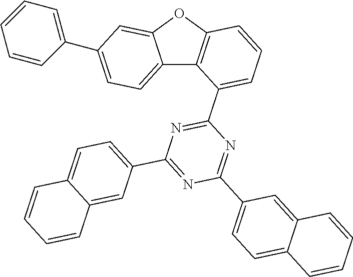 [SMILES: c1ccc(-c2ccc3c(c2)oc2cccc(-c4nc(-c5ccc6ccccc6c5)nc(-c5ccc6ccccc6c5)n4)c23)cc1] | 79% |
| 2-58 | 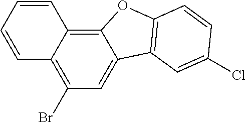 [SMILES: Clc1ccc2oc3c4ccccc4c(Br)cc3c2c1] | 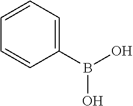 | 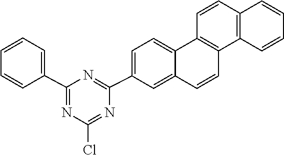 [SMILES: Clc1nc(-c2ccccc2)nc(-c2ccc3c(ccc4c5ccccc5ccc34)c2)n1] | 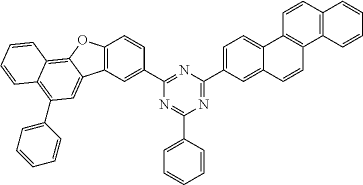 [SMILES: c1ccc(-c2nc(-c3ccc4c(ccc5c6ccccc6ccc45)c3)nc(-c3ccc4oc5c6ccccc6c(-c6ccccc6)cc5c4c3)n2)cc1] | 82% |
| 2-59 | 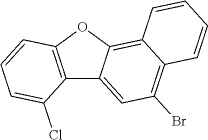 [SMILES: Clc1cccc2oc3c4ccccc4c(Br)cc3c12] | 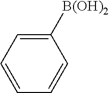 | 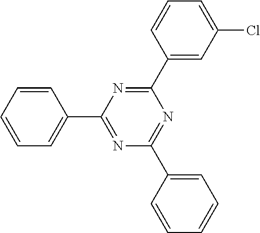 [SMILES: Clc1cccc(-c2nc(-c3ccccc3)nc(-c3ccccc3)n2)c1] | 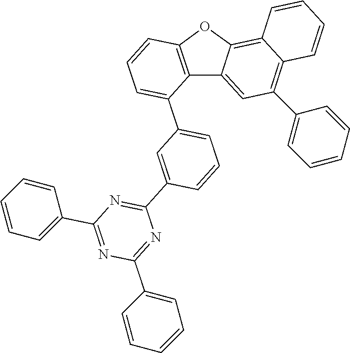 [SMILES: c1ccc(-c2nc(-c3ccccc3)nc(-c3cccc(-c4cccc5oc6c7ccccc7c(-c7ccccc7)cc6c45)c3)n2)cc1] | 86% |

### Preparation Example 6. Preparation of Compound 2-51

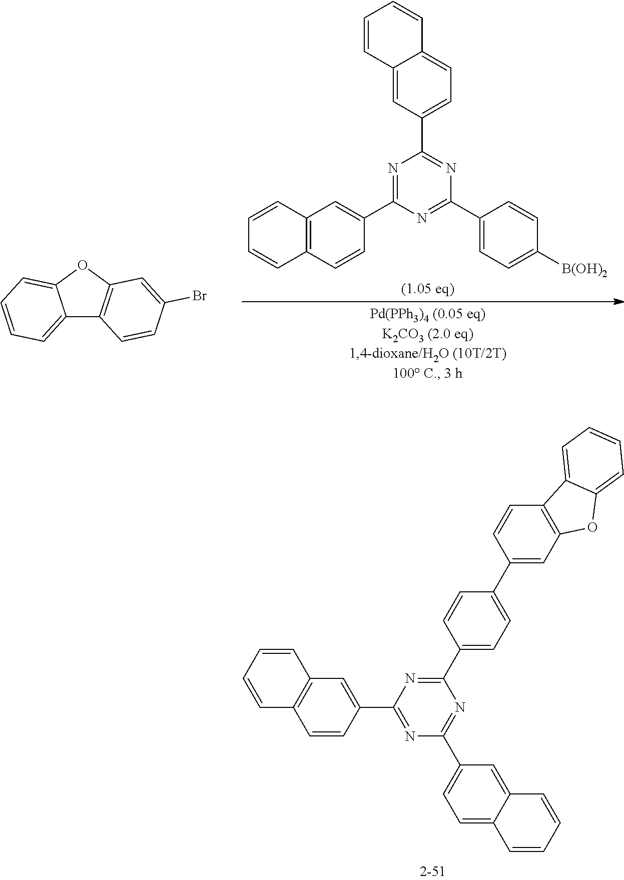 [SMILES: Brc1ccc2c(c1)oc1ccccc12 ; OB(O)c1ccc(-c2nc(-c3ccc4ccccc4c3)nc(-c3ccc4ccccc4c3)n2)cc1 ; c1ccc2cc(-c3nc(-c4ccc(-c5ccc6c(c5)oc5ccccc56)cc4)nc(-c4ccc5ccccc5c4)n3)ccc2c1]

3-Bromodibenzo[b,d]furan (8 g, 32.38 mmol), (4-(4,6-di(naphthalen-2-yl)-1,3,5-triazin-2-yl)phenyl)boronic acid (15.41 g, 63.65 mmol), tetrakis(triphenylphosphine)palladium(0) (Pd(PPh₃)₄) (1.87 g, 1.62 mmol) and potassium carbonate (K₂CO₃) (13.42 g, 97.14 mmol) were dissolved in 1,4-dioxane (80 mL) and water (16 mL), and then the mixture was refluxed for 3 hours at 100° C.

After the reaction was completed, the result was cooled to room temperature, and then precipitated solids were filtered. The obtained solids were dissolved in dichloromethane (DCM), and after removing moisture with MgSO₄, the result was purified using a column to obtain Compound 2-51 (16.78 g, yield 90%).

The following target compounds were synthesized in the same manner as in Preparation Example 6, except that Intermediate J of the following Table 8 was used instead of 3-bromodibenzo[b,d]furan, and Intermediate K of the following Table 8 was used instead of [1,1′-biphenyl]-4-ylboronic acid.

TABLE 8

| Com- | | | | |
|---|---|---|---|---|
| pound | Intermediate | | | |
| No. | J | Intermediate K | Target Compound | Yield |
| 2-47 | 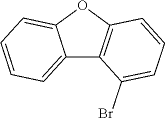 [SMILES: Brc1cccc2oc3ccccc3c12] | 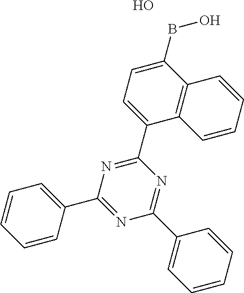 | 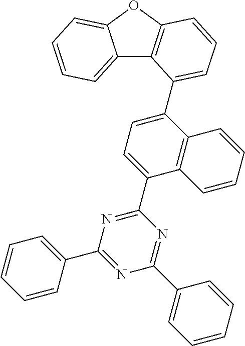 [SMILES: c1ccc(-c2nc(-c3ccccc3)nc(-c3ccc(-c4cccc5oc6ccccc6c45)c4ccccc34)n2)cc1] | 85% |
| 2-51 | 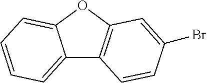 [SMILES: Brc1ccc2c(c1)oc1ccccc12] | 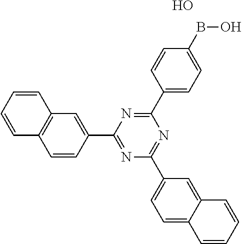 | 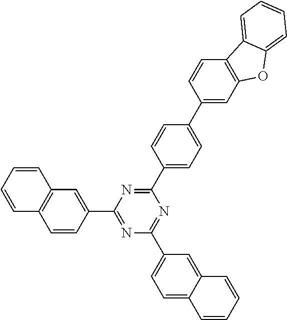 [SMILES: c1ccc2cc(-c3nc(-c4ccc(-c5ccc6c(c5)oc5ccccc56)cc4)nc(-c4ccc5ccccc5c4)n3)ccc2c1] | 90% |

Synthesis results for the compounds described in Preparation Example 5, Preparation Example 6, Table 7 and Table 8 are shown in the following Tables 9 and 10.

The following Table 9 shows measurement values of 1H NMR (CDCl₃, 400 MHz), and the following Table 10 shows measurement values of FD-mass spectrometry (FD-MS: field desorption mass spectrometry).

TABLE 9

| Com- | |
|---|---|
| pound | |
| No. | ¹H NMR (CDCl₃, 400 MHZ) |
| 2-2 | δ = 8.28 (d, 4H), 7.51 (t, 4H), 7.41 (m, 2H) |
| 2-12 | δ = 8.28 (d, 4H), 8.16 (m, 2H), 8.00 (m, 2H), |
| | 7.92 (d, \1H), 7.87 (d, 1H), 7.81 (d, 1H), |
| | 7.73 (d, 1H), 7.67 (m, 2H), 7.59 (m, 2H), |
| | 7.58 (m, 1H), 7.51 (d, 4H), 7.49 (d, 1H), |
| | 7.42 (m, 1H), 7.41 (m, 2H), 7.25 (s, 4H) |
| 2-18 | δ = 8.55 (m, 1H), 8.81 (m, 1H), 8.00 (m, 2H), |
| | 7.95 (t, 1H), 7.92 (d, 1H), 7.80 (m, 2H), 7.75 |
| | (d, 2H), 7.73 (s, 1H), 7. 71 (s, 1H), 7.70 (s, |
| | 1H), 7.67 (m, 2H), 7.64 (m, 1H), 7.59 (m, |
| | 2H), 7.58 (m, 1H), 7.55 (m, 2H), 7.52 (m, |
| | 2H), 7.51 (m, 2H), 7.41 (m, 1H) |
| 2-20 | δ = 8.23 (s, 1H), 8.16 (m, 2H), 7.87 (s, 1H), |
| | 7.85 (d, 2H), 7.81 (d, 1H), 7.79 (t, 6H), 7.67 |
| | (m, 2H), 7.51 (t, 6H) 7.41 (m, 3H), 7.25 (d, |
| | 2H) |
| 2-21 | δ = 8.16 (m, 2H), 8.05 (m, 1H), 8.00 (m, 2H), |
| | 7.98 (m, 1H), 7.92 (d, 1H), 7.87 (d, 1H), 7.81 |
| | (m, 1H), 7.79 (m, 2H), 7.67 (t, 2H), 7.59 (m, |
| | 2H), 7.51 (m, 2H), 7.50 (m, 1H), 7.42 (m, |
| | 1H), 7.41 (m, 1H) |
| 2-36 | δ = 8.54 (t, 1H), 8.28 (d, 4H), 8.16 (m, 1H), |
| | 7.67 (m, 3H), 7.66 (m, 1H), 7.59 (t, 1H), |
| | 7.52 (m, 2H), 7.51 (m, 6H), 7.41 (m, 1H), |
| | 7.25 (m, 4H) |
| 2-53 | δ = 9.09 (s, 1H), 8.49 (d, 1H), 8.28 (d, 2H), |
| | 8.00 (m, 4H), 7.95 (d, 1H), 7.92 (d, 2H), |
| | 7.75 (t, 2H), 7.73 (t, 2H), 7. 64 (m, 1H), |
| | 7.62 (m, 1H), 7.59 (m, 4H), 7.58 (m, 1H), |
| | 7.51 (m, 2H), 7.44 (m 1H), 7.41 (m, 1H) |
| 2-54 | δ = 9.09 (s, 2H), 8.49 (d, 2H), 8.00 (m, 4H), |
| | 7.95 (m, 1H), 7.92 (d, 2H), 7.75 (d, 2H), |
| | 7.64 (s, 1H), 7.62 (m, 1H), 7.59 (m, 4H), |
| | 7.52 (d, 2H), 7.51 (d, 2H), 7.44 (d, 1H), |
| | 7.41 (m, 1H) |
| 2-58 | δ = 8.99 (d, 1H), 8.93 (d, 2H), 8.55 (m, 1H), |
| | 8.34 (s, 1H), 8.28 (d, 2H), 8.18 (m, 1H), |
| | 8.12 (m, 1H), 8.10 (m, 1H), 7.88 (m, 2H), |
| | 7.82 (m, 1H), 8.81 (m, 1H), 7.79 (m, 2H), |
| | 7.72 (d, 1H), 7.71 (d, 4H), 7.55 (m, 2H), |
| | 7.51 (m, 4H), 7.41 (m, 2H) |
| 2-59 | δ = 8.55 (m, 1H), 8.28 (d, 4H), 8.24 (m, 1H), |
| | 8.18 (m, 1H), 7.79 (t, 2H), 7.75 (m, 2H), |
| | 7.71 (s, 1H), 7.70 (s, 1H) |
| 2-47 | δ = 8.55 (m, 2H), 8.28 (d, 4H), 8.01 (s, 2H), |
| | 7.89 (d, 1H), 7.75 (d, 1H), 7.66 (d, 1H), 7.62 |
| | (d, 1H), 7.55 (m, 2H), 7.51 (m, 4H), 7.44 (d, |
| | 1H), 7.41 (m, 2H), 7.38 (m, 1H) 7.32 (t, 1H) |
| 2-51 | δ = 9.09 (s, 2H), 8.49 (d, 2H), 8.00 (m, 4H), |
| | 7.95 (m, 1H), 7.92 (m, 2H), 7.89 (m, 1H), |
| | 7.85 (d, 2 H), 7.75 (s, 1H), 7.66 (d, 1H), 7.64 |
| | (d,), 7.59 (m, 4H), 7.38 (m, 1H), 7.32 (m, |
| | 1H) 7.25 (d, 2H) |

TABLE 10

| Compound | FD-Mass |
|---|---|
| 2-2 | m/z = 618.32 (C₄₃H₁₀D₁₇N₃O, 618.80) |
| 2-12 | m/z = 651.23 (C47H29N3O, 651.75) |
| 2-18 | m/z = 624.22 (C₄₆H₂₈N₂O, 624.73) |
| 2-20 | m/z = 600.22 (C₄₄H₂₈N₂O, 600.71) |
| 2-21 | m/z = 604.16 (C₄₂H₂₄N₂OS, 604.72) |
| 2-47 | m/z = 525.18 (C₃₇H₂₃N₃O, 525.60) |
| 2-36 | m/z = 601.22 (C₄₃H₂₇N₃O, 601.69) |
| 2-53 | m/z = 575.20 (C41H25N3O, 575.66) |
| 2-54 | m/z = 575.20 (C41H25N3O, 575.66) |
| 2-58 | m/z = 675.23 (C49H29N3O, 675.77) |
| 2-59 | m/z = 601.22 (C₄₃H₂₇N₃O, 601.69) |
| 2-51 | m/z = 575.20 (C₄₁H₂₅N₃O, 575.66) |

### Experimental Example 1

### Experimental Example 1-1. Manufacture of Organic Light-Emitting Device

A glass substrate on which ITO was coated as a thin film to a thickness of 1,500 Å was ultrasonic cleaned with distilled water. When the cleaning with distilled water was finished, the substrate was ultrasonic cleaned with solvents such as acetone, methanol and isopropyl alcohol, then dried, and then subjected to ultraviolet ozone (UVO) treatment for 5 minutes using ultraviolet (UV) in a UV cleaner. After that, the substrate was transferred to a plasma cleaner (PT), then subjected to plasma treatment under vacuum for ITO work function increase and residual film removal, and transferred to a thermal deposition apparatus for organic deposition.

On the transparent ITO electrode (positive electrode), 4,4′,4″-tris[2-naphthyl(phenyl)amino]triphenylamine (2-TNATA), a hole injection layer, having a thickness of 600 Å and N,N′-bis(α-naphthyl)-N,N′-diphenyl-4,4′-diamine (NPB), a hole transport layer, having a thickness of 300 Å were deposited.

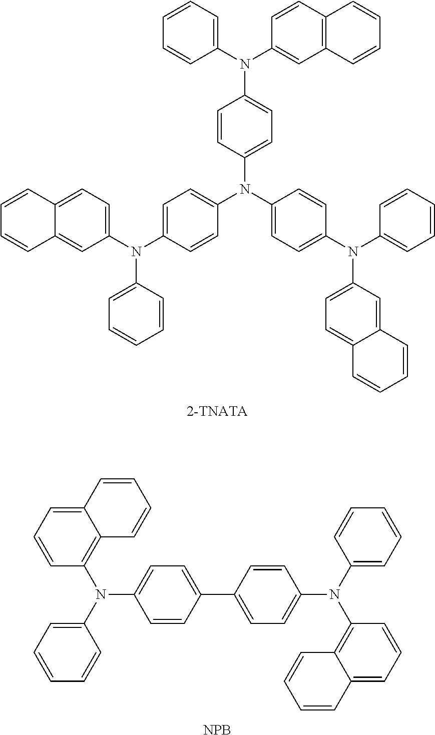 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. The light emitting layer was deposited to a thickness of 500 Å by depositing a compound described in the following Table 11 as a red host (or green host), and, using (piq)₂(Ir) (acac) as a red phosphorescent dopant, doping the (piq)₂(Ir) (acac) to the host by 3 wt %.

After that, BCP was deposited to a thickness of 60 Å as a hole blocking layer, Alq₃ was deposited to a thickness of 200 Å thereon as an electron transport layer. Lastly, lithium fluoride (LiF) was deposited on the electron transport layer to a thickness of 10 Å to form an electron injection layer, and then aluminum (Al) was deposited on the electron injection layer to a thickness of 1,200 Å to form a negative electrode, and as a result, an organic electroluminescent device was manufactured.

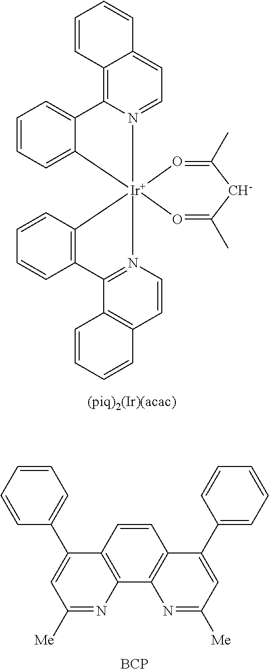

Meanwhile, all the organic compounds required to manufacture the organic light emitting device (OLED) were vacuum sublimation purified under 10⁻⁸ torr to 10⁻⁶ torr for each material to be used in the manufacture of the OLED.

### Experimental Example 1-2. Driving Voltage and Light Emission Efficiency of Organic Light-Emitting Device

For each of the organic light-emitting devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light-emitting device manufactured according to the present invention are shown in the following Table 11.

T₉₀ means a lifetime (unit: hour), a time taken for luminance to become 90% with respect to initial luminance.

TABLE 11

| | | Threshold | Driving | | Color | |
|---|---|---|---|---|---|---|
| | | Voltage | Voltage | Efficiency | Coordinate | Lifetime |
| | Compound | (Vₒₙ) | (Vₒₚ) | (cd/A) | (x, y) | (T₉₀) |
| Example 1 | 5 | 2.51 | 3.06 | 65.13 | (0.684, | 73 |
| | | | | | 0.316) | |
| Example 2 | 8 | 2.49 | 3.09 | 66.56 | (0.685, | 73 |
| | | | | | 0.314) | |
| Example 3 | 13 | 2.47 | 3.02 | 54.33 | (0.684, | 71 |
| | | | | | 0.315) | |
| Example 4 | 14 | 2.46 | 3.12 | 53.14 | (0.684, | 72 |
| | | | | | 0.316) | |
| Example 5 | 15 | 2.47 | 2.96 | 54.05 | (0.684, | 71 |
| | | | | | 0.316) | |
| Example 6 | 45 | 2.48 | 2.93 | 63.67 | (0.684, | 73 |
| | | | | | 0.316) | |
| Example 7 | 76 | 2.48 | 3.08 | 68.54 | (0.684, | 75 |
| | | | | | 0.316) | |
| Example 8 | 94 | 2.51 | 3.03 | 74.33 | (0.684, | 72 |
| | | | | | 0.315) | |
| Example 9 | 140 | 2.46 | 2.98 | 64.07 | (0.683, | 73 |
| | | | | | 0.317) | |
| Example 10 | 156 | 2.47 | 2.91 | 66.55 | (0.684, | 73 |
| | | | | | 0.316) | |
| Example 11 | 157 | 2.47 | 3.10 | 67.81 | (0.683, | 66 |
| | | | | | 0.318) | |
| Example 12 | 221 | 2.49 | 2.84 | 53.80 | (0.685, | 66 |
| | | | | | 0.314) | |
| Example 13 | 226 | 2.48 | 3.11 | 55.20 | (0.684, | 73 |
| | | | | | 0315) | |
| Example 14 | 233 | 2.46 | 2.87 | 52.17 | (0.685, | 71 |
| | | | | | 0.315) | |
| Example 15 | 244 | 2.46 | 2.85 | 55.07 | (0.683, | 71 |
| | | | | | 0.318) | |
| Example 16 | 281 | 2.47 | 2.94 | 77.65 | (0.684, | 71 |
| | | | | | 0315) | |
| Example 17 | 289 | 2.49 | 2.97 | 81.67 | (0.683, | 72 |
| | | | | | 0.318) | |
| Example 18 | 292 | 2.46 | 3.04 | 80.54 | (0.680, | 70 |
| | | | | | 0.319) | |
| Example 19 | 294 | 2.48 | 3.07 | 77.22 | (0.684, | 73 |
| | | | | | 0.316) | |
| Example 20 | 296 | 2.48 | 2.95 | 86.75 | (0.685, | 73 |
| | | | | | 0.315) | |
| Example 21 | 298 | 2.50 | 2.99 | 79.65 | (0.683, | 74 |
| | | | | | 0.317) | |
| Example 22 | 309 | 2.47 | 3.00 | 54.55 | (0.684, | 97 |
| | | | | | 0.315) | |
| Example 23 | 310 | 2.46 | 3.13 | 53.12 | (0.684, | 99 |
| | | | | | 0.316) | |
| Example 24 | 311 | 2.47 | 2.88 | 52.31 | (0.683, | 96 |
| | | | | | 0.318) | |
| Example 25 | 312 | 2.47 | 2.86 | 55.21 | (0.685, | 98 |
| | | | | | 0.314) | |
| Example 26 | 313 | 2.50 | 2.92 | 52.66 | (0.684, | 95 |
| | | | | | 0315) | |
| Example 27 | 315 | 2.49 | 2.89 | 56.56 | (0.685, | 96 |
| | | | | | 0.315) | |
| Example 28 | 317 | 2.47 | 3.05 | 76.55 | (0.683, | 93 |
| | | | | | 0.318) | |
| Example 29 | 318 | 2.49 | 2.90 | 81.56 | (0.684, | 98 |
| | | | | | 0.316) | |
| Example 30 | 319 | 2.47 | 3.08 | 69.88 | (0.684, | 99 |
| | | | | | 0.316) | |
| Example 31 | 320 | 2.49 | 3.01 | 68.21 | (0.685, | 99 |
| | | | | | 0.314) | |
| Comparative | S | 2.23 | 3.85 | 34.15 | (0.684, | 39 |
| Example 1 | | | | | 0.316) | |
| Comparative | T | 2.22 | 3.91 | 38.11 | (0.684, | 38 |
| Example 2 | | | | | 0.315) | |
| Comparative | U | 2.26 | 3.99 | 38.99 | (0.683, | 43 |
| Example 3 | | | | | 0.317) | |
| Comparative | V | 3.55 | 5.90 | 22.00 | (0.684, | 25 |
| Example 4 | | | | | 0.315) | |
| Comparative | W | 3.57 | 5.99 | 26.03 | (0.684, | 36 |
| Example 5 | | | | | 0.315) | |
| Comparative | X | 3.21 | 5.65 | 19.97 | (0.684, | 35 |
| Example 6 | | | | | 0.316) | |
| Comparative | Y | 2.47 | 3.86 | 27.61 | (0.684, | 41 |
| Example 7 | | | | | 0.316) | |
| Comparative | Z | 2.48 | 3.72 | 29.14 | (0.684, | 53 |
| Example 8 | | | | | 0.315) | |

### Comparative Example Compound

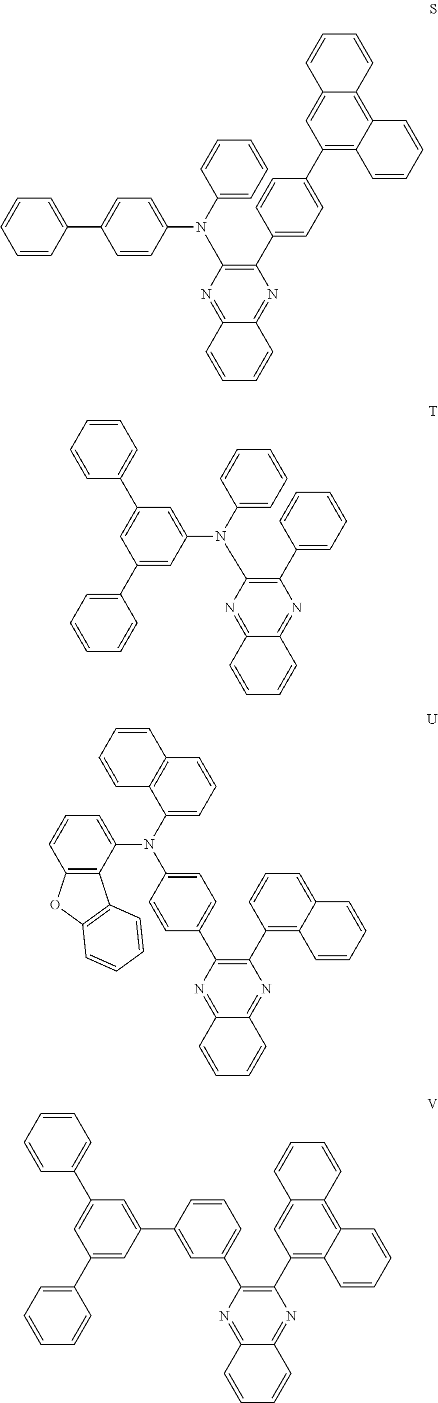 [SMILES: c1ccc(-c2ccc(N(c3ccccc3)c3nc4ccccc4nc3-c3ccc(-c4cc5ccccc5c5ccccc45)cc3)cc2)cc1 ; c1ccc(-c2cc(-c3ccccc3)cc(N(c3ccccc3)c3nc4ccccc4nc3-c3ccccc3)c2)cc1 ; c1ccc2c(-c3nc4ccccc4nc3-c3ccc(N(c4cccc5ccccc45)c4cccc5oc6ccccc6c45)cc3)cccc2c1 ; c1ccc(-c2cc(-c3ccccc3)cc(-c3cccc(-c4nc5ccccc5nc4-c4cc5ccccc5c5ccccc45)c3)c2)cc1]

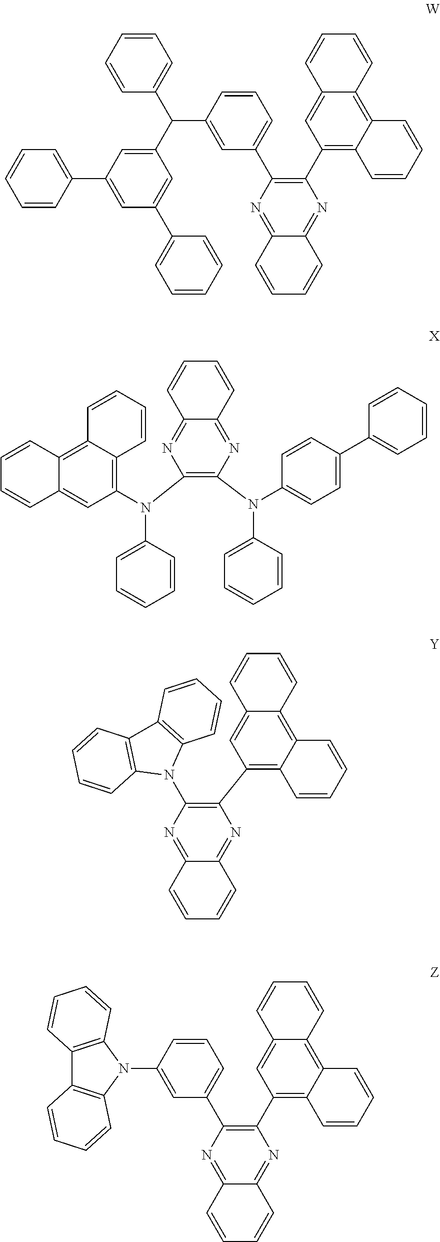 [SMILES: c1ccc(-c2cc(-c3ccccc3)cc(C(c3ccccc3)c3cccc(-c4nc5ccccc5nc4-c4cc5ccccc5c5ccccc45)c3)c2)cc1 ; c1ccc(-c2ccc(N(c3ccccc3)c3nc4ccccc4nc3N(c3ccccc3)c3cc4ccccc4c4ccccc34)cc2)cc1 ; c1ccc2c(c1)cc(-c1nc3ccccc3nc1-n1c3ccccc3c3ccccc31)c1ccccc12 ; c1cc(-c2nc3ccccc3nc2-c2cc3ccccc3c3ccccc23)cc(-n2c3ccccc3c3ccccc32)c1]

From the results of Table 11,
it may be seen that, when the organic material layer of the organic light-emitting device was deposited with the heterocyclic compound represented by Chemical Formula 1 of the present invention, the organic light-emitting device had lower driving voltage, and improved light emission efficiency and lifetime compared to the organic light-emitting devices of Comparative Examples 1 to 8.

Comparative Example 1 has quinoxaline and phenanthrene linked to a phenylene group, Comparative Examples 2 and 3 have phenyl group and naphthyl group substituents introduced to quinoxaline, respectively, Comparative Examples 4 and 5 do not include an arylamine group, Comparative Example 6 does not have a phenanthrene substituent introduced to quinoxaline, and Comparative Examples 7 and 8 have a carbazole group introduced thereto instead of an arylamine group.

Comparative Examples 1 to 8 showed poorer results compared to Examples 1 to 31 in driving voltage, efficiency and lifetime. In other words, it was able to be identified that the threshold voltage was controlled when a phenanthrene substituent is directly introduced to quinoxaline without a linking group. In addition, it was seen that the element had more improved driving, efficiency and lifetime when introducing an arylamine group as a substituent rather than a carbazole group. This is expected to be a result of the compound forming a proper band gap when substituted with an arylamine group, thereby preventing a loss of electrons and holes in the light emitting layer and establishing an effective recombination zone.

It may be identified that Examples 22 to 31 substituted with deuterium have more superior lifetime properties than the compounds of Examples 1 to 21 that do not include deuterium. This is considered to be due to the fact that a carbon-deuterium bond has greater bond dissociation energy than a carbon-hydrogen bond, affecting a lifetime.

### Experimental Example 2

### Experimental Example 2-1. Manufacture of Organic Light-Emitting Device

An organic light-emitting device was manufactured in the same manner as in Experimental Example 1-1 except that, as the host of the light emitting layer, one type of a first host (heterocyclic compound represented by Chemical Formula 1) and one type of a second host (heterocyclic compound represented by Chemical Formula 2) described in the following Table 12 were pre-mixed and then deposited in one source of supply.

### Experimental Example 2-2. Driving Voltage and Light Emission Efficiency of Organic Light-Emitting Device

For each of the organic light-emitting devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light-emitting device manufactured according to the present invention are shown in the following Table 12.

T₉₀ means a lifetime (unit: hour), a time taken for luminance to become 90% with respect to initial luminance.

TABLE 12

| | First | Second | | Driving | | Color | |
|---|---|---|---|---|---|---|---|
| | Host | Host | Ratio | Voltage | Efficiency | Coordinate | Lifetime |
| | (P) | (N) | (P:N) | (Vₒₚ) | (cd/A) | (x, y) | (T₉₀) |
| Example 31 | 5 | 2-12 | 1:1 | 3.09 | 85.03 | (0.684, | 182 |
| | | | | | | 0.316) | |
| Example 32 | 5 | 2-12 | 1:2 | 3.06 | 87.15 | (0.684, | 170 |
| | | | | | | 0.315) | |
| Example 33 | 5 | 2-12 | 2:1 | 3.13 | 83.98 | (0.684, | 188 |
| | | | | | | 0.316) | |
| Example 34 | 8 | 2-53 | 1:1 | 3.12 | 86.22 | (0.684, | 182 |
| | | | | | | 0.316) | |
| Example 35 | 8 | 2-53 | 1:2 | 3.09 | 88.42 | (0.684, | 169 |
| | | | | | | 0.316) | |
| Example 36 | 8 | 2-53 | 2:1 | 3.17 | 84.83 | (0.685, | 187 |
| | | | | | | 0.315) | |
| Example 37 | 45 | 2-58 | 1:1 | 2.97 | 63.52 | (0.684, | 171 |
| | | | | | | 0.316) | |
| Example 38 | 140 | 2-53 | 1:1 | 3.01 | 65.89 | (0.683, | 172 |
| | | | | | | 0.318) | |
| Example 39 | 233 | 2-12 | 1:1 | 2.91 | 72.43 | (0.685, | 178 |
| | | | | | | 0.314) | |
| Example 40 | 233 | 2-12 | 1:2 | 2.87 | 74.56 | (0.684, | 168 |
| | | | | | | 0315) | |
| Example 41 | 233 | 2-12 | 2:1 | 2.95 | 70.87 | (0.685, | 182 |
| | | | | | | 0.315) | |
| Example 42 | 310 | 2-54 | 1:1 | 3.17 | 52.84 | (0.683, | 201 |
| | | | | | | 0.318) | |
| Example 43 | 318 | 2-51 | 1:1 | 2.93 | 101.84 | (0.683, | 203 |
| | | | | | | 0.317) | |
| Comparative | S | 2-53 | 1:1 | 3.85 | 53.24 | (0.684, | 140 |
| Example 9 | | | | | | 0.316) | |
| Comparative | T | 2-58 | 1:1 | 3.91 | 57.79 | (0.683, | 137 |
| Example 10 | | | | | | 0.317) | |
| Comparative | U | 2-51 | 1:1 | 3.99 | 59.02 | (0.684, | 135 |
| Example 11 | | | | | | 0.316) | |
| Comparative | V | 2-54 | 1:1 | 5.90 | 41.76 | (0.683, | 121 |
| Example 12 | | | | | | 0.317) | |
| Comparative | W | 2-12 | 1:1 | 5.99 | 45.88 | (0.684, | 132 |
| Example 13 | | | | | | 0.316) | |
| Comparative | X | 2-53 | 1:1 | 5.65 | 39.79 | (0.683, | 130 |
| Example 14 | | | | | | 0.317) | |
| Comparative | Y | 2-58 | 1:1 | 3.86 | 57.54 | (0.684, | 138 |
| Example 15 | | | | | | 0.316) | |
| Comparative | Z | 2-54 | 1:1 | 3.72 | 50.02 | (0.683, | 142 |
| Example 16 | | | | | | 0.318) | |

Comparing the results of Table 11 and Table 12,
it was able to be identified that efficiency and lifetime were able to be improved when combining two types of hosts compared to the results of Experimental Example 1-2 using a single host. It was able to be identified that, when a donor (P-host) having a favorable hole transport ability and an acceptor (N-host) having a favorable electron transport ability are used at the same time as hosts of a light emitting layer, electrons and holes are readily injected, which leads to the results of enhancing efficiency and lifetime through effective formation of a recombination zone.

### REFERENCE NUMERAL

100: Substrate
200: Positive Electrode
300: Organic Material Layer
301: Hole Injection Layer
302: Hole Transport layer
303: Light Emitting Layer
304: Hole Blocking Layer
305: Electron Transport Layer
306: Electron Injection Layer
400: Negative Electrode

1. A heterocyclic compound represented by the following Chemical Formula 1: 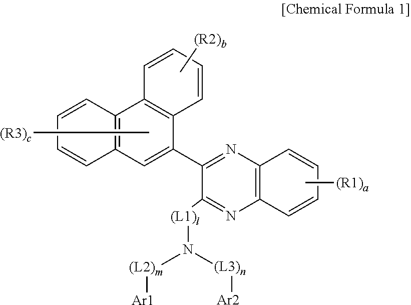
wherein, in Chemical Formula 1,
R1 to R3 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring; or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
a is an integer of 0 to 4, and when a is 2 or greater, R1s are the same as or different from each other;
b is an integer of 0 to 4, and when b is 2 or greater, R2s are the same as or different from each other;
c is an integer of 0 to 5, and when c is 2 or greater, R3s are the same as or different from each other;
Ar1 and Ar2 are the same as or different from each other, and each independently a substituted or unsubstituted C6 to C60 aryl group; or a substituted or unsubstituted C2 to C60 heteroaryl group including one or more of O; or S as a heteroatom;
L1 to L3 are the same as or different from each other, and each independently a direct bond; a substituted or unsubstituted C6 to C60 arylene group; or a substituted or unsubstituted C2 to C60 heteroarylene group including one or more of O; or S as a heteroatom; and
l, m and n are an integer of 0 to 5, and when 1 is 2 or greater, L1s are the same as or different from each other, when m is 2 or greater, L2s are the same as or different from each other, and when n is 2 or greater, L3s are the same as or different from each other.

2. The heterocyclic compound of claim 1, wherein Ar1 and Ar2 are the same as or different from each other, and each independently a substituted or unsubstituted C6 to C30 aryl group; or a substituted or unsubstituted C2 to C30 heteroaryl group including one or more of O; or S as a heteroatom.

3. The heterocyclic compound of claim 1, wherein L1 to L3 are the same as or different from each other, and each independently a direct bond; or a substituted or unsubstituted C6 to C60 arylene group.

4. The heterocyclic compound of claim 1, wherein the heterocyclic compound represented by Chemical Formula 1 does not include deuterium as a substituent, or has a deuterium content of 1% to 100% with respect to a total number of hydrogen atoms and deuterium atoms.

5. The heterocyclic compound of claim 1, wherein the heterocyclic compound represented by Chemical Formula 1 is represented by any one of the following compounds:

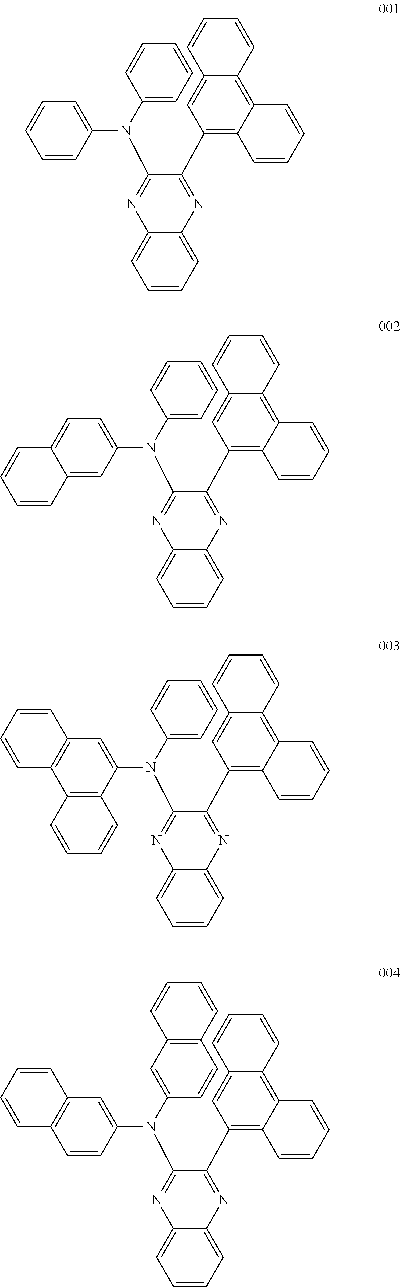 [SMILES: c1ccc(N(c2ccccc2)c2nc3ccccc3nc2-c2cc3ccccc3c3ccccc23)cc1 ; c1ccc(N(c2ccc3ccccc3c2)c2nc3ccccc3nc2-c2cc3ccccc3c3ccccc23)cc1 ; c1ccc(N(c2nc3ccccc3nc2-c2cc3ccccc3c3ccccc23)c2cc3ccccc3c3ccccc23)cc1 ; c1ccc2cc(N(c3ccc4ccccc4c3)c3nc4ccccc4nc3-c3cc4ccccc4c4ccccc34)ccc2c1]

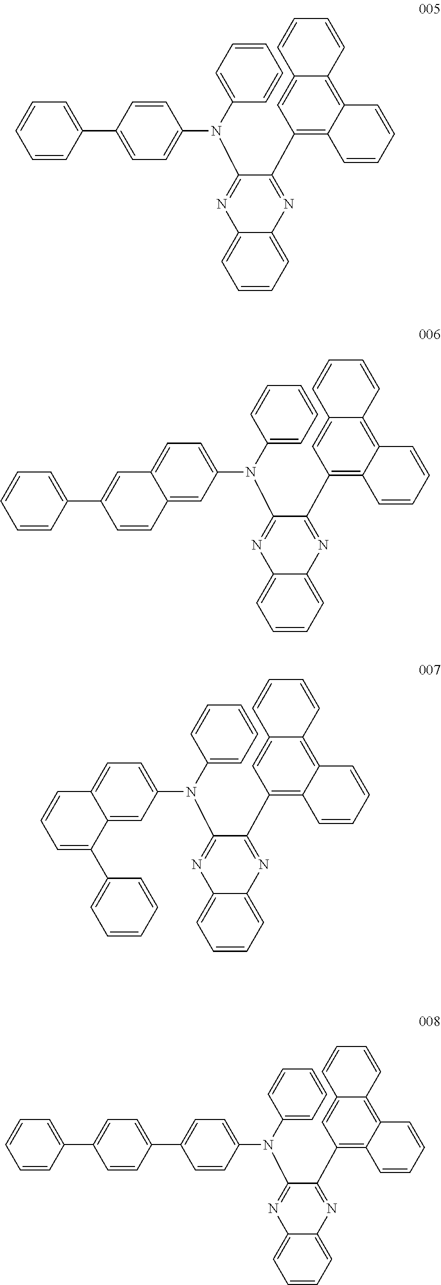 [SMILES: c1ccc(-c2ccc(N(c3ccccc3)c3nc4ccccc4nc3-c3cc4ccccc4c4ccccc34)cc2)cc1 ; c1ccc(-c2ccc3cc(N(c4ccccc4)c4nc5ccccc5nc4-c4cc5ccccc5c5ccccc45)ccc3c2)cc1 ; c1ccc(-c2cccc3ccc(N(c4ccccc4)c4nc5ccccc5nc4-c4cc5ccccc5c5ccccc45)cc23)cc1 ; c1ccc(-c2ccc(-c3ccc(N(c4ccccc4)c4nc5ccccc5nc4-c4cc5ccccc5c5ccccc45)cc3)cc2)cc1]

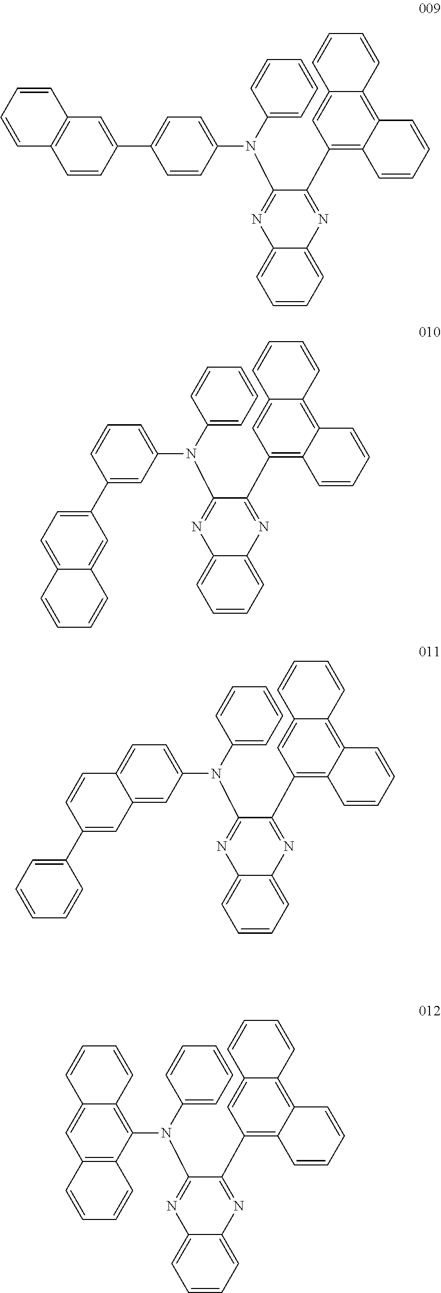

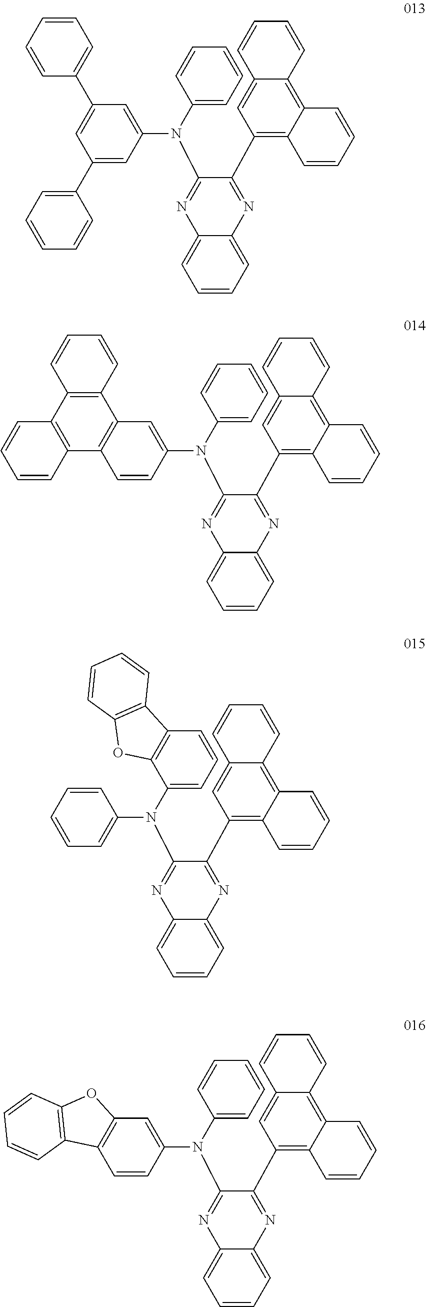 [SMILES: c1ccc(-c2cc(-c3ccccc3)cc(N(c3ccccc3)c3nc4ccccc4nc3-c3cc4ccccc4c4ccccc34)c2)cc1 ; c1ccc(N(c2ccc3c4ccccc4c4ccccc4c3c2)c2nc3ccccc3nc2-c2cc3ccccc3c3ccccc23)cc1 ; c1ccc(N(c2nc3ccccc3nc2-c2cc3ccccc3c3ccccc23)c2cccc3c2oc2ccccc23)cc1 ; c1ccc(N(c2ccc3c(c2)oc2ccccc23)c2nc3ccccc3nc2-c2cc3ccccc3c3ccccc23)cc1]

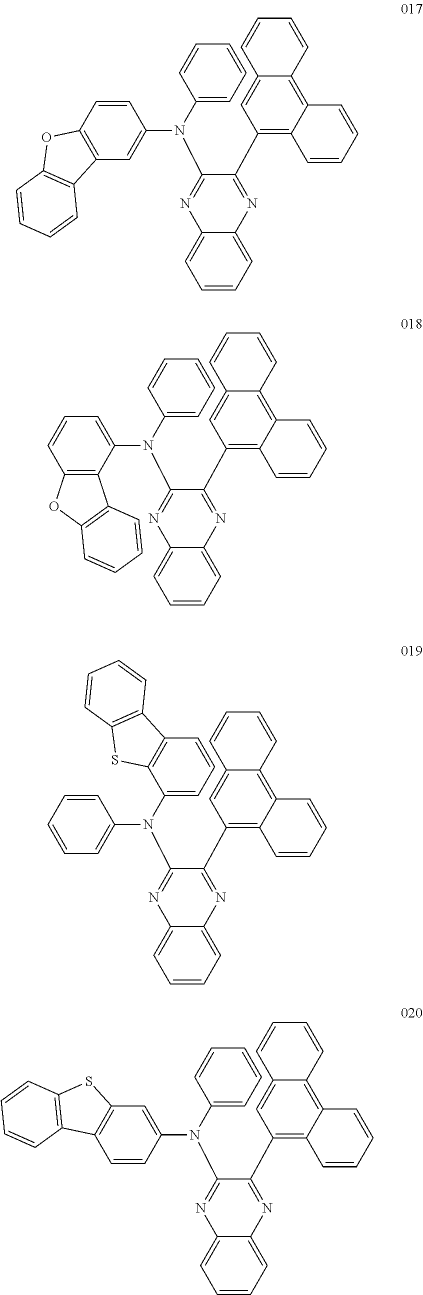

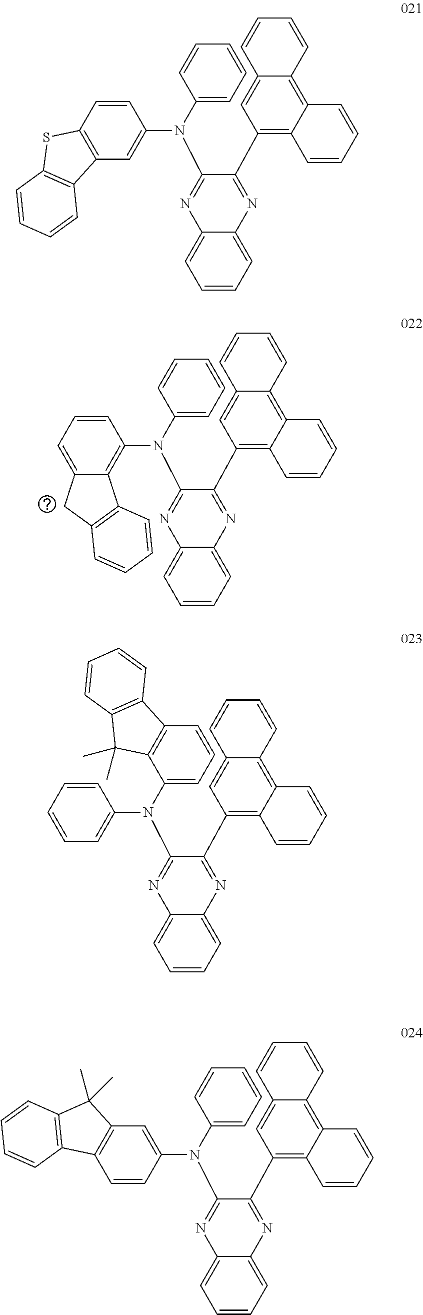

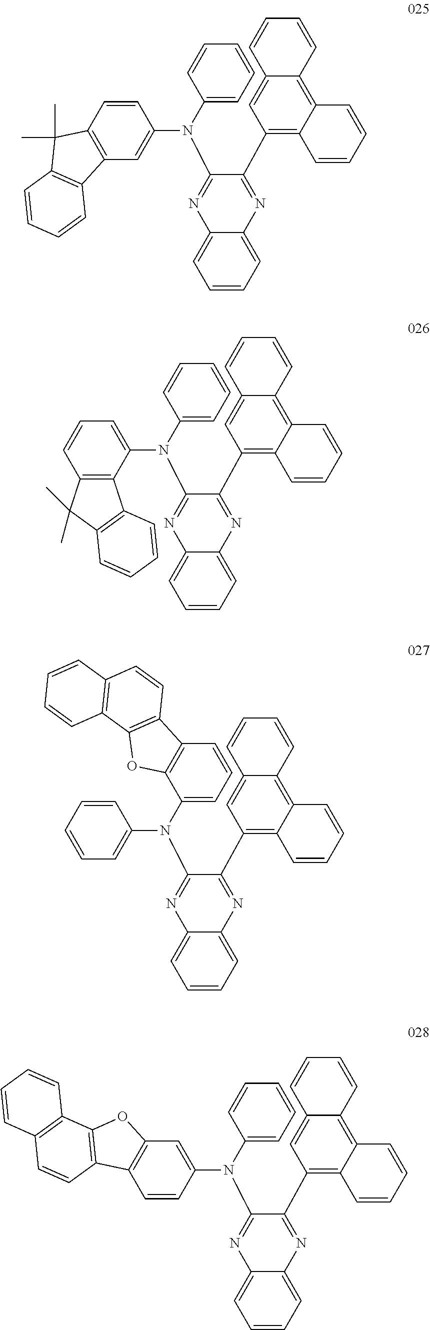 [SMILES: CC1(C)c2ccccc2-c2cc(N(c3ccccc3)c3nc4ccccc4nc3-c3cc4ccccc4c4ccccc34)ccc21 ; CC1(C)c2ccccc2-c2c(N(c3ccccc3)c3nc4ccccc4nc3-c3cc4ccccc4c4ccccc34)cccc21 ; c1ccc(N(c2nc3ccccc3nc2-c2cc3ccccc3c3ccccc23)c2cccc3c2oc2c4ccccc4ccc32)cc1 ; c1ccc(N(c2ccc3c(c2)oc2c4ccccc4ccc32)c2nc3ccccc3nc2-c2cc3ccccc3c3ccccc23)cc1]

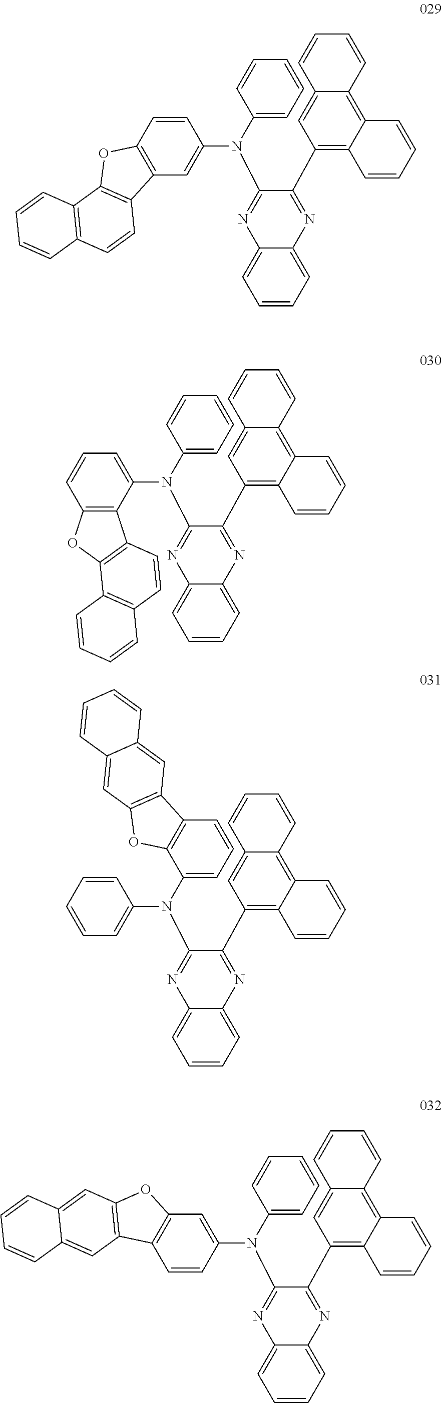

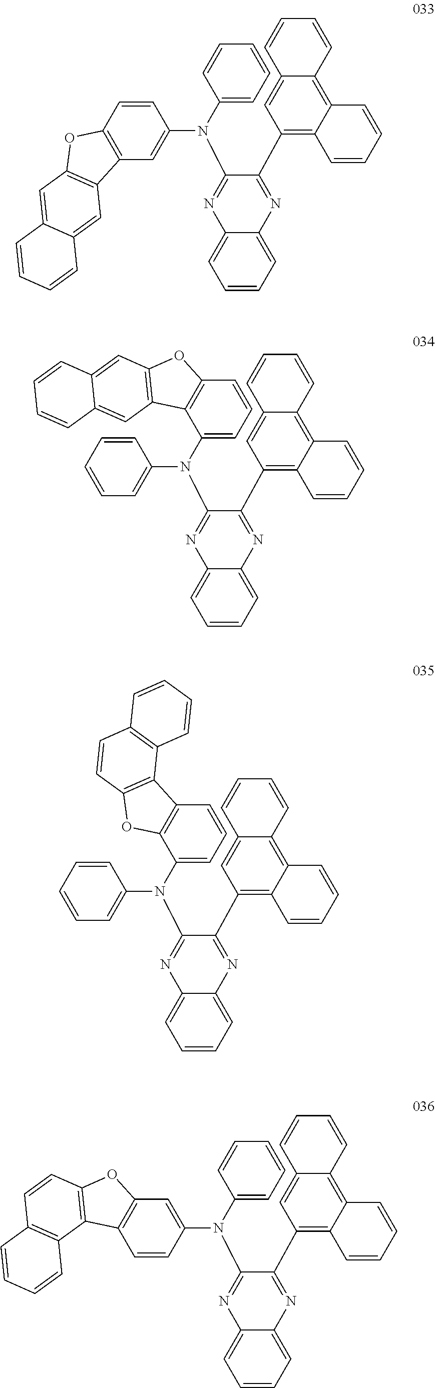 [SMILES: c1ccc(N(c2ccc3oc4cc5ccccc5cc4c3c2)c2nc3ccccc3nc2-c2cc3ccccc3c3ccccc23)cc1 ; c1ccc(N(c2nc3ccccc3nc2-c2cc3ccccc3c3ccccc23)c2cccc3oc4cc5ccccc5cc4c23)cc1 ; c1ccc(N(c2nc3ccccc3nc2-c2cc3ccccc3c3ccccc23)c2cccc3c2oc2ccc4ccccc4c23)cc1 ; c1ccc(N(c2ccc3c(c2)oc2ccc4ccccc4c23)c2nc3ccccc3nc2-c2cc3ccccc3c3ccccc23)cc1]

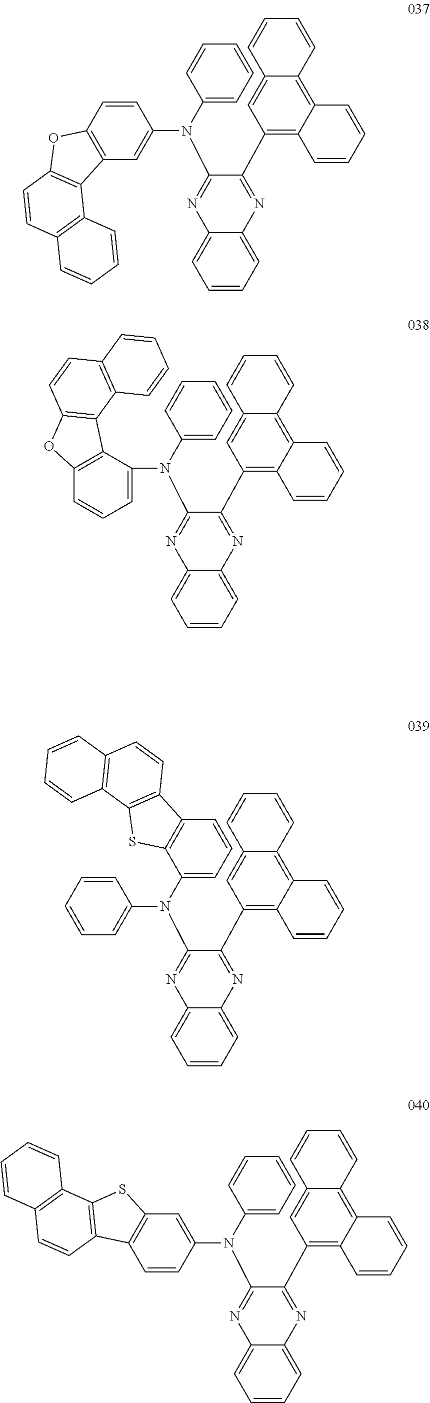 [SMILES: c1ccc(N(c2ccc3oc4ccc5ccccc5c4c3c2)c2nc3ccccc3nc2-c2cc3ccccc3c3ccccc23)cc1 ; c1ccc(N(c2nc3ccccc3nc2-c2cc3ccccc3c3ccccc23)c2cccc3oc4ccc5ccccc5c4c23)cc1 ; c1ccc(N(c2nc3ccccc3nc2-c2cc3ccccc3c3ccccc23)c2cccc3c2sc2c4ccccc4ccc32)cc1 ; c1ccc(N(c2ccc3c(c2)sc2c4ccccc4ccc32)c2nc3ccccc3nc2-c2cc3ccccc3c3ccccc23)cc1]

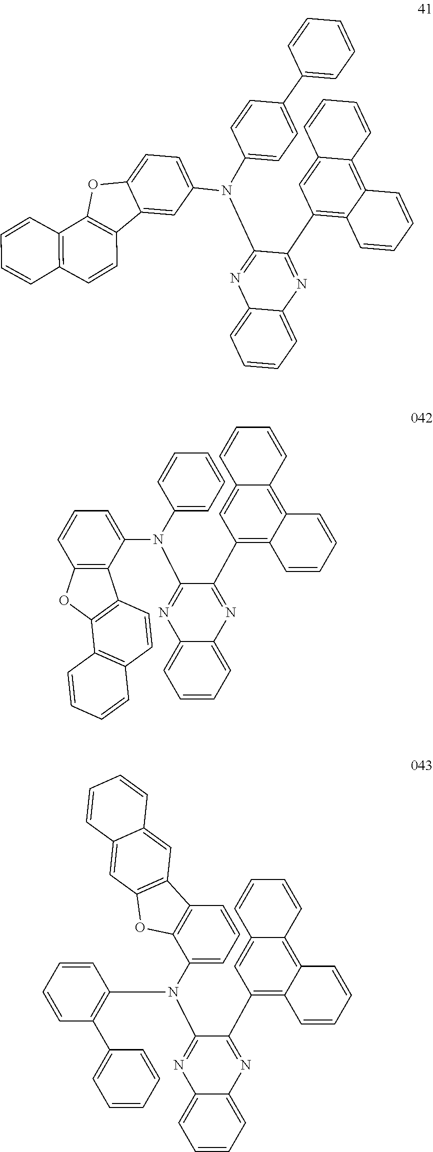 [SMILES: c1ccc(-c2ccc(N(c3ccc4oc5c6ccccc6ccc5c4c3)c3nc4ccccc4nc3-c3cc4ccccc4c4ccccc34)cc2)cc1 ; c1ccc(N(c2nc3ccccc3nc2-c2cc3ccccc3c3ccccc23)c2cccc3oc4c5ccccc5ccc4c23)cc1 ; c1ccc(-c2ccccc2N(c2nc3ccccc3nc2-c2cc3ccccc3c3ccccc23)c2cccc3c2oc2cc4ccccc4cc23)cc1]

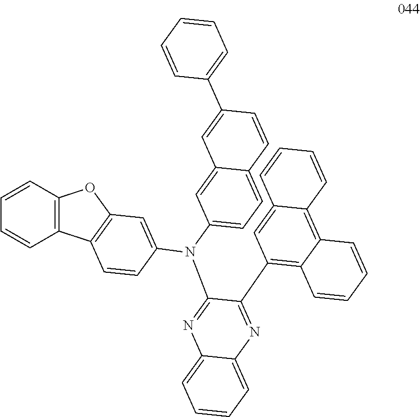 [SMILES: c1ccc(-c2ccc3ccc(N(c4ccc5c(c4)oc4ccccc45)c4nc5ccccc5nc4-c4cc5ccccc5c5ccccc45)cc3c2)cc1]

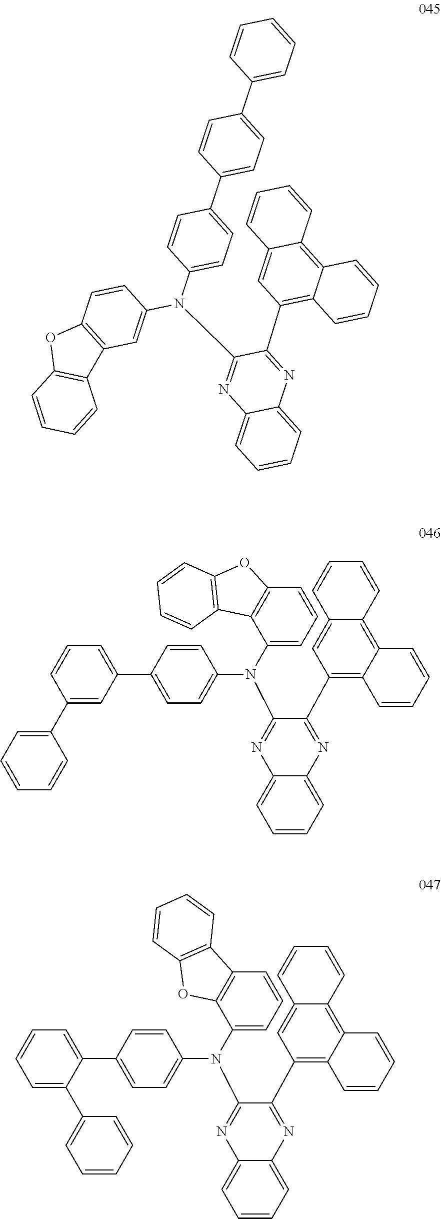 [SMILES: c1ccc(-c2ccc(-c3ccc(N(c4ccc5oc6ccccc6c5c4)c4nc5ccccc5nc4-c4cc5ccccc5c5ccccc45)cc3)cc2)cc1 ; c1ccc(-c2cccc(-c3ccc(N(c4nc5ccccc5nc4-c4cc5ccccc5c5ccccc45)c4cccc5oc6ccccc6c45)cc3)c2)cc1 ; c1ccc(-c2ccccc2-c2ccc(N(c3nc4ccccc4nc3-c3cc4ccccc4c4ccccc34)c3cccc4c3oc3ccccc34)cc2)cc1]

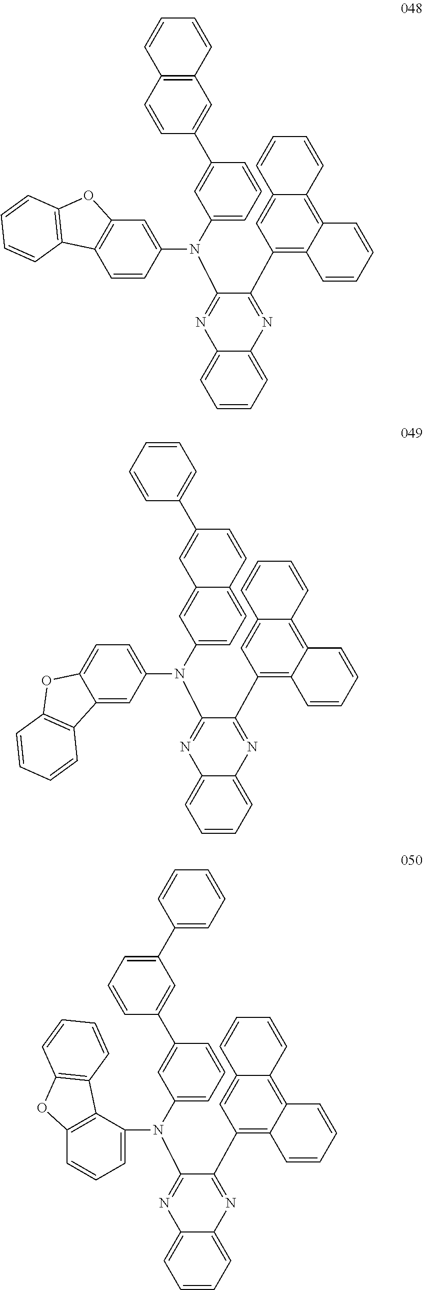

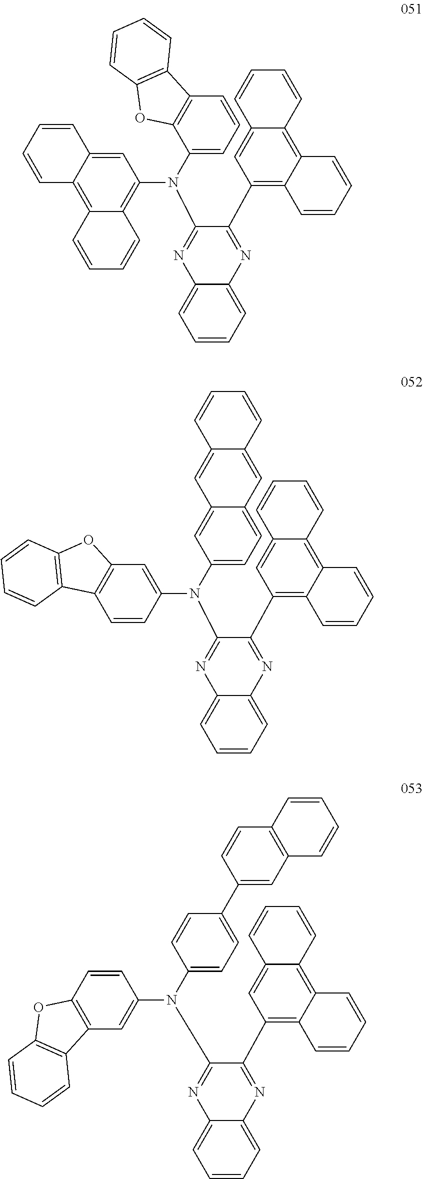 [SMILES: c1ccc2c(c1)cc(-c1nc3ccccc3nc1N(c1cc3ccccc3c3ccccc13)c1cccc3c1oc1ccccc13)c1ccccc12 ; c1ccc2cc3cc(N(c4ccc5c(c4)oc4ccccc45)c4nc5ccccc5nc4-c4cc5ccccc5c5ccccc45)ccc3cc2c1 ; c1ccc2cc(-c3ccc(N(c4ccc5oc6ccccc6c5c4)c4nc5ccccc5nc4-c4cc5ccccc5c5ccccc45)cc3)ccc2c1]

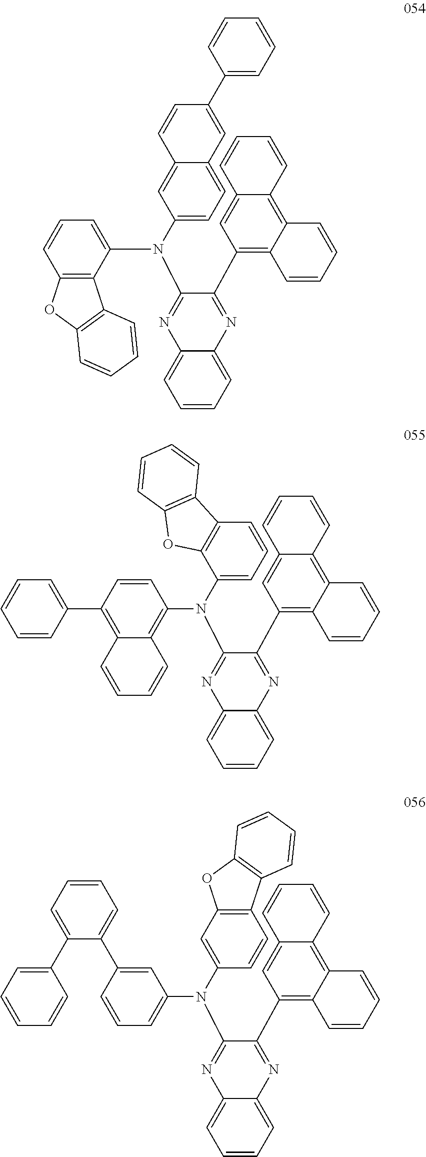

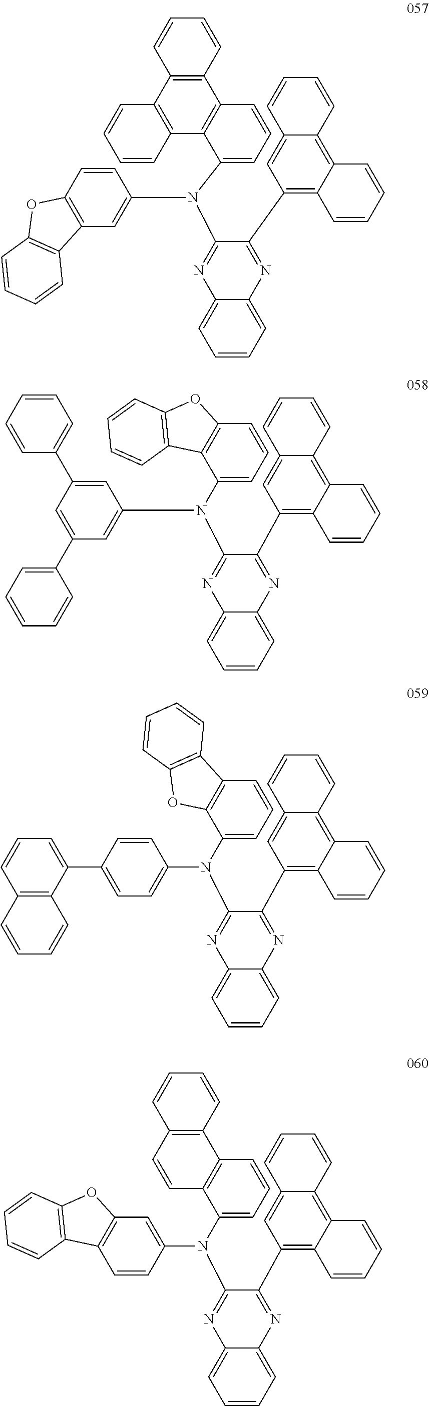

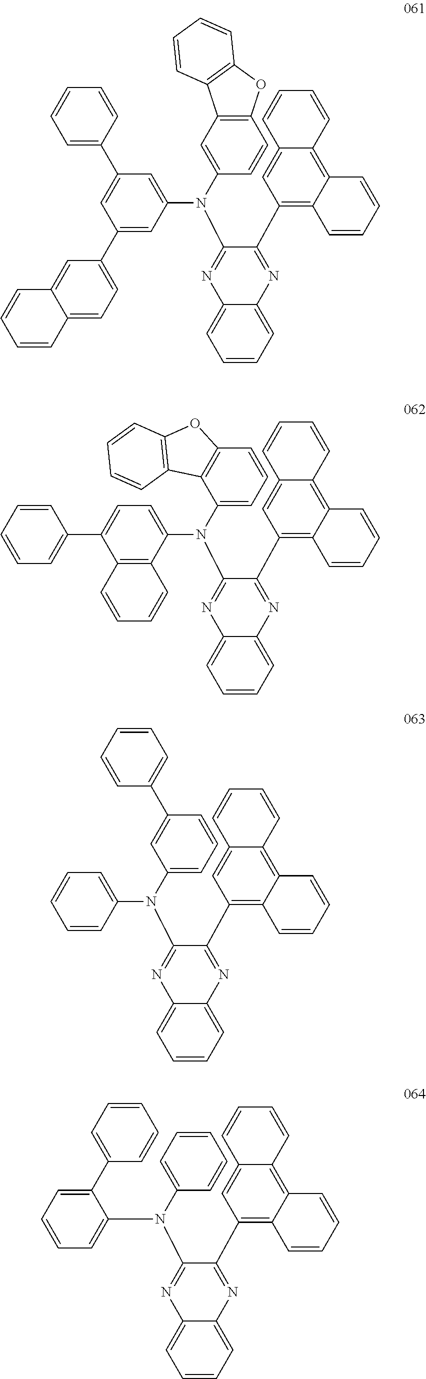 [SMILES: c1ccc(-c2cc(-c3ccc4ccccc4c3)cc(N(c3ccc4oc5ccccc5c4c3)c3nc4ccccc4nc3-c3cc4ccccc4c4ccccc34)c2)cc1 ; c1ccc(-c2ccc(N(c3nc4ccccc4nc3-c3cc4ccccc4c4ccccc34)c3cccc4oc5ccccc5c34)c3ccccc23)cc1 ; c1ccc(-c2cccc(N(c3ccccc3)c3nc4ccccc4nc3-c3cc4ccccc4c4ccccc34)c2)cc1 ; c1ccc(-c2ccccc2N(c2ccccc2)c2nc3ccccc3nc2-c2cc3ccccc3c3ccccc23)cc1]

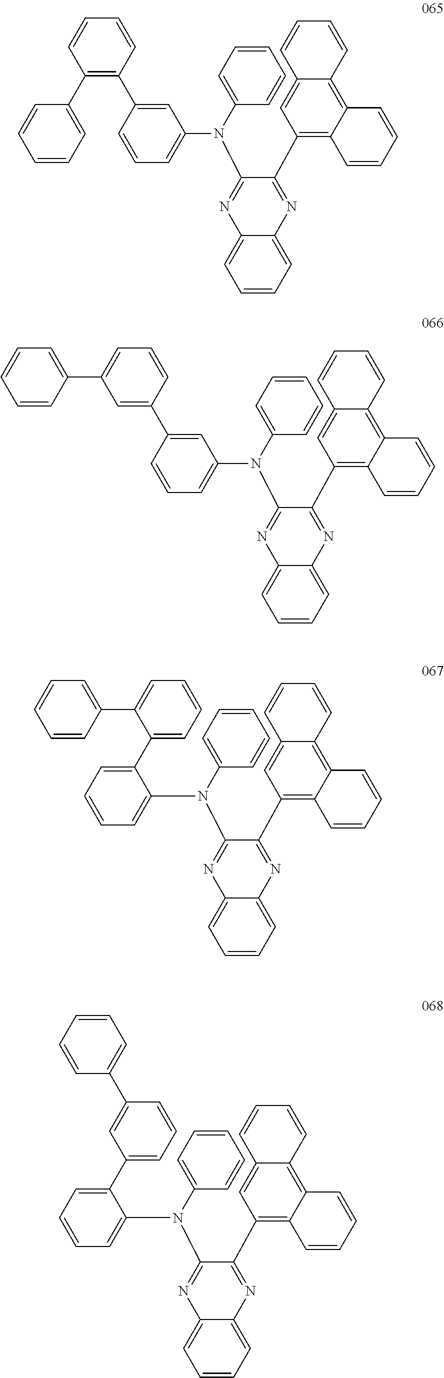 [SMILES: c1ccc(-c2ccccc2-c2cccc(N(c3ccccc3)c3nc4ccccc4nc3-c3cc4ccccc4c4ccccc34)c2)cc1 ; c1ccc(-c2cccc(-c3cccc(N(c4ccccc4)c4nc5ccccc5nc4-c4cc5ccccc5c5ccccc45)c3)c2)cc1 ; c1ccc(-c2ccccc2-c2ccccc2N(c2ccccc2)c2nc3ccccc3nc2-c2cc3ccccc3c3ccccc23)cc1 ; c1ccc(-c2cccc(-c3ccccc3N(c3ccccc3)c3nc4ccccc4nc3-c3cc4ccccc4c4ccccc34)c2)cc1]

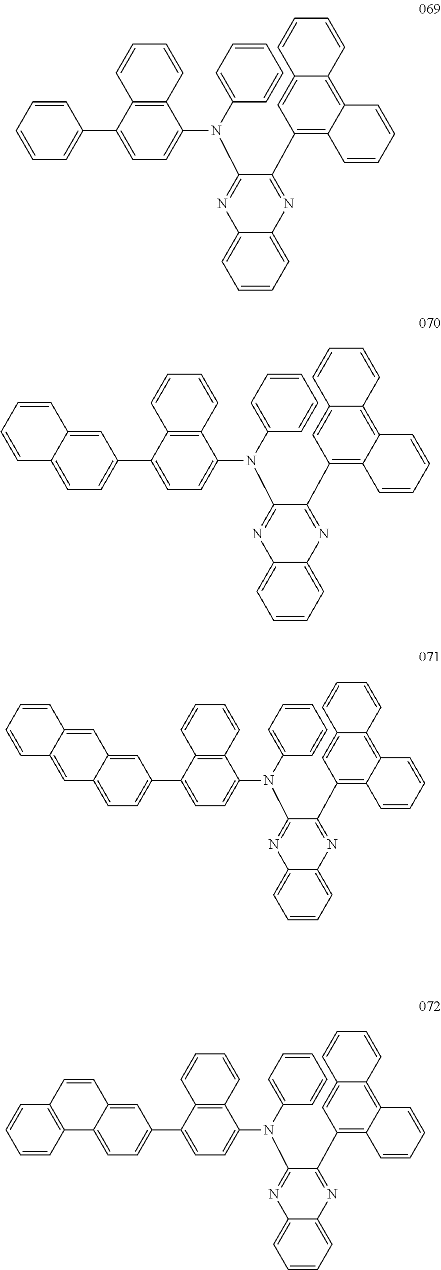 [SMILES: c1ccc(-c2ccc(N(c3ccccc3)c3nc4ccccc4nc3-c3cc4ccccc4c4ccccc34)c3ccccc23)cc1 ; c1ccc(N(c2nc3ccccc3nc2-c2cc3ccccc3c3ccccc23)c2ccc(-c3ccc4ccccc4c3)c3ccccc23)cc1 ; c1ccc(N(c2nc3ccccc3nc2-c2cc3ccccc3c3ccccc23)c2ccc(-c3ccc4cc5ccccc5cc4c3)c3ccccc23)cc1 ; c1ccc(N(c2nc3ccccc3nc2-c2cc3ccccc3c3ccccc23)c2ccc(-c3ccc4c(ccc5ccccc54)c3)c3ccccc23)cc1]

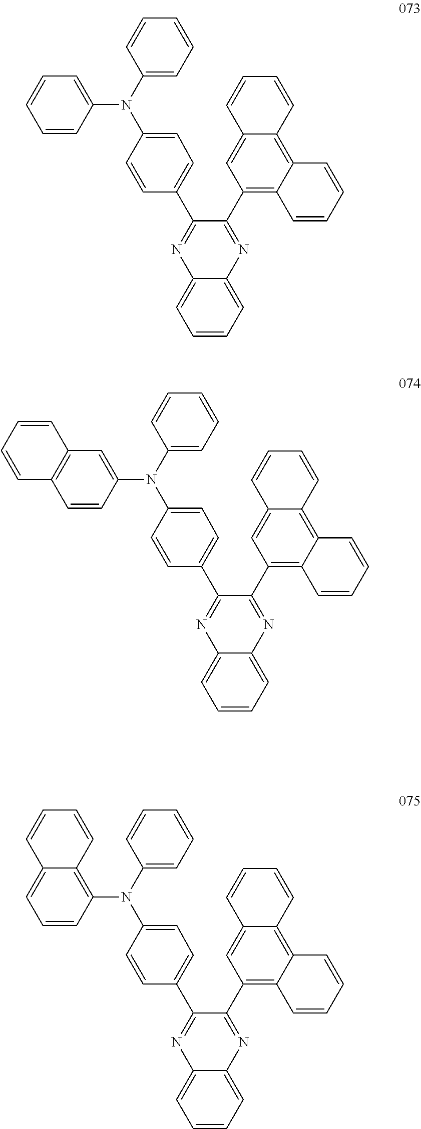 [SMILES: c1ccc(N(c2ccccc2)c2ccc(-c3nc4ccccc4nc3-c3cc4ccccc4c4ccccc34)cc2)cc1 ; c1ccc(N(c2ccc(-c3nc4ccccc4nc3-c3cc4ccccc4c4ccccc34)cc2)c2ccc3ccccc3c2)cc1 ; c1ccc(N(c2ccc(-c3nc4ccccc4nc3-c3cc4ccccc4c4ccccc34)cc2)c2cccc3ccccc23)cc1]

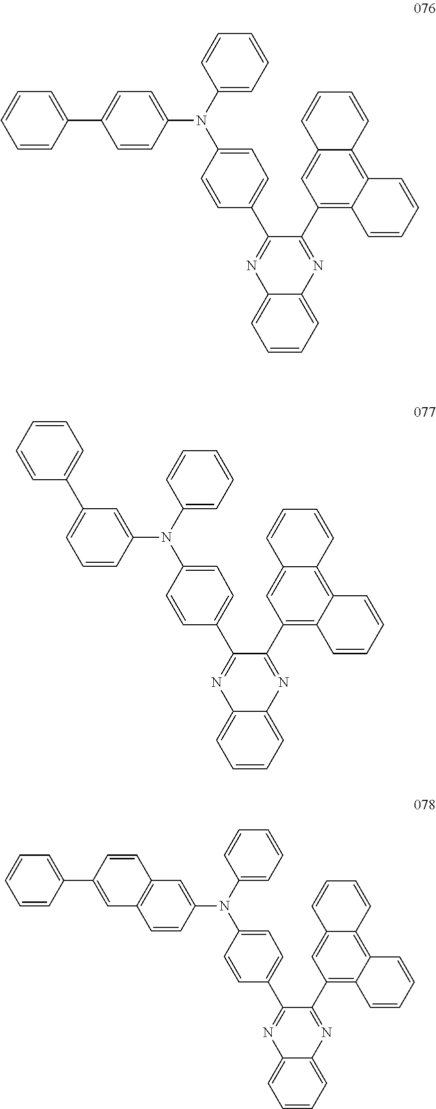 [SMILES: c1ccc(-c2ccc(N(c3ccccc3)c3ccc(-c4nc5ccccc5nc4-c4cc5ccccc5c5ccccc45)cc3)cc2)cc1 ; c1ccc(-c2cccc(N(c3ccccc3)c3ccc(-c4nc5ccccc5nc4-c4cc5ccccc5c5ccccc45)cc3)c2)cc1 ; c1ccc(-c2ccc3cc(N(c4ccccc4)c4ccc(-c5nc6ccccc6nc5-c5cc6ccccc6c6ccccc56)cc4)ccc3c2)cc1]

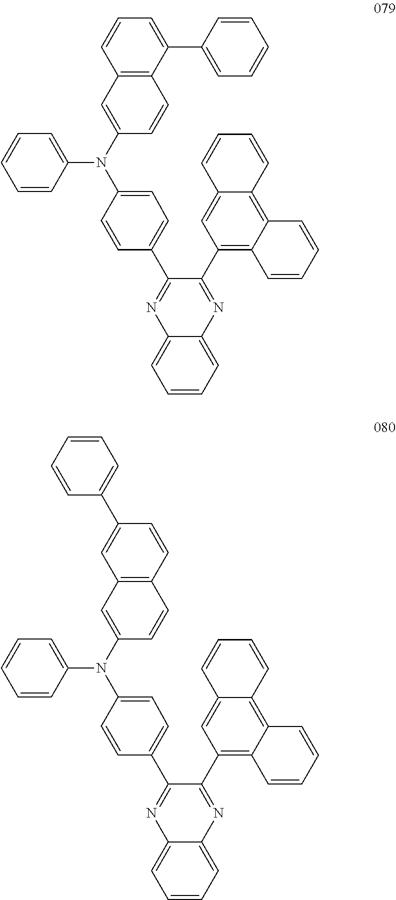 [SMILES: c1ccc(-c2cccc3cc(N(c4ccccc4)c4ccc(-c5nc6ccccc6nc5-c5cc6ccccc6c6ccccc56)cc4)ccc23)cc1 ; c1ccc(-c2ccc3ccc(N(c4ccccc4)c4ccc(-c5nc6ccccc6nc5-c5cc6ccccc6c6ccccc56)cc4)cc3c2)cc1]

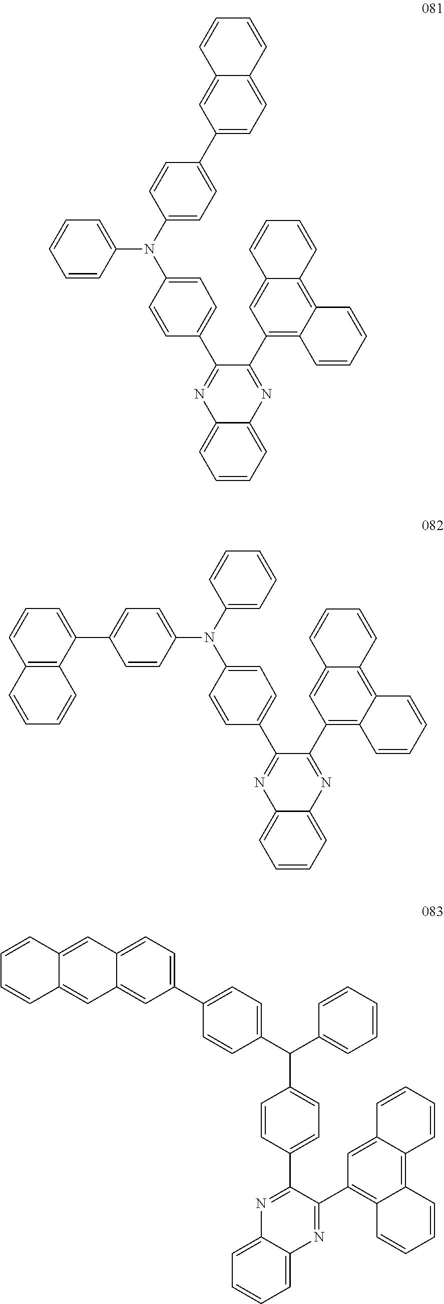 [SMILES: c1ccc(N(c2ccc(-c3ccc4ccccc4c3)cc2)c2ccc(-c3nc4ccccc4nc3-c3cc4ccccc4c4ccccc34)cc2)cc1 ; c1ccc(N(c2ccc(-c3nc4ccccc4nc3-c3cc4ccccc4c4ccccc34)cc2)c2ccc(-c3cccc4ccccc34)cc2)cc1 ; c1ccc(C(c2ccc(-c3ccc4cc5ccccc5cc4c3)cc2)c2ccc(-c3nc4ccccc4nc3-c3cc4ccccc4c4ccccc34)cc2)cc1]

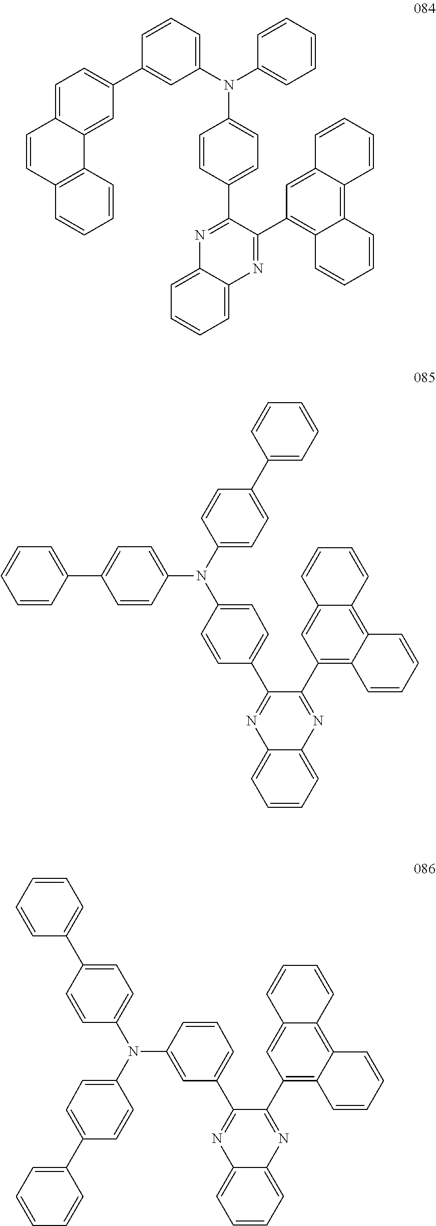 [SMILES: c1ccc(N(c2ccc(-c3nc4ccccc4nc3-c3cc4ccccc4c4ccccc34)cc2)c2cccc(-c3ccc4ccc5ccccc5c4c3)c2)cc1 ; c1ccc(-c2ccc(N(c3ccc(-c4ccccc4)cc3)c3ccc(-c4nc5ccccc5nc4-c4cc5ccccc5c5ccccc45)cc3)cc2)cc1 ; c1ccc(-c2ccc(N(c3ccc(-c4ccccc4)cc3)c3cccc(-c4nc5ccccc5nc4-c4cc5ccccc5c5ccccc45)c3)cc2)cc1]

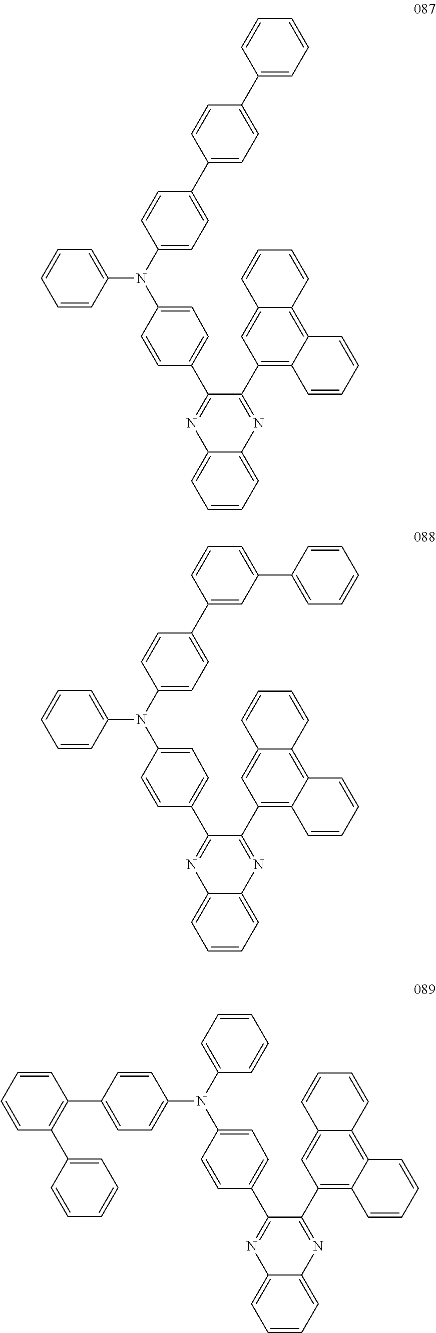

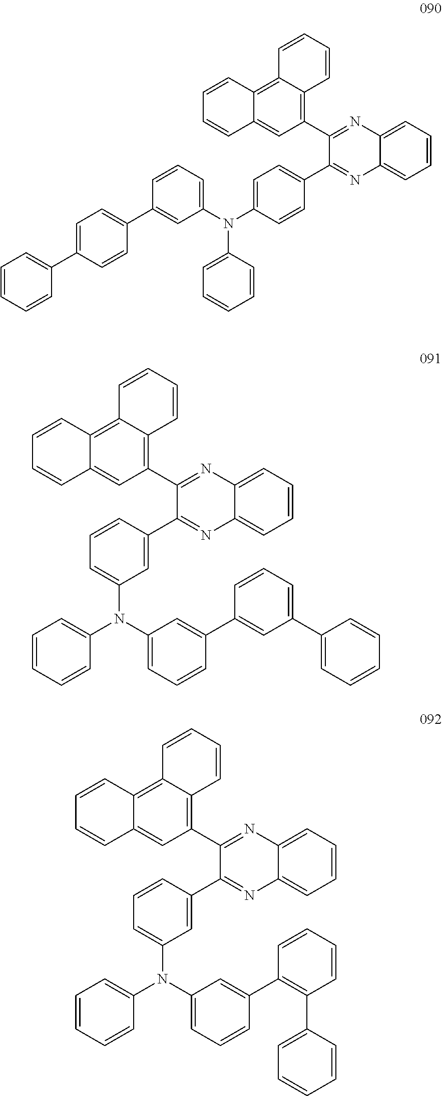

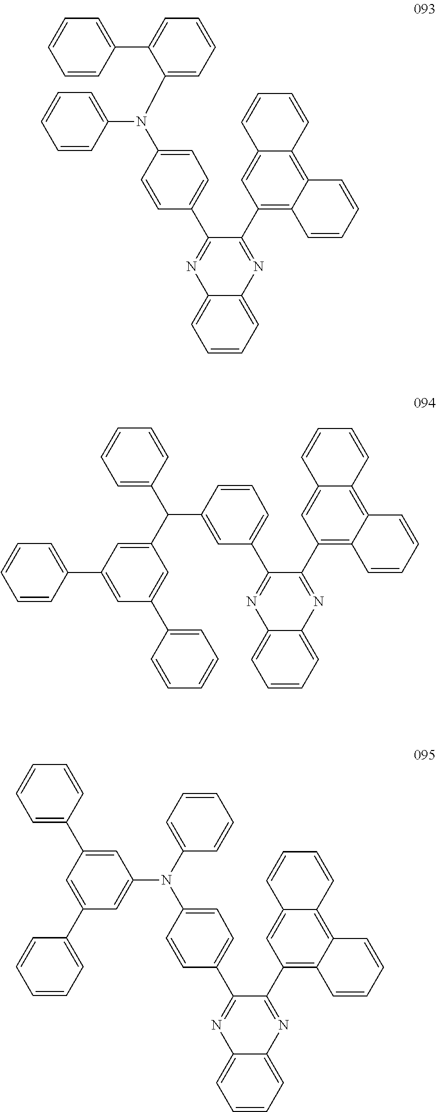 [SMILES: c1ccc(-c2ccccc2N(c2ccccc2)c2ccc(-c3nc4ccccc4nc3-c3cc4ccccc4c4ccccc34)cc2)cc1 ; c1ccc(-c2cc(-c3ccccc3)cc(C(c3ccccc3)c3cccc(-c4nc5ccccc5nc4-c4cc5ccccc5c5ccccc45)c3)c2)cc1 ; c1ccc(-c2cc(-c3ccccc3)cc(N(c3ccccc3)c3ccc(-c4nc5ccccc5nc4-c4cc5ccccc5c5ccccc45)cc3)c2)cc1]

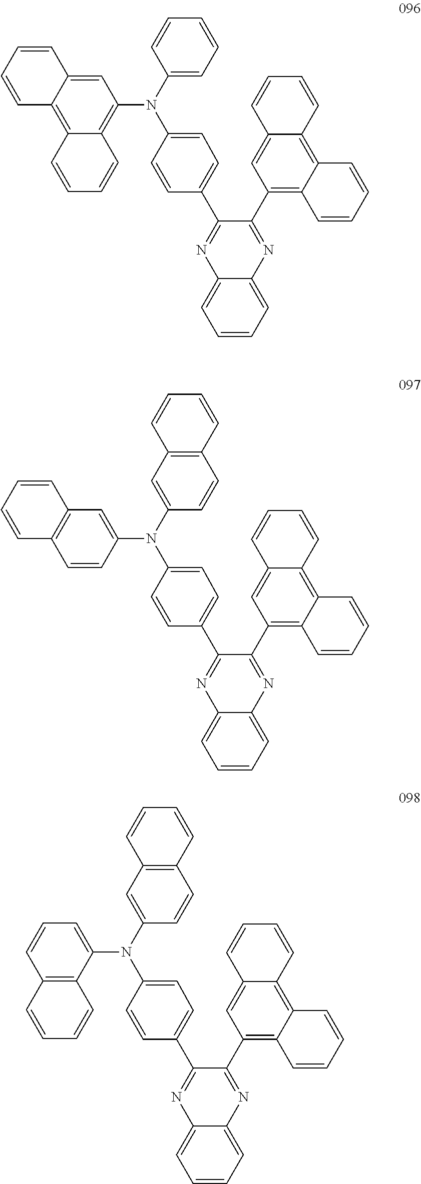 [SMILES: c1ccc(N(c2ccc(-c3nc4ccccc4nc3-c3cc4ccccc4c4ccccc34)cc2)c2cc3ccccc3c3ccccc23)cc1 ; c1ccc2cc(N(c3ccc(-c4nc5ccccc5nc4-c4cc5ccccc5c5ccccc45)cc3)c3ccc4ccccc4c3)ccc2c1 ; c1ccc2cc(N(c3ccc(-c4nc5ccccc5nc4-c4cc5ccccc5c5ccccc45)cc3)c3cccc4ccccc34)ccc2c1]

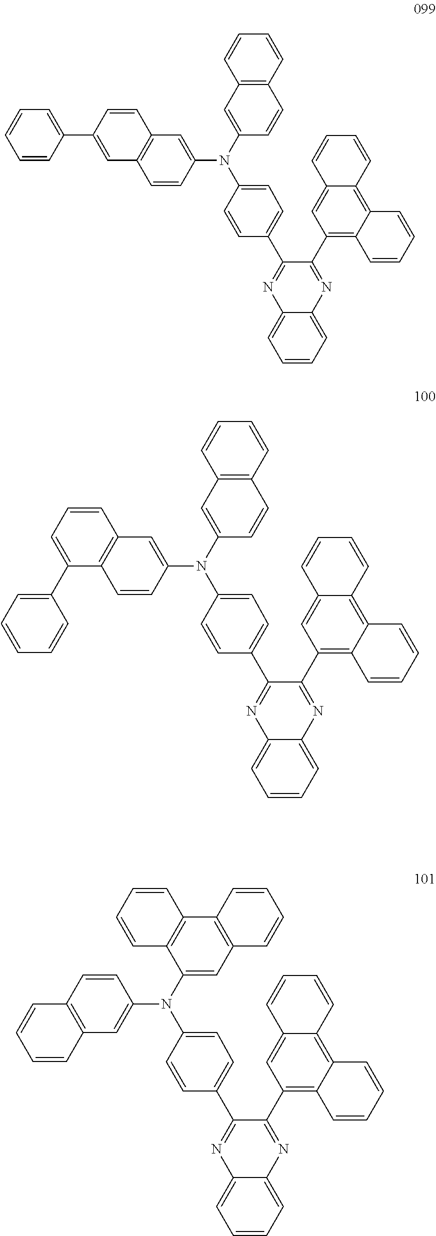 [SMILES: c1ccc(-c2ccc3cc(N(c4ccc(-c5nc6ccccc6nc5-c5cc6ccccc6c6ccccc56)cc4)c4ccc5ccccc5c4)ccc3c2)cc1 ; c1ccc(-c2cccc3cc(N(c4ccc(-c5nc6ccccc6nc5-c5cc6ccccc6c6ccccc56)cc4)c4ccc5ccccc5c4)ccc23)cc1 ; c1ccc2cc(N(c3ccc(-c4nc5ccccc5nc4-c4cc5ccccc5c5ccccc45)cc3)c3cc4ccccc4c4ccccc34)ccc2c1]

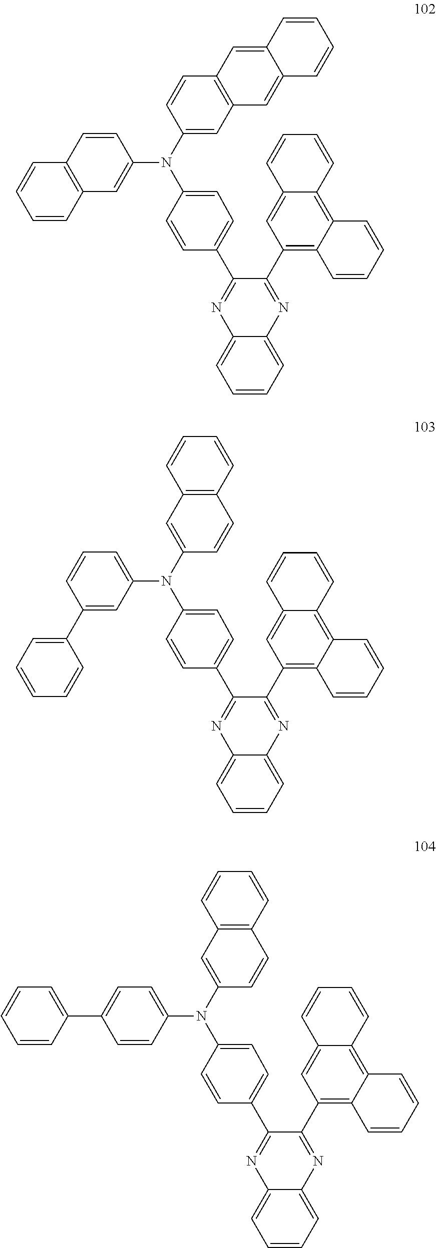

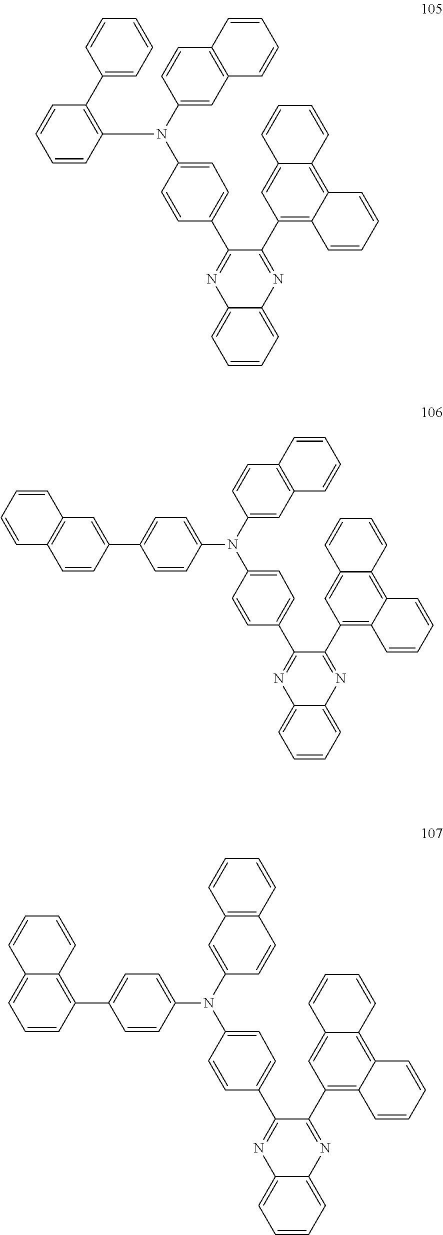 [SMILES: c1ccc(-c2ccccc2N(c2ccc(-c3nc4ccccc4nc3-c3cc4ccccc4c4ccccc34)cc2)c2ccc3ccccc3c2)cc1 ; c1ccc2cc(-c3ccc(N(c4ccc(-c5nc6ccccc6nc5-c5cc6ccccc6c6ccccc56)cc4)c4ccc5ccccc5c4)cc3)ccc2c1 ; c1ccc2cc(N(c3ccc(-c4nc5ccccc5nc4-c4cc5ccccc5c5ccccc45)cc3)c3ccc(-c4cccc5ccccc45)cc3)ccc2c1]

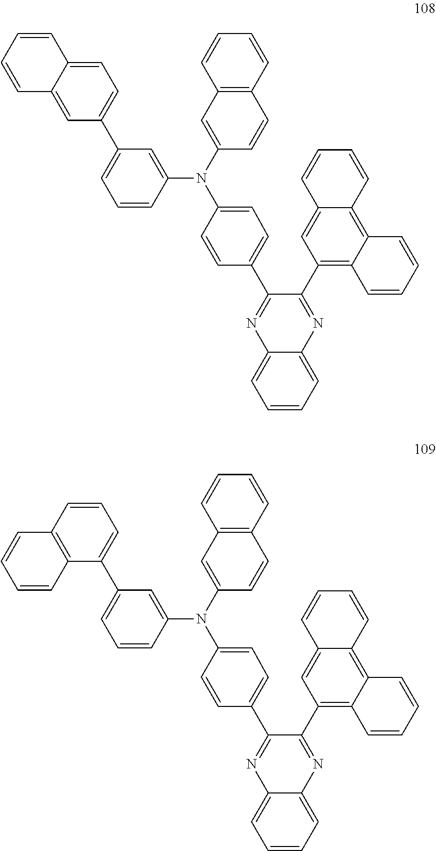 [SMILES: c1cc(-c2ccc3ccccc3c2)cc(N(c2ccc(-c3nc4ccccc4nc3-c3cc4ccccc4c4ccccc34)cc2)c2ccc3ccccc3c2)c1 ; c1cc(-c2cccc3ccccc23)cc(N(c2ccc(-c3nc4ccccc4nc3-c3cc4ccccc4c4ccccc34)cc2)c2ccc3ccccc3c2)c1]

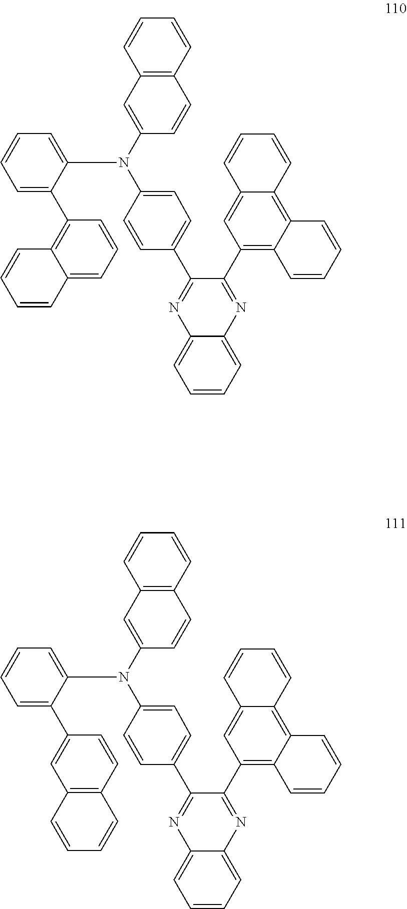 [SMILES: c1ccc(N(c2ccc(-c3nc4ccccc4nc3-c3cc4ccccc4c4ccccc34)cc2)c2ccc3ccccc3c2)c(-c2cccc3ccccc23)c1 ; c1ccc(N(c2ccc(-c3nc4ccccc4nc3-c3cc4ccccc4c4ccccc34)cc2)c2ccc3ccccc3c2)c(-c2ccc3ccccc3c2)c1]

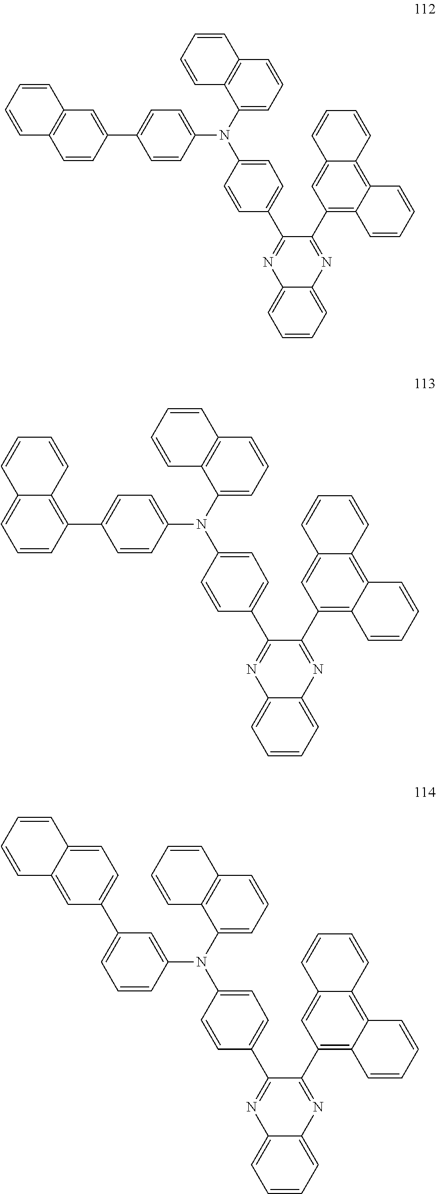 [SMILES: c1ccc2cc(-c3ccc(N(c4ccc(-c5nc6ccccc6nc5-c5cc6ccccc6c6ccccc56)cc4)c4cccc5ccccc45)cc3)ccc2c1 ; c1ccc2c(-c3ccc(N(c4ccc(-c5nc6ccccc6nc5-c5cc6ccccc6c6ccccc56)cc4)c4cccc5ccccc45)cc3)cccc2c1 ; c1cc(-c2ccc3ccccc3c2)cc(N(c2ccc(-c3nc4ccccc4nc3-c3cc4ccccc4c4ccccc34)cc2)c2cccc3ccccc23)c1]

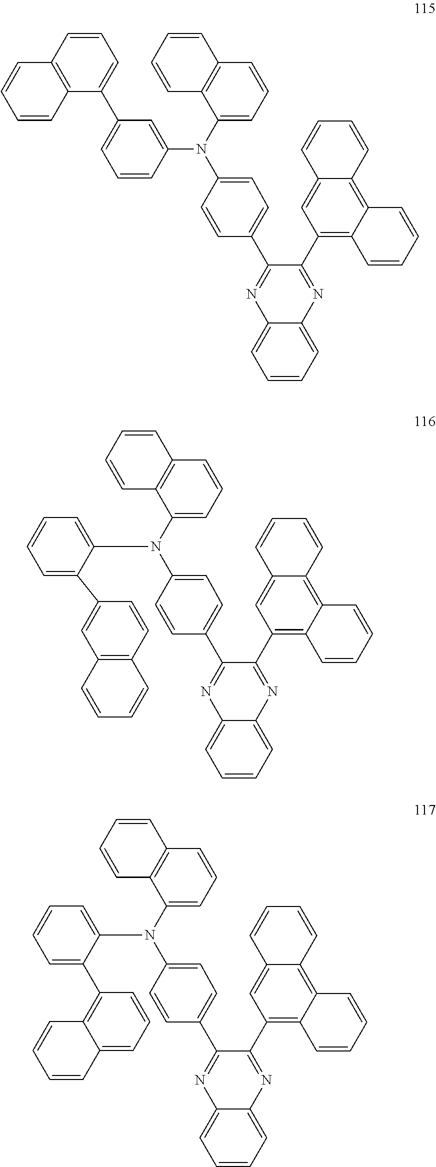 [SMILES: c1cc(-c2cccc3ccccc23)cc(N(c2ccc(-c3nc4ccccc4nc3-c3cc4ccccc4c4ccccc34)cc2)c2cccc3ccccc23)c1 ; c1ccc(N(c2ccc(-c3nc4ccccc4nc3-c3cc4ccccc4c4ccccc34)cc2)c2cccc3ccccc23)c(-c2ccc3ccccc3c2)c1 ; c1ccc(N(c2ccc(-c3nc4ccccc4nc3-c3cc4ccccc4c4ccccc34)cc2)c2cccc3ccccc23)c(-c2cccc3ccccc23)c1]

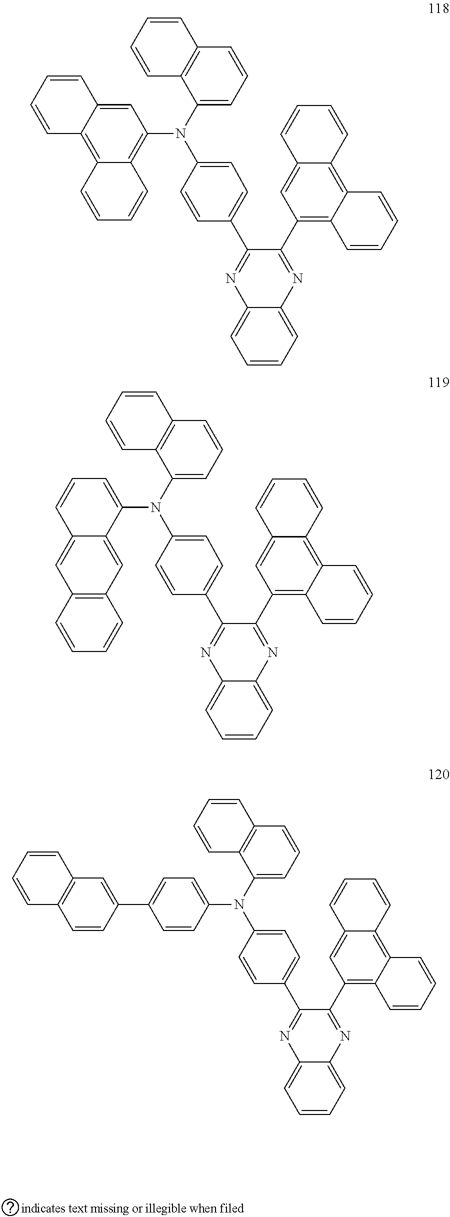 [SMILES: c1ccc2c(N(c3ccc(-c4nc5ccccc5nc4-c4cc5ccccc5c5ccccc45)cc3)c3cc4ccccc4c4ccccc34)cccc2c1 ; c1ccc2cc3c(N(c4ccc(-c5nc6ccccc6nc5-c5cc6ccccc6c6ccccc56)cc4)c4cccc5ccccc45)cccc3cc2c1 ; c1ccc2cc(-c3ccc(N(c4ccc(-c5nc6ccccc6nc5-c5cc6ccccc6c6ccccc56)cc4)c4cccc5ccccc45)cc3)ccc2c1]

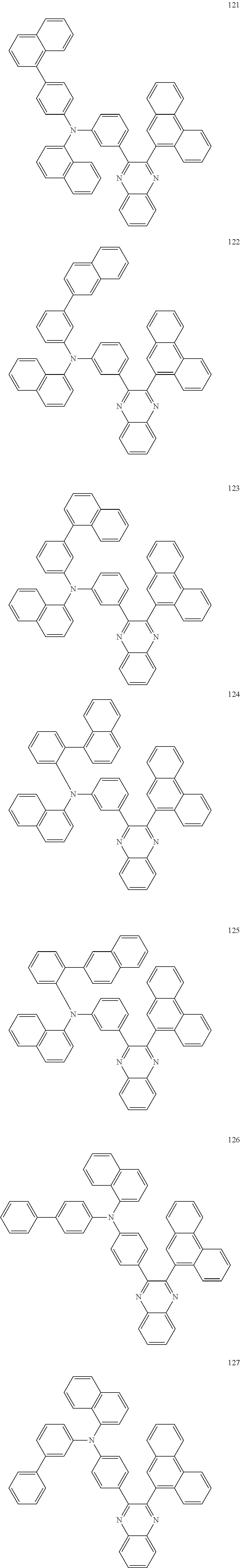

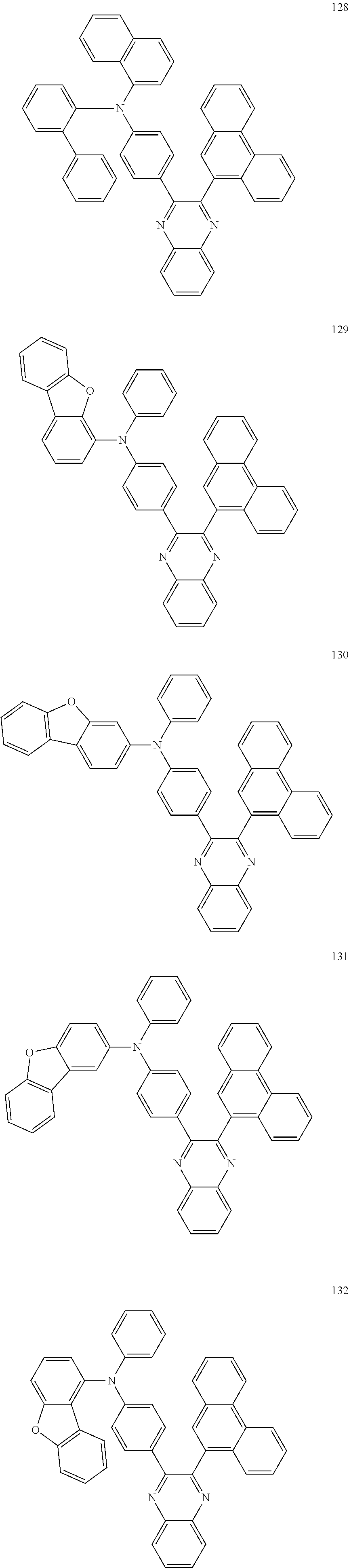 [SMILES: c1ccc(-c2ccccc2N(c2ccc(-c3nc4ccccc4nc3-c3cc4ccccc4c4ccccc34)cc2)c2cccc3ccccc23)cc1 ; c1ccc(N(c2ccc(-c3nc4ccccc4nc3-c3cc4ccccc4c4ccccc34)cc2)c2cccc3c2oc2ccccc23)cc1 ; c1ccc(N(c2ccc(-c3nc4ccccc4nc3-c3cc4ccccc4c4ccccc34)cc2)c2ccc3c(c2)oc2ccccc23)cc1 ; c1ccc(N(c2ccc(-c3nc4ccccc4nc3-c3cc4ccccc4c4ccccc34)cc2)c2ccc3oc4ccccc4c3c2)cc1 ; c1ccc(N(c2ccc(-c3nc4ccccc4nc3-c3cc4ccccc4c4ccccc34)cc2)c2cccc3oc4ccccc4c23)cc1]

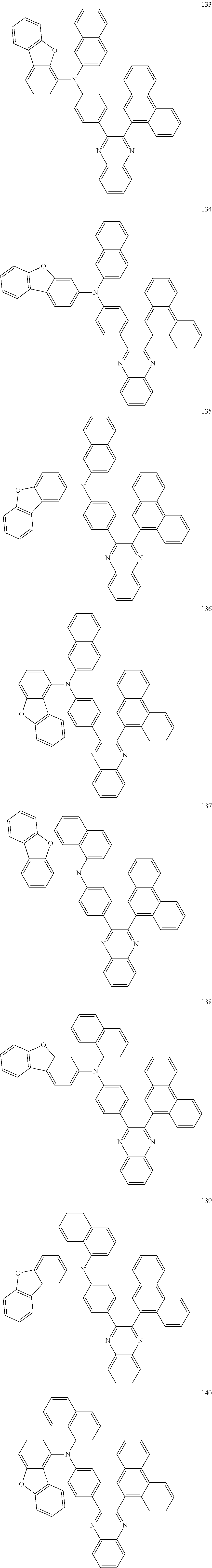

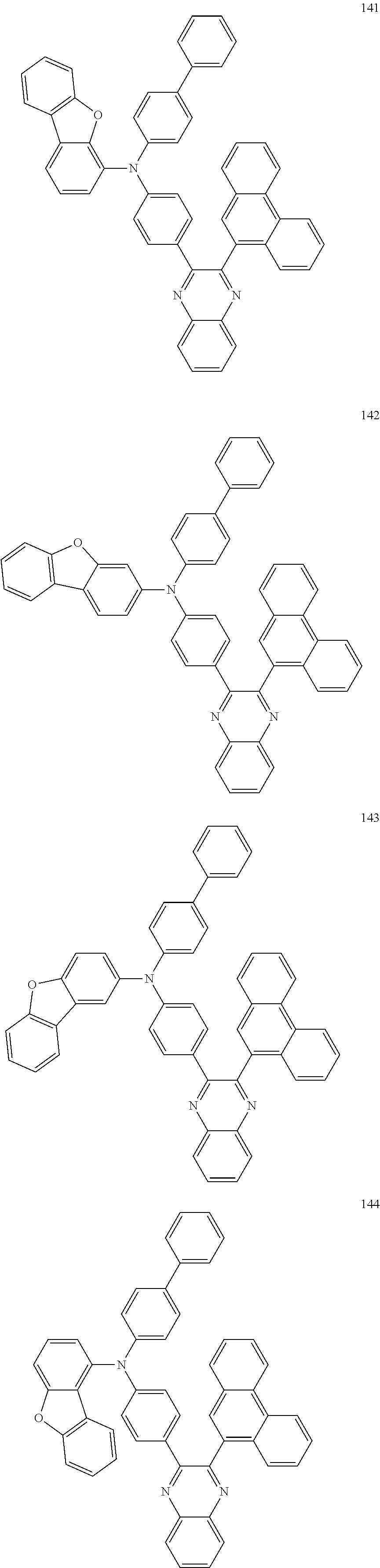

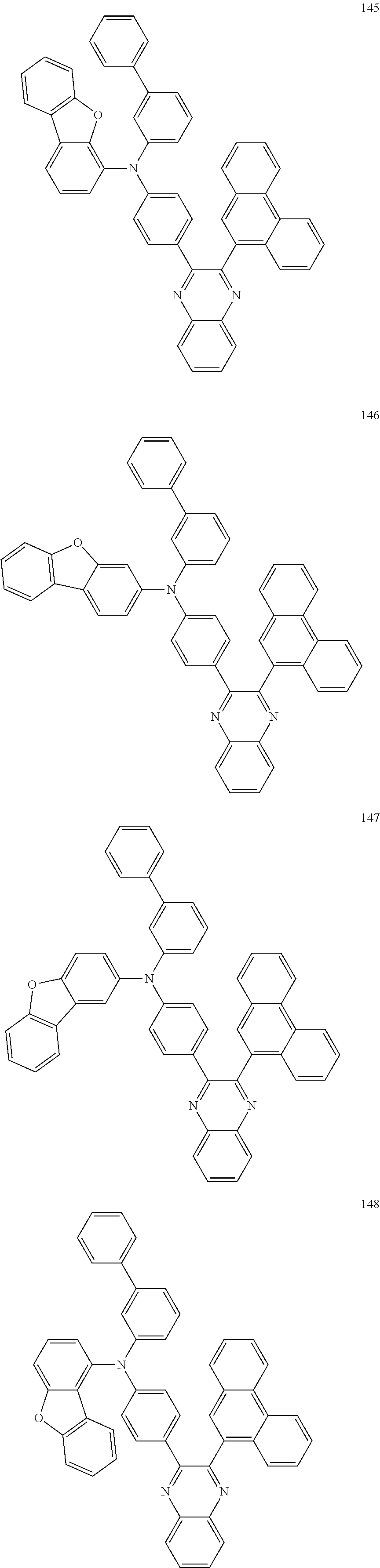 [SMILES: c1ccc(-c2cccc(N(c3ccc(-c4nc5ccccc5nc4-c4cc5ccccc5c5ccccc45)cc3)c3cccc4c3oc3ccccc34)c2)cc1 ; c1ccc(-c2cccc(N(c3ccc(-c4nc5ccccc5nc4-c4cc5ccccc5c5ccccc45)cc3)c3ccc4c(c3)oc3ccccc34)c2)cc1 ; c1ccc(-c2cccc(N(c3ccc(-c4nc5ccccc5nc4-c4cc5ccccc5c5ccccc45)cc3)c3ccc4oc5ccccc5c4c3)c2)cc1 ; c1ccc(-c2cccc(N(c3ccc(-c4nc5ccccc5nc4-c4cc5ccccc5c5ccccc45)cc3)c3cccc4oc5ccccc5c34)c2)cc1]

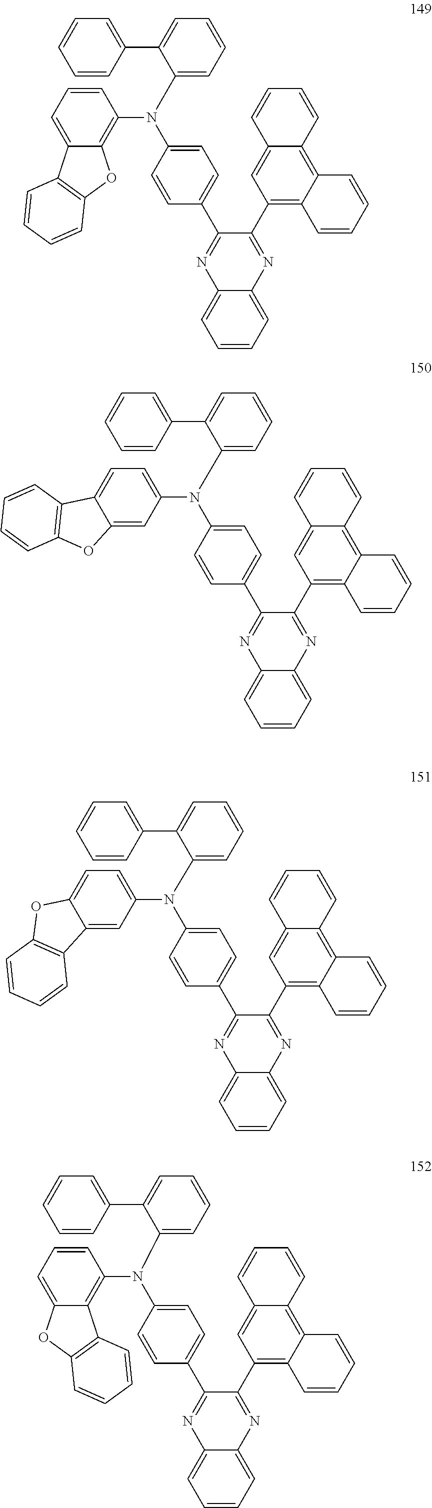

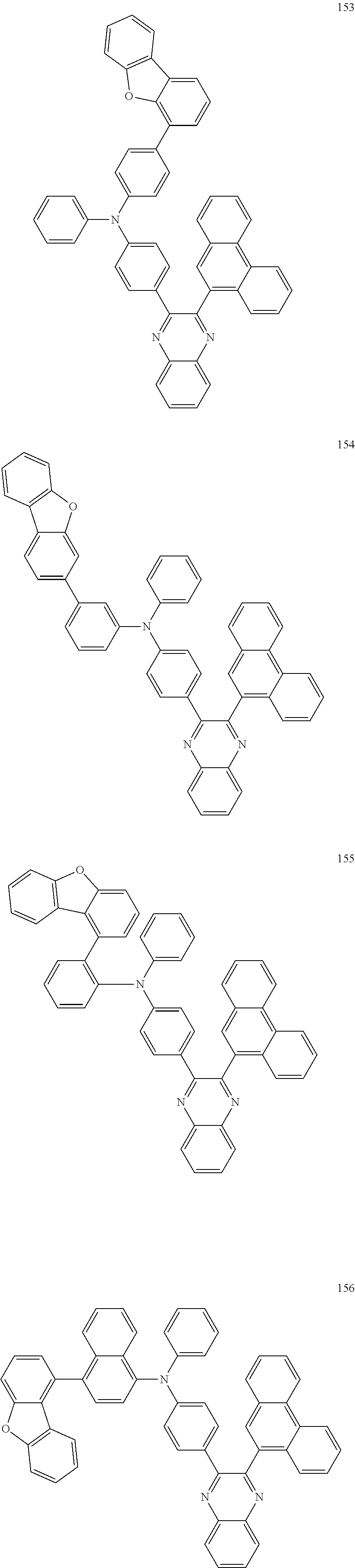 [SMILES: c1ccc(N(c2ccc(-c3nc4ccccc4nc3-c3cc4ccccc4c4ccccc34)cc2)c2ccc(-c3cccc4c3oc3ccccc34)cc2)cc1 ; c1ccc(N(c2ccc(-c3nc4ccccc4nc3-c3cc4ccccc4c4ccccc34)cc2)c2cccc(-c3ccc4c(c3)oc3ccccc34)c2)cc1 ; c1ccc(N(c2ccc(-c3nc4ccccc4nc3-c3cc4ccccc4c4ccccc34)cc2)c2ccccc2-c2cccc3oc4ccccc4c23)cc1 ; c1ccc(N(c2ccc(-c3nc4ccccc4nc3-c3cc4ccccc4c4ccccc34)cc2)c2ccc(-c3cccc4oc5ccccc5c34)c3ccccc23)cc1]

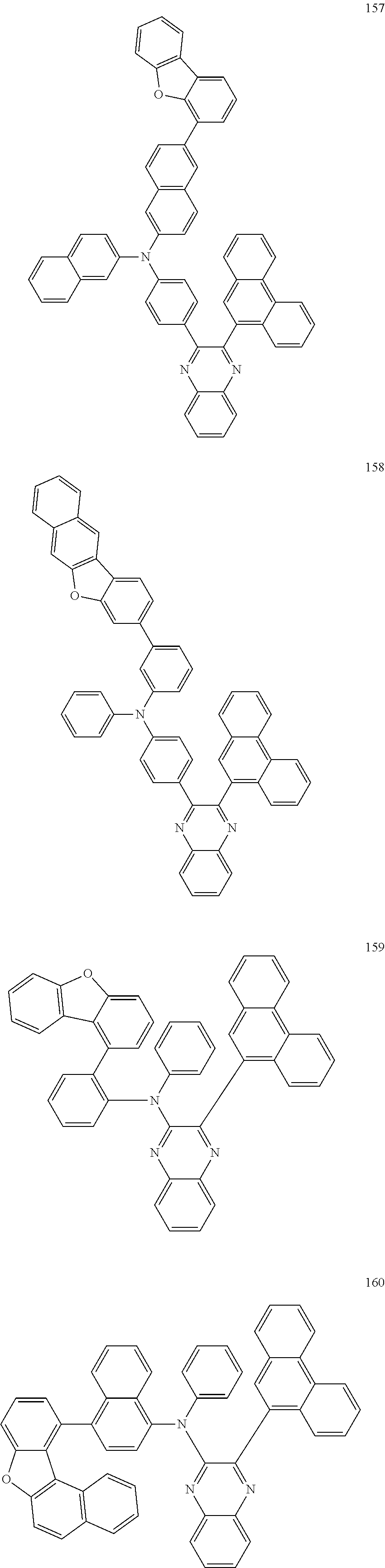 [SMILES: c1ccc2cc(N(c3ccc(-c4nc5ccccc5nc4-c4cc5ccccc5c5ccccc45)cc3)c3ccc4cc(-c5cccc6c5oc5ccccc56)ccc4c3)ccc2c1 ; c1ccc(N(c2ccc(-c3nc4ccccc4nc3-c3cc4ccccc4c4ccccc34)cc2)c2cccc(-c3ccc4c(c3)oc3cc5ccccc5cc34)c2)cc1 ; c1ccc(N(c2ccccc2-c2cccc3oc4ccccc4c23)c2nc3ccccc3nc2-c2cc3ccccc3c3ccccc23)cc1 ; c1ccc(N(c2nc3ccccc3nc2-c2cc3ccccc3c3ccccc23)c2ccc(-c3cccc4oc5ccc6ccccc6c5c34)c3ccccc23)cc1]

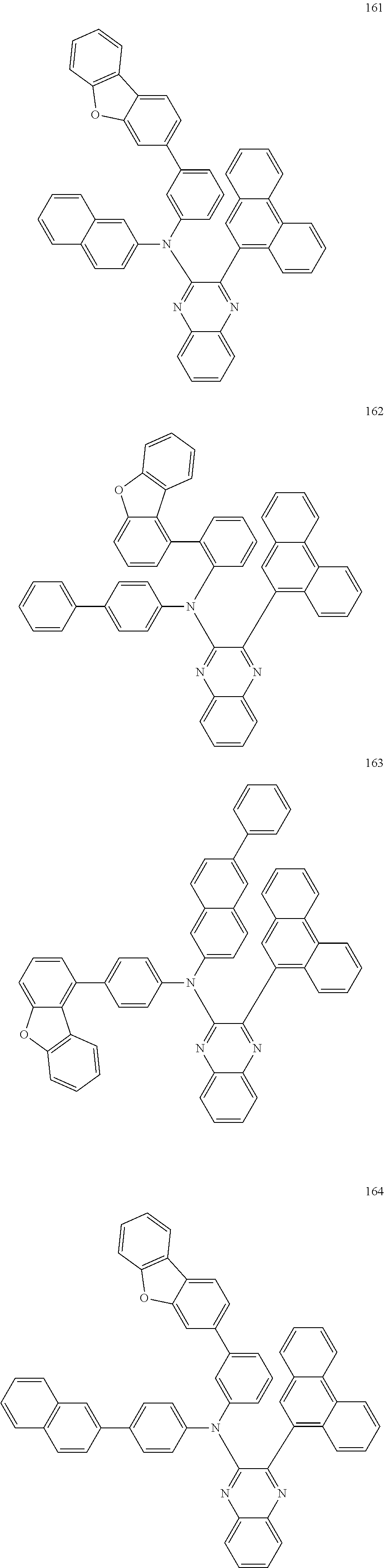

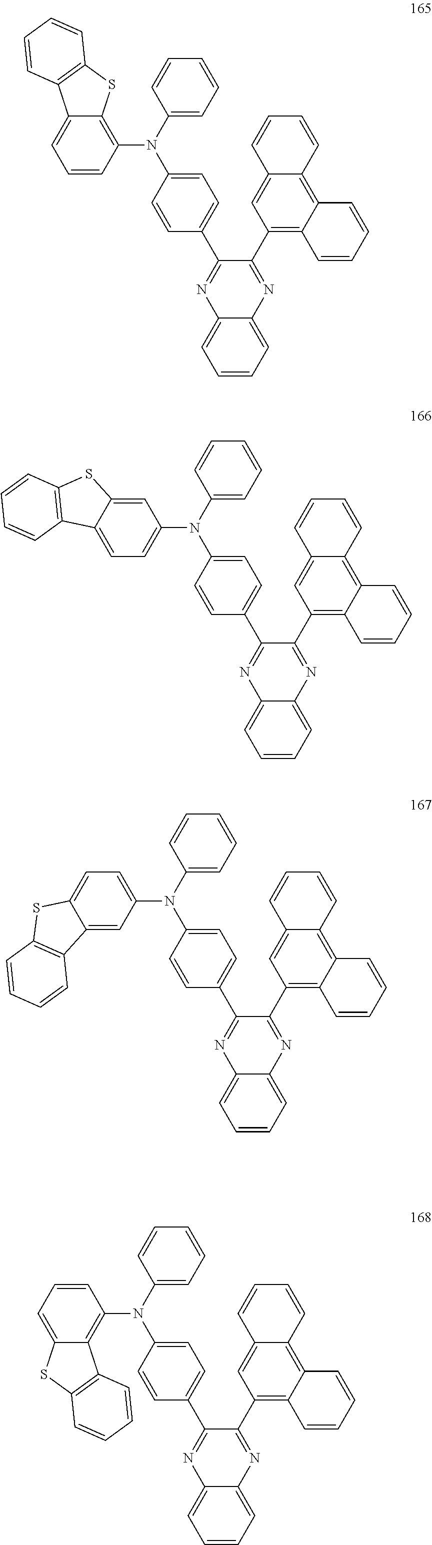 [SMILES: c1ccc(N(c2ccc(-c3nc4ccccc4nc3-c3cc4ccccc4c4ccccc34)cc2)c2cccc3c2sc2ccccc23)cc1 ; c1ccc(N(c2ccc(-c3nc4ccccc4nc3-c3cc4ccccc4c4ccccc34)cc2)c2ccc3c(c2)sc2ccccc23)cc1 ; c1ccc(N(c2ccc(-c3nc4ccccc4nc3-c3cc4ccccc4c4ccccc34)cc2)c2ccc3sc4ccccc4c3c2)cc1 ; c1ccc(N(c2ccc(-c3nc4ccccc4nc3-c3cc4ccccc4c4ccccc34)cc2)c2cccc3sc4ccccc4c23)cc1]

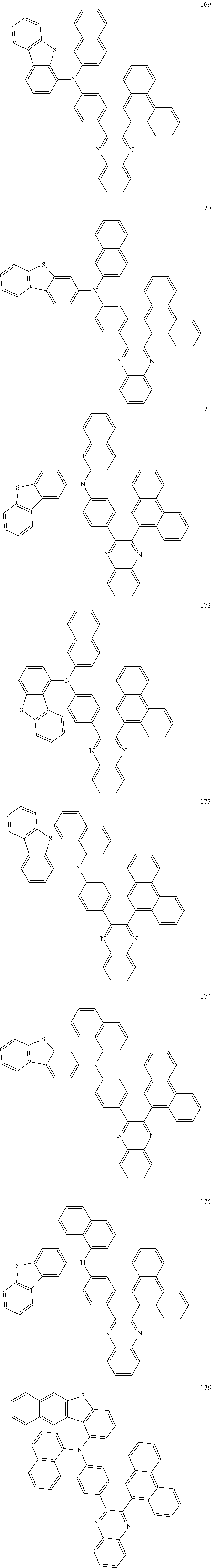

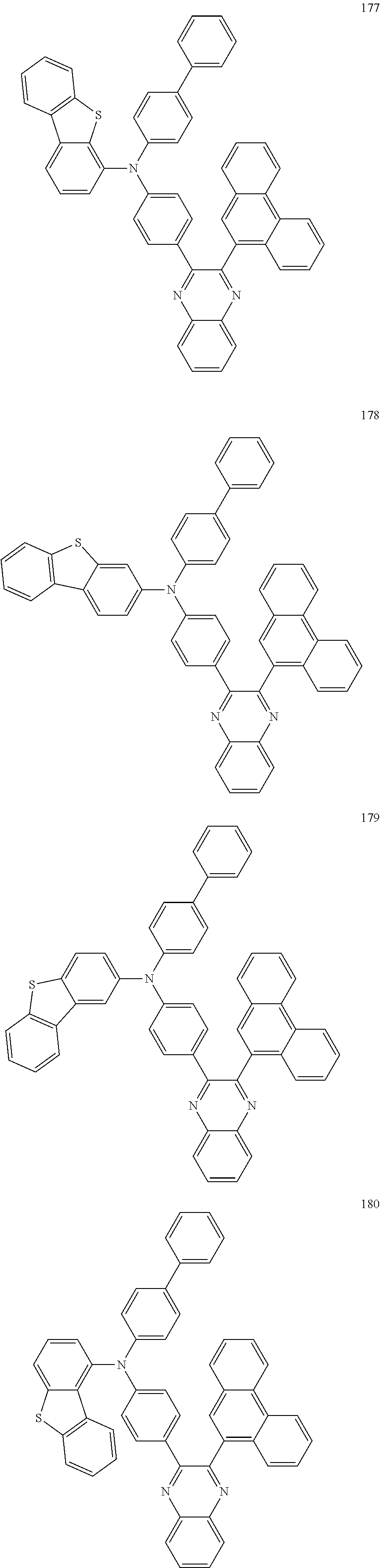

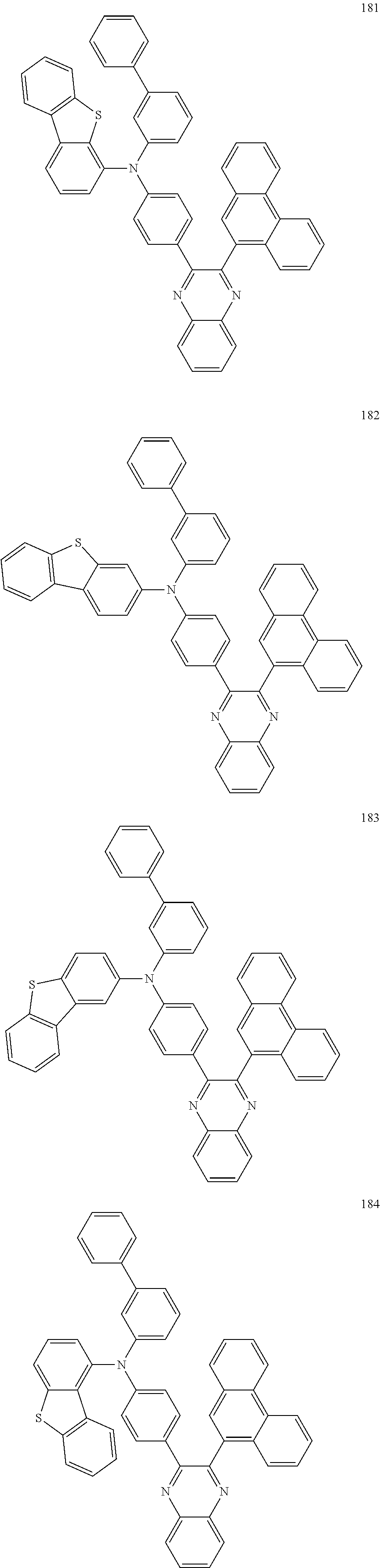 [SMILES: c1ccc(-c2cccc(N(c3ccc(-c4nc5ccccc5nc4-c4cc5ccccc5c5ccccc45)cc3)c3cccc4c3sc3ccccc34)c2)cc1 ; c1ccc(-c2cccc(N(c3ccc(-c4nc5ccccc5nc4-c4cc5ccccc5c5ccccc45)cc3)c3ccc4c(c3)sc3ccccc34)c2)cc1 ; c1ccc(-c2cccc(N(c3ccc(-c4nc5ccccc5nc4-c4cc5ccccc5c5ccccc45)cc3)c3ccc4sc5ccccc5c4c3)c2)cc1 ; c1ccc(-c2cccc(N(c3ccc(-c4nc5ccccc5nc4-c4cc5ccccc5c5ccccc45)cc3)c3cccc4sc5ccccc5c34)c2)cc1]

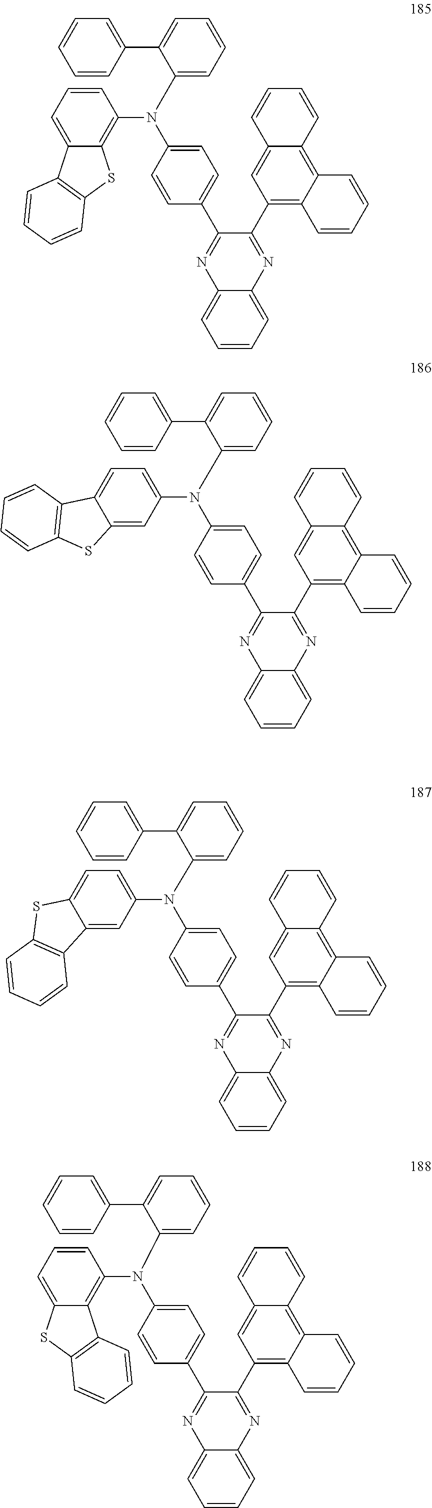

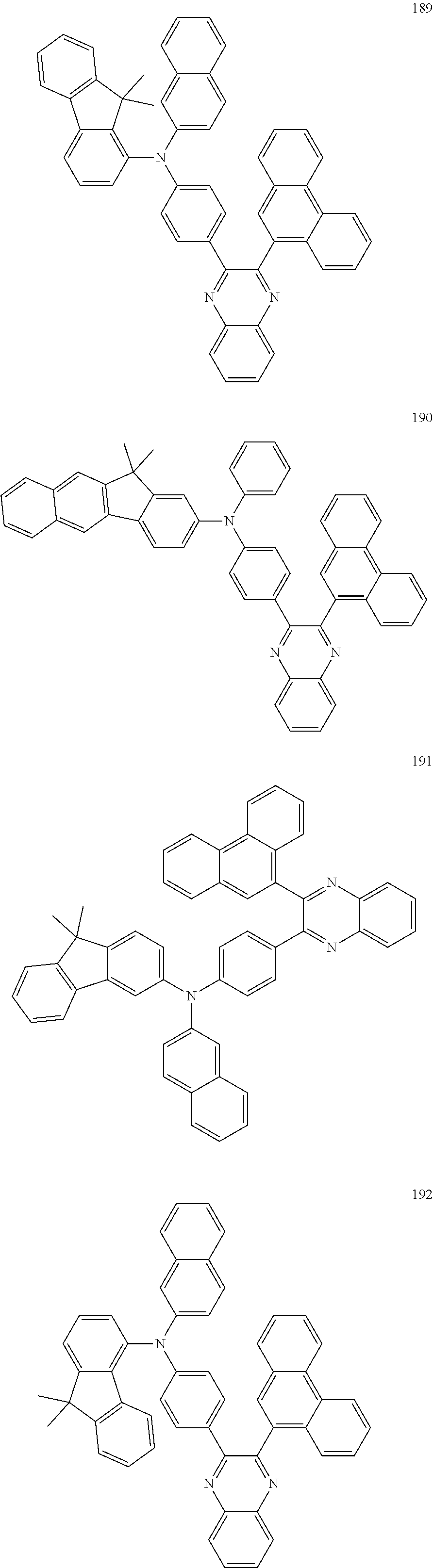 [SMILES: CC1(C)c2ccccc2-c2cccc(N(c3ccc(-c4nc5ccccc5nc4-c4cc5ccccc5c5ccccc45)cc3)c3ccc4ccccc4c3)c21 ; CC1(C)c2cc(N(c3ccccc3)c3ccc(-c4nc5ccccc5nc4-c4cc5ccccc5c5ccccc45)cc3)ccc2-c2cc3ccccc3cc21 ; CC1(C)c2ccccc2-c2cc(N(c3ccc(-c4nc5ccccc5nc4-c4cc5ccccc5c5ccccc45)cc3)c3ccc4ccccc4c3)ccc21 ; CC1(C)c2ccccc2-c2c(N(c3ccc(-c4nc5ccccc5nc4-c4cc5ccccc5c5ccccc45)cc3)c3ccc4ccccc4c3)cccc21]

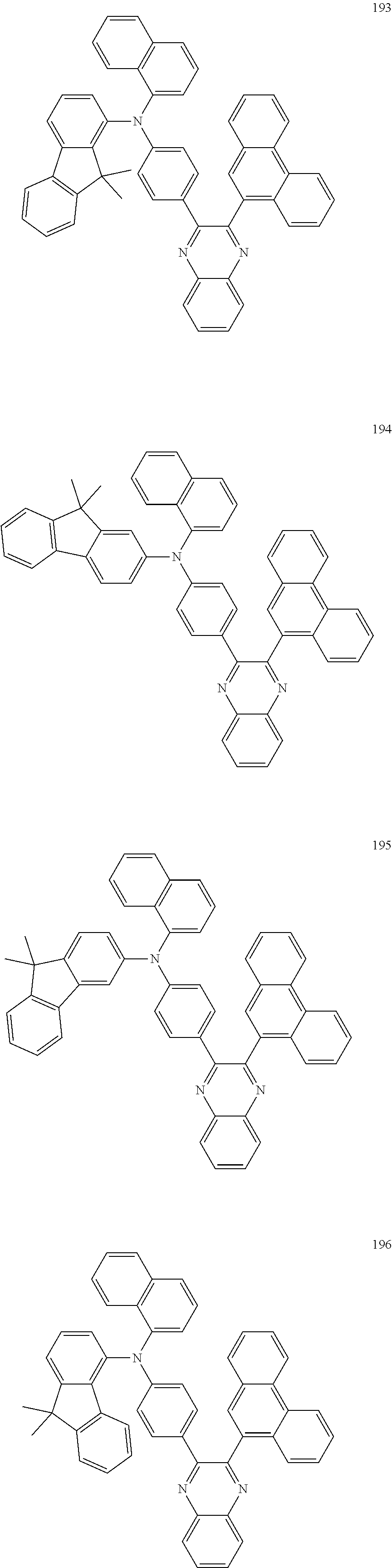 [SMILES: CC1(C)c2ccccc2-c2cccc(N(c3ccc(-c4nc5ccccc5nc4-c4cc5ccccc5c5ccccc45)cc3)c3cccc4ccccc34)c21 ; CC1(C)c2ccccc2-c2ccc(N(c3ccc(-c4nc5ccccc5nc4-c4cc5ccccc5c5ccccc45)cc3)c3cccc4ccccc34)cc21 ; CC1(C)c2ccccc2-c2cc(N(c3ccc(-c4nc5ccccc5nc4-c4cc5ccccc5c5ccccc45)cc3)c3cccc4ccccc34)ccc21 ; CC1(C)c2ccccc2-c2c(N(c3ccc(-c4nc5ccccc5nc4-c4cc5ccccc5c5ccccc45)cc3)c3cccc4ccccc34)cccc21]

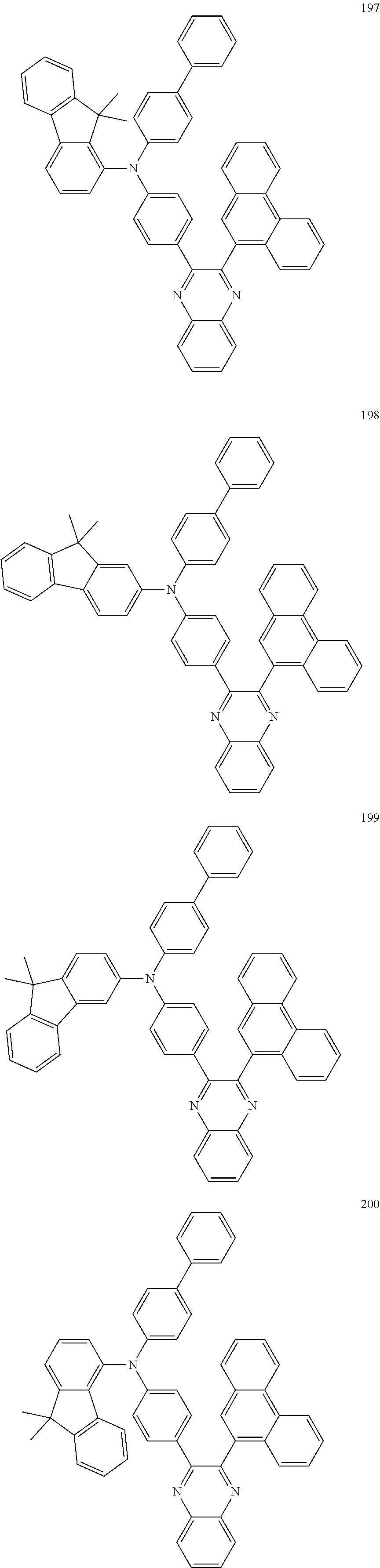

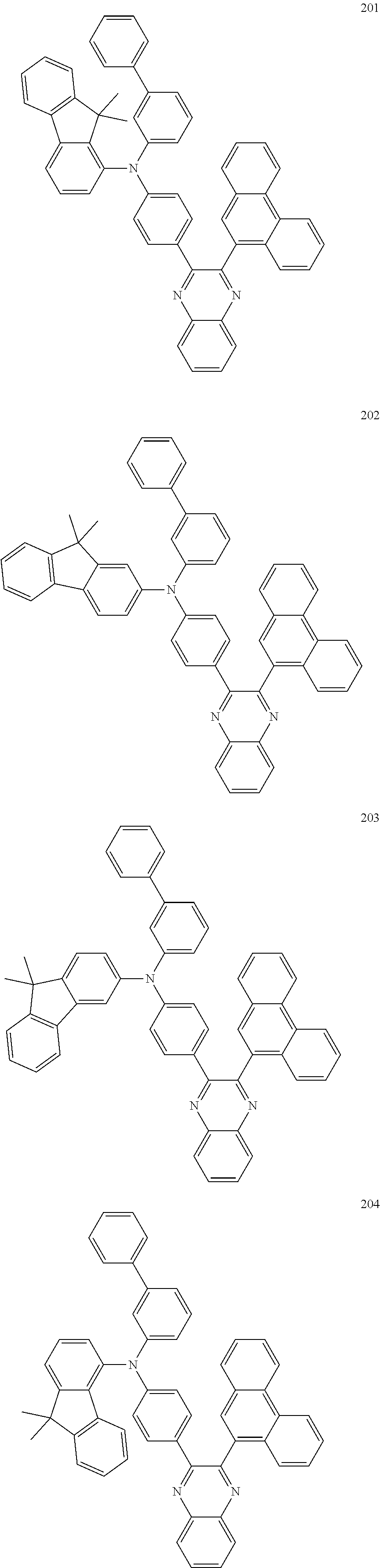 [SMILES: CC1(C)c2ccccc2-c2cccc(N(c3ccc(-c4nc5ccccc5nc4-c4cc5ccccc5c5ccccc45)cc3)c3cccc(-c4ccccc4)c3)c21 ; CC1(C)c2ccccc2-c2ccc(N(c3ccc(-c4nc5ccccc5nc4-c4cc5ccccc5c5ccccc45)cc3)c3cccc(-c4ccccc4)c3)cc21 ; CC1(C)c2ccccc2-c2cc(N(c3ccc(-c4nc5ccccc5nc4-c4cc5ccccc5c5ccccc45)cc3)c3cccc(-c4ccccc4)c3)ccc21 ; CC1(C)c2ccccc2-c2c(N(c3ccc(-c4nc5ccccc5nc4-c4cc5ccccc5c5ccccc45)cc3)c3cccc(-c4ccccc4)c3)cccc21]

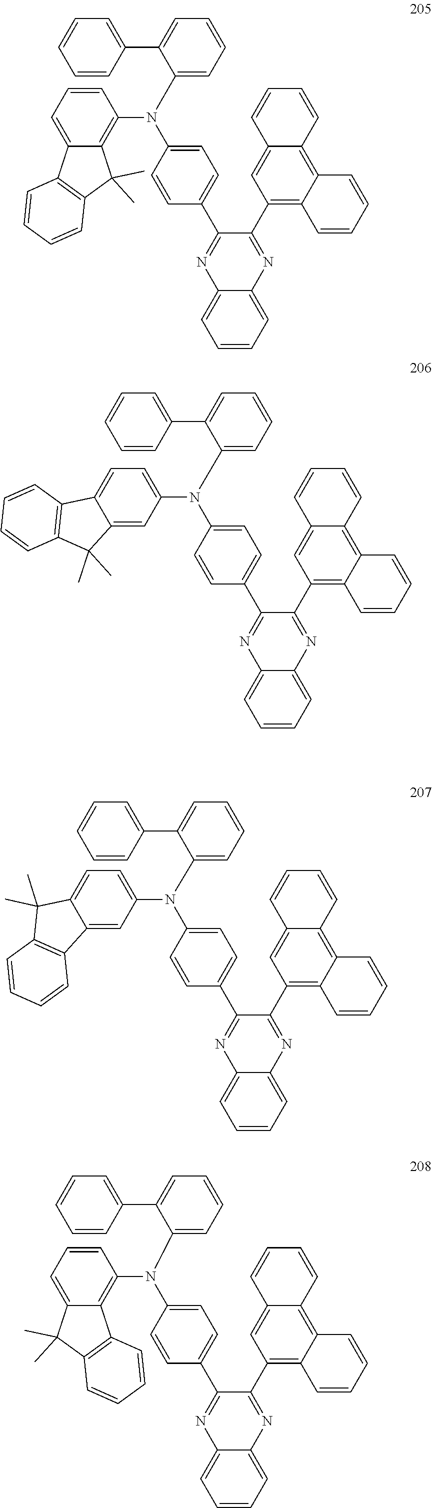

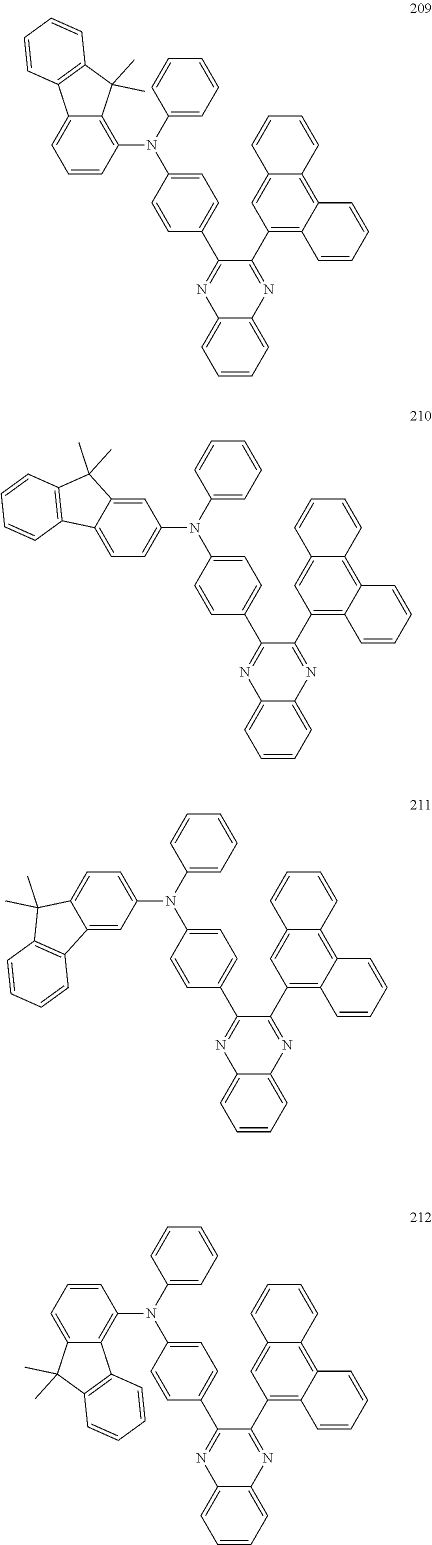 [SMILES: CC1(C)c2ccccc2-c2cccc(N(c3ccccc3)c3ccc(-c4nc5ccccc5nc4-c4cc5ccccc5c5ccccc45)cc3)c21 ; CC1(C)c2ccccc2-c2ccc(N(c3ccccc3)c3ccc(-c4nc5ccccc5nc4-c4cc5ccccc5c5ccccc45)cc3)cc21 ; CC1(C)c2ccccc2-c2cc(N(c3ccccc3)c3ccc(-c4nc5ccccc5nc4-c4cc5ccccc5c5ccccc45)cc3)ccc21 ; CC1(C)c2ccccc2-c2c(N(c3ccccc3)c3ccc(-c4nc5ccccc5nc4-c4cc5ccccc5c5ccccc45)cc3)cccc21]

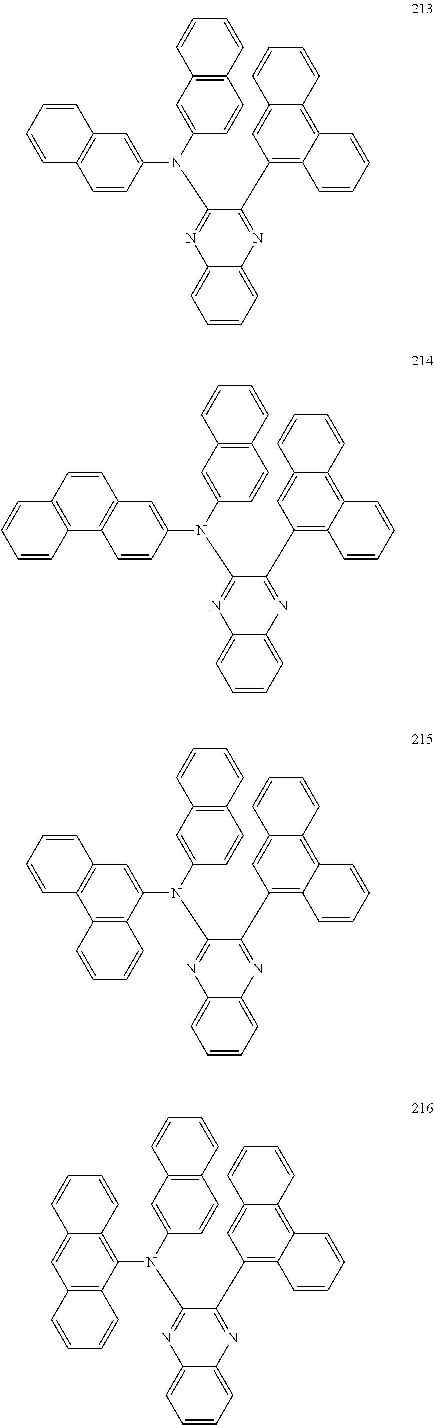 [SMILES: c1ccc2cc(N(c3ccc4ccccc4c3)c3nc4ccccc4nc3-c3cc4ccccc4c4ccccc34)ccc2c1 ; c1ccc2cc(N(c3ccc4c(ccc5ccccc54)c3)c3nc4ccccc4nc3-c3cc4ccccc4c4ccccc34)ccc2c1 ; c1ccc2cc(N(c3nc4ccccc4nc3-c3cc4ccccc4c4ccccc34)c3cc4ccccc4c4ccccc34)ccc2c1 ; c1ccc2cc(N(c3nc4ccccc4nc3-c3cc4ccccc4c4ccccc34)c3c4ccccc4cc4ccccc34)ccc2c1]

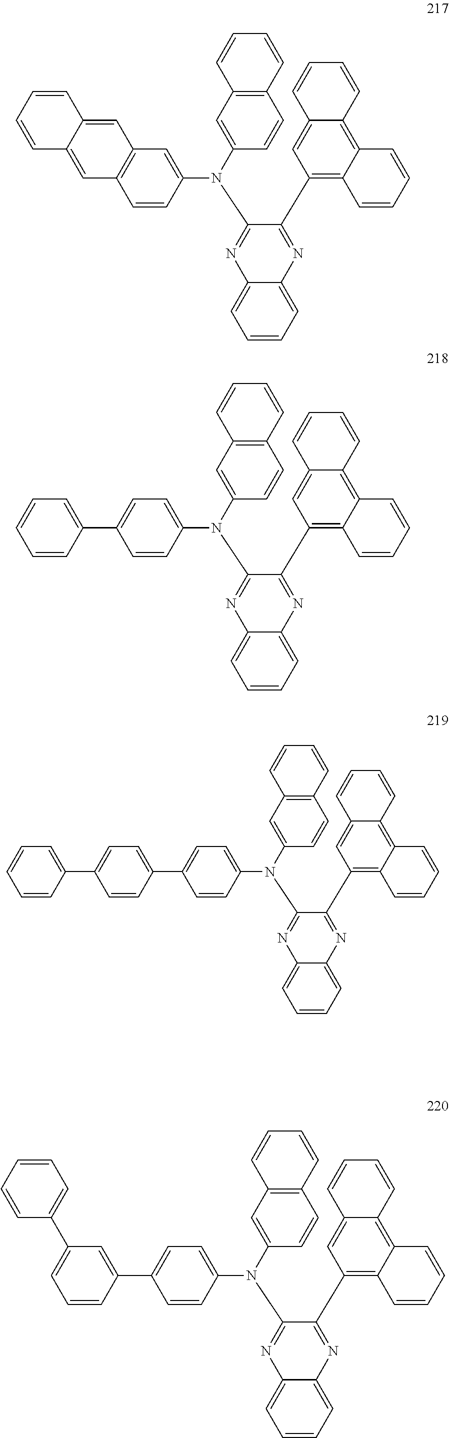

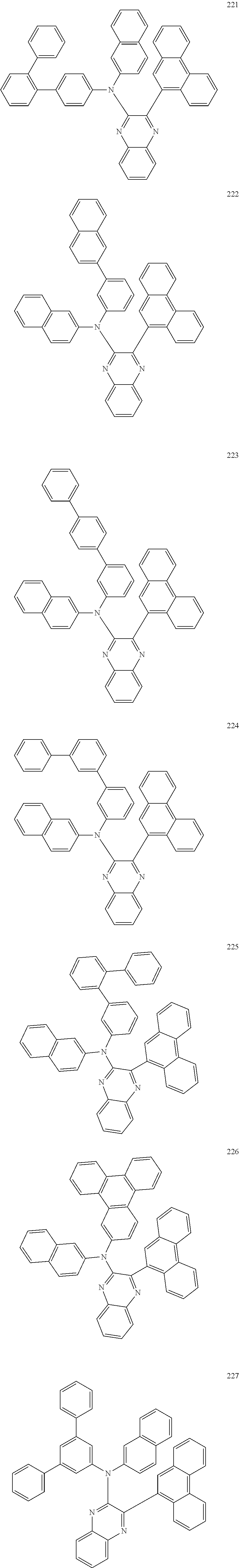

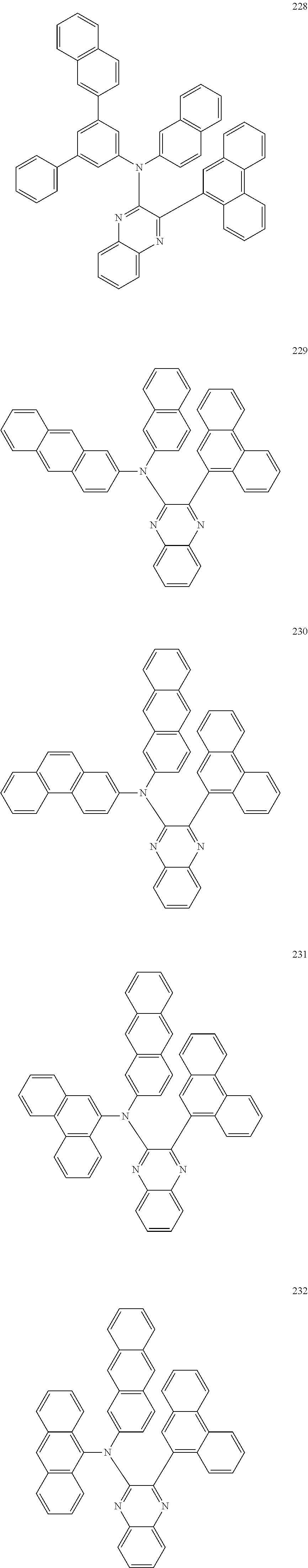 [SMILES: c1ccc(-c2cc(-c3ccc4ccccc4c3)cc(N(c3ccc4ccccc4c3)c3nc4ccccc4nc3-c3cc4ccccc4c4ccccc34)c2)cc1 ; c1ccc2cc(N(c3ccc4cc5ccccc5cc4c3)c3nc4ccccc4nc3-c3cc4ccccc4c4ccccc34)ccc2c1 ; c1ccc2cc3cc(N(c4ccc5c(ccc6ccccc65)c4)c4nc5ccccc5nc4-c4cc5ccccc5c5ccccc45)ccc3cc2c1 ; c1ccc2cc3cc(N(c4nc5ccccc5nc4-c4cc5ccccc5c5ccccc45)c4cc5ccccc5c5ccccc45)ccc3cc2c1 ; c1ccc2cc3cc(N(c4nc5ccccc5nc4-c4cc5ccccc5c5ccccc45)c4c5ccccc5cc5ccccc45)ccc3cc2c1]

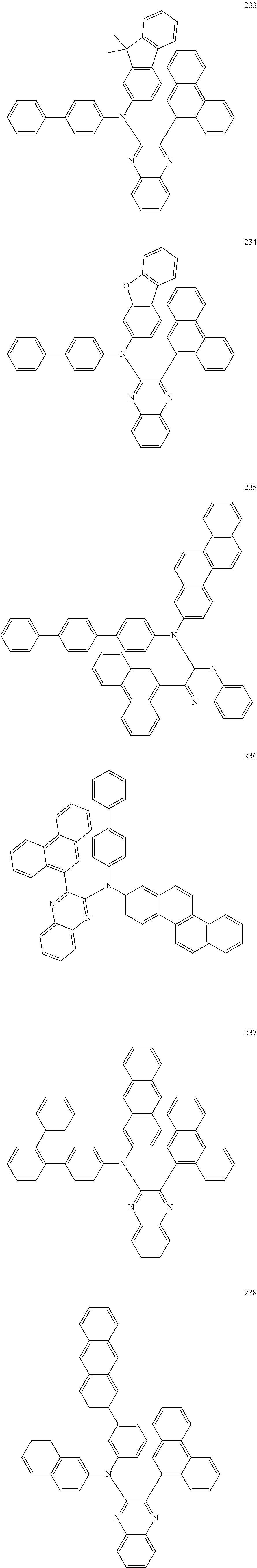

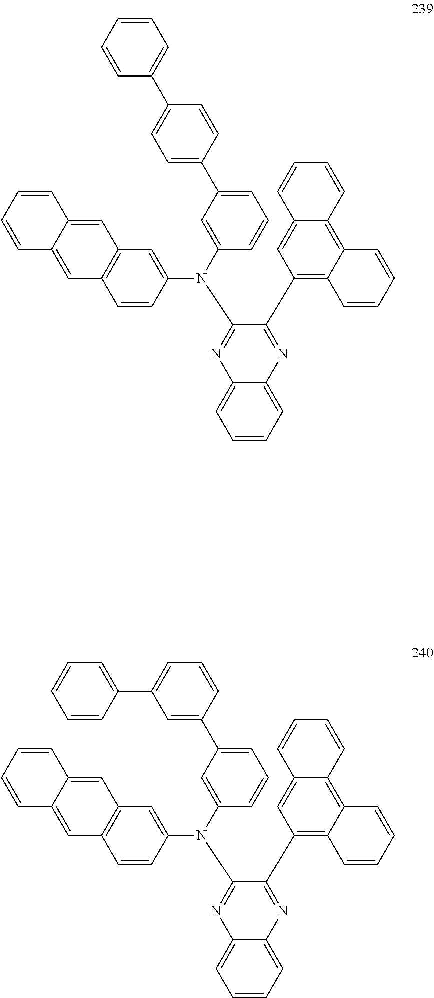 [SMILES: c1ccc(-c2ccc(-c3cccc(N(c4ccc5cc6ccccc6cc5c4)c4nc5ccccc5nc4-c4cc5ccccc5c5ccccc45)c3)cc2)cc1 ; c1ccc(-c2cccc(-c3cccc(N(c4ccc5cc6ccccc6cc5c4)c4nc5ccccc5nc4-c4cc5ccccc5c5ccccc45)c3)c2)cc1]

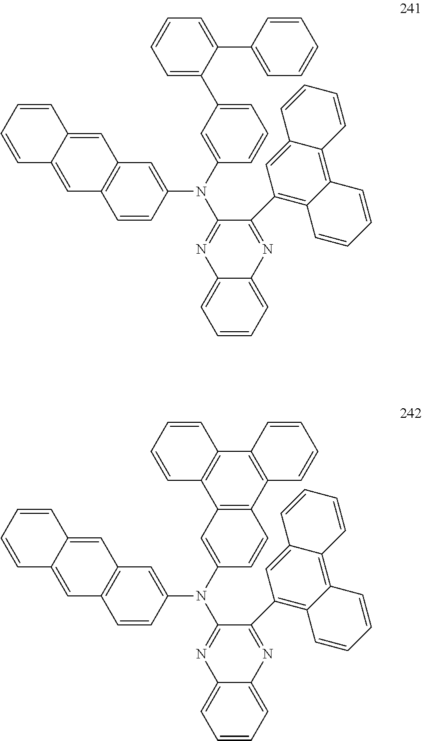 [SMILES: c1ccc(-c2ccccc2-c2cccc(N(c3ccc4cc5ccccc5cc4c3)c3nc4ccccc4nc3-c3cc4ccccc4c4ccccc34)c2)cc1 ; c1ccc2cc3cc(N(c4ccc5c6ccccc6c6ccccc6c5c4)c4nc5ccccc5nc4-c4cc5ccccc5c5ccccc45)ccc3cc2c1]

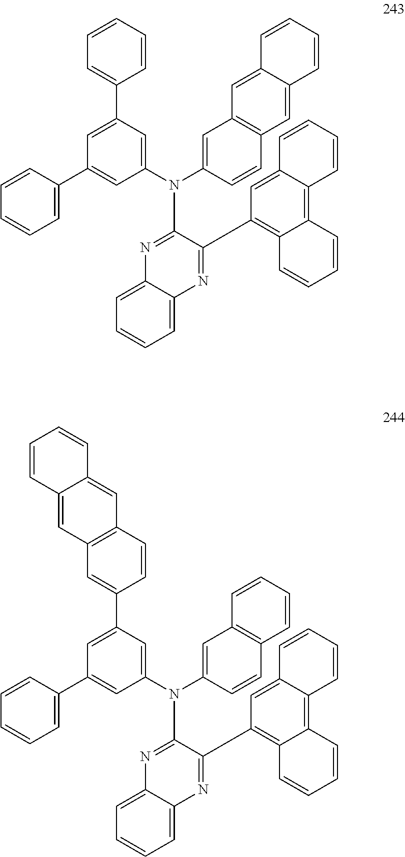 [SMILES: c1ccc(-c2cc(-c3ccccc3)cc(N(c3ccc4cc5ccccc5cc4c3)c3nc4ccccc4nc3-c3cc4ccccc4c4ccccc34)c2)cc1 ; c1ccc(-c2cc(-c3ccc4cc5ccccc5cc4c3)cc(N(c3ccc4ccccc4c3)c3nc4ccccc4nc3-c3cc4ccccc4c4ccccc34)c2)cc1]

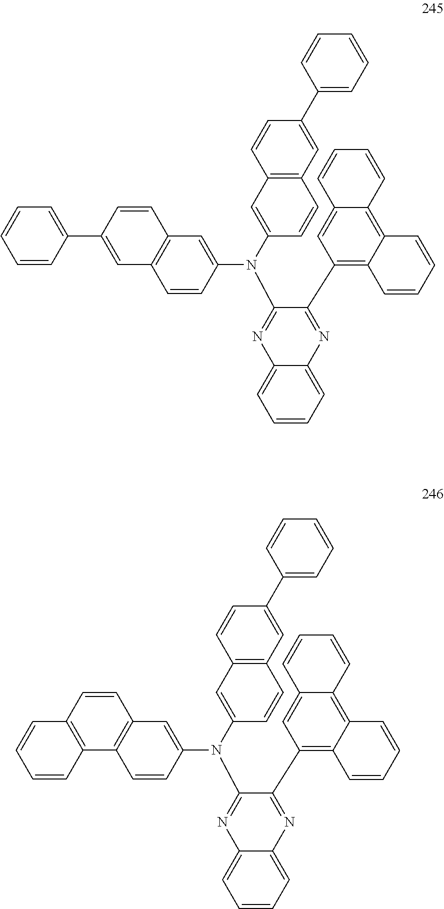 [SMILES: c1ccc(-c2ccc3cc(N(c4ccc5cc(-c6ccccc6)ccc5c4)c4nc5ccccc5nc4-c4cc5ccccc5c5ccccc45)ccc3c2)cc1 ; c1ccc(-c2ccc3cc(N(c4ccc5c(ccc6ccccc65)c4)c4nc5ccccc5nc4-c4cc5ccccc5c5ccccc45)ccc3c2)cc1]

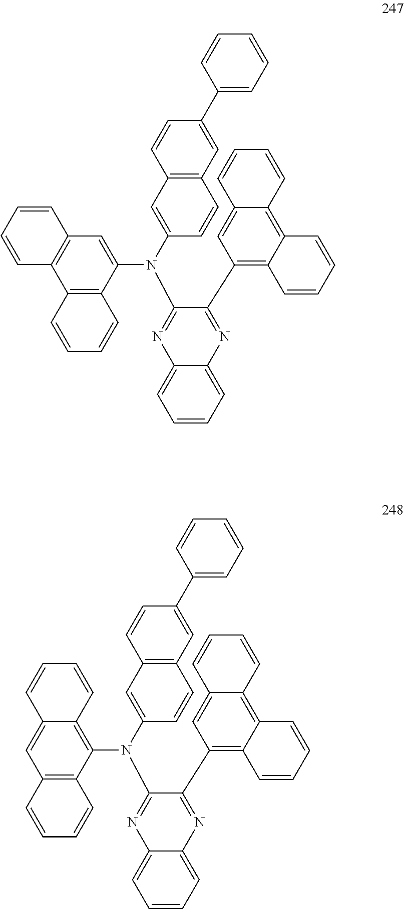 [SMILES: c1ccc(-c2ccc3cc(N(c4nc5ccccc5nc4-c4cc5ccccc5c5ccccc45)c4cc5ccccc5c5ccccc45)ccc3c2)cc1 ; c1ccc(-c2ccc3cc(N(c4nc5ccccc5nc4-c4cc5ccccc5c5ccccc45)c4c5ccccc5cc5ccccc45)ccc3c2)cc1]

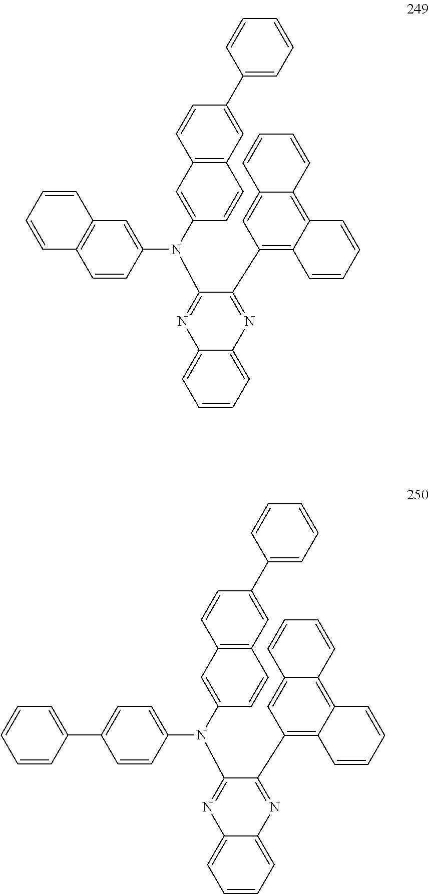 [SMILES: c1ccc(-c2ccc3cc(N(c4ccc5ccccc5c4)c4nc5ccccc5nc4-c4cc5ccccc5c5ccccc45)ccc3c2)cc1 ; c1ccc(-c2ccc(N(c3ccc4cc(-c5ccccc5)ccc4c3)c3nc4ccccc4nc3-c3cc4ccccc4c4ccccc34)cc2)cc1]

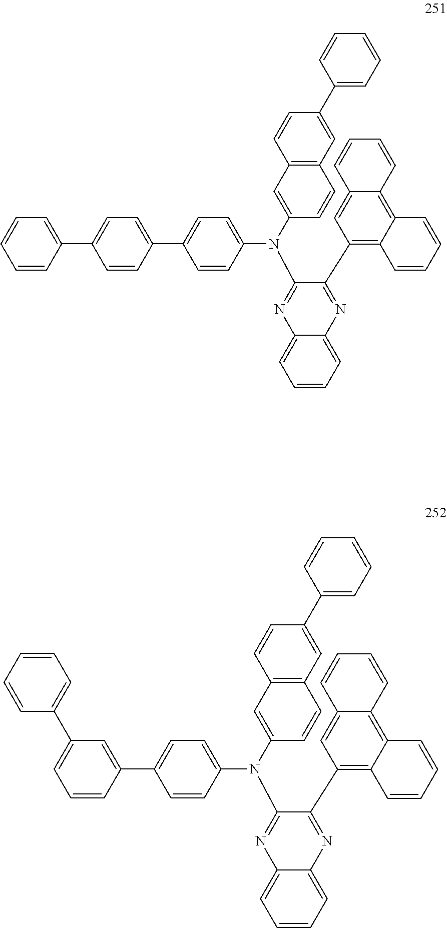 [SMILES: c1ccc(-c2ccc(-c3ccc(N(c4ccc5cc(-c6ccccc6)ccc5c4)c4nc5ccccc5nc4-c4cc5ccccc5c5ccccc45)cc3)cc2)cc1 ; c1ccc(-c2cccc(-c3ccc(N(c4ccc5cc(-c6ccccc6)ccc5c4)c4nc5ccccc5nc4-c4cc5ccccc5c5ccccc45)cc3)c2)cc1]

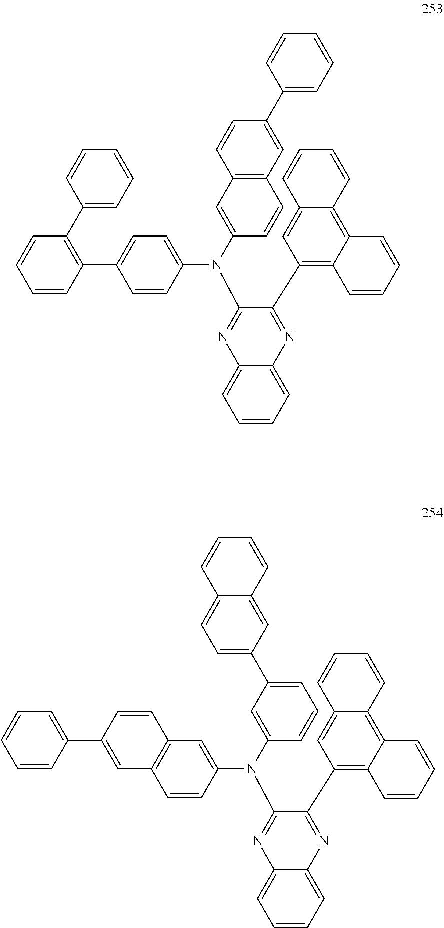 [SMILES: c1ccc(-c2ccc3cc(N(c4ccc(-c5ccccc5-c5ccccc5)cc4)c4nc5ccccc5nc4-c4cc5ccccc5c5ccccc45)ccc3c2)cc1 ; c1ccc(-c2ccc3cc(N(c4cccc(-c5ccc6ccccc6c5)c4)c4nc5ccccc5nc4-c4cc5ccccc5c5ccccc45)ccc3c2)cc1]

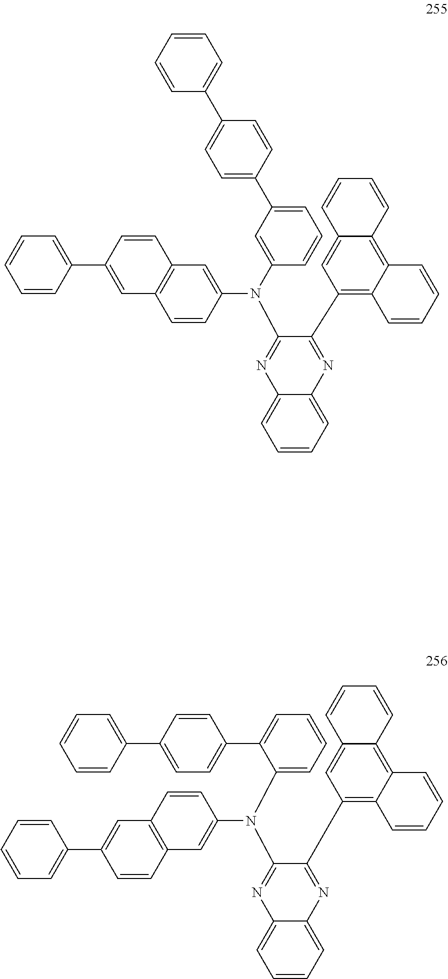 [SMILES: c1ccc(-c2ccc(-c3cccc(N(c4ccc5cc(-c6ccccc6)ccc5c4)c4nc5ccccc5nc4-c4cc5ccccc5c5ccccc45)c3)cc2)cc1 ; c1ccc(-c2ccc(-c3ccccc3N(c3ccc4cc(-c5ccccc5)ccc4c3)c3nc4ccccc4nc3-c3cc4ccccc4c4ccccc34)cc2)cc1]

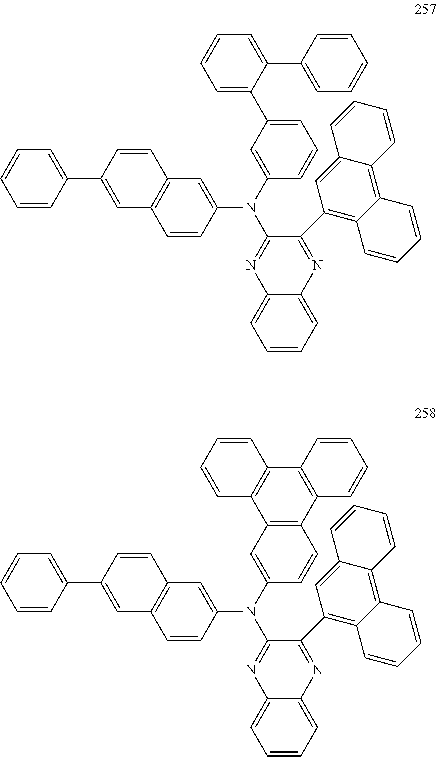 [SMILES: c1ccc(-c2ccc3cc(N(c4cccc(-c5ccccc5-c5ccccc5)c4)c4nc5ccccc5nc4-c4cc5ccccc5c5ccccc45)ccc3c2)cc1 ; c1ccc(-c2ccc3cc(N(c4ccc5c6ccccc6c6ccccc6c5c4)c4nc5ccccc5nc4-c4cc5ccccc5c5ccccc45)ccc3c2)cc1]

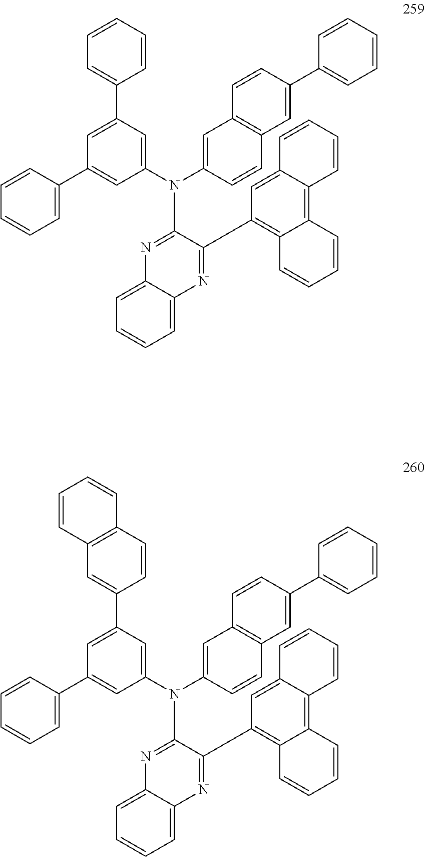 [SMILES: c1ccc(-c2cc(-c3ccccc3)cc(N(c3ccc4cc(-c5ccccc5)ccc4c3)c3nc4ccccc4nc3-c3cc4ccccc4c4ccccc34)c2)cc1 ; c1ccc(-c2cc(-c3ccc4ccccc4c3)cc(N(c3ccc4cc(-c5ccccc5)ccc4c3)c3nc4ccccc4nc3-c3cc4ccccc4c4ccccc34)c2)cc1]

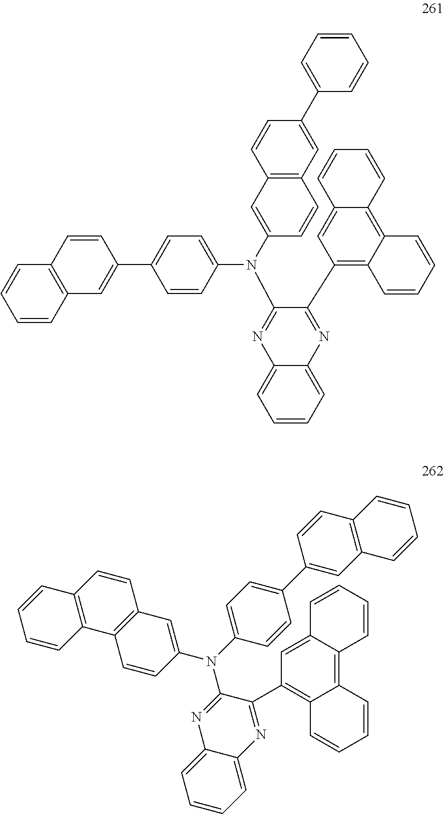 [SMILES: c1ccc(-c2ccc3cc(N(c4ccc(-c5ccc6ccccc6c5)cc4)c4nc5ccccc5nc4-c4cc5ccccc5c5ccccc45)ccc3c2)cc1 ; c1ccc2cc(-c3ccc(N(c4ccc5c(ccc6ccccc65)c4)c4nc5ccccc5nc4-c4cc5ccccc5c5ccccc45)cc3)ccc2c1]

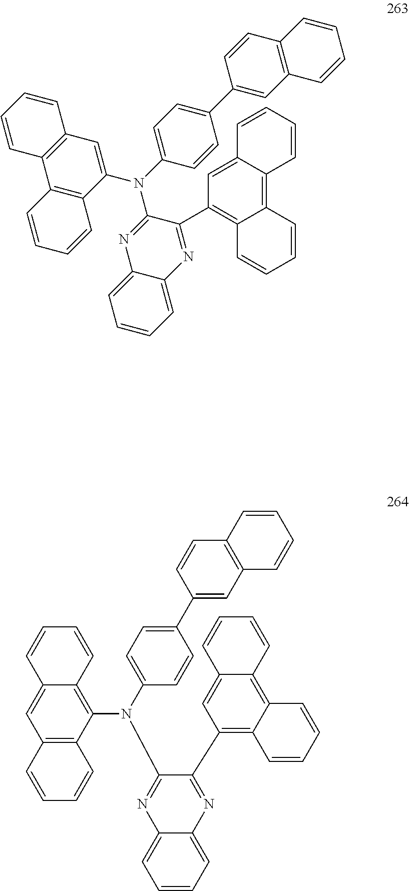 [SMILES: c1ccc2cc(-c3ccc(N(c4nc5ccccc5nc4-c4cc5ccccc5c5ccccc45)c4cc5ccccc5c5ccccc45)cc3)ccc2c1 ; c1ccc2cc(-c3ccc(N(c4nc5ccccc5nc4-c4cc5ccccc5c5ccccc45)c4c5ccccc5cc5ccccc45)cc3)ccc2c1]

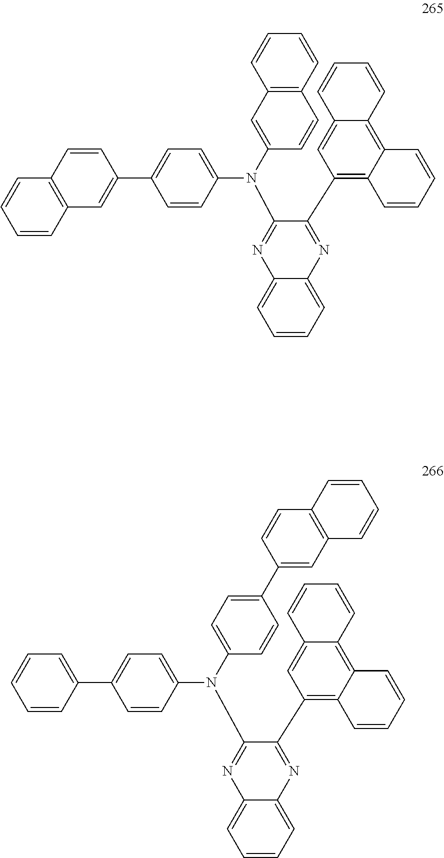 [SMILES: c1ccc2cc(-c3ccc(N(c4ccc5ccccc5c4)c4nc5ccccc5nc4-c4cc5ccccc5c5ccccc45)cc3)ccc2c1 ; c1ccc(-c2ccc(N(c3ccc(-c4ccc5ccccc5c4)cc3)c3nc4ccccc4nc3-c3cc4ccccc4c4ccccc34)cc2)cc1]

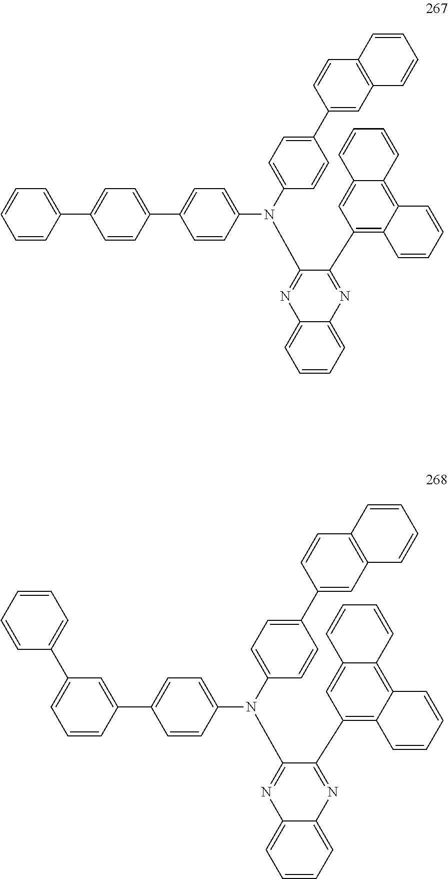 [SMILES: c1ccc(-c2ccc(-c3ccc(N(c4ccc(-c5ccc6ccccc6c5)cc4)c4nc5ccccc5nc4-c4cc5ccccc5c5ccccc45)cc3)cc2)cc1 ; c1ccc(-c2cccc(-c3ccc(N(c4ccc(-c5ccc6ccccc6c5)cc4)c4nc5ccccc5nc4-c4cc5ccccc5c5ccccc45)cc3)c2)cc1]

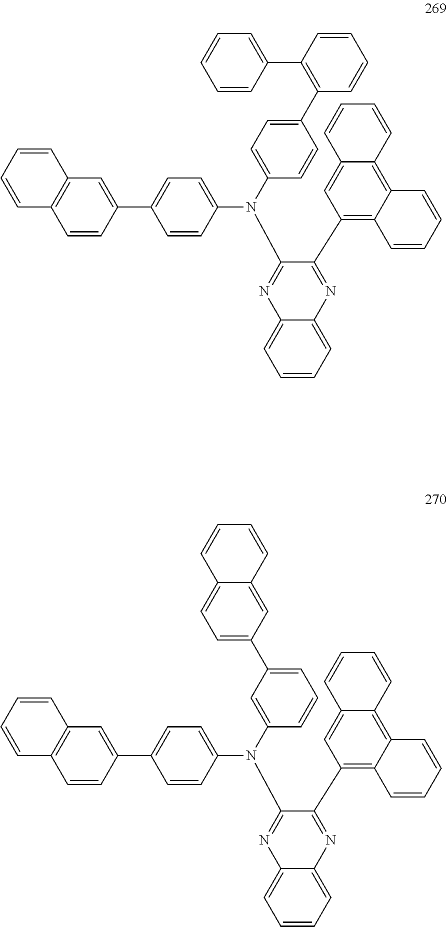 [SMILES: c1ccc(-c2ccccc2-c2ccc(N(c3ccc(-c4ccc5ccccc5c4)cc3)c3nc4ccccc4nc3-c3cc4ccccc4c4ccccc34)cc2)cc1 ; c1cc(-c2ccc3ccccc3c2)cc(N(c2ccc(-c3ccc4ccccc4c3)cc2)c2nc3ccccc3nc2-c2cc3ccccc3c3ccccc23)c1]

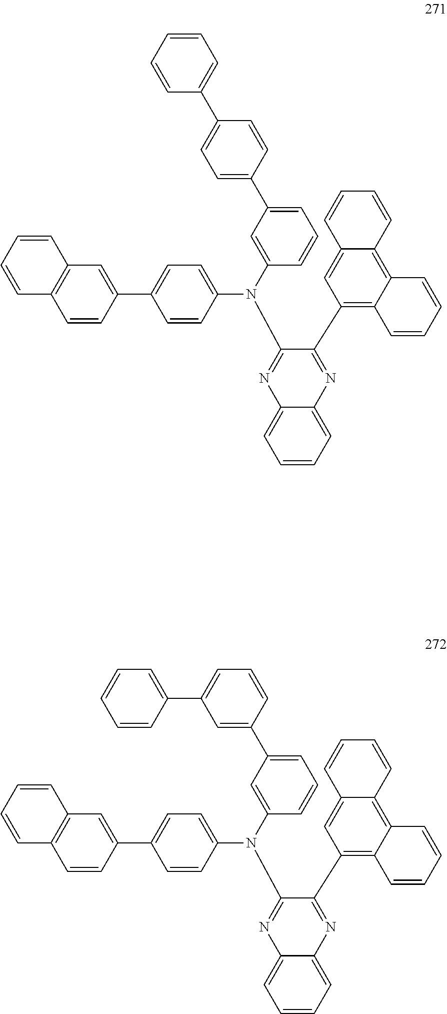 [SMILES: c1ccc(-c2ccc(-c3cccc(N(c4ccc(-c5ccc6ccccc6c5)cc4)c4nc5ccccc5nc4-c4cc5ccccc5c5ccccc45)c3)cc2)cc1 ; c1ccc(-c2cccc(-c3cccc(N(c4ccc(-c5ccc6ccccc6c5)cc4)c4nc5ccccc5nc4-c4cc5ccccc5c5ccccc45)c3)c2)cc1]

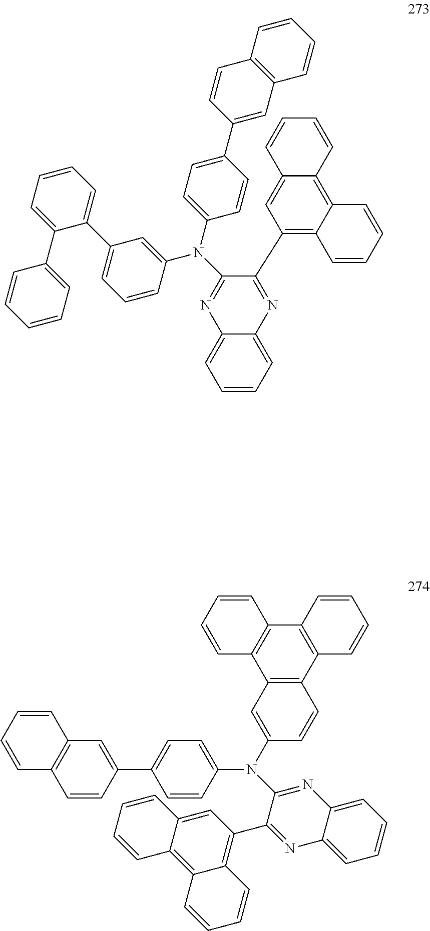 [SMILES: c1ccc(-c2ccccc2-c2cccc(N(c3ccc(-c4ccc5ccccc5c4)cc3)c3nc4ccccc4nc3-c3cc4ccccc4c4ccccc34)c2)cc1 ; c1ccc2cc(-c3ccc(N(c4ccc5c6ccccc6c6ccccc6c5c4)c4nc5ccccc5nc4-c4cc5ccccc5c5ccccc45)cc3)ccc2c1]

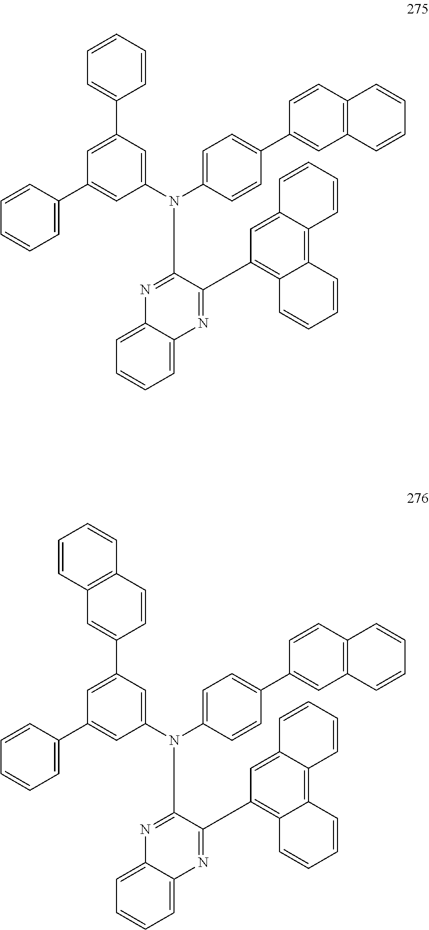 [SMILES: c1ccc(-c2cc(-c3ccccc3)cc(N(c3ccc(-c4ccc5ccccc5c4)cc3)c3nc4ccccc4nc3-c3cc4ccccc4c4ccccc34)c2)cc1 ; c1ccc(-c2cc(-c3ccc4ccccc4c3)cc(N(c3ccc(-c4ccc5ccccc5c4)cc3)c3nc4ccccc4nc3-c3cc4ccccc4c4ccccc34)c2)cc1]

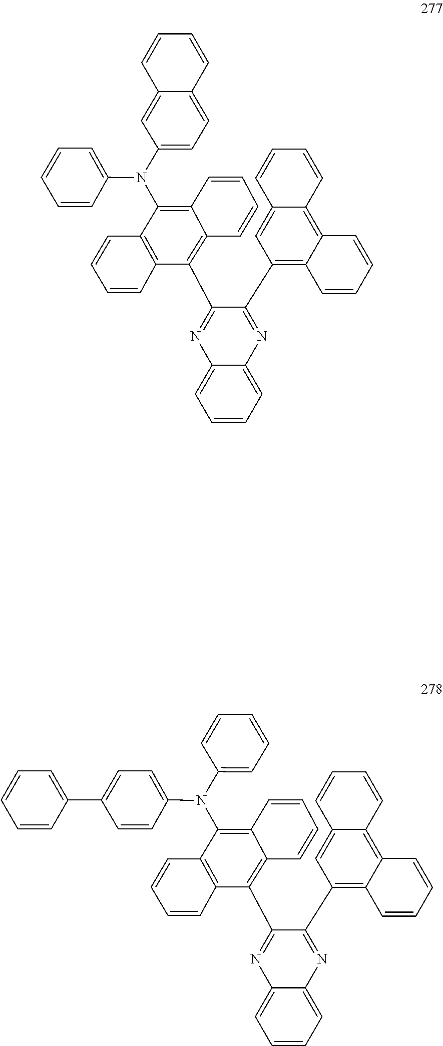 [SMILES: c1ccc(N(c2ccc3ccccc3c2)c2c3ccccc3c(-c3nc4ccccc4nc3-c3cc4ccccc4c4ccccc34)c3ccccc23)cc1 ; c1ccc(-c2ccc(N(c3ccccc3)c3c4ccccc4c(-c4nc5ccccc5nc4-c4cc5ccccc5c5ccccc45)c4ccccc34)cc2)cc1]

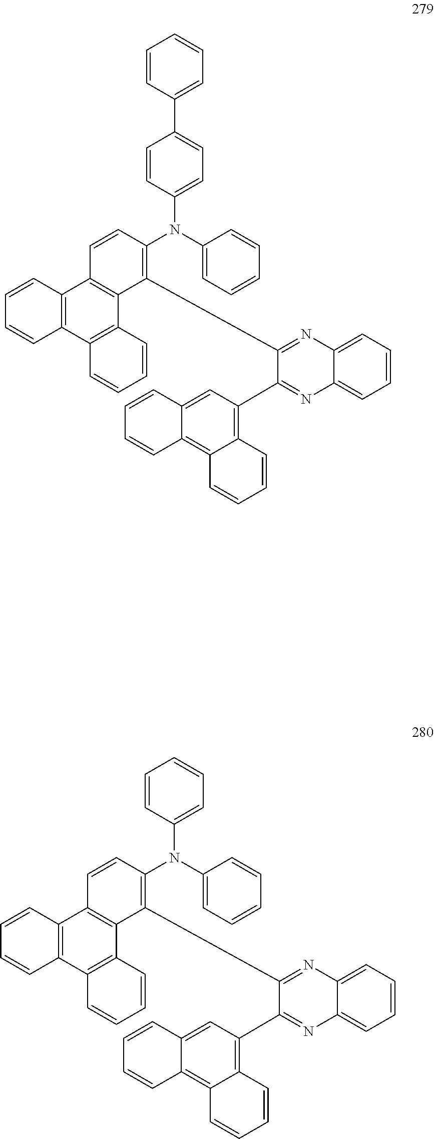 [SMILES: c1ccc(-c2ccc(N(c3ccccc3)c3ccc4c5ccccc5c5ccccc5c4c3-c3nc4ccccc4nc3-c3cc4ccccc4c4ccccc34)cc2)cc1 ; c1ccc(N(c2ccccc2)c2ccc3c4ccccc4c4ccccc4c3c2-c2nc3ccccc3nc2-c2cc3ccccc3c3ccccc23)cc1]

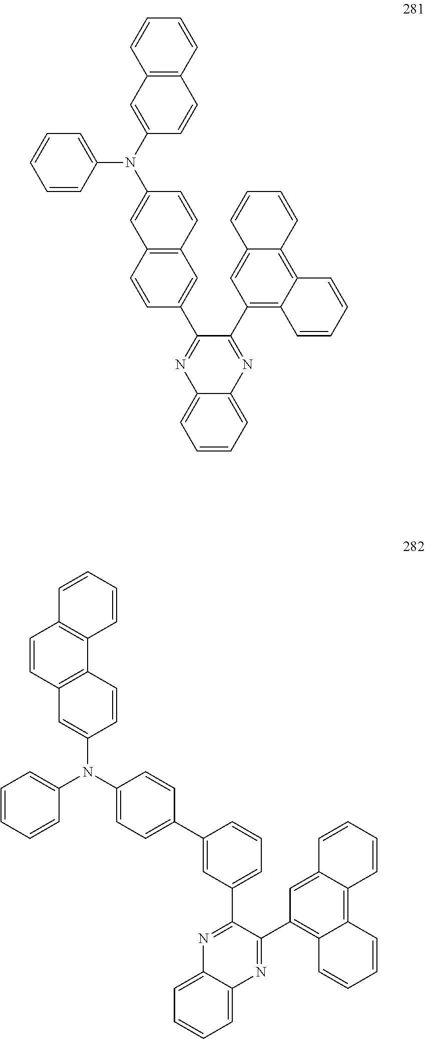 [SMILES: c1ccc(N(c2ccc3ccccc3c2)c2ccc3cc(-c4nc5ccccc5nc4-c4cc5ccccc5c5ccccc45)ccc3c2)cc1 ; c1ccc(N(c2ccc(-c3cccc(-c4nc5ccccc5nc4-c4cc5ccccc5c5ccccc45)c3)cc2)c2ccc3c(ccc4ccccc43)c2)cc1]

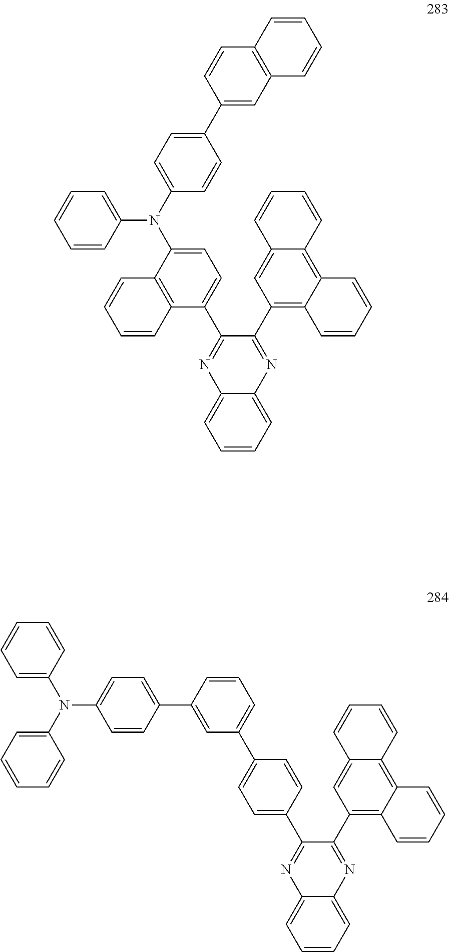 [SMILES: c1ccc(N(c2ccc(-c3ccc4ccccc4c3)cc2)c2ccc(-c3nc4ccccc4nc3-c3cc4ccccc4c4ccccc34)c3ccccc23)cc1 ; c1ccc(N(c2ccccc2)c2ccc(-c3cccc(-c4ccc(-c5nc6ccccc6nc5-c5cc6ccccc6c6ccccc56)cc4)c3)cc2)cc1]

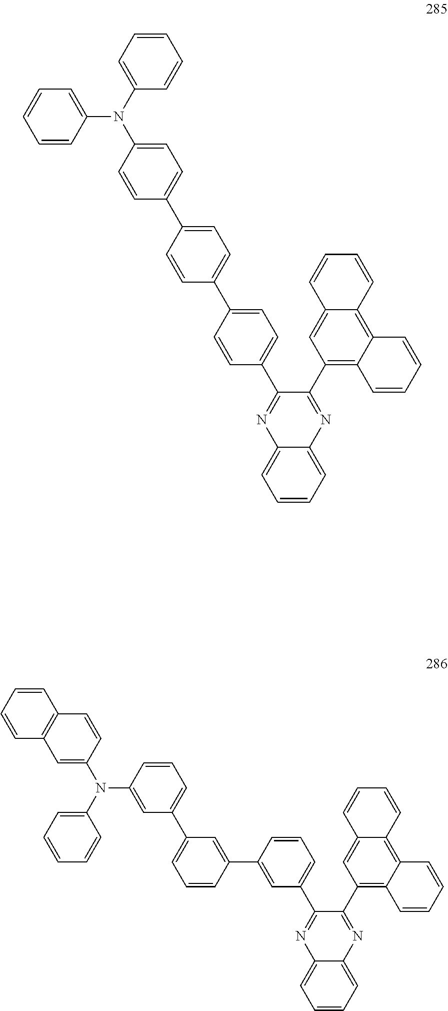 [SMILES: c1ccc(N(c2ccccc2)c2ccc(-c3ccc(-c4ccc(-c5nc6ccccc6nc5-c5cc6ccccc6c6ccccc56)cc4)cc3)cc2)cc1 ; c1ccc(N(c2cccc(-c3cccc(-c4cccc(-c5nc6ccccc6nc5-c5cc6ccccc6c6ccccc56)c4)c3)c2)c2ccc3ccccc3c2)cc1]

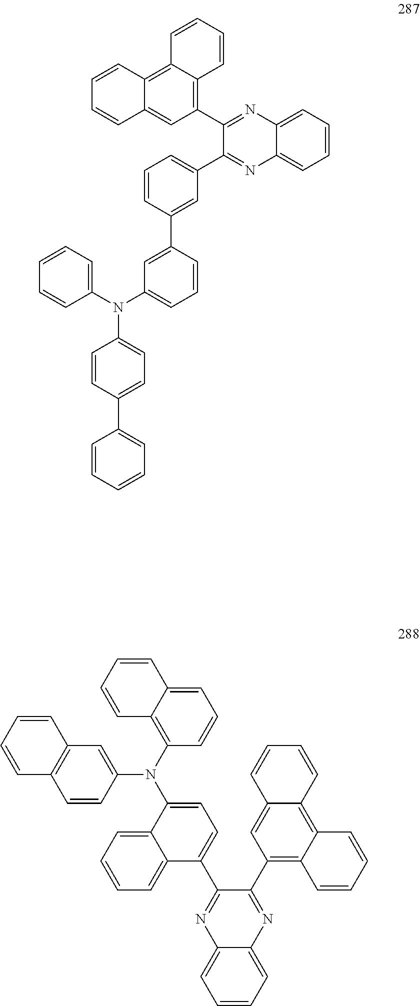 [SMILES: c1ccc(-c2ccc(N(c3ccccc3)c3cccc(-c4cccc(-c5nc6ccccc6nc5-c5cc6ccccc6c6ccccc56)c4)c3)cc2)cc1 ; c1ccc2cc(N(c3cccc4ccccc34)c3ccc(-c4nc5ccccc5nc4-c4cc5ccccc5c5ccccc45)c4ccccc34)ccc2c1]

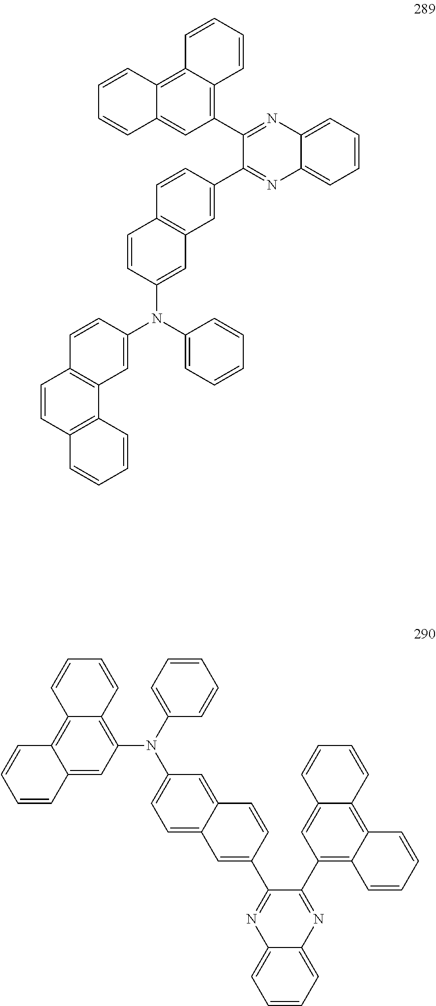 [SMILES: c1ccc(N(c2ccc3ccc(-c4nc5ccccc5nc4-c4cc5ccccc5c5ccccc45)cc3c2)c2ccc3ccc4ccccc4c3c2)cc1 ; c1ccc(N(c2ccc3cc(-c4nc5ccccc5nc4-c4cc5ccccc5c5ccccc45)ccc3c2)c2cc3ccccc3c3ccccc23)cc1]

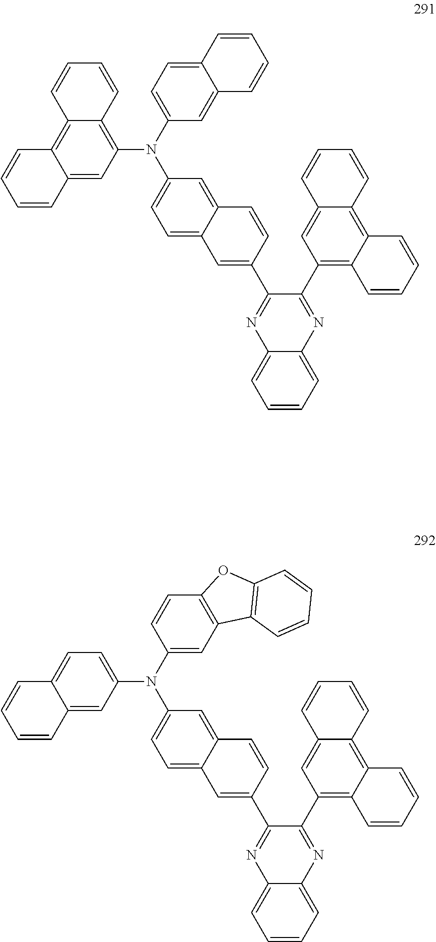 [SMILES: c1ccc2cc(N(c3ccc4cc(-c5nc6ccccc6nc5-c5cc6ccccc6c6ccccc56)ccc4c3)c3cc4ccccc4c4ccccc34)ccc2c1 ; c1ccc2cc(N(c3ccc4cc(-c5nc6ccccc6nc5-c5cc6ccccc6c6ccccc56)ccc4c3)c3ccc4oc5ccccc5c4c3)ccc2c1]

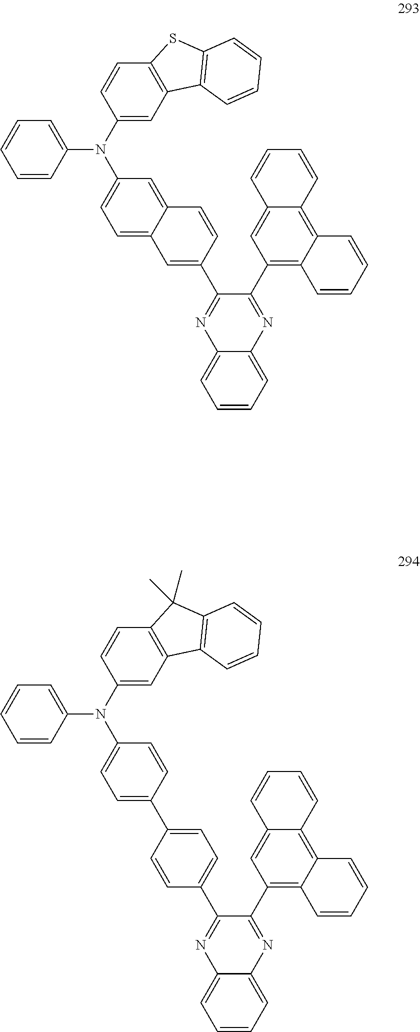 [SMILES: c1ccc(N(c2ccc3cc(-c4nc5ccccc5nc4-c4cc5ccccc5c5ccccc45)ccc3c2)c2ccc3sc4ccccc4c3c2)cc1 ; CC1(C)c2ccccc2-c2cc(N(c3ccccc3)c3ccc(-c4ccc(-c5nc6ccccc6nc5-c5cc6ccccc6c6ccccc56)cc4)cc3)ccc21]

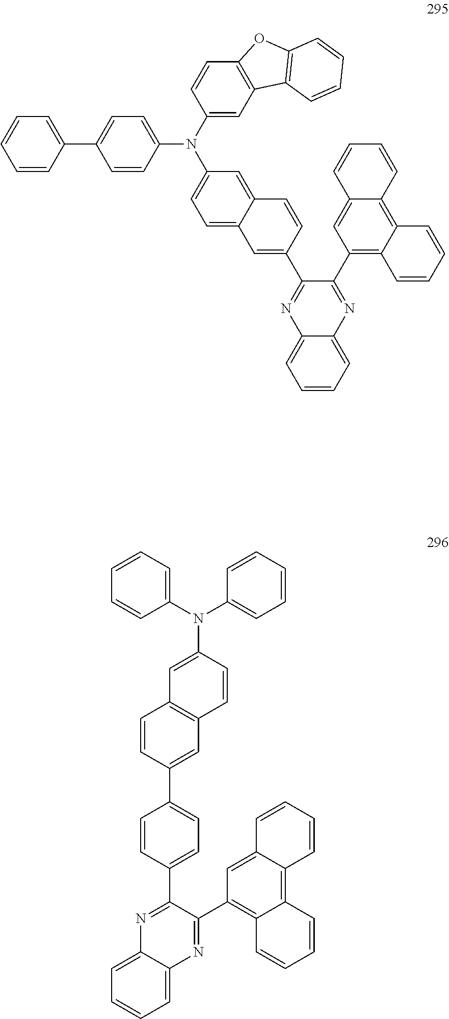 [SMILES: c1ccc(-c2ccc(N(c3ccc4cc(-c5nc6ccccc6nc5-c5cc6ccccc6c6ccccc56)ccc4c3)c3ccc4oc5ccccc5c4c3)cc2)cc1 ; c1ccc(N(c2ccccc2)c2ccc3cc(-c4ccc(-c5nc6ccccc6nc5-c5cc6ccccc6c6ccccc56)cc4)ccc3c2)cc1]

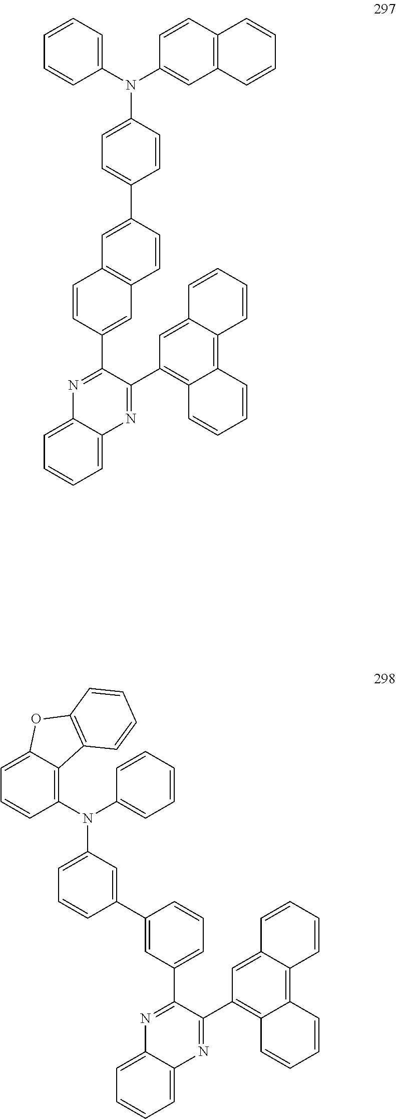 [SMILES: c1ccc(N(c2ccc(-c3ccc4cc(-c5nc6ccccc6nc5-c5cc6ccccc6c6ccccc56)ccc4c3)cc2)c2ccc3ccccc3c2)cc1 ; c1ccc(N(c2cccc(-c3cccc(-c4nc5ccccc5nc4-c4cc5ccccc5c5ccccc45)c3)c2)c2cccc3oc4ccccc4c23)cc1]

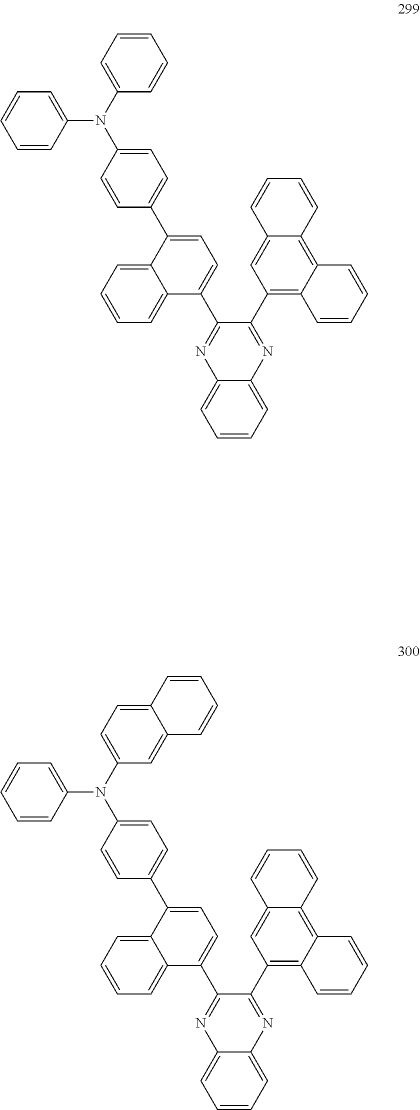 [SMILES: c1ccc(N(c2ccccc2)c2ccc(-c3ccc(-c4nc5ccccc5nc4-c4cc5ccccc5c5ccccc45)c4ccccc34)cc2)cc1 ; c1ccc(N(c2ccc(-c3ccc(-c4nc5ccccc5nc4-c4cc5ccccc5c5ccccc45)c4ccccc34)cc2)c2ccc3ccccc3c2)cc1]

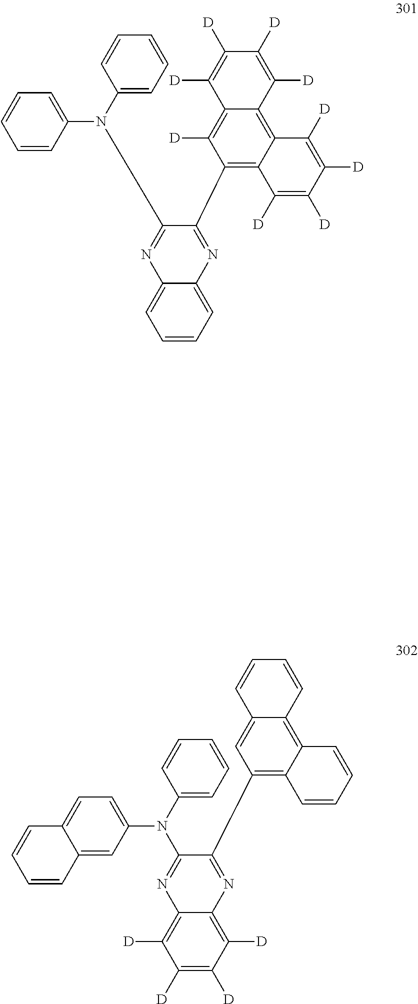 [SMILES: [2H]c1c([2H])c([2H])c2c(c1[2H])c([2H])c(-c1nc3ccccc3nc1N(c1ccccc1)c1ccccc1)c1c([2H])c([2H])c([2H])c([2H])c12 ; [2H]c1c([2H])c([2H])c2nc(N(c3ccccc3)c3ccc4ccccc4c3)c(-c3cc4ccccc4c4ccccc34)nc2c1[2H]]

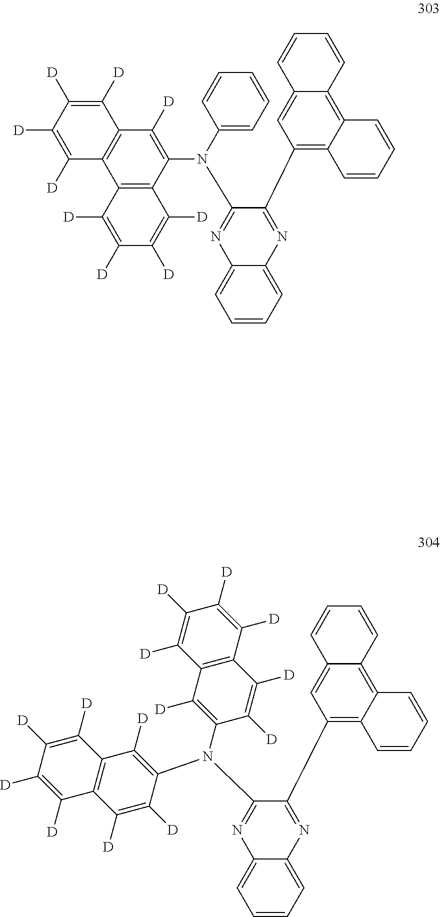 [SMILES: [2H]c1c([2H])c([2H])c2c(c1[2H])c([2H])c(N(c1ccccc1)c1nc3ccccc3nc1-c1cc3ccccc3c3ccccc13)c1c([2H])c([2H])c([2H])c([2H])c12 ; [2H]c1c([2H])c([2H])c2c([2H])c(N(c3nc4ccccc4nc3-c3cc4ccccc4c4ccccc34)c3c([2H])c([2H])c4c([2H])c([2H])c([2H])c([2H])c4c3[2H])c([2H])c([2H])c2c1[2H]]

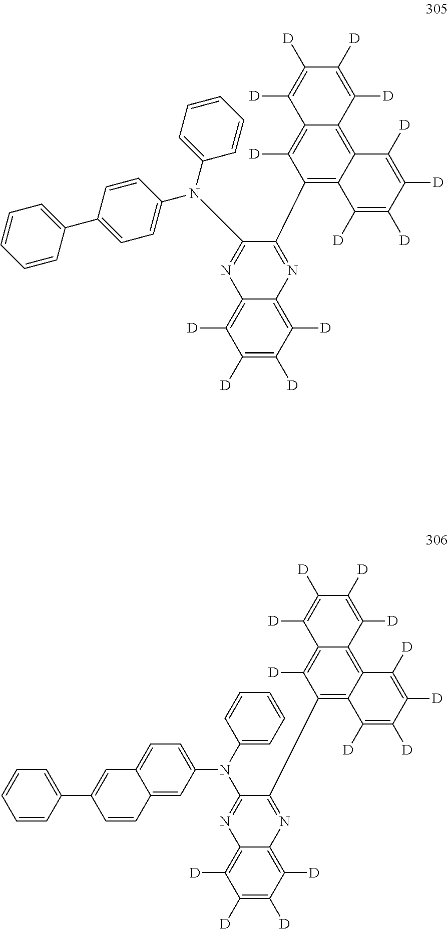 [SMILES: [2H]c1c([2H])c([2H])c2nc(N(c3ccccc3)c3ccc(-c4ccccc4)cc3)c(-c3c([2H])c4c([2H])c([2H])c([2H])c([2H])c4c4c([2H])c([2H])c([2H])c([2H])c34)nc2c1[2H] ; [2H]c1c([2H])c([2H])c2nc(N(c3ccccc3)c3ccc4cc(-c5ccccc5)ccc4c3)c(-c3c([2H])c4c([2H])c([2H])c([2H])c([2H])c4c4c([2H])c([2H])c([2H])c([2H])c34)nc2c1[2H]]

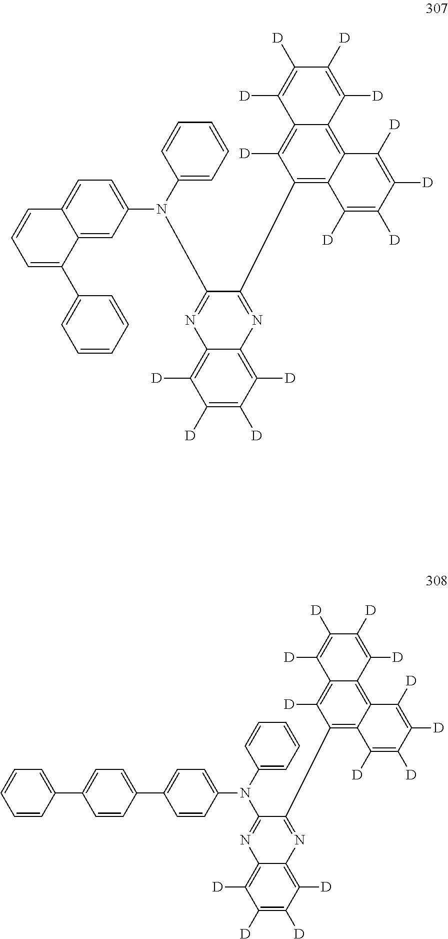 [SMILES: [2H]c1c([2H])c([2H])c2nc(N(c3ccccc3)c3ccc4cccc(-c5ccccc5)c4c3)c(-c3c([2H])c4c([2H])c([2H])c([2H])c([2H])c4c4c([2H])c([2H])c([2H])c([2H])c34)nc2c1[2H] ; [2H]c1c([2H])c([2H])c2nc(N(c3ccccc3)c3ccc(-c4ccc(-c5ccccc5)cc4)cc3)c(-c3c([2H])c4c([2H])c([2H])c([2H])c([2H])c4c4c([2H])c([2H])c([2H])c([2H])c34)nc2c1[2H]]

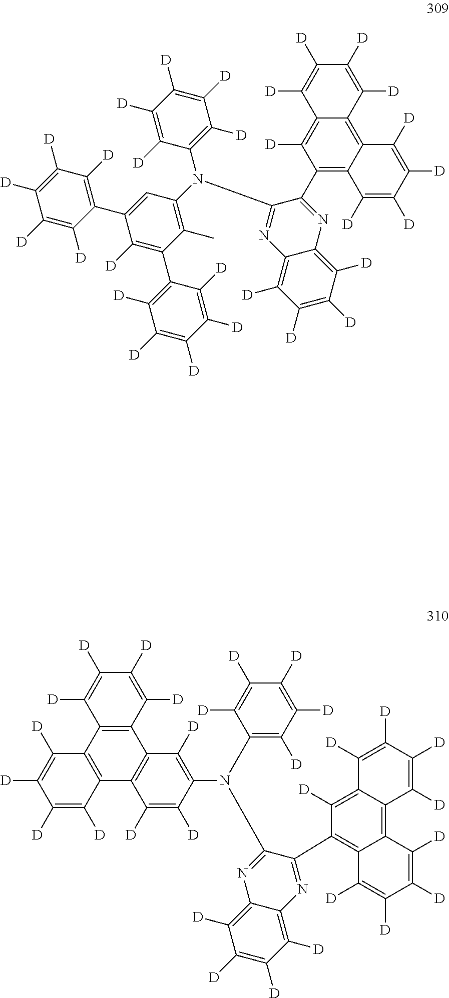 [SMILES: [2H]c1c([2H])c([2H])c(-c2cc(N(c3nc4c([2H])c([2H])c([2H])c([2H])c4nc3-c3c([2H])c4c([2H])c([2H])c([2H])c([2H])c4c4c([2H])c([2H])c([2H])c([2H])c34)c3c([2H])c([2H])c([2H])c([2H])c3[2H])c(C)c(-c3c([2H])c([2H])c([2H])c([2H])c3[2H])c2[2H])c([2H])c1[2H] ; [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c3c4c([2H])c([2H])c([2H])c([2H])c4c4c([2H])c([2H])c([2H])c([2H])c4c3c2[2H])c([2H])c1[2H]]

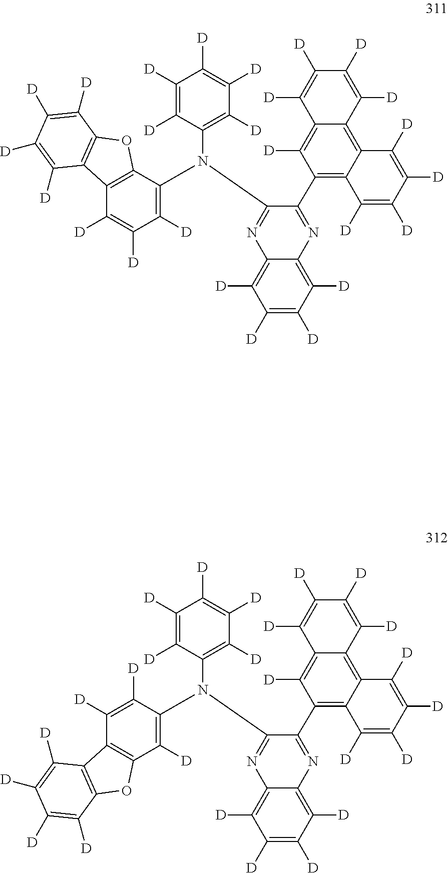 [SMILES: [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c([2H])c3c2oc2c([2H])c([2H])c([2H])c([2H])c23)c([2H])c1[2H] ; [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c3c(oc4c([2H])c([2H])c([2H])c([2H])c43)c2[2H])c([2H])c1[2H]]

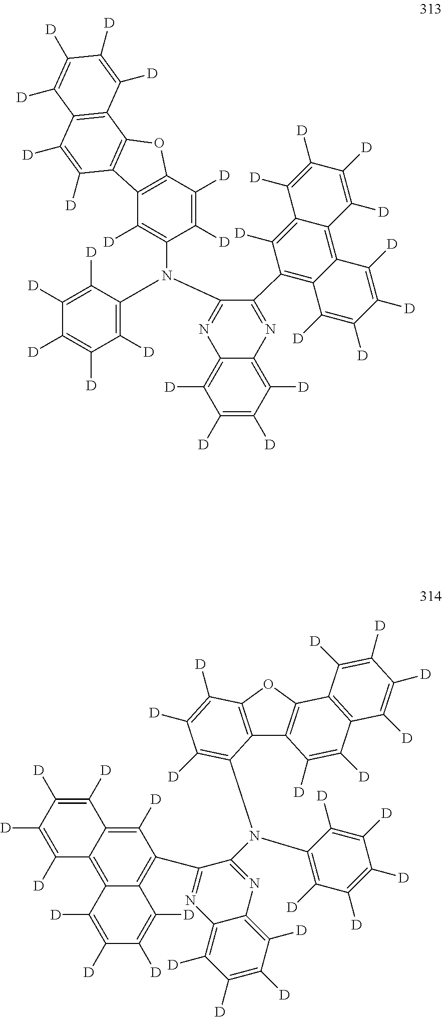 [SMILES: [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c3oc4c5c([2H])c([2H])c([2H])c([2H])c5c([2H])c([2H])c4c3c2[2H])c([2H])c1[2H] ; [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c([2H])c3oc4c5c([2H])c([2H])c([2H])c([2H])c5c([2H])c([2H])c4c23)c([2H])c1[2H]]

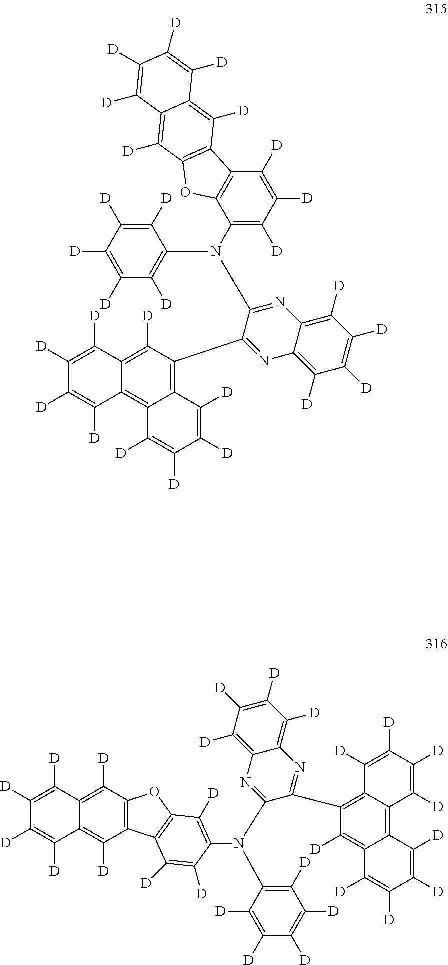 [SMILES: [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c([2H])c3c2oc2c([2H])c4c([2H])c([2H])c([2H])c([2H])c4c([2H])c23)c([2H])c1[2H] ; [2H]c1c([2H])c([2H])c(N(c2nc3c([2H])c([2H])c([2H])c([2H])c3nc2-c2c([2H])c3c([2H])c([2H])c([2H])c([2H])c3c3c([2H])c([2H])c([2H])c([2H])c23)c2c([2H])c([2H])c3c(oc4c([2H])c5c([2H])c([2H])c([2H])c([2H])c5c([2H])c43)c2[2H])c([2H])c1[2H]]

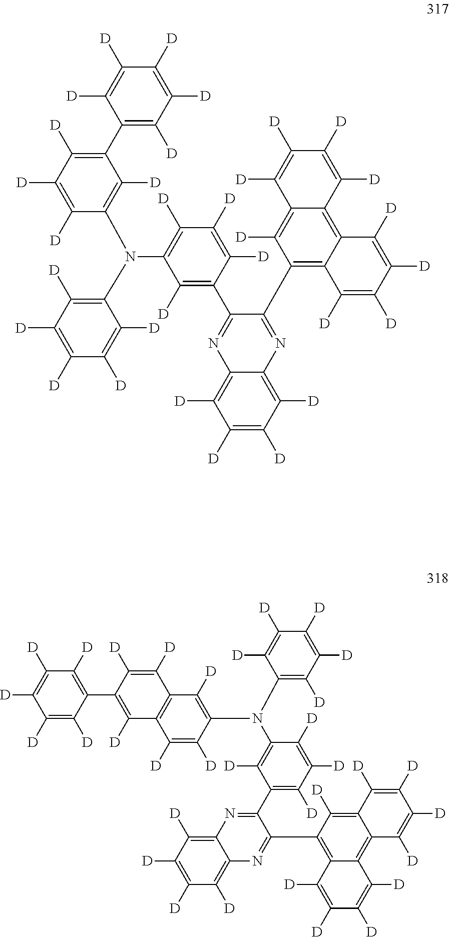

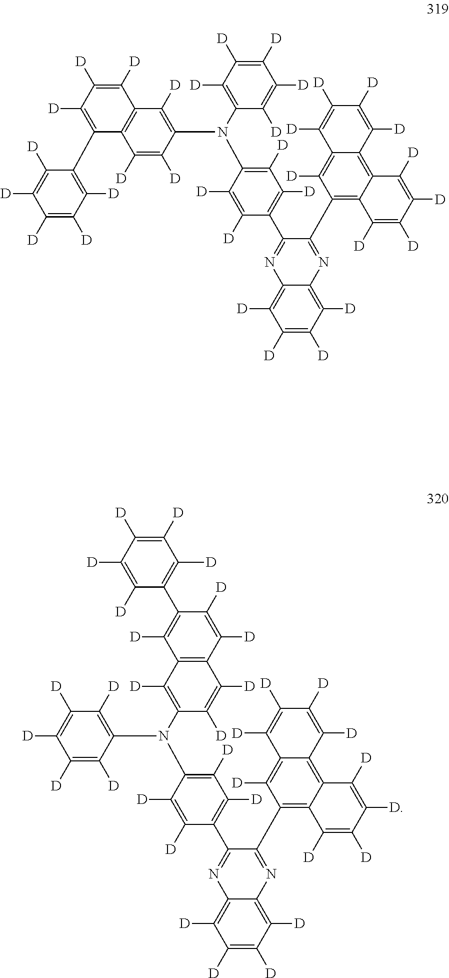

6. An organic light-emitting device comprising:
a first electrode;
a second electrode provided opposite to the first electrode; and
one or more organic material layers provided between the first electrode and the second electrode,
wherein at least one or more organic material layers comprises the heterocyclic compound of claim 1.

7. The organic light-emitting device of claim 6, wherein the organic material layer includes a light emitting layer, and the light emitting layer includes the heterocyclic compound.

8. The organic light-emitting device of claim 6, wherein the organic material layer includes a light emitting layer, the light emitting layer includes a host material, and the host material includes the heterocyclic compound.

9. The organic light-emitting device of claim 6, wherein the organic material layer further includes a heterocyclic compound represented by the following Chemical Formula 2: 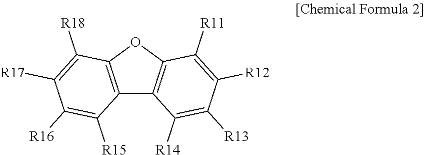
in Chemical Formula 2
R11 to R18 are the same as or different from each other, and each independently selected from the group consisting of hydrogen; deuterium; halogen: a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; —NR201R202; and the following Chemical Formula 3, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
at least one of R11 to R18 is the following Chemical Formula 3, 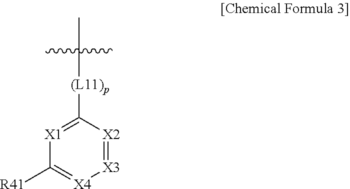
in Chemical Formula 3
X1 is N; or CRa;
X2 is N; or CRb;
X3 is N; or CRc;
X4 is N; or CRd;
at least two of X1 to X4 are N;
R41 and Ra to Rd are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted (C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R301R302; —SiR301R302R303; and —NR301R302, or two or more groups adjacent to each other bond to each other to form a substituted or unsubstituted C6 to C60 aromatic hydrocarbon ring; or a substituted or unsubstituted C2 to C60 heteroring, and R301, R302 and R303 are the same as or different from each other and each independently a substituted or unsubstituted C1 to C60 alkyl group; a substituted or unsubstituted C6 to C60 aryl group; or a substituted or unsubstituted C2 to C60 heteroaryl group;
L11 is a direct bond; a substituted or unsubstituted C6 to C60 arylene group; or a substituted or unsubstituted C2 to C60 heteroarylene group; and
p is an integer of 0 to 5, and when p is 2 or greater, L1 is are the same as or different from each other.

10. The organic light-emitting device of claim 9, wherein Chemical Formula 3 is represented by any one of the following Chemical Formulae 3-1 to 3-6: 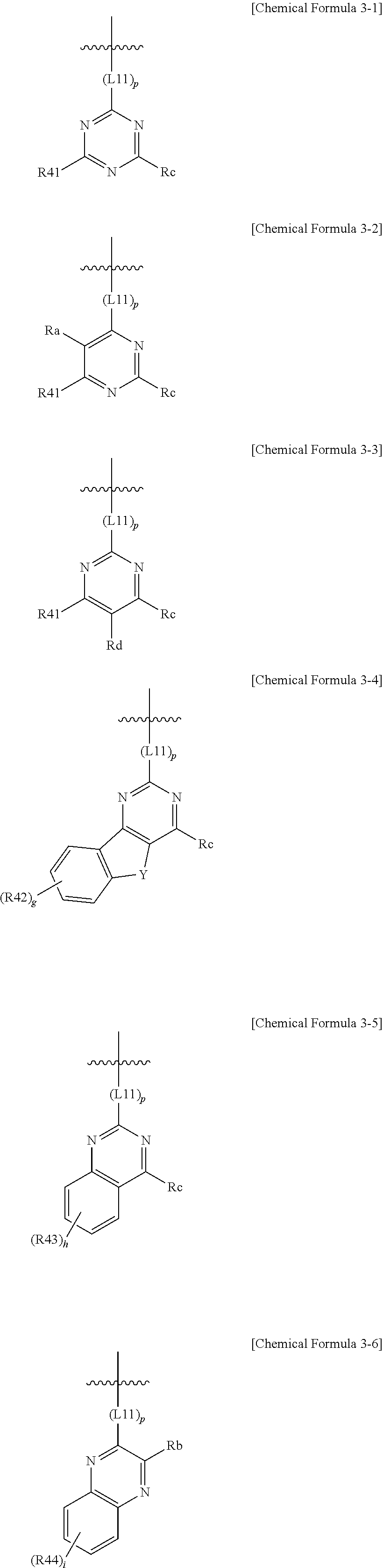
in Chemical Formulae 3-1 to 3-6,
Y is O; or S;
R42 to R44 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group: a substituted or unsubstituted C2 to C60 heteroaryl group; —P(═O)R301R302; —SiR301R302R303; and —NR301R302, and R301, R302 and R303 are the same as or different from each other and each independently a substituted or unsubstituted C1 to C60 alkyl group; a substituted or unsubstituted C6 to C60 aryl group; or a substituted or unsubstituted C2 to C60 heteroaryl group;
g is an integer of 0 to 4, and when g is 2 or greater, R42s are the same as or different from each other;
h is an integer of 0 to 4, and when h is 2 or greater, R43s are the same as or different from each other;
i is an integer of 0 to 4, and when i is 2 or greater, R44s are the same as or different from each other; and
R41, Ra to Rd, L11 and p have the same definitions as in Chemical Formula 3.

11. The organic light-emitting device of claim 9, wherein the heterocyclic compound represented by Chemical Formula 2 does not include deuterium as a substituent, or has a deuterium content of 1% to 100% with respect to a total number of hydrogen atoms and deuterium atoms.

12. The organic light-emitting device of claim 9, wherein at least one of the heterocyclic compound represented by Chemical Formula 1 and the heterocyclic compound represented by Chemical Formula 2 does not include deuterium as a substituent, or has a deuterium content of 1% to 100% with respect to a total number of hydrogen atoms and deuterium atoms.

13. The organic light-emitting device of claim 9, wherein the heterocyclic compound represented by Chemical Formula 2 is represented by any one of the following compounds:

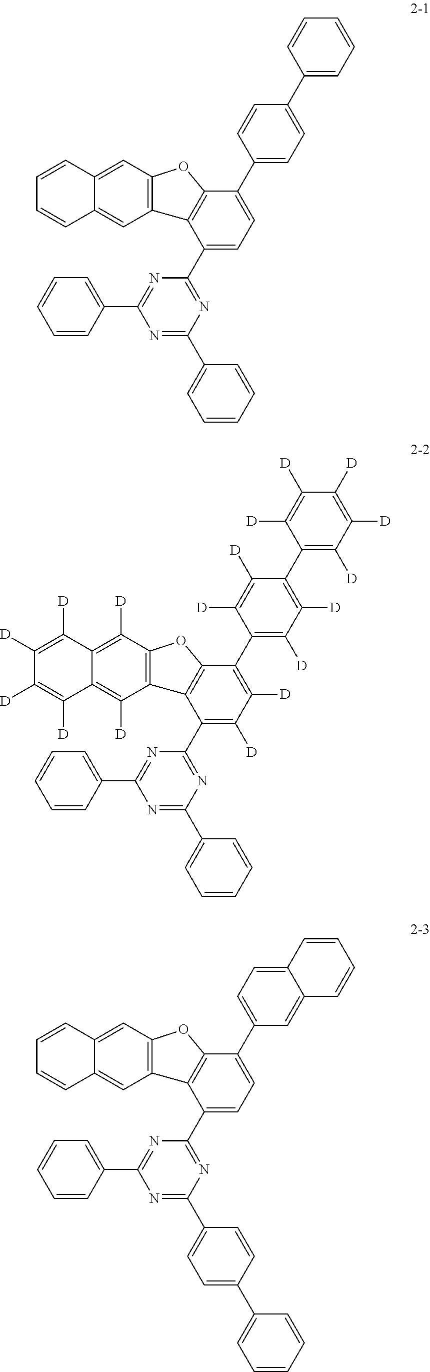 [SMILES: c1ccc(-c2ccc(-c3ccc(-c4nc(-c5ccccc5)nc(-c5ccccc5)n4)c4c3oc3cc5ccccc5cc34)cc2)cc1 ; [2H]c1c([2H])c([2H])c(-c2c([2H])c([2H])c(-c3c([2H])c([2H])c(-c4nc(-c5ccccc5)nc(-c5ccccc5)n4)c4c3oc3c([2H])c5c([2H])c([2H])c([2H])c([2H])c5c([2H])c34)c([2H])c2[2H])c([2H])c1[2H] ; c1ccc(-c2ccc(-c3nc(-c4ccccc4)nc(-c4ccc(-c5ccc6ccccc6c5)c5oc6cc7ccccc7cc6c45)n3)cc2)cc1]

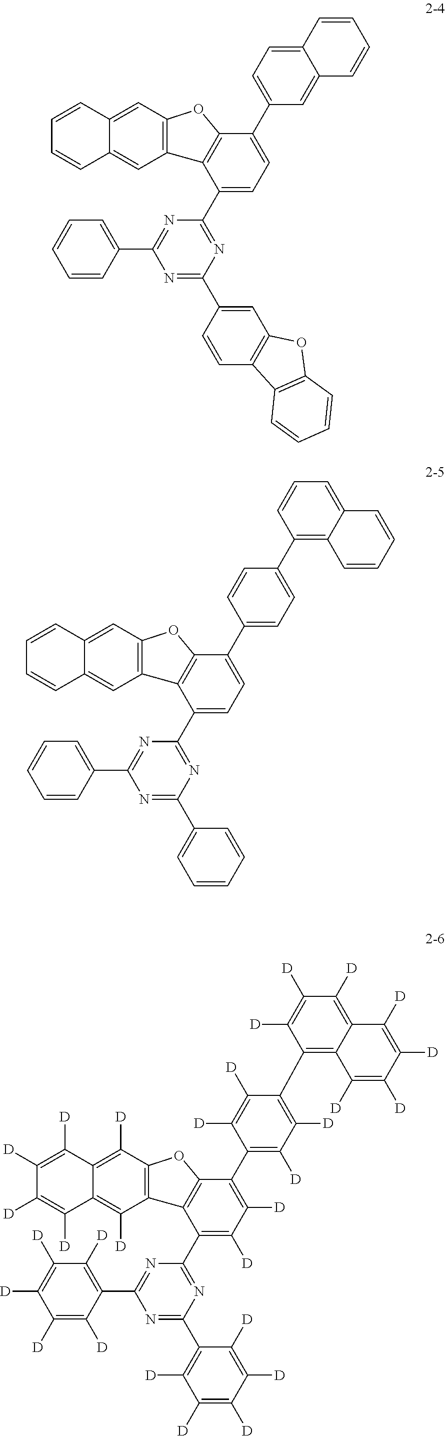

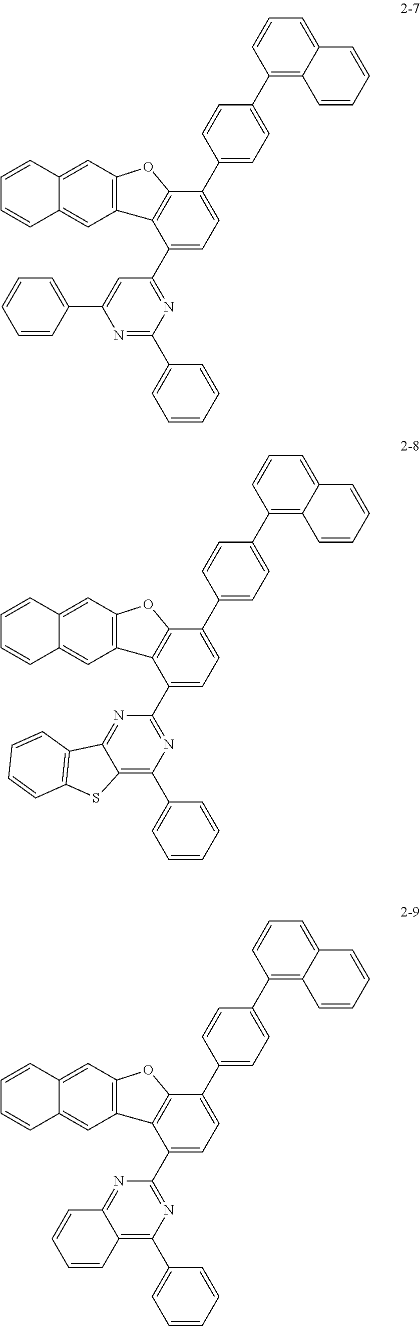 [SMILES: c1ccc(-c2cc(-c3ccc(-c4ccc(-c5cccc6ccccc56)cc4)c4oc5cc6ccccc6cc5c34)nc(-c3ccccc3)n2)cc1 ; c1ccc(-c2nc(-c3ccc(-c4ccc(-c5cccc6ccccc56)cc4)c4oc5cc6ccccc6cc5c34)nc3c2sc2ccccc23)cc1 ; c1ccc(-c2nc(-c3ccc(-c4ccc(-c5cccc6ccccc56)cc4)c4oc5cc6ccccc6cc5c34)nc3ccccc23)cc1]

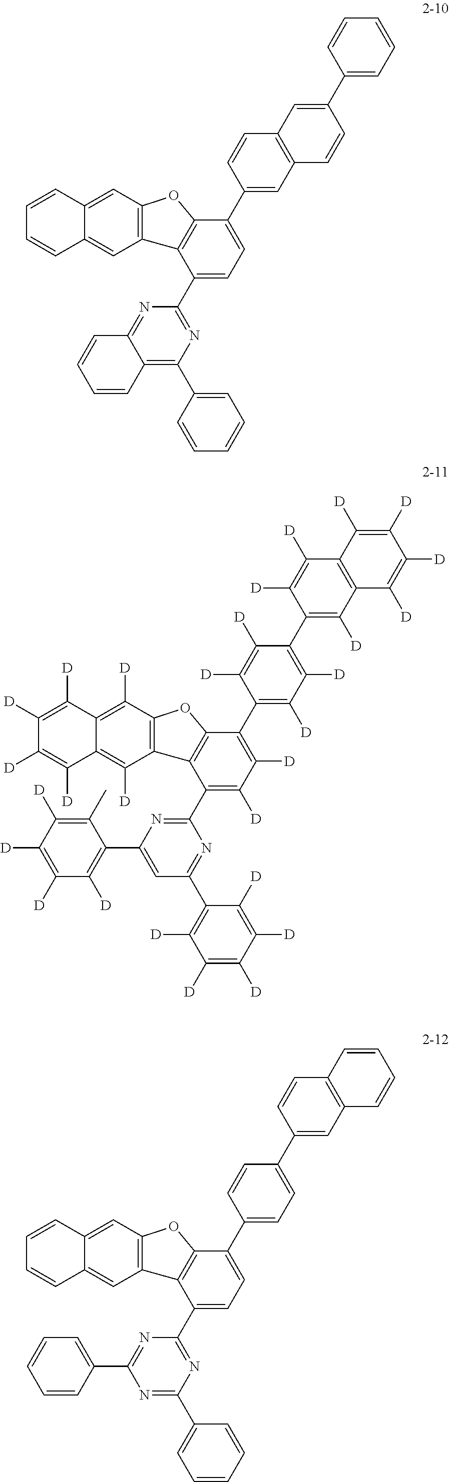 [SMILES: c1ccc(-c2ccc3cc(-c4ccc(-c5nc(-c6ccccc6)c6ccccc6n5)c5c4oc4cc6ccccc6cc45)ccc3c2)cc1 ; [2H]c1c([2H])c([2H])c(-c2cc(-c3c([2H])c([2H])c([2H])c([2H])c3C)nc(-c3c([2H])c([2H])c(-c4c([2H])c([2H])c(-c5c([2H])c([2H])c6c([2H])c([2H])c([2H])c([2H])c6c5[2H])c([2H])c4[2H])c4oc5c([2H])c6c([2H])c([2H])c([2H])c([2H])c6c([2H])c5c34)n2)c([2H])c1[2H] ; c1ccc(-c2nc(-c3ccccc3)nc(-c3ccc(-c4ccc(-c5ccc6ccccc6c5)cc4)c4oc5cc6ccccc6cc5c34)n2)cc1]

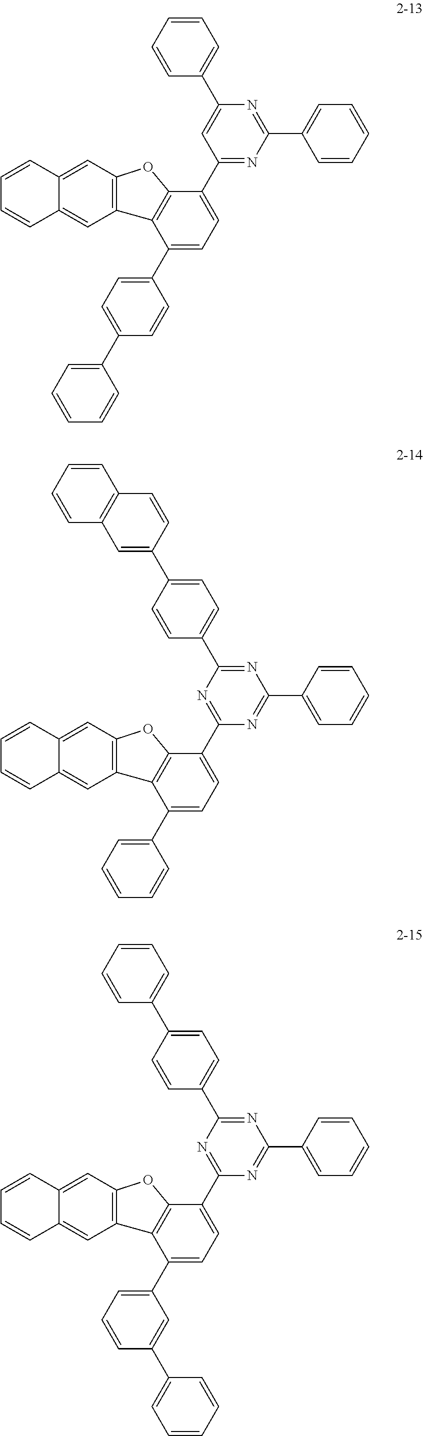

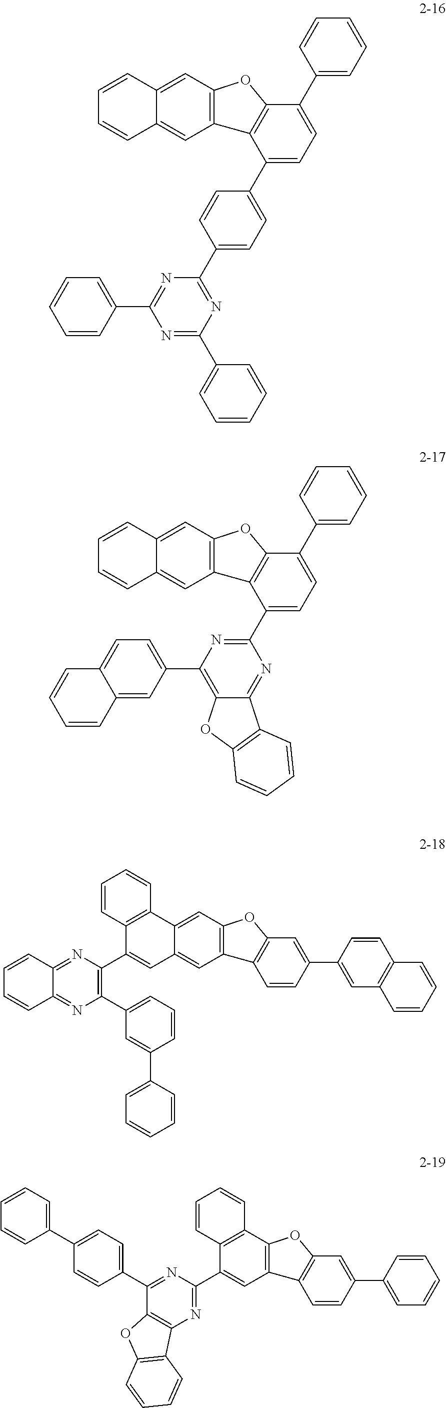 [SMILES: c1ccc(-c2nc(-c3ccccc3)nc(-c3ccc(-c4ccc(-c5ccccc5)c5oc6cc7ccccc7cc6c45)cc3)n2)cc1 ; c1ccc(-c2ccc(-c3nc(-c4ccc5ccccc5c4)c4oc5ccccc5c4n3)c3c2oc2cc4ccccc4cc23)cc1 ; c1ccc(-c2cccc(-c3nc4ccccc4nc3-c3cc4cc5c(cc4c4ccccc34)oc3cc(-c4ccc6ccccc6c4)ccc35)c2)cc1 ; c1ccc(-c2ccc(-c3nc(-c4cc5c6ccc(-c7ccccc7)cc6oc5c5ccccc45)nc4c3oc3ccccc34)cc2)cc1]

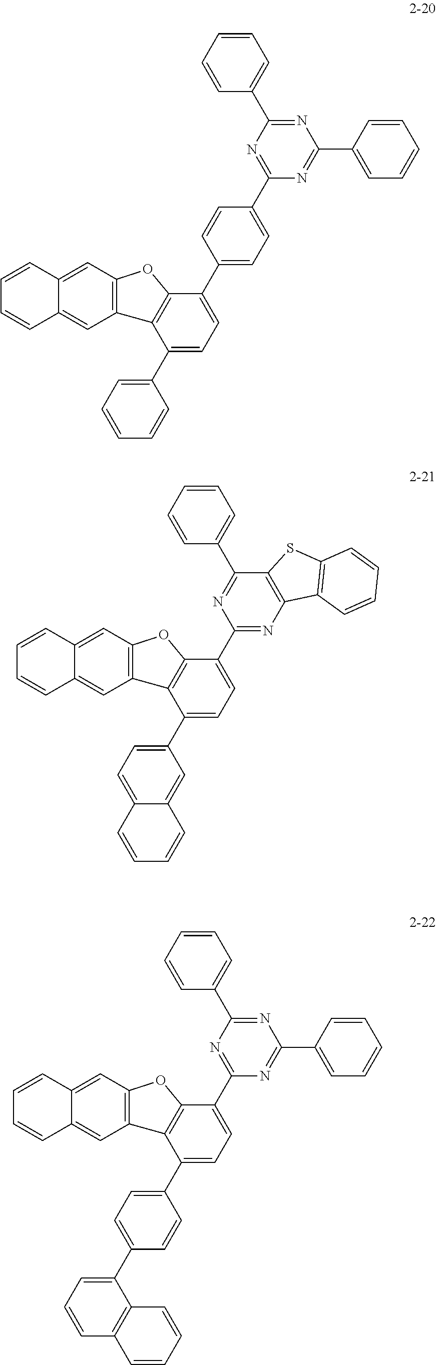 [SMILES: c1ccc(-c2nc(-c3ccccc3)nc(-c3ccc(-c4ccc(-c5ccccc5)c5c4oc4cc6ccccc6cc45)cc3)n2)cc1 ; c1ccc(-c2nc(-c3ccc(-c4ccc5ccccc5c4)c4c3oc3cc5ccccc5cc34)nc3c2sc2ccccc23)cc1 ; c1ccc(-c2nc(-c3ccccc3)nc(-c3ccc(-c4ccc(-c5cccc6ccccc56)cc4)c4c3oc3cc5ccccc5cc34)n2)cc1]

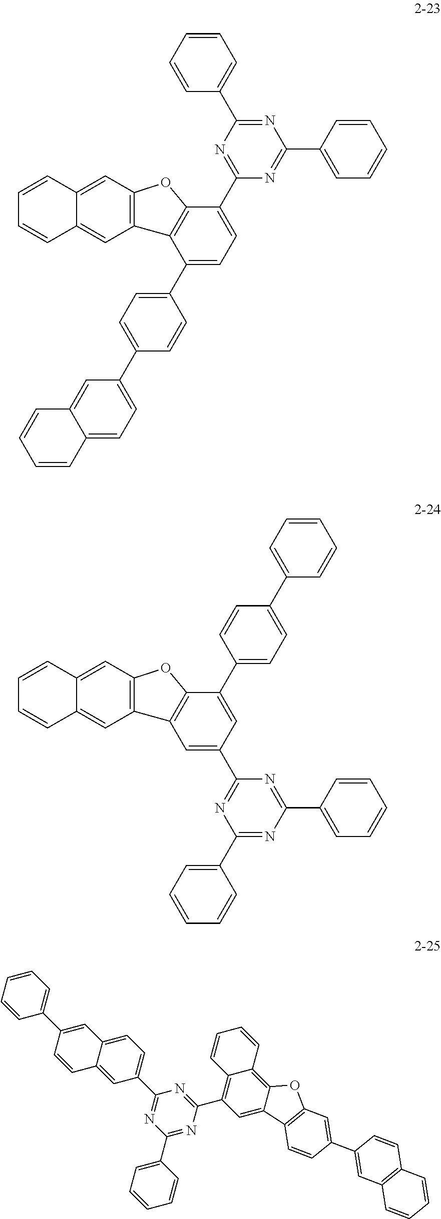 [SMILES: c1ccc(-c2nc(-c3ccccc3)nc(-c3ccc(-c4ccc(-c5ccc6ccccc6c5)cc4)c4c3oc3cc5ccccc5cc34)n2)cc1 ; c1ccc(-c2ccc(-c3cc(-c4nc(-c5ccccc5)nc(-c5ccccc5)n4)cc4c3oc3cc5ccccc5cc34)cc2)cc1 ; c1ccc(-c2ccc3cc(-c4nc(-c5ccccc5)nc(-c5cc6c7ccc(-c8ccc9ccccc9c8)cc7oc6c6ccccc56)n4)ccc3c2)cc1]

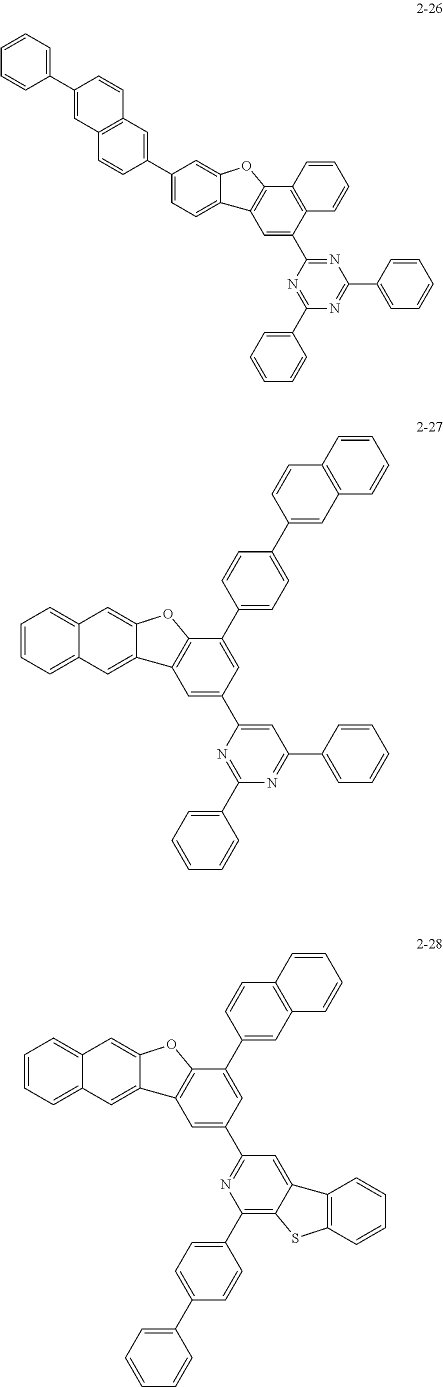 [SMILES: c1ccc(-c2ccc3cc(-c4ccc5c(c4)oc4c6ccccc6c(-c6nc(-c7ccccc7)nc(-c7ccccc7)n6)cc54)ccc3c2)cc1 ; c1ccc(-c2cc(-c3cc(-c4ccc(-c5ccc6ccccc6c5)cc4)c4oc5cc6ccccc6cc5c4c3)nc(-c3ccccc3)n2)cc1 ; c1ccc(-c2ccc(-c3nc(-c4cc(-c5ccc6ccccc6c5)c5oc6cc7ccccc7cc6c5c4)cc4c3sc3ccccc34)cc2)cc1]

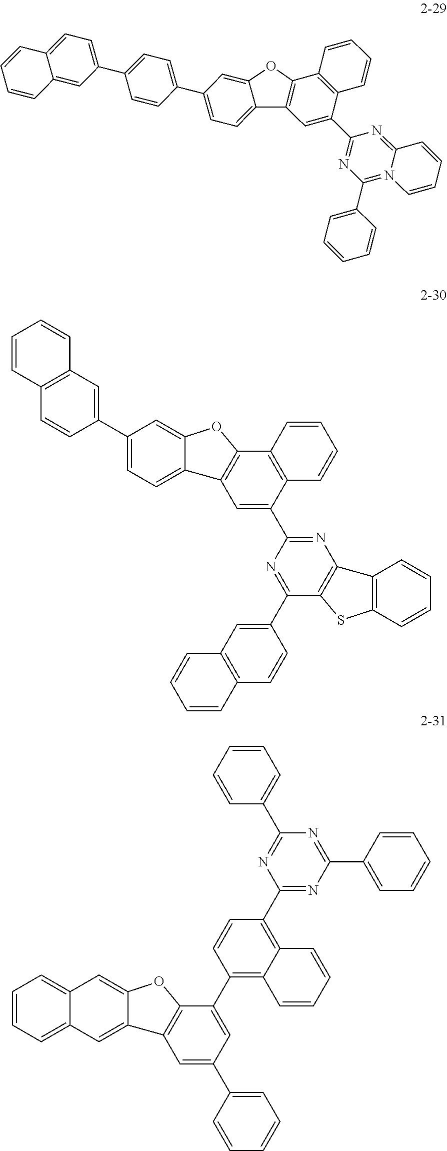

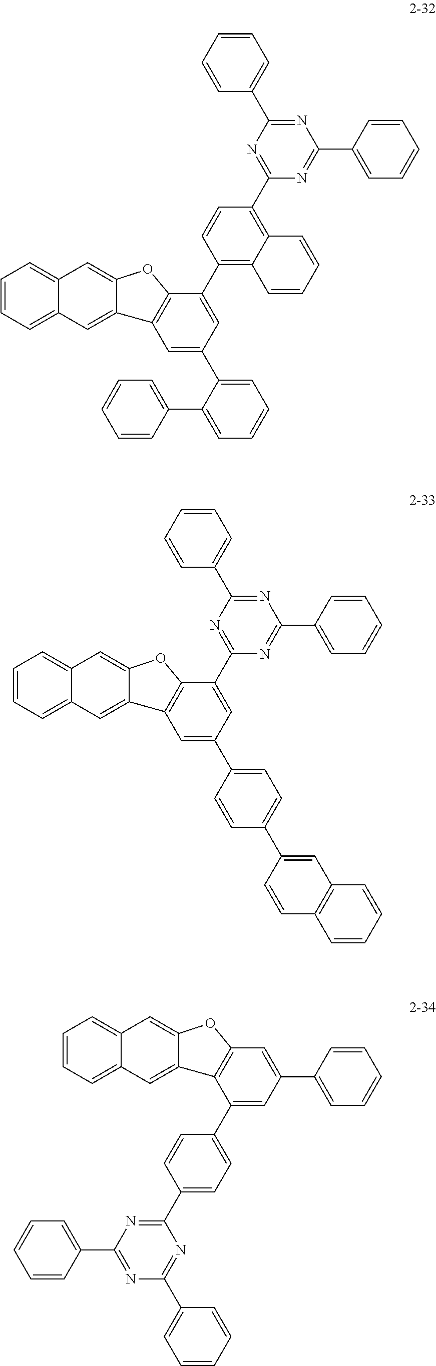 [SMILES: c1ccc(-c2nc(-c3ccccc3)nc(-c3ccc(-c4cc(-c5ccccc5-c5ccccc5)cc5c4oc4cc6ccccc6cc45)c4ccccc34)n2)cc1 ; c1ccc(-c2nc(-c3ccccc3)nc(-c3cc(-c4ccc(-c5ccc6ccccc6c5)cc4)cc4c3oc3cc5ccccc5cc34)n2)cc1 ; c1ccc(-c2cc(-c3ccc(-c4nc(-c5ccccc5)nc(-c5ccccc5)n4)cc3)c3c(c2)oc2cc4ccccc4cc23)cc1]

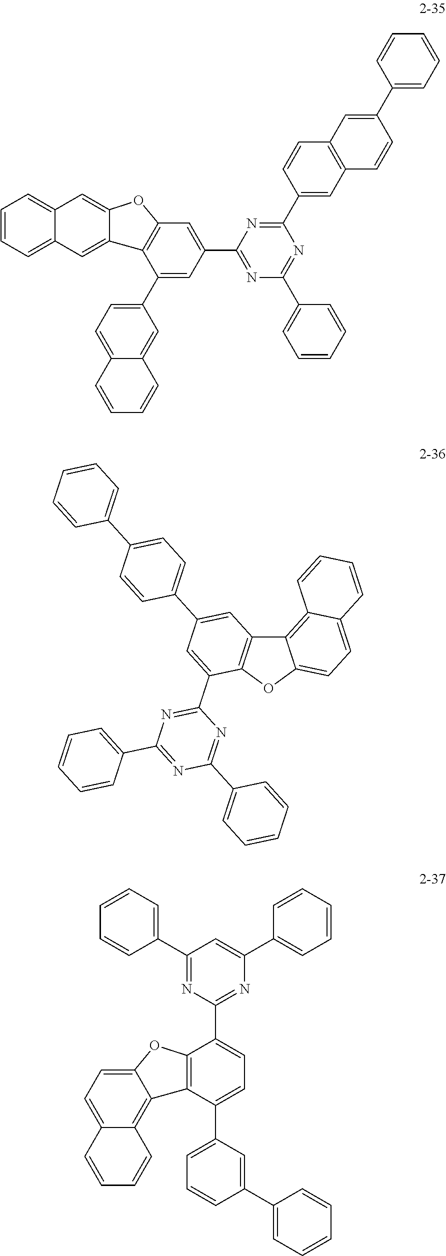 [SMILES: c1ccc(-c2ccc3cc(-c4nc(-c5ccccc5)nc(-c5cc(-c6ccc7ccccc7c6)c6c(c5)oc5cc7ccccc7cc56)n4)ccc3c2)cc1 ; c1ccc(-c2ccc(-c3cc(-c4nc(-c5ccccc5)nc(-c5ccccc5)n4)c4oc5ccc6ccccc6c5c4c3)cc2)cc1 ; c1ccc(-c2cccc(-c3ccc(-c4nc(-c5ccccc5)cc(-c5ccccc5)n4)c4oc5ccc6ccccc6c5c34)c2)cc1]

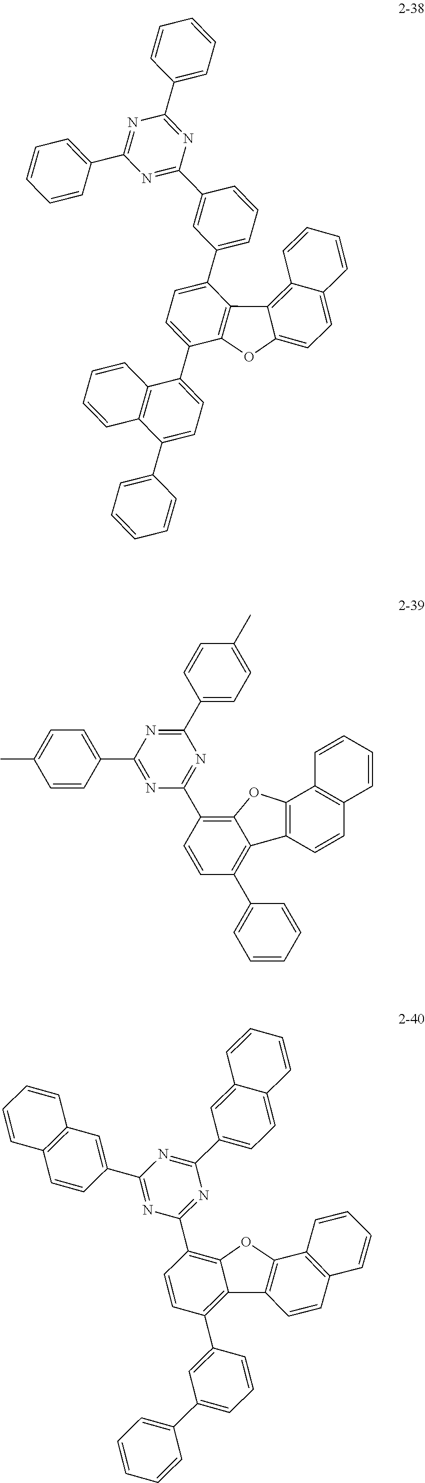 [SMILES: c1ccc(-c2nc(-c3ccccc3)nc(-c3cccc(-c4ccc(-c5ccc(-c6ccccc6)c6ccccc56)c5oc6ccc7ccccc7c6c45)c3)n2)cc1 ; Cc1ccc(-c2nc(-c3ccc(C)cc3)nc(-c3ccc(-c4ccccc4)c4c3oc3c5ccccc5ccc34)n2)cc1 ; c1ccc(-c2cccc(-c3ccc(-c4nc(-c5ccc6ccccc6c5)nc(-c5ccc6ccccc6c5)n4)c4oc5c6ccccc6ccc5c34)c2)cc1]

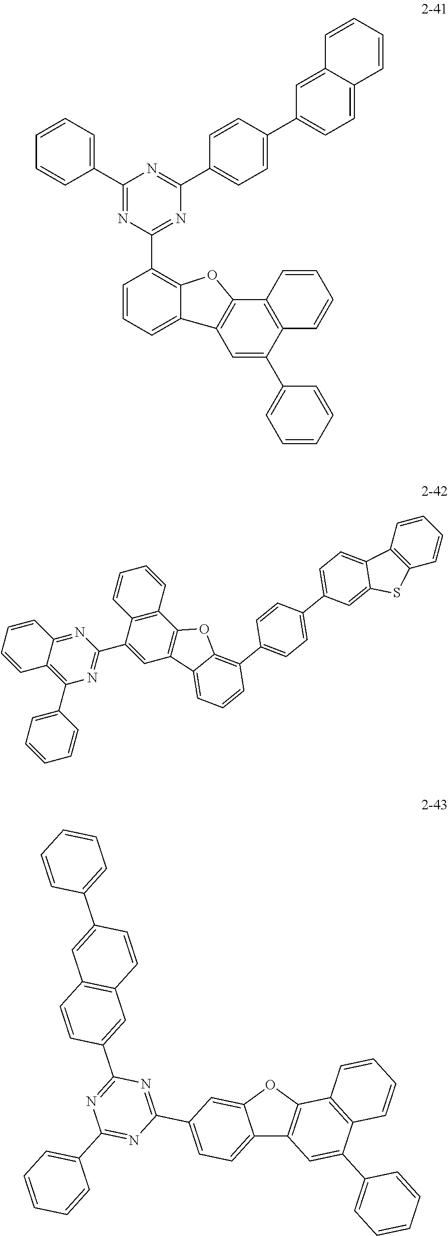 [SMILES: c1ccc(-c2nc(-c3ccc(-c4ccc5ccccc5c4)cc3)nc(-c3cccc4c3oc3c5ccccc5c(-c5ccccc5)cc43)n2)cc1 ; c1ccc(-c2nc(-c3cc4c5cccc(-c6ccc(-c7ccc8c(c7)sc7ccccc78)cc6)c5oc4c4ccccc34)nc3ccccc23)cc1 ; c1ccc(-c2ccc3cc(-c4nc(-c5ccccc5)nc(-c5ccc6c(c5)oc5c7ccccc7c(-c7ccccc7)cc65)n4)ccc3c2)cc1]

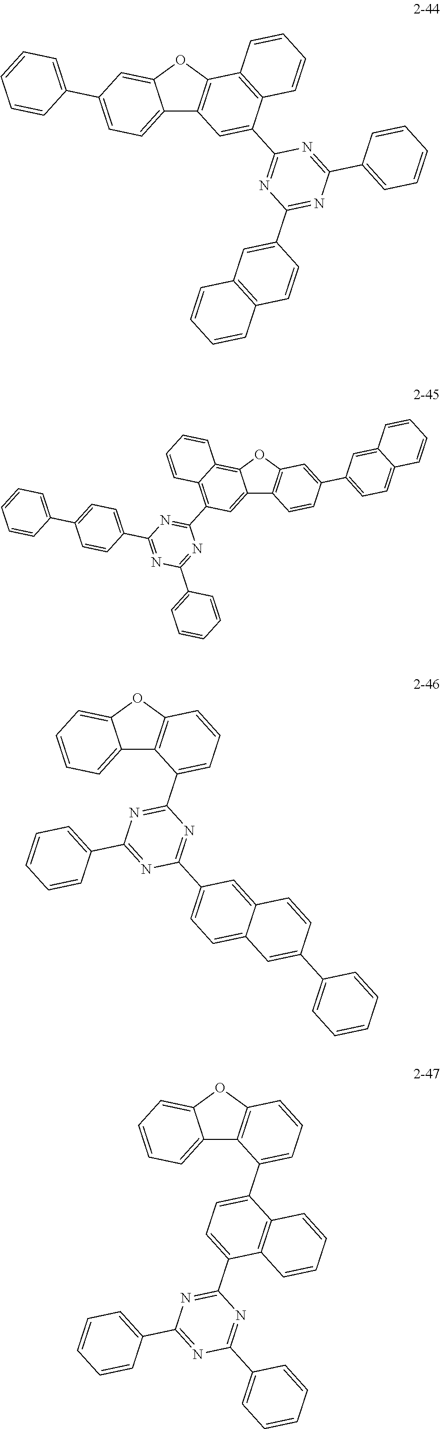 [SMILES: c1ccc(-c2ccc3c(c2)oc2c4ccccc4c(-c4nc(-c5ccccc5)nc(-c5ccc6ccccc6c5)n4)cc32)cc1 ; c1ccc(-c2ccc(-c3nc(-c4ccccc4)nc(-c4cc5c6ccc(-c7ccc8ccccc8c7)cc6oc5c5ccccc45)n3)cc2)cc1 ; c1ccc(-c2ccc3cc(-c4nc(-c5ccccc5)nc(-c5cccc6oc7ccccc7c56)n4)ccc3c2)cc1 ; c1ccc(-c2nc(-c3ccccc3)nc(-c3ccc(-c4cccc5oc6ccccc6c45)c4ccccc34)n2)cc1]

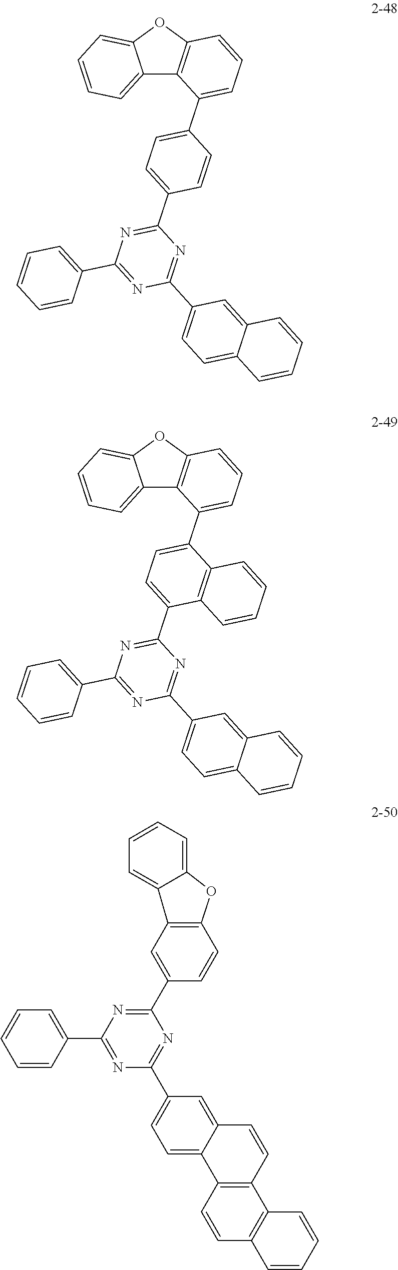 [SMILES: c1ccc(-c2nc(-c3ccc(-c4cccc5oc6ccccc6c45)cc3)nc(-c3ccc4ccccc4c3)n2)cc1 ; c1ccc(-c2nc(-c3ccc4ccccc4c3)nc(-c3ccc(-c4cccc5oc6ccccc6c45)c4ccccc34)n2)cc1 ; c1ccc(-c2nc(-c3ccc4c(ccc5c6ccccc6ccc45)c3)nc(-c3ccc4oc5ccccc5c4c3)n2)cc1]

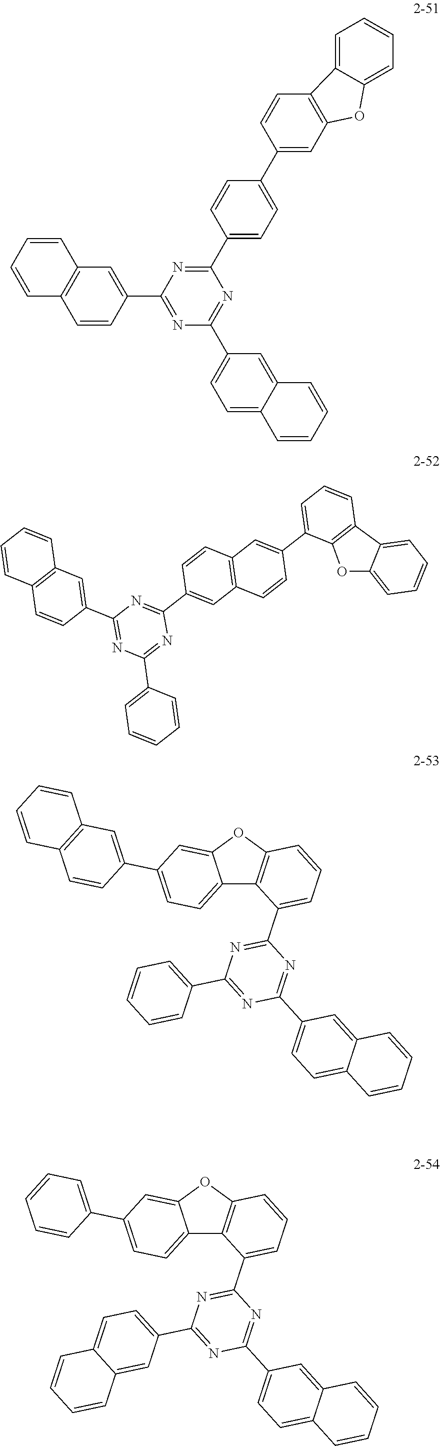 [SMILES: c1ccc2cc(-c3nc(-c4ccc(-c5ccc6c(c5)oc5ccccc56)cc4)nc(-c4ccc5ccccc5c4)n3)ccc2c1 ; c1ccc(-c2nc(-c3ccc4ccccc4c3)nc(-c3ccc4cc(-c5cccc6c5oc5ccccc56)ccc4c3)n2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2ccc3c(c2)oc2cccc(-c4nc(-c5ccc6ccccc6c5)nc(-c5ccc6ccccc6c5)n4)c23)cc1]

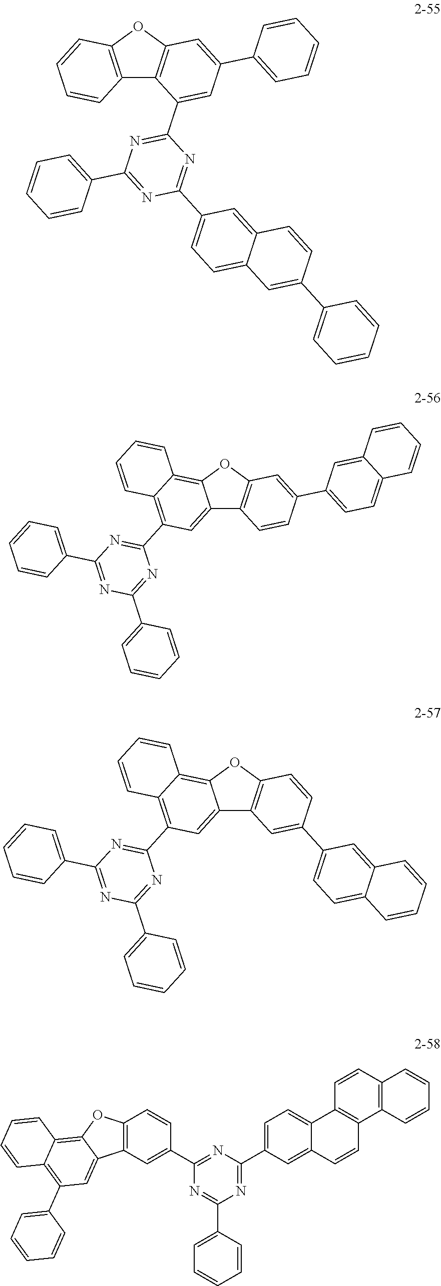 [SMILES: c1ccc(-c2ccc3cc(-c4nc(-c5ccccc5)nc(-c5cc(-c6ccccc6)cc6oc7ccccc7c56)n4)ccc3c2)cc1 ; c1ccc(-c2nc(-c3ccccc3)nc(-c3cc4c5ccc(-c6ccc7ccccc7c6)cc5oc4c4ccccc34)n2)cc1 ; c1ccc(-c2nc(-c3ccccc3)nc(-c3cc4c5cc(-c6ccc7ccccc7c6)ccc5oc4c4ccccc34)n2)cc1 ; c1ccc(-c2nc(-c3ccc4c(ccc5c6ccccc6ccc45)c3)nc(-c3ccc4oc5c6ccccc6c(-c6ccccc6)cc5c4c3)n2)cc1]

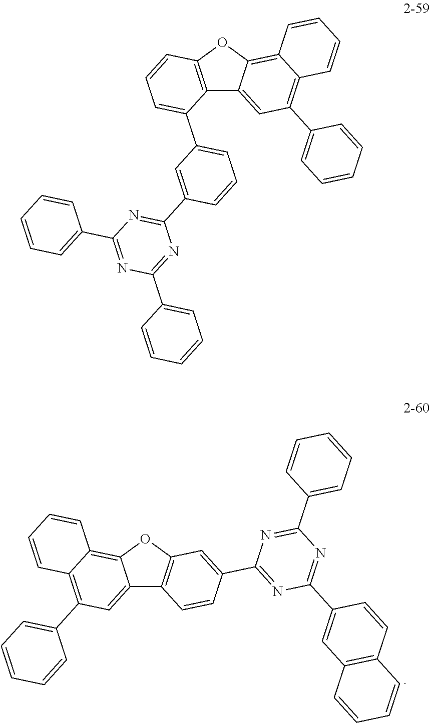 [SMILES: c1ccc(-c2nc(-c3ccccc3)nc(-c3cccc(-c4cccc5oc6c7ccccc7c(-c7ccccc7)cc6c45)c3)n2)cc1 ; c1ccc(-c2nc(-c3ccc4ccccc4c3)nc(-c3ccc4c(c3)oc3c5ccccc5c(-c5ccccc5)cc43)n2)cc1]

14. The organic light-emitting device of claim 6, further comprising one, or two or more layers selected from the group consisting of a light emitting layer, a hole injection layer, a hole transport layer, an electron injection layer, an electron transport layer, an electron blocking layer and a hole blocking layer.

15. A composition for an organic material layer, the composition comprising:
the heterocyclic compound of claim 1, and a heterocyclic compound represented by the following Chemical Formula 2: 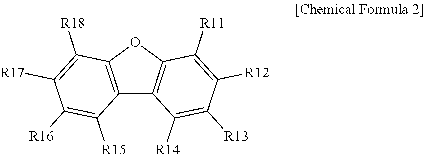
wherein, in Chemical Formula 2,
R11 to R18 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; —NR201R202; and the following Chemical Formula 3, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
at least one of R11 to R18 is the following Chemical Formula 3, 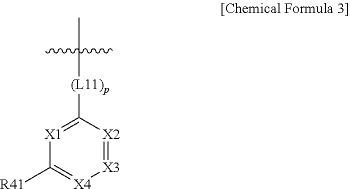
in Chemical Formula 3,
X1 is N; or CRa;
X2 is N; or CRb;
X3 is N; or CRc;
X4 is N; or CRd;
at least two of X1 to X4 are N;
R41 and Ra to Rd are the same as or different from each other, and each independently selected from the group consisting of hydrogen; deuterium; halogen: a cyano group; a substituted or unsubstituted C1 to C60 alkyl group; a substituted or unsubstituted C2 to C60 alkenyl group, a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group, —P(═O)R301R302; —SiR301R302R303; and —NR301R302, or two or more groups adjacent to each other bond to each other to form a substituted or unsubstituted C6 to C60 aromatic hydrocarbon ring; or a substituted or unsubstituted C2 to C60 heteroring, and R301, R302 and R303 are the same as or different from each other and each independently a substituted or unsubstituted C1 to C60 alkyl group; a substituted or unsubstituted C6 to C60 aryl group; or a substituted or unsubstituted C2 to C60 heteroaryl group;
L11 is a direct bond; a substituted or unsubstituted C6 to C60 arylene group; or a substituted or unsubstituted C2 to C60 heteroarylene group; and
p is an integer of 0 to 5, and when p is 2 or greater, L1 is are the same as or different from each other.

16. The composition of claim 15, wherein the heterocyclic compound represented by Chemical Formula 1 and the heterocyclic compound represented by Chemical Formula 2 have a weight ratio of 1:9 to 9:1.
